(19) United States
(12) Patent Application Publication
(10) Pub. No.: US 2026/0297581 A1
(43) Pub. Date: Oct. 1, 2026

(54) SHORT INTERFERING NUCLEIC ACID (SINA) MOLECULES TARGETING ANGPTL 3 AND ANGPTL 8 AND METHODS OF USING THE SAME

(71) Applicant: Aligos Therapeutics, Inc., South San Francisco, CA (US)

(72) Inventors: Leonid Beigelman, San Mateo, CA (US); Vivek Kumar Rajwanshi, Cupertino, CA (US); David Bernard Smith, San Mateo, CA (US); Jin Hong, Pacifica, CA (US); Xuan Luong, South San Francisco, CA (US); Saul Martinez Montero, San Bruno, CA (US); Antitsa Dimitrova STOYCHEVA, South San Francisco, CA (US); Aneerban Bhattacharya, South San Francisco, CA (US)

(73) Assignee: Aligos Therapeutics, Inc., South San Francisco, CA (US)

(21) Appl. No.: 19/478,343

(22) PCT Filed: Apr. 26, 2024
(86) PCT No.: PCT/US2024/026619

§ 371 Date: Oct. 24, 2025

Related U.S. Application Data
(60) Provisional application No. 63/462,456, filed on Apr. 27, 2023.

(51) Int. Cl. C12N 15/113; A61K 31/712; A61K 31/7125; A61K 31/713; A61P 1/16; A61P 3/06

(52) U.S. Cl. CPC C12N 15/113; A61K 31/712; A61K 31/7125; A61K 31/713; A61P 1/16; A61P 3/06; C12N 2310/11; C12N 2310/14; C12N 2310/312; C12N 2310/314; C12N 2310/315; C12N 2310/321; C12N 2310/322; C12N 2310/3515

(57) ABSTRACT

The present disclosure is in the field of pharmaceutical compounds and preparations and method of their use in the treatment of disease. Described are short interfering nucleic acid (siNA) molecules comprising modified nucleotides that target ANGPTL3 and ANGPTL8 as well as optimized co-formulation ratio or linker strategy in combining the ANGPTL3 siNA and ANGPTL8 siNA, compositions containing the same, and uses thereof for treating or preventing diseases. In particular, the present disclosure is in the field of siNA molecules that target ANGPTL3 and ANGPTL8 for treatment of cardiovascular disease and nonalcoholic fatty liver.

### TECHNICAL FIELD

The present disclosure is in the field of pharmaceutical compounds and preparations and method of their use in the treatment of disease. Described are short interfering nucleic acid (siNA) molecules comprising modified nucleotides that target ANGPTL3 or ANGPTL8, compositions containing the same, and uses thereof for treating or preventing diseases. In particular, the present disclosure is in the field of siNA molecules targeting ANGPTL3 and ANGPTL8 for treatment of cardiovascular disease and nonalcoholic fatty liver, among other diseases.

### BACKGROUND

The following discussion is merely provided to aid the reader in understanding the disclosure and is not admitted to describe or constitute prior art thereto.

Triglycerides are the main form of lipid that provides and stores energy in the human body. Lipoprotein lipase (LPL) breaks down triglycerides in lipoproteins, such as chylomicrons (CM) and very low-density lipoproteins (VLDL), into two free fatty acids and one monoacylglycerol molecule.

ANGPTL3, also known as Angiopoietin-like protein 3, is a protein exclusively and constitutively expressed in the liver. ANGPTL8, also known as lipasin, is expressed in the liver and adipose tissues after fed conditions. ANGPTL3, as well as ANGTL4, are inhibitors of LPL and they work in coordination with the cofactor ANGPTL8. The inhibition of LPL subsequently increases plasma triglyceride (TG), LDL cholesterol (LDL-c), Total Cholesterol (TC), and HDL-cholesterol (HDL-c).

ANGPTL3 can form a complex with the co-factor ANGPTL8 (ANGPTL3/8). The ANGPTL3/8 complex is a much stronger inhibitor of LPL in oxidative tissues such as heart and muscle (i.e., >100 fold) than ANGPTL3 alone, and ANGPTL8 is not an inhibitor of LPL. Reducing the ANGPTL3/8 complex by applying a combination of ANGPTL3 siNA and ANGPTL8 siNA treatments could achieve greater TG, TC, and LDL-c reduction than ANGPTL3 siNA treatment alone.

ANGPTL3 also inhibits endothelial lipase (EL) which results in elevation of HDL-c. ANGPTL8 cofactor is not required in inhibition of EL by ANGPTL3. ANGPTL3 siNA treatment alone has been shown to reduce “good” cholesterol HDL-c which can be a problem. Applying a combination of ANGPTL3 and ANGPTL8 siNA at optimum ratio could maximally reduce the abundance of the ANGPTL3/8 complex in heart and muscles, resulting in maximum reduction of LPL inhibition and maximum TG, TC, and LDL-c reduction in circulation, while leaving sufficient free ANGPTL3 to inhibit EL. Compared to ANGPTL3 siNA monotherapy, the amount of ANGPTL3 siNA dosed in combo therapy (ANGPTL3 siNA+ANGPTL8 siNA) is going to be lower, resulting in greater inhibition of EL by free ANGPTL3 and a better HDL-c profile. By optimizing the ANGPTL3 siNA vs ANGPTL8 siNA ratio, is possible to achieve lower HDL-c reduction than with ANGPTL3 siNA mono-therapy, or no HDL-c reduction, or even slight elevation of HDL-c in circulation.

Hypertriglyceridemia is associated with increased risk of atherosclerotic cardiovascular diseases and with fatty acid liver disease. Treatments that target the production and clearance of triglyceride-rich lipoproteins (TRLs) mitigate these complications. ANGPTL3 inhibition also lowers LDL particle concentration and may therefore have an overall advantage as a lipid-regulating agent.

ANGPTL3 controls LPL activity and has previously been treated as a target for inhibition by siNA, antisense oligonucleotide (ASO) and monoclonal antibodies. But the dual inhibition of ANGPTL3 and ANGPTL8 by siNA or ASO and the effects of dual inhibition on LPL and EL activities have not been previously explored.

RNA interference (RNAi) is a biological response to double-stranded RNA that mediates resistance to both endogenous parasitic and exogenous pathogenic nucleic acids, and regulates the expression of protein-coding genes. The short interfering nucleic acids (siNA), such as siRNA, have been developed for RNAi therapy to treat a variety of diseases. For instance, RNAi therapy has been proposed for the treatment of metabolic diseases, neurodegenerative diseases, cancer, and pathogenic infections.

### SUMMARY

The present disclosure provides siNA molecules useful for targeting and inhibiting ANGPTL3 and ANGPTL8 in optimized combination ratios, which subsequently increases LPL activity in oxidative tissues, such as, heart and muscles resulting in decreased TG, TC, and LDL-c in circulation. The combination therapy can maintain or increase HDL-c by maintaining a high level of free ANGPTL3 and subsequence inhibition of EL. Compared with bi-specific monoclonal antibodies that target the ANGPTL3/8 complex, the dual targeting siNA approach reduces dosing frequency and results in better patient compliance.

In one aspect, the present disclosure provides a short interfering nucleic acid (siNA) molecule comprising:
(a) a sense strand comprising a first nucleotide sequence that is at least about 60%, 65%, 70%, 75%, 80%, 85%, 90%, 95%, or 100% identical to an RNA corresponding to a target gene, wherein the first nucleotide sequence:

is 15 to 30 nucleotides in length; and
comprises 15 or more modified nucleotides independently selected from a 2′-O-methyl nucleotide and a 2′-fluoro nucleotide, wherein at least one modified nucleotide is a 2′-O-methyl nucleotide and the nucleotide at position 3, 5, 7, 8, 9, 10, 11, 12, 14, 17, and/or 19 from the 5′ end of the first nucleotide sequence is a 2′-fluoro nucleotide or wherein at least one modified nucleotide is a 2′-O-methyl nucleotide and at least one modified nucleotide is a 2′-fluoro nucleotide; and
an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence:
is 15 to 30 nucleotides in length; and
comprises 15 or more modified nucleotides independently selected from a 2′-O-methyl nucleotide and a 2′-fluoro nucleotide, wherein at least one modified nucleotide is a 2′-O-methyl nucleotide and at least one modified nucleotide is a 2′-fluoro nucleotide; or
(b) a sense strand comprising a first nucleotide sequence that is at least about 60%, 65%, 70%, 75%, 80%, 85%, 90%, 95%, or 100% identical to an RNA corresponding to a target gene, wherein the first nucleotide sequence:
(i) is 15 to 30 nucleotides in length; and
(ii) comprises 15 or more modified nucleotides independently selected from a 2′-O-methyl nucleotide and a 2′-fluoro nucleotide, wherein at least one modified nucleotide is a 2′-O-methyl nucleotide and at least one modified nucleotide is a 2′-fluoro nucleotide; and an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence:
(iii) is 15 to 30 nucleotides in length; and (iv) comprises 15 or more modified nucleotides independently selected from a 2′-O-methyl nucleotide and a 2′-fluoro nucleotide, wherein at least one modified nucleotide is a 2′-O-methyl nucleotide and the nucleotide at position 2, 5, 6, 8, 10, 14, 16, 17, and/or 18 from the 5′ end of the second nucleotide sequence is a 2′-fluoro nucleotide;
wherein the target gene is a gene encoding ANGPTL8 or a gene that controls expression of ANGPTL8.

In one aspect, the present disclosure provides a short interfering nucleic acid (siNA) molecule comprising:
(a) a sense strand comprising a first nucleotide sequence that is at least about 60%, 65%, 70%, 75%, 80%, 85%, 90%, 95%, or 100% identical to an RNA corresponding to a target gene, wherein the first nucleotide sequence:
is 15 to 30 nucleotides in length; and
comprises 15 or more modified nucleotides independently selected from a 2′-O-methyl nucleotide and a 2′-fluoro nucleotide, wherein at least one modified nucleotide is a 2′-O-methyl nucleotide and the nucleotide at position 3, 5, 7, 8, 9, 10, 11, 12, 14, 17, and/or 19 from the 5′ end of the first nucleotide sequence is a 2′-fluoro nucleotide or wherein at least one modified nucleotide is a 2′-O-methyl nucleotide and at least one modified nucleotide is a 2′-fluoro nucleotide; and
an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence:
is 15 to 30 nucleotides in length; and
comprises 15 or more modified nucleotides independently selected from a 2′-O-methyl nucleotide and a 2′-fluoro nucleotide, wherein at least one modified nucleotide is a 2′-O-methyl nucleotide and at least one modified nucleotide is a 2′-fluoro nucleotide; or
(b) a sense strand comprising a first nucleotide sequence that is at least about 60%, 65%, 70%, 75%, 80%, 85%, 90%, 95%, or 100% identical to an RNA corresponding to a target gene, wherein the first nucleotide sequence:
(i) is 15 to 30 nucleotides in length; and
(ii) comprises 15 or more modified nucleotides independently selected from a 2′-O-methyl nucleotide and a 2′-fluoro nucleotide, wherein at least one modified nucleotide is a 2′-O-methyl nucleotide and at least one modified nucleotide is a 2′-fluoro nucleotide; and an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence:
(iii) is 15 to 30 nucleotides in length; and (iv) comprises 15 or more modified nucleotides independently selected from a 2′-O-methyl nucleotide and a 2′-fluoro nucleotide, wherein at least one modified nucleotide is a 2′-O-methyl nucleotide and the nucleotide at position 2, 5, 6, 8, 10, 14, 16, 17, and/or 18 from the 5′ end of the second nucleotide sequence is a 2′-fluoro nucleotide;
wherein the target gene is a gene encoding ANGPTL3 or a gene that controls expression of ANGPTL3.

In another aspect, the present disclosure provides double-stranded short interfering nucleic acid (ds-siNA) molecules comprising:
(a) a sense strand comprising a first nucleotide sequence, wherein the first nucleotide sequence is 15 to 30 nucleotides in length and comprises a nucleotide sequence as set forth in in any one of SEQ ID NOs: 1-316, and
an antisense strand comprising a second nucleotide sequence, wherein the second nucleotide sequence is 15 to 30 nucleotides in length and comprises a nucleotide sequence that is a substantial reverse complement of the first nucleotide sequence; or
(b) a sense strand comprising a first nucleotide sequence, wherein the first nucleotide sequence is 15 to 30 nucleotides in length and comprises a nucleotide sequence that is a substantial reverse complement of a second nucleotide sequence; and
an antisense strand comprising the second nucleotide sequence, wherein the second nucleotide sequence is 15 to 30 nucleotides in length and comprises a nucleotide sequence as set forth in in any one of SEQ ID NOs: 317-632.

In another aspect, the present disclosure provides double-stranded short interfering nucleic acid (ds-siNA) molecules comprising:
(a) a sense strand comprising a first nucleotide sequence, wherein the first nucleotide sequence is 15 to 30 nucleotides in length and comprises a nucleotide sequence as set forth in in any one of SEQ ID NOs: 633-878, and
an antisense strand comprising a second nucleotide sequence, wherein the second nucleotide sequence is 15 to 30 nucleotides in length and comprises a nucleotide sequence that is a substantial reverse complement of the first nucleotide sequence; or
(b) a sense strand comprising a first nucleotide sequence, wherein the first nucleotide sequence is 15 to 30 nucleotides in length and comprises a nucleotide sequence that is a substantial reverse complement of a second nucleotide sequence; and
an antisense strand comprising the second nucleotide sequence, wherein the second nucleotide sequence is 15 to 30 nucleotides in length and comprises a nucleotide sequence as set forth in any one of SEQ ID NOs: 879-1125.

In another aspect, the present disclosure provides double-stranded short interfering nucleic acid (ds-siNA) molecules comprising: (a) a sense strand comprising a nucleotide sequence as set forth in any one of SEQ ID NOs: 1-316, and (b) an antisense strand. In another aspect, the present disclosure provides double-stranded short interfering nucleic acid (ds-siNA) molecules comprising (a) a sense strand comprising a nucleotide sequence as set forth in any one of SEQ ID NOs: 633-878, and (b) an antisense strand. In another aspect, the present disclosure provides double-stranded short interfering nucleic acid (ds-siNA) molecules comprising (a) an antisense strand comprising a nucleotide sequence as set forth in any one of SEQ ID NOs: 317-632 (b) a sense strand. In another aspect, the present disclosure provides double-stranded short interfering nucleic acid (ds-siNA) molecules comprising (a) an antisense strand comprising a nucleotide sequence as set forth in any one of SEQ ID NOS: 879-1125 (b) a sense strand.

In some embodiments of any of the foregoing aspects, the sense strand comprises SEQ ID NOs: 633, 634, 642, 653, 654, 674, 687, 688, 690-694, 708-713, 756-757, 765, 776, 777, 797, 810, 811, 813-817, or 831-836 and the antisense strand comprises SEQ ID NOs: 879, 880, 888, 899, 900, 920, 933, 934, 936-940, 954-959, 1003-1004, 1012, 1023, 1024, 1044, 1057, 1058, 1060-1064, or 1078-1083, respectively. In some embodiments, the sense strand comprises SEQ ID NOs: 633, 654, 688, 690, 708, 713, 756, 777, 811, 813, 831, or 836, and the antisense strand comprises SEQ ID NOs: 879, 900, 934, 936, 954, 959, 1003, 1024, 1058, 1060, 1078, or 1083, respectively.

In some embodiments of any of the foregoing aspects, the sense and/or antisense strand further comprises a TT sequence at 3′ end.

In some embodiments of any of the foregoing aspects, at least one end of the ds-siNA is a blunt end.

In some embodiments of any of the foregoing aspects, at least one end of the ds-siNA comprises an overhang, wherein the overhang comprises at least one nucleotide, optionally wherein the at least one nucleotide comprises a 2′-O-methyl nucleotide, a deoxy nucleotide, or a uracil analogue or derivative.

In some embodiments of any of the foregoing aspects, both ends of the ds-siNA comprise an overhang, wherein the overhang comprises at least one nucleotide, optionally wherein the at least one nucleotide comprises a 2′-O-methyl nucleotide, a deoxy nucleotide, or a uracil analogue or derivative.

In some embodiments of any of the foregoing aspects or embodiments, the sense strand and/or the antisense strand independently comprise 1 or more mesyl phosphoramidate internucleoside linkages, or 1 or more phosphorothioate internucleoside linkages.

In some embodiments of any of the foregoing aspects or embodiments, the sense strand comprises at least 1, 2, 3, 4, 5, 6, 7, 8, 9, 10, 11, 12, 13, 14, 15 or more phosphorothioate internucleoside linkages. In some embodiments, (i) at least one phosphorothioate internucleoside linkage in the sense strand is between the nucleotides at positions 1 and 2 from the 5′ end of the sense strand sequence; (ii) at least one phosphorothioate internucleoside linkage is between the nucleotides at positions 2 and 3 from the 5′ end of the sense strand sequence.

In some embodiments of any of the foregoing aspects or embodiments, the antisense strand further comprises at least 1, 2, 3, 4, 5, 6, 7, 8, 9, 10, 11, 12, 13, 14, 15 or more phosphorothioate internucleoside linkages. In some embodiments: (i) at least one phosphorothioate internucleoside linkage in the antisense strand is between the nucleotides at positions 1 and 2 from the 5′ end of the antisense strand sequence; (ii) at least one phosphorothioate internucleoside linkage in the antisense strand is between the nucleotides at positions 2 and 3 from the 5′ end of the antisense strand sequence; (iii) at least one phosphorothioate internucleoside linkage in the antisense strand is between the nucleotides at positions 1 and 2 from the 3′ end of the antisense strand sequence; and/or (iv) at least one phosphorothioate internucleoside linkage is between the nucleotides at positions 2 and 3 from the 3′ end of the antisense strand sequence.

In some embodiments of any of the foregoing aspects or embodiments, the sense strand comprises at least 1, 2, 3, 4, 5, 6, 7, 8, 9, 10, 11, 12, 13, 14, 15 or more mesyl phosphoramidate internucleoside linkages. In some embodiments: (i) at least one mesyl phosphoramidate internucleoside linkage in the sense strand is between the nucleotides at positions 1 and 2 from the 5′ end of the sense strand sequence; (ii) at least one mesyl phosphoramidate internucleoside linkage is between the nucleotides at positions 2 and 3 from the 5′ end of the sense strand sequence.

In some embodiments of any of the foregoing aspects or embodiments, the antisense strand further comprises at least 1, 2, 3, 4, 5, 6, 7, 8, 9, 10, 11, 12, 13, 14, 15 or more mesyl phosphoramidate internucleoside linkages. In some embodiments: (i) at least one mesyl phosphoramidate internucleoside linkage in the antisense strand is between the nucleotides at positions 1 and 2 from the 5′ end of the antisense strand sequence; (ii) at least one mesyl phosphoramidate internucleoside linkage in the antisense strand is between the nucleotides at positions 2 and 3 from the 5′ end of the antisense strand sequence; (iii) at least one mesyl phosphoramidate internucleoside linkage in the antisense strand is between the nucleotides at positions 1 and 2 from the 3′ end of the antisense strand sequence; and/or (iv) at least one mesyl phosphoramidate internucleoside linkage is between the nucleotides at positions 2 and 3 from the 3′ end of the antisense strand sequence.

In some embodiments of any of the foregoing aspects or embodiments, the antisense strand comprises one or more modified nucleotides. In some embodiments of any of the foregoing aspects or embodiments, the sense strand comprises one or more modified nucleotides. In some embodiments of any of the foregoing aspects or embodiments, the sense strand and the antisense strand each independently comprise one or more modified nucleotides. In some embodiments, the modified nucleotides are independently selected from 2′-O-methyl nucleotides and 2′-fluoro nucleotides. Other modified nucleotides that may be included in the sense strand, antisense strand, or both are disclosed herein.

In some embodiments of any of the foregoing aspects or embodiments, the antisense strand, sense strand, first nucleotide sequence, and/or second nucleotide sequence comprises at least one modified nucleotide selected from:

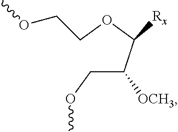

wherein Rx is a nucleobase, aryl, heteroaryl, or H,

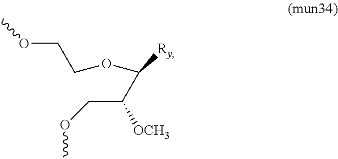

wherein R_{y} is a nucleobase,

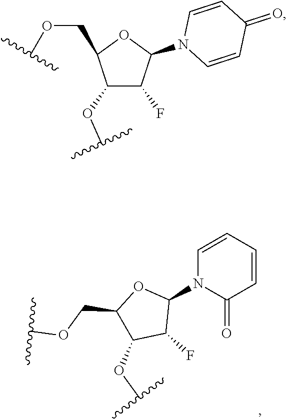

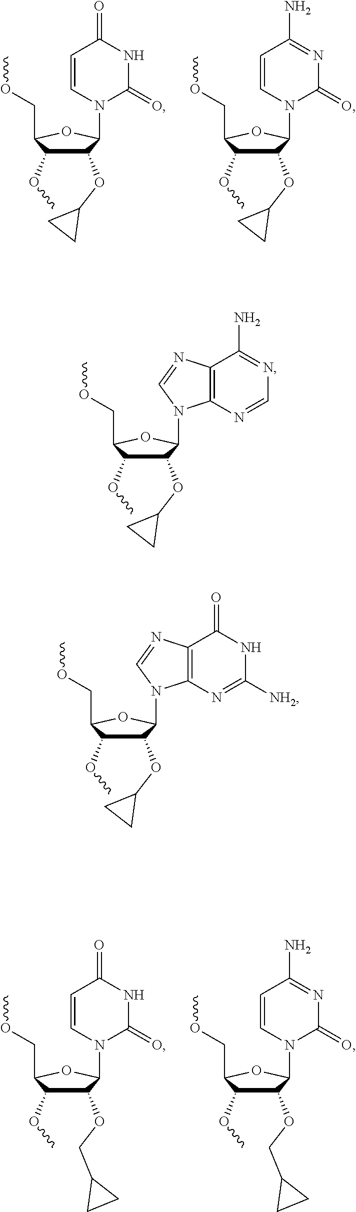

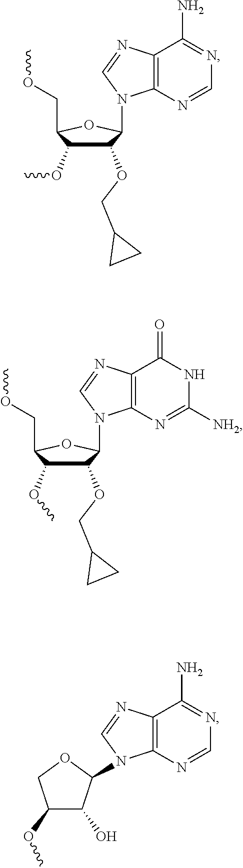

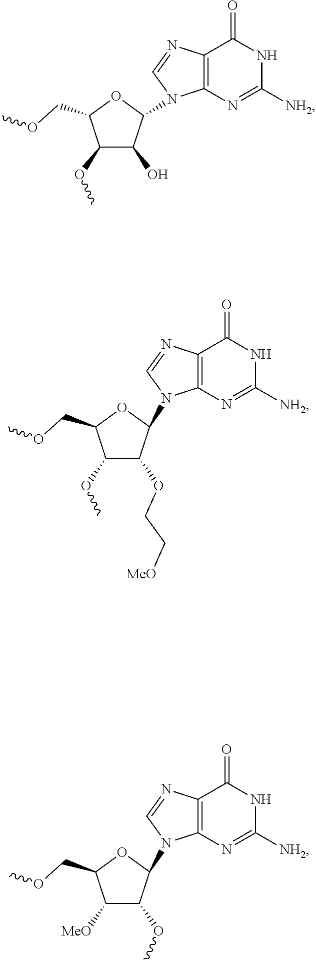

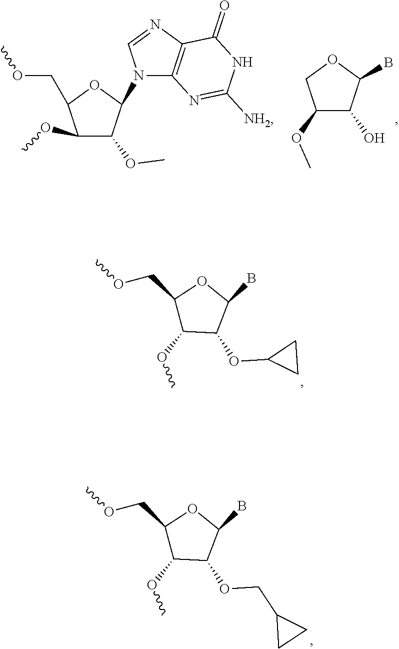

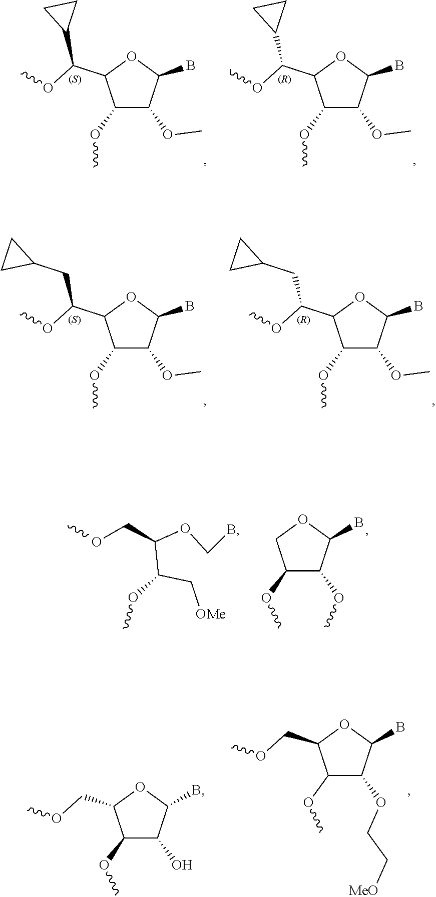

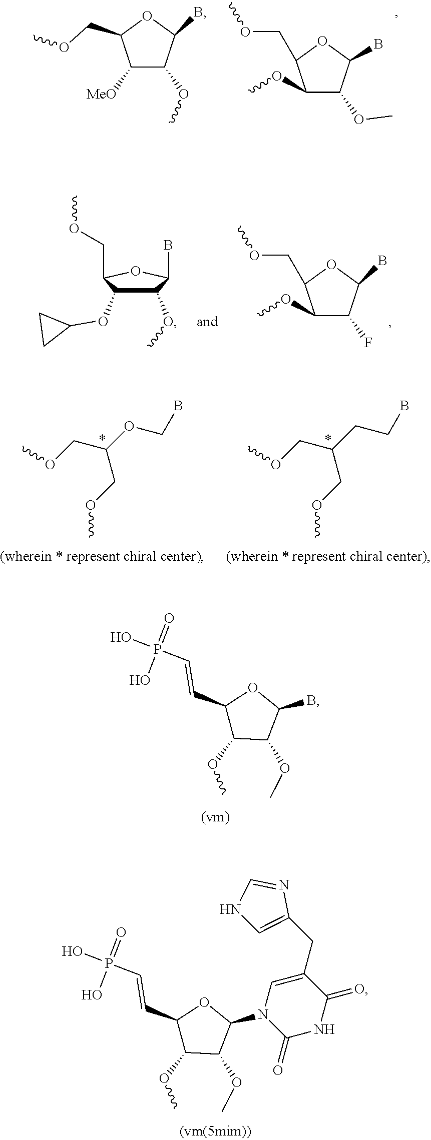

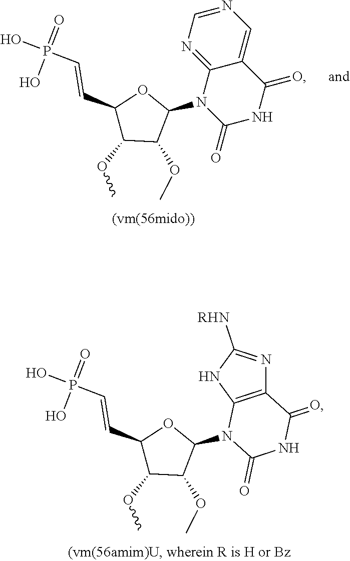

wherein B is a nucleobase, aryl, heteroaryl, or H, and wherein [symbol] or [symbol] represents a phosphodiester linkage, a phosphorothioate linkage, a mesyl phosphoramidate linkage, or H.

In some embodiments of any of the foregoing aspects or embodiments, the ds-siNA further comprises a phosphorylation blocker, a 5′-stabilized end cap, or a combination thereof. In some embodiments, the phosphorylation blocker is attached to the 5′ end of the sense strand, and optionally, wherein the phosphorylation blocker is attached to the 5′ end of the sense strand via one or more of a phosphodiester linkage, a phosphorothioate linkage, a mesyl phosphoramidate linkage, or a phosphorodithioate linkage. In some embodiments, the 5′-stabilized end cap is a 5′ vinyl phosphonate (e.g., vm). In some embodiments, the 5′-stabilized end cap is attached to the 5′ end of the antisense strand, and optionally, wherein the 5′-stabilized end cap is attached to the 5′ end of the antisense strand one or more of a phosphodiester linkage, a phosphorothioate linkage, a mesyl phosphoramidate linkage, or a phosphorodithioate linkage. In some embodiments, the 5′-stabilized end cap comprises a structure of:

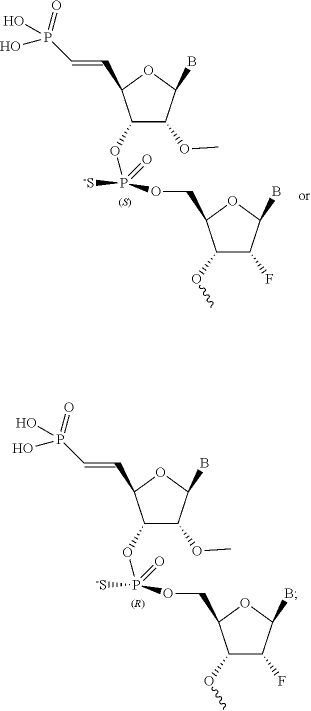

wherein B is a nucleobase, aryl, heteroaryl, or H; wherein [symbol] represents a phosphodiester linkage, a phosphorothioate linkage, or a mesyl phosphoramidate linkage.

In some embodiments of any of the foregoing aspects or embodiments, the ds-siNA further comprises a galactosamine. In some embodiments, the galactosamine is an N-acetylgalactosamine comprising a structure of:

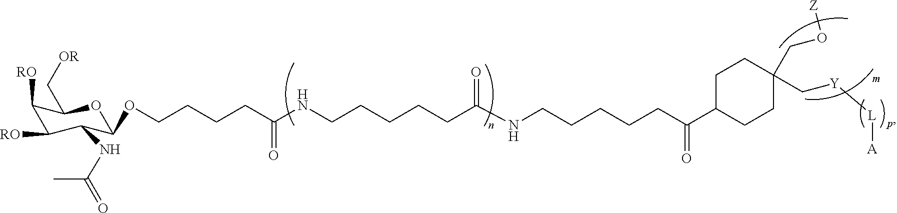

wherein m is 1, 2, 3, 4, or 5; each n is independently 1 or 2; p is 0 or 1; each R is independently H; each Y is independently selected from —O—P(═O)(SH)—, —O—P(═O)(O)—, —O—P(═O)(OH)—, and —O—P(S)S—; Z is H or a second protecting group; either L is a linker or L and Y in combination are a linker; and A is H, OH, a third protecting group, an activated group, or an oligonucleotide. In some embodiments, the galactosamine is an N-acetylgalactosamine comprising a structure of:

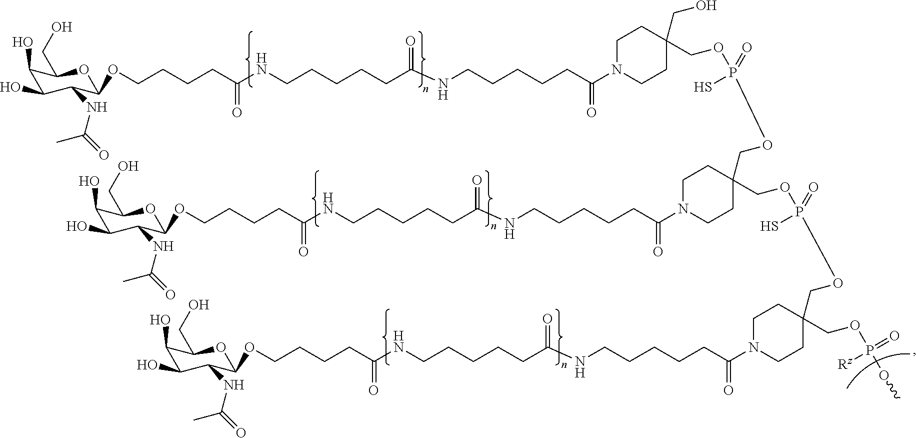

wherein R^{z} is OH or SH; and each n is independently 1 or 2.

In some embodiments of any of the foregoing aspects or embodiments, the sense and/or anti-sense strand of the ds-siNA is conjugated to GalNAc, Folate, Cholesterol, or Palmitic Acid.

In some embodiments of any of the foregoing aspects or embodiments, the ds-siNA reduces ANGPTL8 or ANGPTL3 expression by more than about 10%, more than about 20% more than about 30%, more than about 40%, more than about 50%, more than about 60%, more than about 70%, more than about 80%, more than about 90%, or more than about 95%. In some embodiments of any of the foregoing aspects or embodiments, the ds-siNA reduces ANGPTL8 or ANGPTL3 expression by 100%.

In some embodiments of any of the foregoing aspects or embodiments, the ds-siNA reduces ANGPTL8 or ANGPTL3 expression or activity with a EC₅₀ value of 50 μM or less, of 40 μM or less, of 30 μM or less, of 20 μM or less, 15 μM or less, 10 μM or less, 5 μM or less, 2 μM or less, or 1 μM or less.

In some embodiments of any of the foregoing aspects or embodiments, the ds-siNA has a CC₅₀ value of more than 1 μM.

In some embodiments of any of the foregoing aspects or embodiments, the ds-siNA comprises RNA nucleotides.

In another aspect, the present disclosure provides ds-siNA, comprising a sense strand and an antisense strand, wherein the antisense strand comprises at its 5′ end one of the following:

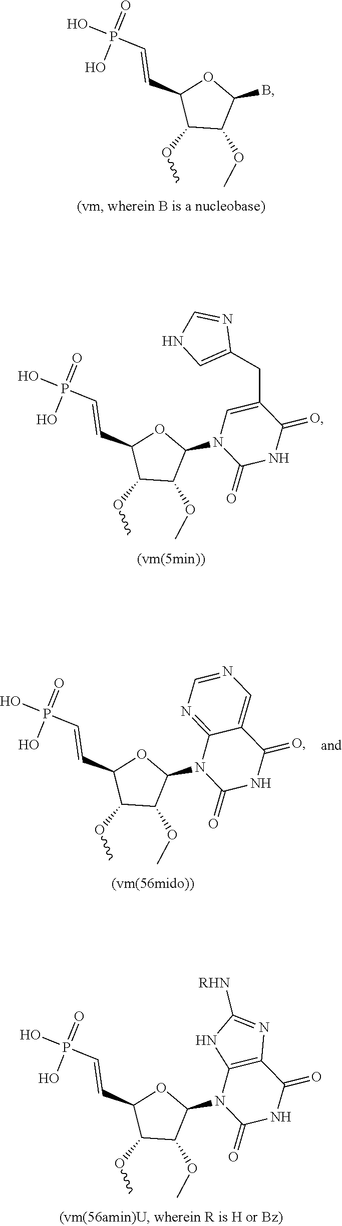

In another aspect, the present disclosure provides pharmaceutical compositions comprising at least one ds-siNA disclosed herein and a pharmaceutically acceptable carrier or diluent. In another aspect, the present disclosure provides pharmaceutical compositions comprising two or more ds-siNA disclosed herein. For the purposes of pharmaceutical compositions that combine the disclosed ds-siNA, the combination of two or more ds-siNA disclosed herein will generally comprise at least one ds-siNA that reduces expression of ANGPTL3 and at least one ds-siNA that reduces expression of ANGPTL8.

In another aspect, the present disclosure provides pharmaceutical compositions comprising at least a first siNA that reduces ANGPTL3 expression and a second siNA that reduces ANGPTL8 expression and a pharmaceutically acceptable carrier. In some embodiments, the first siNA that reduces ANGPTL3 expression comprises a nucleic acid sequence disclosed in Table 3, and the second siNA that reduces ANGPTL8 expression comprises a nucleic acid sequence disclosed in Table 1. In some embodiments, the first siNA that reduces ANGPTL3 expression is a ds-siNA disclosed in Table 4, and the second siNA that reduces ANGPTL8 expression is a ds-siNA disclosed in Table 2. In some embodiments, a weight ratio of the first siNA and the second siNA is selected from 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1. In some embodiments, a molar ration of the first siNA and the second siNA is selected from 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1. In some embodiments, the first siNA and the second siNA are connected by a linker, which may be a nucleotide-based linker or a non-nucleotide-based linker.

The pharmaceutical compositions disclosed herein may be formulated for parenteral, ocular, nasal, transdermal, pulmonary, or topical administration or comprises a liposome that encapsulates the ds-siNA.

The present disclosure also provides uses of the ds-siNA disclosed herein in the manufacture of a medicament for treating cardiovascular disease or nonalcoholic fatty liver disease.

The present disclosure also provides uses of two or more ds-siNA disclosed herein in the manufacture of a medicament for treating cardiovascular disease or nonalcoholic fatty liver disease.

The present disclosure also provides ds-siNA disclosed herein or the pharmaceutical compositions disclosed herein for use in treating cardiovascular disease or nonalcoholic fatty liver disease.

The present disclosure also provides two or more ds-siNA disclosed herein for use in treating cardiovascular disease or nonalcoholic fatty liver disease.

In another aspect the present disclosure provides methods of treating a disease in a subject in need thereof, comprising administering to the subject a ds-siNA disclosed herein or a pharmaceutical composition disclosed herein, wherein the disease is cardiovascular disease or nonalcoholic fatty liver disease.

In another aspect the present disclosure provides methods of treating a disease in a subject in need thereof, comprising administering to the subject two or more ds-siNA disclosed herein, wherein the disease is cardiovascular disease or nonalcoholic fatty liver disease.

In another aspect the present disclosure provides methods of treating a disease in a subject in need thereof, comprising administering to the subject a first pharmaceutical composition comprising at least a first ds-siNA that reduces ANGPTL3 expression and a second pharmaceutical composition comprising at least a second ds-siNA that reduces ANGPTL8 expression, wherein the disease is cardiovascular disease or nonalcoholic fatty liver disease. In some embodiments, the first siNA that reduces ANGPTL3 expression comprises a nucleic acid sequence disclosed in Table 3, and the second siNA that reduces ANGPTL8 expression comprises a nucleic acid sequence disclosed in Table 1. In some embodiments, the first siNA that reduces ANGPTL3 expression is a ds-siNA disclosed in Table 4, and the second siNA that reduces ANGPTL8 expression is a ds-siNA disclosed in Table 2.

In some embodiments of the disclosed methods, the cardiovascular disease is Hypertriglyceridemia (HTG) or Familial Hypercholesterolemia (FH).

In some embodiments of the disclosed methods, wherein the subject is a mammal, and optionally wherein the mammal is a human or a non-human primate.

In some embodiments of the disclosed methods, wherein the ds-siNA or pharmaceutical composition(s) are administered intravenously, subcutaneously, or via inhalation.

In some embodiments of the disclosed methods, wherein the subject has been treated with one or more additional therapeutic agents.

In another aspect the present disclosure provides methods of reducing ANGPTL8 and/or ANGPTL3 expression or activity in a tissue, an organ, or a cell of a subject in need thereof, wherein the method comprises delivering to the subject one or more of the ds-siNA disclosed herein or the pharmaceutical compositions disclosed herein.

In another aspect the present disclosure provides modified nucleosides selected from

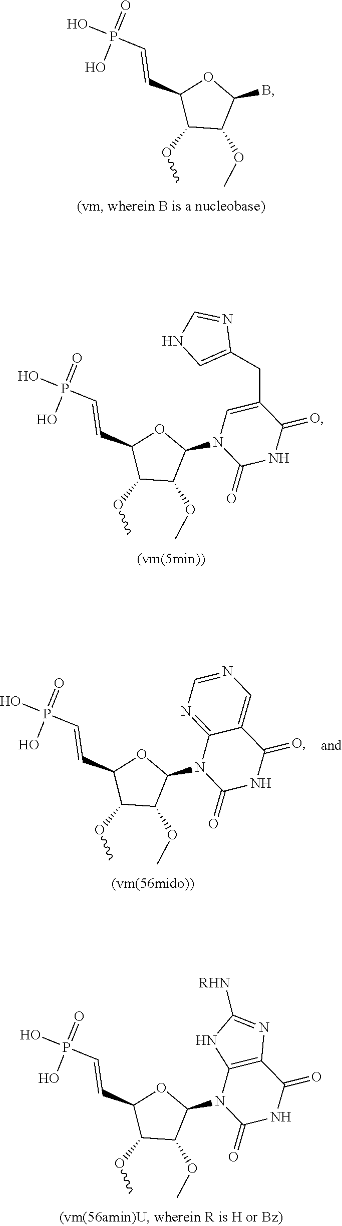

It should be appreciated that all combinations of the foregoing concepts and additional concepts discussed in greater detail below are provided as being part of the inventive subject matter disclosed herein and may be employed in any combination to achieve the benefits described herein.

### BRIEF DESCRIPTION OF THE DRAWINGS

FIG. 1A-IF depicts examples of siNA molecules that target ANGPTL3.

FIG. 2A-2B provides the effects of exemplary siNAs after treatment of COS-7 cells with a dose response curve. FIG. 2A shows the percentage of viable COS-7 cells after treatment. FIG. 2B shows percentage inhibition of cynomolgous monkey ANGPTL3 after treatment with 6 exemplary siNAs as determined by luciferase assay.

FIG. 3 depicts the percentage change in expression of ANGPTL3 protein from serum samples in groups treated with the ANGPTL3 siNAs relative to the expression levels from serum samples in the group treated with the negative vehicle control in the AAV-hANGPTL3 mice. Serum samples were taken at Day 28 post-dose.

FIG. 4 depicts the percentage change in expression of ANGPTL3 protein from serum samples in groups treated with the ANGPTL3 siNAs relative to the expression levels from serum samples in a group treated with the negative vehicle control in the hANGPTL3 knock-in mice. Serum samples were taken at Day 28 post-dose.

FIG. 5 depicts the percentage change in expression of ANGPTL3 RNA of the liver samples in groups treated with ANGPTL3 siNAs relative to the expression levels of the same RNA in the livers of the group treated with the negative vehicle control in the hANGPTL3 knock-in mice. Liver samples were collected at Day 28 post-dose.

FIG. 6 depicts the results of the plasmid based on- and off-target luciferase assay for siNAs having destabilized seed regions.

FIG. 7 depicts the percentage change in expression of ANGPTL8 RNA in the livers of the groups treated with ANGPTL8 siNAs relative to the expression levels of the same RNA in the livers of the group treated with the negative vehicle control in the AAV-hANGPTL8 mice. Livers samples were collected at Day 14 post-dose.

FIGS. 8A, 8B, 8C, 8D, and 8E depict percent reduction in various lipids following single or combination hANGPTL3 siNA and hANGPTL8 siNA treatment in the human ANGPTL3/8 double knock-in mouse study. FIG. 8A shows the percent reduction in total cholesterol levels Day 7 post-dose. FIG. 8B shows the percent reduction in total cholesterol levels Day 14 post-dose. FIG. 8C shows the percent reduction in total cholesterol levels Day 21 post-dose. FIG. 8D shows the percent reduction in total cholesterol levels Day 28 post-dose. FIG. 8E shows the percent reduction in triglyceride levels Day 14 post-dose.

### DETAILED DESCRIPTION

The present disclosure provides siNA that target and decrease expression of ANGPTL3 or ANGPTL8. ANGPTL3 (along with ANGPTL4) work in coordination with cofactor ANGPTL8 to regulate lipid and triglyceride metabolism. ANGPTL3 is exclusively and constitutively expressed in the liver, and its inhibition lowers LDL particle concentration. ANGPTL8 is expressed in the liver and adipose tissues and is induced under the fed conditions. Inhibiting or lowering expression of either or both of ANGPTL3 and ANGPTL8 could treat cardiovascular disease, nonalcoholic fatty liver disease (NAFLD), metabolic dysfunction-associated steatohepatitis (MASH), and other diseases associated with high triglyceride levels.

The present disclosure provides combinations of ANGPTL3-targeting siNA and ANGPTL8-targeting siNA that can be used together to treatments as disclosed in more detail herein.

### Definitions

It is to be understood that methods are not limited to the particular embodiments described, and as such may vary. It is also to be understood that the terminology used herein is for the purpose of describing particular embodiments only and is not intended to be limiting. The scope of the present technology will be limited only by the appended claims.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

As used in the specification and claims, the singular form “a,” “an” and “the” include singular and plural references unless the context clearly dictates otherwise.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As used herein, “about” means plus or minus 10% as well as the specified number. For example, “about 10” should be understood as both “10” and “9-11.”

As used herein, “optional” or “optionally” means that the subsequently described event or circumstance may or may not occur, and that the description includes instances where said event or circumstance occurs and instances where it does not.

The terms “individual,” “subject,” and “patient” are used interchangeably herein, and refer to any individual mammal, e.g., bovine, canine, feline, equine, simian, porcine, camelid, bat, or human, being treated according to the disclosed methods or uses. In preferred embodiments, the subject is a human.

As used herein, the phrases “effective amount,” “therapeutically effective amount,” and “therapeutic level” mean the siNA dosage or concentration in a subject that provides the specific pharmacological effect for which the siNA is administered in a subject in need of such treatment, i.e. to treat or prevent a cardiovascular disease, nonalcoholic fatty liver disease, etc. It is emphasized that a therapeutically effective amount or therapeutic level of an siNA will not always be effective in treating the diseases described herein, even though such dosage is deemed to be a therapeutically effective amount by those of skill in the art. For convenience only, exemplary dosages, drug delivery amounts, therapeutically effective amounts, and therapeutic levels are provided below. Those skilled in the art can adjust such amounts in accordance with standard practices as needed to treat a specific subject and/or condition. The therapeutically effective amount may vary based on the route of administration and dosage form, the age and weight of the subject, and/or the subject's condition, including the type and severity of the disease or disorder.

The terms “ANGPTL3” and “angiopoietin like 3” refer to nucleic acid sequences encoding a ANGPTL3 protein, peptide, or polypeptide. An example of an ANGPTL3 transcript is Genbank Accession No. NM_014495.4, which is incorporated by reference in its entirety. The term “ANGPTL3” may also include other ANGPTL3 encoding sequences, such as ANGPTL3 isoforms, mutant ANGPTL3 genes, splice variants of ANGPTL3 genes, and ANGPTL3 gene polymorphisms. The term “ANGPTL3” may also refer to a nucleic acid sequence that encodes the polypeptide gene product of a ANGPTL3 gene/transcript, e.g., a ANGPTL3 protein, peptide, or polypeptide according to for example Genbank Accession No. NM 014495.4 of which is incorporated by reference in its entirety.

The terms “ANGPTL8,” “angiopoietin like 8,” and “lipasin” refer to nucleic acid sequences encoding a ANGPTL8 protein, peptide, or polypeptide. An example of an ANGPTL8 transcript is Genbank Accession No. NM_018687.7, which is incorporated by reference in its entirety. The term “ANGPTL8” may also include other ANGPTL8 encoding sequences, such as ANGPTL8 isoforms, mutant ANGPTL8 genes, splice variants of ANGPTL8 genes, and ANGPTL8 gene polymorphisms. The term “ANGPTL8” may also refer to a nucleic acid sequence that encodes the polypeptide gene product of a ANGPTL8 gene/transcript, e.g., a ANGPTL8 protein, peptide, or polypeptide according to for example Genbank Accession No. NM_018687.7 of which is incorporated by reference in its entirety.

The terms “treatment” or “treating” as used herein with reference to reducing or eliminating the disease and/or improving or ameliorating one or more symptoms of the disease.

The terms “prevent” or “preventing” as used herein with reference to a disease refer to precluding a disease from developing in a subject at risk of developing cardiovascular disease and nonalcoholic fatty liver disease.

As used herein, the term “pharmaceutical composition” refers to the combination of at least one active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see, for example, Martin, Remington's Pharmaceutical Sciences, 15th Ed., Mack Publ. Co., Easton, PA [1975].

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

As used herein, the term “nucleobase” refers to a nitrogen-containing biological compound that forms a nucleoside. Examples of nucleobases include, but are not limited to, thymine, uracil, adenine, cytosine, guanine, and an analogue or derivative thereof.

For the purposes of the present disclosure, a DNA sequence that replaces all the U residues of an RNA sequence with T residues is “identical” to the RNA sequence, and vice versa. Accordingly, a sequence that is “identical to an RNA corresponding to” a DNA sequence constitutes the DNA sequence with all T replaced by U. The presence of modified nucleotides or 2′-deoxynucleotides in a sequence does not make a sequence not “identical to an RNA” but rather a modified RNA.

As used herein, “modified nucleotide” includes any nucleic acid or nucleic acid analogue residue that contains a modification or substitution in the chemical structure of an unmodified nucleotide base, sugar (including, but not limited to, 2′-substitution), or phosphate (including, but not limited to, alternate internucleotide linkers, such as phosphorothioates or the substitution of bridging oxygens in phosphate linkers with bridging sulfurs), or a combination thereof. Non-limiting examples of modified nucleotides are shown herein.

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

As a general matter, compositions specifying a percentage are by weight unless otherwise specified. Further, if a variable is not accompanied by a definition, then the previous definition of the variable controls.

### ANGPTL3, ANGPTL8, and Associated Diseases

The siNA molecules and compositions described herein may be administered to a subject to treat a disease. Further disclosed herein are uses of any of the siNA molecules or compositions disclosed herein in the manufacture of a medicament for treating a disease.

The disease being treated may be a disease associated with ANGPTL3 expression, ANGPTL8 expression, or both. ANGPTL3 is an inhibitor of LPL and functions in combination with the cofactor ANGPTL8. ANGPTL3 is exclusively and constitutively expressed in the liver, where ANGPTL8 is expressed in the adipose tissue and liver and is induced after fed conditions. LPL breaks down fat in the form triglyceride in lipoproteins such as chylomicrons (CM) and very low-density lipoproteins (VLDL), into two free fatty acids and one monoacylglycerol molecule.

As used herein, “a disease or disorder associated with ANGPTL3,” “a disease or disorder associated with ANGPTL8,” and “a disease or disorder associated with ANGPTL3 and ANGPTL8” refers to a disease or disorder known in the art to be associated with altered LPL levels, activity, or both. A disease or disorder associated with ANGPTL3, ANGPTL8 or ANGPTL3 and ANGPTL8 may include cardiovascular disease (CVD) and nonalcoholic fatty liver disease (NAFLD), such as metabolic dysfunction-associated steatohepatitis (MASH). Non-limiting examples of cardiovascular disease include hypertriglyceridemia (HTG) and Familial Hypercholesterolemia (FM).

Assessment of siNA molecules targeting ANGPTL3 and ANGPTL8

In some embodiments, a reporter system is used to assess the effectiveness of the siNA molecules of the siNA molecules of the present disclosure in modulating, reducing or inhibiting ANGPTL3 and ANGPTL8. In some embodiments, the reporter system is a luciferase assay. The expression of luciferase can be measured by methods well known in the art. In other embodiments, siNA targeting of a ANGPTL3 and ANGPTL8 target sequence is assessed by measuring ANGPTL3 and ANGPTL8 RNA or protein levels. In such embodiments, ANGPTL3 and ANGPTL8 RNA levels can be assessed by art-recognized methods such as RT-PCR, Northern blot, expression array, etc., and protein levels can be measured by immunoblotting, immunofluorescence, or other antibody-based methods.

In some embodiments, the potency of the disclosed siNA molecules is determined by using a reporter assay. In some embodiments, the ds-siNA of the present disclosure reduces the level or activity of ANGPTL3 by more than about 10%, more than about 20% more than about 30%, more than about 40%, more than about 50%, more than about 60%, more than about 70%, more than about 80%, more than about 90%, or more than about 95%. In some embodiments, the ds-siNA of the present disclosure reduces the level or activity of ANGPTL3 by 100%. In some embodiments, the ds-siNA of the present disclosure reduces the level or activity ANGPTL8 by more than about 10%, more than about 20% more than about 30%, more than about 40%, more than about 50%, more than about 60%, more than about 70%, more than about 80%, more than about 90%, or more than about 95%. In some embodiments, the ds-siNA of the present disclosure reduces the level or activity of ANGPTL8 by 100%.

In some embodiments, the level or activity of ANGPTL3 and ANGPTL8 can be determined by directly measuring ANGPTL3 and ANGPTL8 expression with for example RT-PCR followed by quantitative PCR. In some embodiments, the ds-siNA of the present disclosure reduces the expression level of ANGPTL3 and ANGPTL8 by more than about 10%, more than about 20% more than about 30%, more than about 40%, more than about 50%, more than about 60%, more than about 70%, more than about 80%, more than about 90%, or more than about 95%. In some embodiments, the ds-siNA of the present disclosure reduces the expression level of ANGPTL3 and ANGPTL8 by 100%. In some embodiments, the ds-siNA of the present disclosure reduces the expression level of a ANGPTL3 and/or ANGPTL8 target genes by more than about 10%, more than about 20% more than about 30%, more than about 40%, more than about 50%, more than about 60%, more than about 70%, more than about 80%, more than about 90%, or more than about 95%. In some embodiments, the ds-siNA of the present disclosure reduces the expression of a ANGPTL3 and/or ANGPTL8 target genes by 100%. In some embodiments, the ANGPTL3 target gene comprises NG_028169.1:5005-13798. In some embodiments, the ANGPTL8 target gene comprises NC_000019.10:11239619-11241943.

By measuring percentage inhibition of ANGPTL3 and ANGPTL8 as determined by a reporter system after increasing amounts of ds-siNA, the EC₅₀ (half maximum effective dosage) value of the ds-siNA molecules can be determined. In some embodiments, the ds-siNA has EC₅₀ value of 50 μM or less, of 40 μM or less, of 30 μM or less, of 20 μM or less, 15 μM or less, 10 μM or less, 5 μM or less, 2 μM or less, or 1 μM or less.

By measuring cell viability upon inhibition of ANGPTL3 and ANGPTL8, the CC₅₀ (i.e. the concentration that is required for reducing cell viability by 50%) of the ds-siNA can be determined. In some embodiments, the ds-siNA has a CC₅₀ value of more than about 0.5 μM, more than about 1 μM, more than about 1.5 μM, more than about 2 μM, more than about 2.5 μM, more than about 3 μM, more than about 4 μM, or more than about 5 μM.

In some embodiments, the effectiveness of the ds-siNA can be determined by measuring the expression level of ANGPTL3 and ANGPTL8, and/or a target gene of ANGPTL3 and ANGPTL8. In some embodiments, the effectiveness of the ds-siNA can be determined by measuring the expression level of ANGPTL3. In some embodiments, the effectiveness of the ds-siNA can be determined by measuring the expression level of ANGPTL8.

In some embodiments, the pharmacodynamics of a GalNAc conjugated ANGPTL3 siNA, GalNAc conjugated ANGPTL8 siNA or combination regimen comprising GalNAc conjugated ANGPTL3 siNA and GalNAc conjugated ANGPTL8 siNA as disclosed here in can be determined by in vivo mice studies infected with recombinant viruses AAV-human ANGPTL3 or AAV-human ANGPTL8 or both. Serial and terminal blood collections can be tested for target protein knock down through human ANGPTL3 ELISA, or human ANGPTL8 ELISA or both. Mouse livers can be tested for human NA knock down (human ANGPTL3 qPCR, or human ANMGPTL8 qPCR or both) or human target protein knockdown.

In some embodiments, in vivo efficacy studies can be determined using knock in mice containing human ANGPTL3, human ANGPTL8 or both human ANGPTL3 and ANGPTL8. Mice can be fed with high fat diet and treated with GalNAc conjugated ANGPTL3 siNA, GalNAc conjugated ANGPTL8 siNA or combination regimen comprising GalNAc conjugated ANGPTL3 siNA and GalNAc conjugated ANGPTL8 siNA as disclosed herein. Serial and terminal blood collections can be tested for target protein knock down using ANGPTL3 ELISA, or human ANGPTL8 ELISA or both. Blood LDL-c, TG, HDL-c and total cholesterol can also be measured as efficacy end points. Liver TG and other liver lipid measurements can also be applied.

In some embodiments, the GalNAc conjugated ANGPTL3 siNA, GalNAc conjugated ANGPTL8 siNA or combination regimen comprising GalNAC conjugated ANGPTL3 siNA and GalNAc conjugated ANGPTL8 siNA as disclosed herein can also be tested in monkey dyslipidemia model and monitor blood LDL-c, total Cholesterol, HDL-c, TG and liver fat.

Any of the siNA molecules disclosed herein may further comprise one or more linkers independently selected from a phosphodiester (PO) linker, phosphorothioate (PS) linker, phosphorodithioate linker, mesyl phosphoramidate (Ms), and PS-mimic linker. In some embodiments, the PS-mimic linker is a sulfur linker. In some embodiments, the linkers are internucleoside linkers. Alternatively or additionally, the linkers may connect a nucleotide of the siNA molecule to at least one phosphorylation blocker, conjugated moiety, or 5′-stabilized end cap. In some embodiments, the linkers connect a conjugated moiety to a phosphorylation blocker or 5′-stabilized end cap.

As disclosed in more detail here, the present disclosure provides empirical evidence showing that reducing expression of ANGPTL8 alone with ds-siNA, such as the ds-siNA disclosed here, can reduce cholesterol and triglyceride levels in an animal. As such, combinations of ds-siNA targeting ANGPTL3 and ds-siNA targeting ANGPTL8 can be used in combination to provide further therapeutic benefits.

Short Interfering Nucleic Acid (siNA) Molecules

As indicated above, the present disclosure provides siNA molecules comprising modified nucleotides. Any of the siNA molecules described herein may be double-stranded siNA (ds-siNA) molecules. The terms “siNA molecules” and “ds-siNA molecules” may be used interchangeably. In some embodiments, the ds-siNA molecules comprise a sense strand and an antisense strand. Tables 1~4 details sequences of the present disclosure useful for sense and antisense strands, disclosed in SEQ ID NOs: 1-632 (ANGPTL8) and 633-1125 (ANGPTL3).

In some embodiments, the double-stranded short interfering nucleic acid (ds-siNA) molecule comprises (a) a sense strand comprising a first nucleotide sequence, wherein the first nucleotide sequence is 15 to 30 nucleotides in length and comprises or consists of a nucleotide sequence as set forth in SEQ ID NOs: 1-316; and (b) an antisense strand comprising a second nucleotide sequence, wherein the second nucleotide sequence is 15 to 30 nucleotides in length and comprises or consists of a nucleotide sequence that is the reverse complement of the first nucleotide sequence or has a nucleic acid sequence with sufficient complementarity (i.e., not necessarily 100%) to hybridize to the first nucleotide sequence. In some embodiments, the first nucleotide sequence comprises or consists of a nucleotide sequence as set forth in SEQ ID NOs: 1-316; and the second nucleotide sequence comprises or consists of a nucleotide sequence according to SEQ ID NOs: 317-632.

In some embodiments, the double-stranded short interfering nucleic acid (ds-siNA) molecule comprises (a) a sense strand comprising a first nucleotide sequence, wherein the first nucleotide sequence is 15 to 30 nucleotides in length and comprises or consists of a nucleotide sequence as set forth in SEQ ID NOs: 633-878; and (b) an antisense strand comprising a second nucleotide sequence, wherein the second nucleotide sequence is 15 to 30 nucleotides in length and comprises or consists of a nucleotide sequence that is the reverse complement of the first nucleotide sequence or has a nucleic acid sequence with sufficient complementarity (i.e., not necessarily 100%) to hybridize to the first nucleotide sequence. In some embodiments, the first nucleotide sequence comprises or consists of a nucleotide sequence as set forth in SEQ ID NOs: 633-878; and the second nucleotide sequence comprises or consists of a nucleotide sequence according to SEQ ID NOs: 879-1125.

In some embodiments, the double-stranded short interfering nucleic acid (ds-siNA) molecule comprises (a) a sense strand comprising a first nucleotide sequence, wherein the first nucleotide sequence is 15 to 30 nucleotides in length and comprises a nucleotide sequence with 75%, 80%, 85%, 90%, 91%, 92%, 93%, 94%, 95%, 99%, or 100% similarity to SEQ ID NOs: 1-316; and (b) an antisense strand comprising a second nucleotide sequence, wherein the second nucleotide sequence is 15 to 30 nucleotides in length and comprises a nucleotide sequence that is the reverse complement of the first nucleotide sequence. In some embodiments, the first nucleotide sequence comprises a nucleotide sequence with 75%, 80%, 85%, 90%, 91%, 92%, 93%, 94%, 95%, 99%, or 100% similarity to SEQ ID NOs: 1-316; and the second nucleotide sequence comprises a nucleotide sequence with 75%, 80%, 85%, 90%, 91%, 92%, 93%, 94%, 95%, 99%, or 100% similarity to SEQ ID NOs: 317-632.

In some embodiments, the double-stranded short interfering nucleic acid (ds-siNA) molecule comprises (a) a sense strand comprising a first nucleotide sequence, wherein the first nucleotide sequence is 15 to 30 nucleotides in length and comprises a nucleotide sequence with 75%, 80%, 85%, 90%, 91%, 92%, 93%, 94%, 95%, 99%, or 100% similarity to SEQ ID NOs: 633-878; and (b) an antisense strand comprising a second nucleotide sequence, wherein the second nucleotide sequence is 15 to 30 nucleotides in length and comprises a nucleotide sequence that is the reverse complement of the first nucleotide sequence. In some embodiments, the first nucleotide sequence comprises a nucleotide sequence with 75%, 80%, 85%, 90%, 91%, 92%, 93%, 94%, 95%, 99%, or 100% similarity to SEQ ID NOs: 633-878; and the second nucleotide sequence comprises a nucleotide sequence with 75%, 80%, 85%, 90%, 91%, 92%, 93%, 94%, 95%, 99%, or 100% similarity to SEQ ID NOs: 879-1125.

In some embodiments, the double-stranded short interfering nucleic acid (ds-siNA) molecule comprises (a) a sense strand comprising a first nucleotide sequence, wherein the first nucleotide sequence is 15 to 30 nucleotides in length and comprises a nucleotide sequence that differs by 1, 2, 3, 4, or 5 nucleotides from any one of SEQ ID NOs: 1-316; and (b) an antisense strand comprising a second nucleotide sequence, wherein the second nucleotide sequence is 15 to 30 nucleotides in length and comprises a nucleotide sequence that is the reverse complement of the first nucleotide sequence. In some embodiments, the second nucleotide sequence comprises a nucleotide sequence that differs by 1, 2, 3, 4, or 5 nucleotides from any one of SEQ ID NOs: 317-632.

In some embodiments, the double-stranded short interfering nucleic acid (ds-siNA) molecule comprises (a) a sense strand comprising a first nucleotide sequence, wherein the first nucleotide sequence is 15 to 30 nucleotides in length and comprises a nucleotide sequence that differs by 1, 2, 3, 4, or 5 nucleotides from any one of SEQ ID NOs: 633-878; and (b) an antisense strand comprising a second nucleotide sequence, wherein the second nucleotide sequence is 15 to 30 nucleotides in length and comprises a nucleotide sequence that is the reverse complement of the first nucleotide sequence. In some embodiments, the second nucleotide sequence comprises a nucleotide sequence that differs by 1, 2, 3, 4, or 5 nucleotides from any one of SEQ ID NOs: 879-1125.

In some embodiments, the present disclosure provides a double-stranded short interfering nucleic acid (ds-siNA) molecule comprising (a) a sense strand comprising a nucleotide sequence identical to SEQ ID NOs: 1-316, and (b) an antisense strand. In some embodiments, the present disclosure provides a double-stranded short interfering nucleic acid (ds-siNA) molecule comprising (a) an antisense strand comprising a nucleotide sequence identical to SEQ ID NOs: 317-632; and (b) a sense strand.

In some embodiments, the present disclosure provides a double-stranded short interfering nucleic acid (ds-siNA) molecule comprising (a) a sense strand comprising a nucleotide sequence identical to SEQ ID NOs: 633-878, and (b) an antisense strand. In some embodiments, the present disclosure provides a double-stranded short interfering nucleic acid (ds-siNA) molecule comprising (a) an antisense strand comprising a nucleotide sequence identical to SEQ ID NOs: 879-1125; and (b) a sense strand.

In some embodiments, the present disclosure relates to a double-stranded short interfering nucleic acid (ds-siNA) molecule comprising:
(a) a sense strand comprising a first nucleotide sequence, wherein the first nucleotide sequence is 15 to 30 nucleotides in length and comprises a nucleotide sequence as set forth in SEQ ID NOs: 1-316, and an antisense strand comprising a second nucleotide sequence, wherein the second nucleotide sequence is 15 to 30 nucleotides in length and comprises a nucleotide sequence that is a substantial reverse complement of the first nucleotide sequence; or
(b) a sense strand comprising a first nucleotide sequence, wherein the first nucleotide sequence is 15 to 30 nucleotides in length and comprises a nucleotide sequence that is a substantial reverse complement of a second nucleotide sequence; and
an antisense strand comprising the second nucleotide sequence, wherein the second nucleotide sequence is 15 to 30 nucleotides in length and comprises a nucleotide sequence as set forth in SEQ ID NOs: 317-632.

In some embodiments, the present disclosure relates to a double-stranded short interfering nucleic acid (ds-siNA) molecule comprising:
(a) a sense strand comprising a first nucleotide sequence, wherein the first nucleotide sequence is 15 to 30 nucleotides in length and comprises a nucleotide sequence as set forth in SEQ ID NOs: 633-878, and
an antisense strand comprising a second nucleotide sequence, wherein the second nucleotide sequence is 15 to 30 nucleotides in length and comprises a nucleotide sequence that is a substantial reverse complement of the first nucleotide sequence; or
(b) a sense strand comprising a first nucleotide sequence, wherein the first nucleotide sequence is 15 to 30 nucleotides in length and comprises a nucleotide sequence that is a substantial reverse complement of a second nucleotide sequence; and
an antisense strand comprising the second nucleotide sequence, wherein the second nucleotide sequence is 15 to 30 nucleotides in length and comprises a nucleotide sequence as set forth in SEQ ID NOs: 879-1125.

In some embodiments, the present disclosure relates to a double-stranded short interfering nucleic acid (ds-siNA) molecule comprising (a) a sense strand comprising a nucleotide sequence identical to SEQ ID NOs: 1-316, and (b) an antisense strand.

In some embodiments, the present disclosure relates to a double-stranded short interfering nucleic acid (ds-siNA) molecule comprising (a) a sense strand comprising a nucleotide sequence identical to SEQ ID NOs: 633-878, and (b) an antisense strand.

In some embodiments, the present disclosure relates to a double-stranded short interfering nucleic acid (ds-siNA) molecule comprising (a) an antisense strand comprising a nucleotide sequence identical to SEQ ID NOs: 317-632, and (b) a sense strand.

In some embodiments, the present disclosure relates to a double-stranded short interfering nucleic acid (ds-siNA) molecule comprising (a) an antisense strand comprising a nucleotide sequence identical to SEQ ID NOs: 879-1125, and (b) a sense strand.

In some embodiments, the present disclosure provides a ds-siNA comprising a sense strand comprising any one of SEQ ID NOs: 633, 634, 643, 653, 654, 674, 687, 688, 690-693, 694, 708-713, 756-757, 756, 776, 777, 797, 810, 811, 813-817, or 831-836; and an antisense strand comprising any one of SEQ ID NOs: 879, 880, 888, 899, 900, 920, 933, 934, 936-940, 954-959, 1003-1004, 1012, 1023, 1024, 1044, 1057-1064, or 1078-1083, respectively.

In some embodiments, the present disclosure provides a ds-siNA comprising a sense strand comprising any one of SEQ ID NOs: 633, 654, 688, 690, 708, 713, 756, 777, 811, 813, 831, or 836, and an antisense strand comprising any one of SEQ ID NOs: 879, 900, 934, 936, 954, 959, 1003, 1024, 1058, 1060, 1078, or 1083, respectively.

The disclosed siNA molecules may comprise (a) at least one phosphorylation blocker, conjugated moiety, or 5′-stabilized end cap; and (b) a short interfering nucleic acid (siNA). In some embodiments, the phosphorylation blocker is a phosphorylation blocker disclosed herein. In some embodiments, the 5′-stabilized end cap is a 5′-stabilized end cap disclosed herein. The siNA may comprise any of the first nucleotide, second nucleotide, sense strand, or antisense strand sequences disclosed herein. The siNA may comprise 5 to 100, 5 to 90, 10 to 100, 10 to 90, 10 to 80, 10 to 70, 10 to 60, 10 to 50, 10 to 30, 10 to 25, 15 to 100, 15 to 90, 15 to 80, 15 to 70, 15 to 60, 15 to 50, 15 to 30, or 15 to 25 nucleotides. The siNA may comprise at least 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides. The siNA may comprise less than or equal to 50, 45, 40, 39, 38, 37, 36, 35, 34, 33, 32, 31, 30, 29, 28, 27, 26, 25, 24, 23, 22, 21, 20, or 19 nucleotides. The nucleotides may be modified nucleotides. The siNA may be single stranded. The siNA may be double stranded. The siNA may comprise (a) a sense strand comprising 15 to 30, 15 to 25, 15 to 24, 15 to 23, 15 to 22, 15 to 21, 17 to 30, 17 to 25, 17 to 24, 17 to 23, 17 to 22, 17 to 21, 18 to 30, 18 to 25, 18 to 24, 18 to 23, 18 to 22, 18 to 21, 19 to 30, 19 to 25, 19 to 24, 19 to 23, 19 to 22, 19 to 21, 20 to 25, 20 to 24, 20 to 23, 21 to 25, 21 to 24, or 21 to 23 nucleotides; and (b) an antisense strand comprising 15 to 30, 15 to 25, 15 to 24, 15 to 23, 15 to 22, 15 to 21, 17 to 30, 17 to 25, 17 to 24, 17 to 23, 17 to 22, 17 to 21, 18 to 30, 18 to 25, 18 to 24, 18 to 23, 18 to 22, 18 to 21, 19 to 30, 19 to 25, 19 to 24, 19 to 23, 19 to 22, 19 to 21, 20 to 25, 20 to 24, 20 to 23, 21 to 25, 21 to 24, or 21 to 23 nucleotides. The siNA may comprise (a) a sense strand comprising about 15, 16, 17, 18, 19, 20, 21, 22, or 23 nucleotides; and (b) an antisense strand comprising about 15, 16, 17, 18, 19, 20, 21, 22, or 23 nucleotides. The siNA may comprise (a) a sense strand comprising about 19 nucleotides; and (b) an antisense strand comprising about 21 nucleotides. The siNA may comprise (a) a sense strand comprising about 21 nucleotides; and (b) an antisense strand comprising about 23 nucleotides.

Any of the siNA molecules disclosed herein may further comprise one or more linkers independently selected from a phosphodiester (PO) linker, phosphorothioate (PS) linker, phosphorodithioate linker, mesyl phosphoramidate (Ms), and PS-mimic linker. In some embodiments, the PS-mimic linker is a 3′ or 5′ sulfur linker. In some embodiments, the linkers are internucleoside linkers. Alternatively or additionally, the linkers may connect a nucleotide of the siNA molecule to at least one phosphorylation blocker, conjugated moiety, or 5′-stabilized end cap. In some embodiments, the linkers connect a conjugated moiety to a phosphorylation blocker or 5′-stabilized end cap.

In some embodiments, at least one end of the ds-siNA comprises an overhang, wherein the overhang comprises at least one nucleotide, preferably wherein the at least one nucleotide comprises a 2′-O-methyl nucleotide, a deoxy nucleotide, uracil, or a uracil analogue or derivative.

In some embodiments, both ends of the ds-siNA comprise an overhang, wherein the overhang comprises at least one nucleotide, preferably wherein the at least one nucleotide comprises a 2′-O-methyl nucleotide, a deoxy nucleotide, uracil, or a uracil analogue or derivative.

For the purposes of the present disclosure, a ds-siNA molecule may have the following formula:

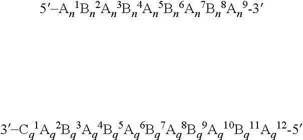

wherein:
the top strand is a sense strand comprising a first nucleotide sequence that is at least about 60%, 65%, 70%, 75%, 80%, 85%, 90%, 91%, 92%, 93%, 94%, 95%, or 100% identical to an RNA corresponding to a target gene, wherein the first nucleotide sequence comprises 15 to 30 nucleotides;
the bottom strand is an antisense strand comprising a second nucleotide sequence that is at least about 60%, 65%, 70%, 75%, 80%, 85%, 90%, 91%, 92%, 93%, 94%, 95%, or 100% complementary to the RNA corresponding to the target gene, wherein the second nucleotide sequence comprises 15 to 30 nucleotides;
each A is independently a 2′-O-methyl nucleotide or a nucleotide comprising a 5′ stabilized end cap or phosphorylation blocker;
B is a 2′-fluoro nucleotide;
C represents overhanging nucleotides and is a 2′-O-methyl nucleotide, a deoxy nucleotide, or uracil;
n¹=1-6 nucleotides in length;
each n², n⁶, n⁸, q³, q⁵, q⁷, q⁹, q¹¹, and q¹² is independently 0-1 nucleotides in length;
each n³ and n⁴ is independently 1-3 nucleotides in length;
n⁵ is 1-10 nucleotides in length;
n⁷ is 0-4 nucleotides in length;
each n⁹, q¹, and q² is independently 0-2 nucleotides in length;
q⁴ is 0-3 nucleotides in length;
q⁶ is 0-5 nucleotides in length;
q⁸ is 2-7 nucleotides in length; and
q¹⁰ is 2-11 nucleotides in length.

The ds-siNA may further comprise a conjugated moiety. The conjugated moiety may comprise any of the galactosamines disclosed herein. The ds-siNA may further comprise (i) phosphorothioate internucleoside linkages between the nucleotides at positions 1 and 2 and positions 2 and 3 from the 5′ end of the sense strand; and (ii) phosphorothioate internucleoside linkages between any nucleotide position, for example at positions 1 and 2; positions 2 and 3; positions 3 and 4 etc. The ds-siNA may further comprise a 5′-stabilizing end cap. The 5′-stabilizing end cap may be a 2′-OMe-vinyl phosphonate (vm). The 5′-stabilizing end cap may feature a modified base (e.g., vm(5mim), and vm(56mido)), as disclosed herein. The 5′-stabilizing end cap may be attached to the 5′ end of the antisense strand. In some embodiments, the 2′-O-methyl nucleotide at position 1 from the 5′ end of the sense strand is further modified to contain a 5′ stabilizing end cap. In some embodiments, the 2′-O-methyl nucleotide at position 1 from the 5′ end of the antisense strand is further modified to contain a 5′ stabilizing end cap. In some embodiments, the 2′-O-methyl nucleotide at position 1 from the 5′ end of the sense strand is further modified to contain a phosphorylation blocker. In some embodiments, the 2′-O-methyl nucleotide at position 1 from the 3′ end of the sense strand is further modified to contain a phosphorylation blocker. In some embodiments, the 2′-O-methyl nucleotide at position 1 from the 5′ end of the antisense strand is further modified to contain a phosphorylation blocker. In some embodiments, the 2′-O-methyl nucleotide at position 1 from the 3′ end of the antisense strand is further modified to contain a phosphorylation blocker. An exemplary ds-siNA molecule may have the following formula:

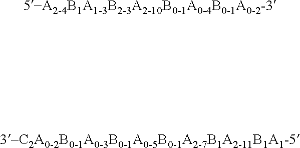

wherein:
the top strand is a sense strand comprising a first nucleotide sequence that is at least about 60%, 65%, 70%, 75%, 80%, 85%, 90%, 91%, 92%, 93%, 94%, 95%, or 100% identical to an RNA corresponding to a target gene, wherein the first nucleotide sequence comprises 15 to 30 nucleotides;
the bottom strand is an antisense strand comprising a second nucleotide sequence that is at least about 60%, 65%, 70%, 75%, 80%, 85%, 90%, 91%, 92%, 93%, 94%, 95%, or 100% complementary to the RNA corresponding to the target gene, wherein the second nucleotide sequence comprises 15 to 30 nucleotides;
each A is independently a 2′-O-methyl nucleotide or a nucleotide comprising a 5′ stabilized end cap or phosphorylation blocker;
B is a 2′-fluoro nucleotide;
C represents overhanging nucleotides and is a 2′-O-methyl nucleotide, a deoxy nucleotide, or uracil (or uracil containing nucleotide).

The ds-siNA may further comprise a conjugated moiety. The conjugated moiety may comprise any of the galactosamines disclosed herein. The ds-siNA may further comprise (i) phosphorothioate internucleoside linkages between the nucleotides at positions 1 and 2 and positions 2 and 3 from the 5′ end of the sense strand; and (ii) phosphorothioate internucleoside linkages between the nucleotides at positions 1 and 2; positions 2 and 3; positions 19 and 20; and positions 20 and 21 from the 5′ end of the antisense strand. The ds-siNA may further comprise a 5′-stabilizing end cap. The 5′-stabilizing end cap may include a vinyl phosphonate. The vinyl phosphonate may be a deuterated vinyl phosphonate. The deuterated vinyl phosphonate may be a mono-deuterated vinyl phosphonate. The deuterated vinyl phosphonate may be a mono- or di-deuterated vinyl phosphonate. In some embodiments, the 5′ end of the antisense strand may comprise a

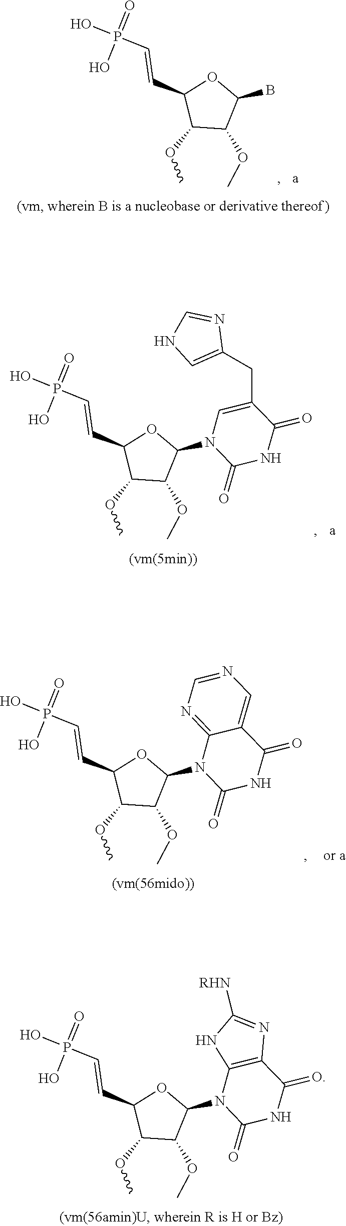

The 5′-stabilizing end cap may be attached to the 5′ end of the antisense strand. The 5′-stabilizing end cap may be attached to the 3′ end of the antisense strand. The 5′-stabilizing end cap may be attached to the 5′ end of the sense strand. The 5′-stabilizing end cap may be attached to the 3′ end of the sense strand. In some embodiments, the 2′-O-methyl nucleotide at position 1 from the 5′ end of the sense strand is further modified to contain a 5′ stabilizing end cap. In some embodiments, the 2′-O-methyl nucleotide at position 1 from the 5′ end of the antisense strand is further modified to contain a 5′ stabilizing end cap. In some embodiments, the 2′-O-methyl nucleotide at position 1 from the 5′ end of the sense strand is further modified to contain a phosphorylation blocker. In some embodiments, the 2′-O-methyl nucleotide at position 1 from the 3′ end of the sense strand is further modified to contain a phosphorylation blocker. In some embodiments, the 2′-O-methyl nucleotide at position 1 from the 5′ end of the antisense strand is further modified to contain a phosphorylation blocker. In some embodiments, the 2′-O-methyl nucleotide at position 1 from the 3′ end of the antisense strand is further modified to contain a phosphorylation blocker.

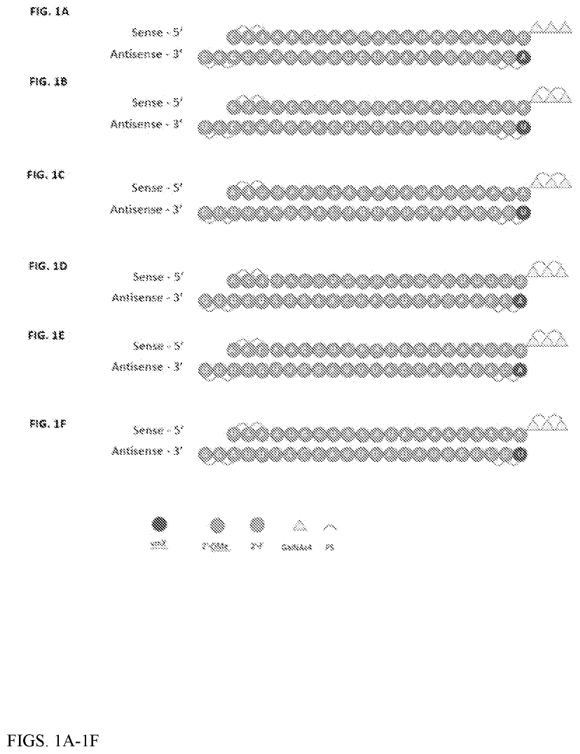

The exemplary ds-siNA shown in FIG. 1A FIG. 1F comprise (i) a sense strand comprising 19-25 nucleotides (e.g., 21 nucleotides); and (ii) an antisense strand comprising 21-25 nucleotides (e.g., 23 nucleotides). The ds-siNA may optionally further comprise (iii) a conjugated moiety, wherein the conjugated moiety (e.g GalNAc4 is attached to the 3′ end or the 5′ end of the sense strand or the antisense strand. The ds-siNA may comprise a 2 nucleotide overhang at positions 22 and 23 from the 5′ end of the antisense strand. The ds-siNA may further comprise 1, 2, 3, 4, 5, 6 or more phosphorothioate (ps) internucleoside linkages or mesyl phosphoramidate internucleoside linkage (Ms). At least one phosphorothioate internucleoside linkage or mesyl phosphoramidate internucleoside linkage (Ms) may be between the nucleotides at positions 1 and 2 or positions 2 and 3 from the 5′ end of the sense strand. At least one phosphorothioate internucleoside linkage or mesyl phosphoramidate internucleoside linkage (Ms) may be between the nucleotides at positions 1 and 2 or positions 2 and 3 from the 5′ end of the antisense strand. At least one phosphorothioate internucleoside linkage or mesyl phosphoramidate internucleoside linkage (Ms) may be between the nucleotides at positions 19 and 20, positions 20 and 21, positions 21 and 22, or positions 22 and 23 from the 5′ end of the antisense strand or any combination thereof. As shown in FIGS. 1A-1F, about 4 nucleotides (e.g., 1, 2, 3, 4, 5, or 6) in the sense strand may be 2′-fluoro nucleotides. As shown in FIGS. 1A-1F, about 4 nucleotides (e.g., 1, 2, 3, 4, 5, or 6) in the antisense strand may be 2′-fluoro nucleotides. As shown in FIGS. 1A-1F, 15-17 nucleotides in the sense strand may be 2′-O-methyl nucleotides. As shown in FIGS. 1A-1F 16-21 nucleotides in the antisense strand may be 2′-O-methyl nucleotides.

In some embodiments, the 2′-O-methyl nucleotide at position 1 from the 5′ end of the sense strand can be further modified to contain a 5′ stabilizing end cap. In some embodiments, the 2′-O-methyl nucleotide at position 1 from the 5′ end of the antisense strand can be further modified to contain a 5′ stabilizing end cap. In some embodiments, the 2′-O-methyl nucleotide at position 1 from the 5′ end of the sense strand can be further modified to contain a phosphorylation blocker. In some embodiments, the 2′-O-methyl nucleotide at position 1 from the 3′ end of the sense strand can be further modified to contain a phosphorylation blocker. In some embodiments, the 2′-O-methyl nucleotide at position 1 from the 5′ end of the antisense strand can be further modified to contain a phosphorylation blocker. In some embodiments, the 2′-O-methyl nucleotide at position 1 from the 3′ end of the antisense strand can be further modified to contain a phosphorylation blocker.

In some embodiments, the 2′-O-methyl nucleotide at position 1 from the 5′ end of the sense strand is a d2vd3 nucleotide, a d2vd3U nucleotide, a d2vd3A nucleotide, an omeco-d3 nucleotide, an omeco-d3U nucleotide, an omeco-d3A nucleotide, a 4 h nucleotide, a 4 hU nucleotide, a 4 hA nucleotide, a v-mun nucleotide, a c2o-4 h nucleotide, an omeco-munb nucleotide, a d2vm nucleotide, or a d2vmA nucleotide. In some embodiments, the 2′-O-methyl nucleotide at position 1 from the 5′ end of the antisense strand is a d2vd3 nucleotide, a d2vd3U nucleotide, a d2vd3A nucleotide, an omeco-d3 nucleotide, an omeco-d3U nucleotide, an omeco-d3A nucleotide, a 4 h nucleotide, a 4 hU nucleotide, a 4 hA nucleotide, a v-mun nucleotide, a c2o-4 h nucleotide, an omeco-munb nucleotide, a d2vm nucleotide, or a d2vmA nucleotide. In some embodiments, the 2′-O-methyl nucleotide at position 1 from the 3′ end of the sense strand is a d2vd3 nucleotide, a d2vd3U nucleotide, a d2vd3A nucleotide, an omeco-d3 nucleotide, an omeco-d3U nucleotide, an omeco-d3A nucleotide, a 4h nucleotide, a 4 hU nucleotide, a 4 hA nucleotide, a v-mun nucleotide, a c20-4 h nucleotide, an omeco-munb nucleotide, a d2vm nucleotide, or a d2vmA nucleotide. In some embodiments, the 2′-O-methyl nucleotide at position 1 from the 3′ end of the antisense strand is a d2vd3 nucleotide, a d2vd3U nucleotide, d2vd3A nucleotide, an omeco-d3 nucleotide, an omeco-d3U nucleotide, an omeco-d3A nucleotide, a 4 h nucleotide, a 4 hU nucleotide, a v-mun nucleotide, a c20-4 h nucleotide, an omeco-munb nucleotide, a d2vm nucleotide, or a d2vmA nucleotide. In some embodiments, at least 1, 2, 3, 4 or more 2′-fluoro nucleotides on the sense strand or antisense strand is a 2′-fluoro nucleotide mimic. In some embodiments, at least 1, 2, 3, 4 or more 2′-fluoro nucleotides on the sense strand or antisense strand is a fB, fN, f (4nh) Q, f4P, f2P, or fX nucleotide. In some embodiments, at least 1, 2, 3, 4 or more 2′-O-methyl nucleotide on the sense or antisense strand is a 2′-O-methyl nucleotide mimic. In some embodiments, one or more nucleotides in the sense strand and/or the antisense strand may be a 3′,4′ seco modified nucleotide in which the bond between the 3′ and 4′ positions of the furanose ring is broken (e.g., mun34).

In some embodiments, the antisense strand, sense strand, first nucleotide sequence, and/or second nucleotide sequence comprises at least one modified nucleotide comprising

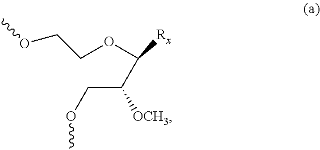

wherein R^{x} is a nucleobase, aryl, heteroaryl, or H,

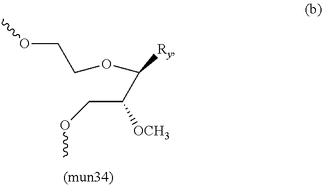

wherein R^{y} is a nucleobase,

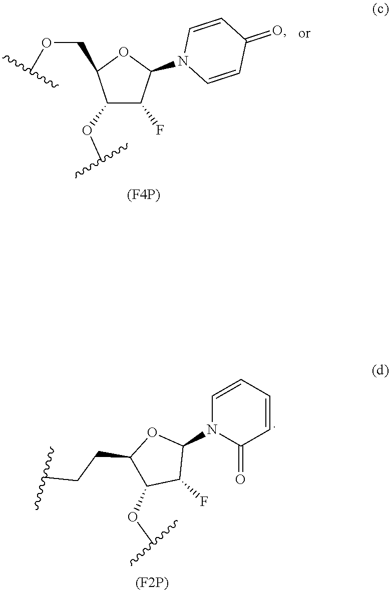

In some embodiments, the second nucleotide sequence (or antisense strand) comprises a modified nucleotide comprising at least

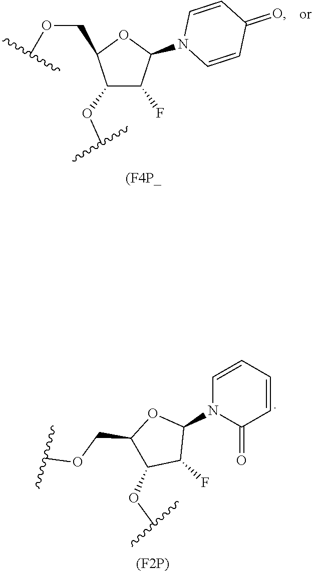

In some embodiments, the sense strand of the siNA, the antisense strand or the siNA, or both comprise at least 1, at least 2, at least 3, at least 4, or at least 5 nucleotide(s) comprising a structure independently selected from:

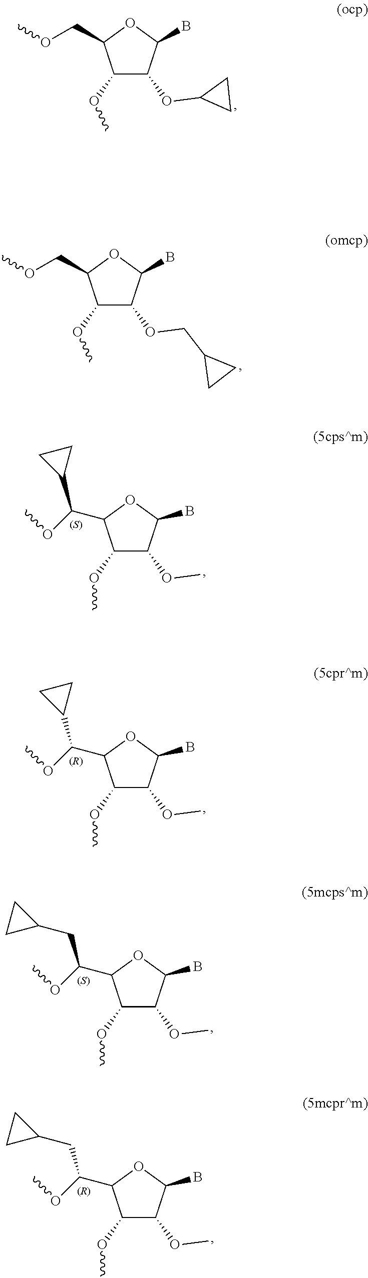

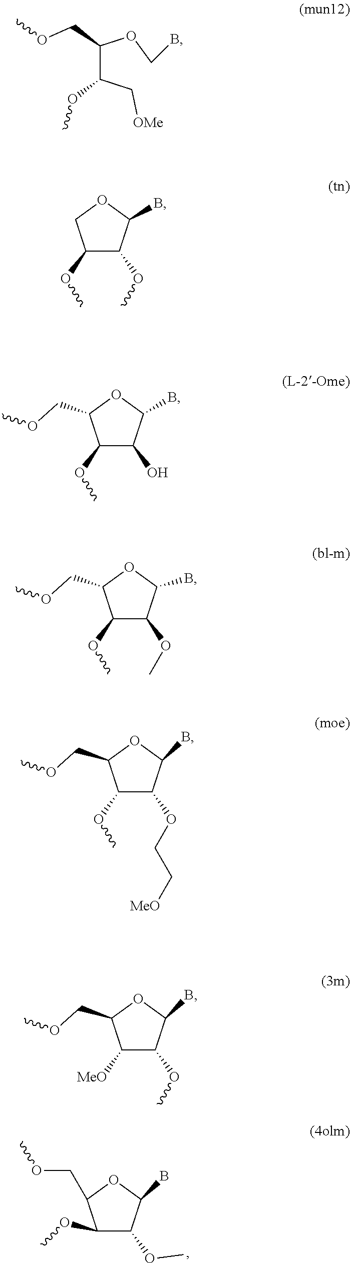

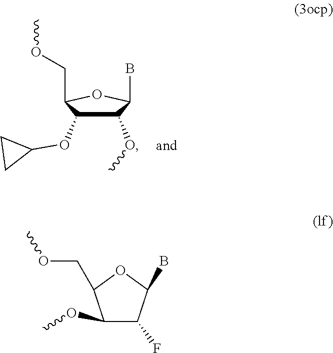

or at least 1, at least 2, at least 3, at least 4, or at least 5 nucleotide analog(s) comprising a structure independently selected from:

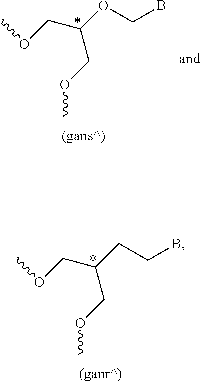

wherein B is a nucleobase, an aryl, heteroaryl, or H; wherein [symbol] represents a phosphodiester linkage, a phosphorothioate linkage, or H; and wherein * represent chiral center (e.g., R or S isomer). In some embodiments, the sense strand, the antisense strand, or both each independently comprise 1 or more phosphorothioate internucleoside linkages. In some embodiments, the siNA further comprises a phosphorylation blocker.

In some embodiments, the sense strand of the siNA, the antisense strand of the siNA, or both each independently comprise at least 1, at least 2, at least 3, at least 4, or at least 5 or more of

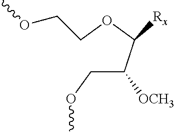

wherein Rₓ is a nucleobase, aryl, heteroaryl, or H,

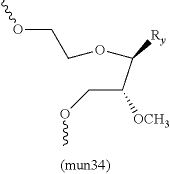

wherein R_{y} is a nucleobase,

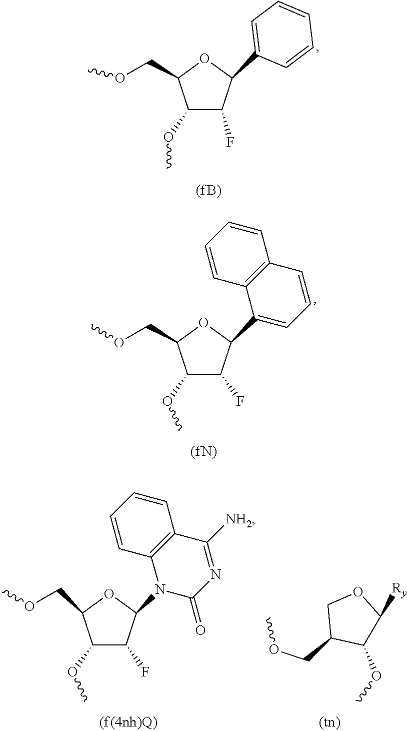

wherein R_{y} is a nucleobase, or combinations thereof.

In some embodiments, the siNA further comprises a galactosamine. In some embodiments, the galactosamine is N-acetylgalactosamine (GalNAc) of Formula (VI):

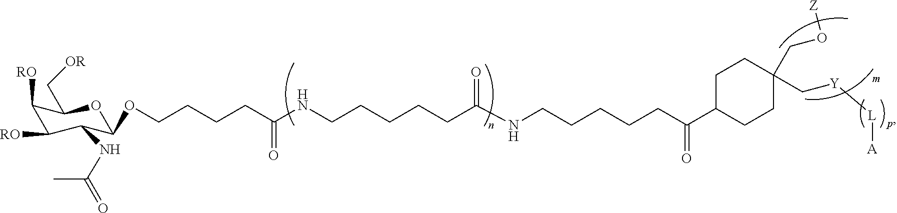

wherein
m is 1, 2, 3, 4, or 5;
each n is independently 1 or 2;
p is 0 or 1;
each R is independently H;
each Y is independently selected from —O—P(═O)(SH)—, —O—P(═O)(O)—, —O—P(═O)(OH)—, and —O—P(S)S—;
Z is H or a second protecting group;
either L is a linker or L and Y in combination are a linker; and
A is H, OH, a third protecting group, an activated group, or an oligonucleotide. In some embodiments, the galactosamine is N-acetylgalactosamine (GalNAc) of Formula (VII):

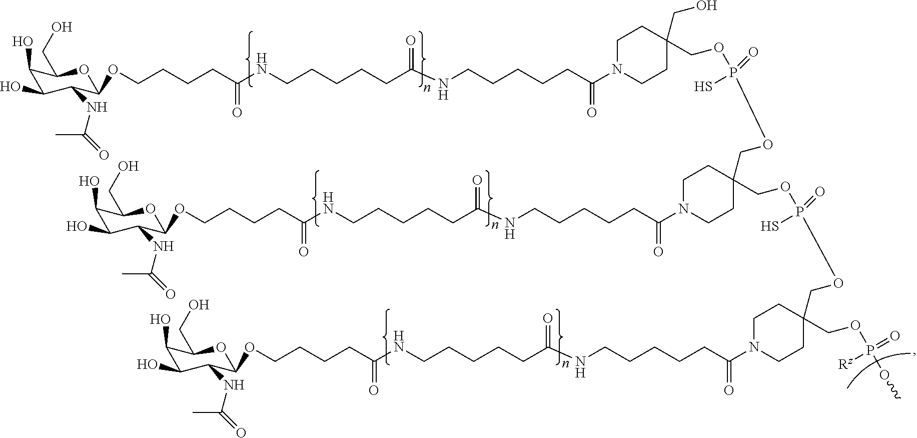

wherein R^{z} is OH or SH; and each n is independently 1 or 2.
   A. siNA Sense Strand

Any of the siNA molecules described herein may comprise a sense strand. The sense strand may comprise a first nucleotide sequence. The first nucleotide sequence may be 15 to 30, 15 to 25, 15 to 23, 17 to 23, 19 to 23, or 19 to 21 nucleotides in length. In some embodiments, the first nucleotide sequence is 15, 16, 17, 18, 19, 20, 21, 22, 23, 24, 25, 26, 27, 28, 29, or 30 nucleotides in length. In some embodiments, the first nucleotide sequence is at least 19 nucleotides in length. In some embodiments, the first nucleotide sequence is at least 21 nucleotides in length.

In some embodiments, the sense strand is the same length as the first nucleotide sequence. In some embodiments, the sense strand is longer than the first nucleotide sequence. In some embodiments, the sense strand may further comprise 1, 2, 3, 4, or 5 or more nucleotides than the first nucleotide sequence. In some embodiments, the sense strand may further comprise a deoxyribonucleic acid (DNA). In some embodiments, the DNA is thymine (T). In some embodiments, the sense strand may further comprise a TT sequence. In some embodiments, the sense strand may further comprise one or more modified nucleotides that are adjacent to the first nucleotide sequence. In some embodiments, the one or more modified nucleotides are independently selected from any of the modified nucleotides disclosed herein (e.g., 2′-fluoro nucleotide, 2′-O-methyl nucleotide, 2′-fluoro nucleotide mimic, 2′-O-methyl nucleotide mimic, or a nucleotide comprising a modified nucleobase).

In some embodiments, the first nucleotide sequence comprises 15, 16, 17, 18, 19, 20, 21, 22, 23, or more modified nucleotides independently selected from a 2′-O-methyl nucleotide and a 2′-fluoro nucleotide. In some embodiments, 70%, 75%, 80%, 85%, 90%, 91%, 92%, 93%, 94%, 95% or 100% of the nucleotides in the first nucleotide sequence are modified nucleotides independently selected from a 2′-O-methyl nucleotide and a 2′-fluoro nucleotide. In some embodiments, 100% of the nucleotides in the first nucleotide sequence are modified nucleotides independently selected from a 2′-O-methyl nucleotide and a 2′-fluoro nucleotide. In some embodiments, the 2′-O-methyl nucleotide is a 2′-O-methyl nucleotide mimic. In some embodiments, the 2′-fluoro nucleotide is a 2′-fluoro nucleotide mimic.

In some embodiments, between about 15 to 30, 15 to 25, 15 to 24, 15 to 23, 15 to 22, 15 to 21, 17 to 30, 17 to 25, 17 to 24, 17 to 23, 17 to 22, 17 to 21, 18 to 30, 18 to 25, 18 to 24, 18 to 23, 18 to 22, 18 to 21, 19 to 30, 19 to 25, 19 to 24, 19 to 23, 19 to 22, 19 to 21, 20 to 25, 20 to 24, 20 to 23, 21 to 25, 21 to 24, or 21 to 23 modified nucleotides of the first nucleotide sequence are 2′-O-methyl nucleotides. In some embodiments, between about 2 to 20 modified nucleotides of the first nucleotide sequence are 2′-O-methyl nucleotides. In some embodiments, between about 5 to 25 modified nucleotides of the first nucleotide sequence are 2′-O-methyl nucleotides. In some embodiments, between about 10 to 25 modified nucleotides of the first nucleotide sequence are 2′-O-methyl nucleotides. In some embodiments, between about 12 to 25 modified nucleotides of the first nucleotide sequence are 2′-O-methyl nucleotides. In some embodiments, at least about 10, 11, 12, 13, 14, 15, 16, 17, 18, 19, 20, 21, or 22 modified nucleotides of the first nucleotide sequence are 2′-O-methyl nucleotides. In some embodiments, at least about 12 modified nucleotides of the first nucleotide sequence are 2′-O-methyl nucleotides. In some embodiments, at least about 13 modified nucleotides of the first nucleotide sequence are 2′-O-methyl nucleotides. In some embodiments, at least about 14 modified nucleotides of the first nucleotide sequence are 2′-O-methyl nucleotides. In some embodiments, at least about 15 modified nucleotides of the first nucleotide sequence are 2′-O-methyl nucleotides. In some embodiments, at least about 16 modified nucleotides of the first nucleotide sequence are 2′-O-methyl nucleotides. In some embodiments, at least about 17 modified nucleotides of the first nucleotide sequence are 2′-O-methyl nucleotides. In some embodiments, at least about 18 modified nucleotides of the first nucleotide sequence are 2′-O-methyl nucleotides. In some embodiments, at least about 19 modified nucleotides of the first nucleotide sequence are 2′-O-methyl nucleotides. In some embodiments, less than or equal to 25, 24, 23, 22, 21, 20, 19, 18, 17, 16, 15, 14, 13, 12, 11, 10, 9, 8, 7, 6, 5, 4, 3, or 2 modified nucleotides of the first nucleotide sequence are 2′-O-methyl nucleotides. In some embodiments, less than or equal to 21 modified nucleotides of the first nucleotide sequence are 2′-O-methyl nucleotides. In some embodiments, less than or equal to 20 modified nucleotides of the first nucleotide sequence are 2′-O-methyl nucleotides. In some embodiments, less than or equal to 19 modified nucleotides of the first nucleotide sequence are 2′-O-methyl nucleotides. In some embodiments, less than or equal to 18 modified nucleotides of the first nucleotide sequence are 2′-O-methyl nucleotides. In some embodiments, less than or equal to 17 modified nucleotides of the first nucleotide sequence are 2′-O-methyl nucleotides. In some embodiments, less than or equal to 16 modified nucleotides of the first nucleotide sequence are 2′-O-methyl nucleotides. In some embodiments, less than or equal to 15 modified nucleotides of the first nucleotide sequence are 2′-O-methyl nucleotides. In some embodiments, less than or equal to 14 modified nucleotides of the first nucleotide sequence are 2′-O-methyl nucleotides. In some embodiments, less than or equal to 13 modified nucleotides of the first nucleotide sequence are 2′-O-methyl nucleotides. In some embodiments, at least one modified nucleotide of the first nucleotide sequence is a 2′-O-methyl pyrimidine. In some embodiments, at least 5, 6, 7, 8, 9, or 10 modified nucleotides of the first nucleotide sequence are 2′-O-methyl pyrimidines. In some embodiments, at least one modified nucleotide of the first nucleotide sequence is a 2′-O-methyl purine. In some embodiments, at least 5, 6, 7, 8, 9, or 10 modified nucleotides of the first nucleotide sequence are 2′-O-methyl purines. In some embodiments, the 2′-O-methyl nucleotide is a 2′-O-methyl nucleotide mimic.

In some embodiments, between 2 to 15 modified nucleotides of the first nucleotide sequence are 2′-fluoro nucleotides. In some embodiments, between 2 to 10 modified nucleotides of the first nucleotide sequence are 2′-fluoro nucleotides. In some embodiments, between 2 to 6 modified nucleotides of the first nucleotide sequence are 2′-fluoro nucleotides. In some embodiments, 1 to 6, 1 to 5, 1 to 4, or 1 to 3 modified nucleotides of the first nucleotide sequence are 2′-fluoro nucleotides. In some embodiments, at least 1, 2, 3, 4, 5, or 6 modified nucleotides of the first nucleotide sequence are 2′-fluoro nucleotides. In some embodiments, at least 1 modified nucleotide of the first nucleotide sequence is a 2′-fluoro nucleotide. In some embodiments, at least 2 modified nucleotides of the first nucleotide sequence are 2′-fluoro nucleotides. In some embodiments, at least 3 modified nucleotides of the first nucleotide sequence are 2′-fluoro nucleotides. In some embodiments, at least 4 modified nucleotides of the first nucleotide sequence are 2′-fluoro nucleotides. In some embodiments, at least 5 modified nucleotides of the first nucleotide sequence are 2′-fluoro nucleotides. In some embodiments, at least 6 modified nucleotides of the first nucleotide sequence are 2′-fluoro nucleotides. In some embodiments, 10, 9, 8, 7, 6, 5, 4, 3 or fewer modified nucleotides of the first nucleotide sequence are 2′-fluoro nucleotides. In some embodiments, 10 or fewer modified nucleotides of the first nucleotide sequence are 2′-fluoro nucleotides. In some embodiments, 7 or fewer modified nucleotides of the first nucleotide sequence are 2′-fluoro nucleotides. In some embodiments, 6 or fewer modified nucleotides of the first nucleotide sequence are 2′-fluoro nucleotides. In some embodiments, 5 or fewer modified nucleotides of the first nucleotide sequence are 2′-fluoro nucleotides. In some embodiments, 4 or fewer modified nucleotides of the first nucleotide sequence are 2′-fluoro nucleotides. In some embodiments, 3 or fewer modified nucleotides of the first nucleotide sequence are 2′-fluoro nucleotides. In some embodiments, 2 or fewer modified nucleotides of the first nucleotide sequence are 2′-fluoro nucleotides. In some embodiments, at least one modified nucleotide of the first nucleotide sequence is a 2′-fluoro pyrimidine. In some embodiments, 1, 2, 3, 4, 5, or 6 modified nucleotides of the first nucleotide sequence are 2′-fluoro pyrimidines. In some embodiments, at least one modified nucleotide of the first nucleotide sequence is a 2′-fluoro purine. In some embodiments, 1, 2, 3, 4, 5, or 6 modified nucleotides of the first nucleotide sequence are 2′-fluoro purines. In some embodiments, the 2′-fluoro nucleotide is a 2′-fluoro nucleotide mimic.

In some embodiments, the nucleotide at position 3, 5, 7, 8, 9, 10, 11, 12, 14, 17, and/or 19 from the 5′ end of the first nucleotide sequence is a 2′-fluoro nucleotide. In some embodiments, at least two nucleotides at positions 3, 5, 7, 8, 9, 10, 11, 12, 14, 17, and/or 19 from the 5′ end of the first nucleotide sequence are 2′-fluoro nucleotides. In some embodiments, at least three nucleotides at positions 3, 5, 7, 8, 9, 10, 11, 12, 14, 17, and/or 19 from the 5′ end of the first nucleotide sequence are 2′-fluoro nucleotides. In some embodiments, at least four nucleotides at positions 3, 5, 7, 8, 9, 10, 11, 12, 14, 17, and/or 19 from the 5′ end of the first nucleotide sequence are 2′-fluoro nucleotides. In some embodiments, at least five nucleotides at positions 3, 5, 7, 8, 9, 10, 11, 12, 14, 17, and/or 19 from the 5′ end of the first nucleotide sequence are 2′-fluoro nucleotides. In some embodiments, the nucleotides at positions 3, 5, 7, 8, 9, 10, 11, 12, 14, 17, and/or 19 from the 5′ end of the first nucleotide sequence are 2′-fluoro nucleotides. In some embodiments, the nucleotide at position 3 from the 5′ end of the first nucleotide sequence is a 2′-fluoro nucleotide. In some embodiments, the nucleotide at position 7 from the 5′ end of the first nucleotide sequence is a 2′-fluoro nucleotide. In some embodiments, the nucleotide at position 8 from the 5′ end of the first nucleotide sequence is a 2′-fluoro nucleotide. In some embodiments, the nucleotide at position 9 from the 5′ end of the first nucleotide sequence is a 2′-fluoro nucleotide. In some embodiments, the nucleotide at position 12 from the 5′ end of the first nucleotide sequence is a 2′-fluoro nucleotide. In some embodiments, the nucleotide at position 17 from the 5′ end of the first nucleotide sequence is a 2′-fluoro nucleotide. In some embodiments, the 2′-fluoro nucleotide is a 2′-fluoro nucleotide mimic.

In some embodiments, at least 1, 2, 3, 4, 5, 6, or 7 nucleotides at position 3, 5, 7, 8, 9, 10, 11, 12, 14, 17, and/or 19 from the 5′ end of the first nucleotide sequence is a 2′-fluoro nucleotide. In some embodiments, the nucleotide at positions 3, 5, 7, 8, 9, 10, 11, 12, 14, 17, and/or 19 from the 5′ end of the first nucleotide sequence is a 2′-fluoro nucleotide. In some embodiments, at least two nucleotides at positions 3, 5, 7, 8, 9, 10, 11, 12, 14, 17, and/or 19 from the 5′ end of the first nucleotide sequence are 2′-fluoro nucleotides. In some embodiments, at least three nucleotides at positions 3, 5, 7, 8, 9, 10, 11, 12, 14, 17, and/or 19 from the 5′ end of the first nucleotide sequence are 2′-fluoro nucleotides. In some embodiments, the nucleotides at positions 3, 5, 7, 8, 9, 10, 11, 12, 14, 17, and/or 19 from the 5′ end of the first nucleotide sequence are 2′-fluoro nucleotides. In some embodiments, the nucleotide at position 3 from the 5′ end of the first nucleotide sequence is a 2′-fluoro nucleotide. In some embodiments, the nucleotide at position 5 from the 5′ end of the first nucleotide sequence is a 2′-fluoro nucleotide. In some embodiments, the nucleotide at position 7 from the 5′ end of the first nucleotide sequence is a 2′-fluoro nucleotide. In some embodiments, the nucleotide at position 8 from the 5′ end of the first nucleotide sequence is a 2′-fluoro nucleotide. In some embodiments, the nucleotide at position 9 from the 5′ end of the first nucleotide sequence is a 2′-fluoro nucleotide. In some embodiments, the nucleotide at position 10 from the 5′ end of the first nucleotide sequence is a 2′-fluoro nucleotide. In some embodiments, the nucleotide at position 11 from the 5′ end of the first nucleotide sequence is a 2′-fluoro nucleotide. In some embodiments, the nucleotide at position 12 from the 5′ end of the first nucleotide sequence is a 2′-fluoro nucleotide. In some embodiments, the nucleotide at position 14 from the 5′ end of the first nucleotide sequence is a 2′-fluoro nucleotide. In some embodiments, the nucleotide at position 17 from the 5′ end of the first nucleotide sequence is a 2′-fluoro nucleotide. In some embodiments, the nucleotide at position 19 from the 5′ end of the first nucleotide sequence is a 2′-fluoro nucleotide. In some embodiments, the nucleotide at position 3, 7, 8, 9, 12, and/or 17 from the 5′ end of the first nucleotide sequence is a 2′-fluoro nucleotide. In some embodiments, the nucleotide at position 3, 7, 8, and/or 17 from the 5′ end of the first nucleotide sequence is a 2′-fluoro nucleotide. In some embodiments, the nucleotide at position 3, 7, 8, 9, 12, and/or 17 from the 5′ end of the first nucleotide sequence is a 2′-fluoro nucleotide. In some embodiments, the nucleotide at position 5, 7, 8, and/or 9 from the 5′ end of the first nucleotide sequence is a 2′-fluoro nucleotide. In some embodiments, the nucleotide at position 5, 9, 10, 11, 12, and/or 19 from the 5′ end of the first nucleotide sequence is a 2′-fluoro nucleotide. In some embodiments, the 2′-fluoro nucleotide is a 2′-fluoro nucleotide mimic.

In some embodiments, the 2′-fluoro or 2′-O-methyl nucleotide mimic is a nucleotide mimic of Formula (V):

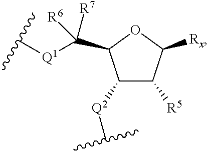

wherein Rₓ is independently a nucleobase, aryl, heteroaryl, or H, Q¹ and Q² are independently S or O, R⁵ is independently -OCD₃, —F, or —OCH₃, and R⁶ and R⁷ are independently H, D, or CD₃. In some embodiments, the nucleobase is selected from thymine, cytosine, guanine, adenine, uracil, and an analogue or derivative thereof.

In some embodiments, the 2′-fluoro or 2′-O-methyl nucleotide mimic is a nucleotide mimic of Formula (16)-Formula (20):

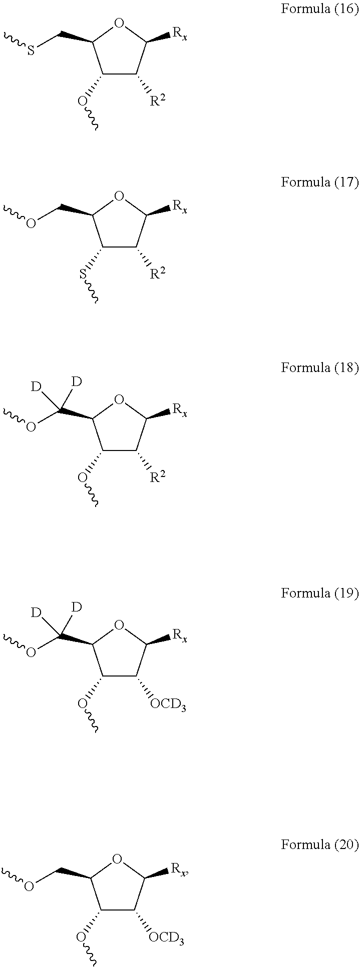

wherein Rₓ is independently a nucleobase, aryl, heteroaryl, or H and R² is F or —OCH₃. In some embodiments, the nucleobase is selected from thymine, cytosine, guanine, adenine, uracil, and an analogue or derivative thereof.

In some embodiments, the sense strand may comprise 1, 2, 3, 4, or 5 or more modified nucleotide(s) selected from:

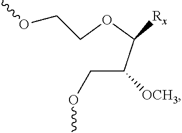

wherein Rx is a nucleobase, aryl, heteroaryl, or H,

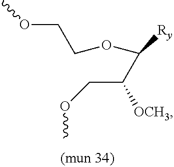

wherein R_{y} is a nucleobase,

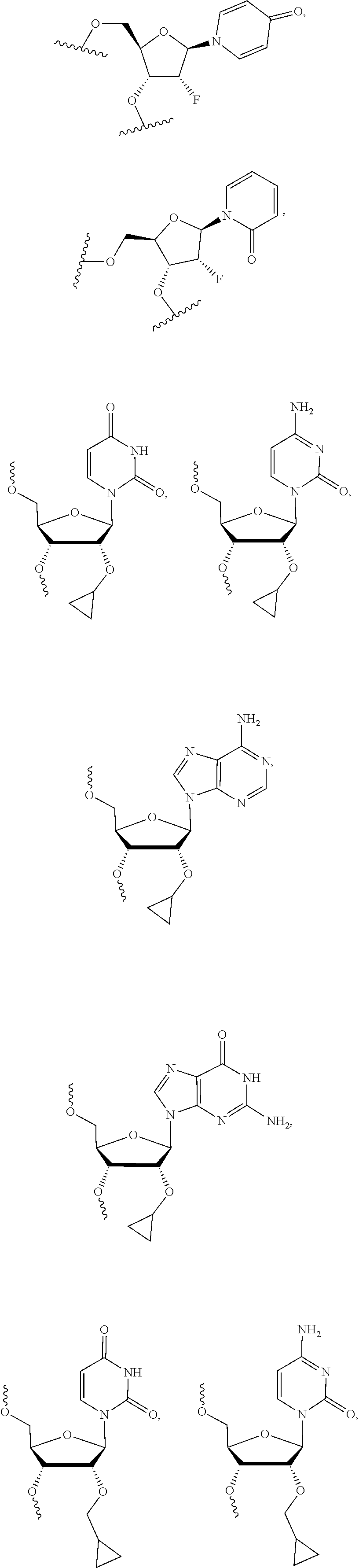

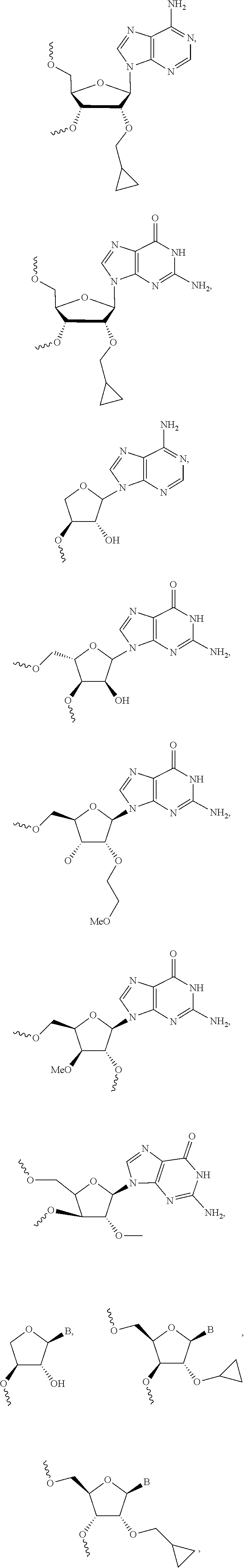

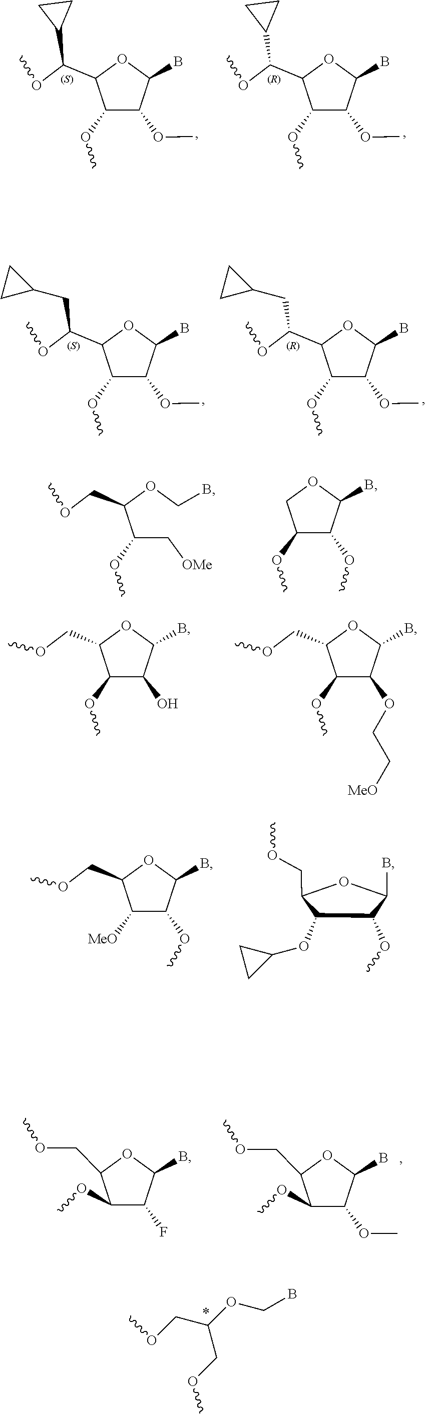

(wherein * represent chiral center), and

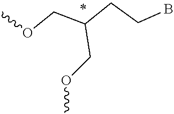

(wherein * represent chiral center), wherein B is a nucleobase, aryl, heteroaryl, or H, and wherein [symbol] or [symbol] represents a phosphodiester linkage, a phosphorothioate linkage, a mesyl phosphoramidate linkage, or H. In some embodiments, the nucleobase is selected from thymine, cytosine, guanine, adenine, uracil, and an analogue or derivative thereof.

In some embodiments, the first nucleotide sequence comprises, consists of, or consists essentially of ribonucleic acids (RNAs). In some embodiments, the first nucleotide sequence comprises, consists of, or consists essentially of modified RNAs. In some embodiments, the modified RNAs are selected from a 2′-O-methyl RNA and 2′-fluoro RNA. In some embodiments, 15, 16, 17, 18, 19, 20, 21, 22, or 23 modified nucleotides of the first nucleotide sequence are independently selected from 2′-O-methyl RNA and 2′-fluoro RNA.

In some embodiments, the sense strand may further comprise one or more internucleoside linkages independently selected from a phosphodiester (PO) internucleoside linkage, phosphorothioate (PS) internucleoside linkage, mesyl phosphoramidate internucleoside linkage (Ms), phosphorodithioate internucleoside linkage, and PS-mimic internucleoside linkage. In some embodiments, the PS-mimic internucleoside linkage is a sulfo internucleoside linkage.

In some embodiments, the sense strand may further comprise at least 1, 2, 3, 4, 5, 6, 7, 8, 9, 10, 11, 12, 13, 14, or 15 or more phosphorothioate internucleoside linkages. In some embodiments, the sense strand comprises 20, 19, 18, 17, 16, 15, 14, 13, 12, 11, 10, 9, 8, 7, 6, 5, 4, or 3 or fewer phosphorothioate internucleoside linkages. In some embodiments, the sense strand comprises 2 to 10, 2 to 8, 2 to 6, 1 to 5, 1 to 4, 1 to 3, or 1 to 2 phosphorothioate internucleoside linkages. In some embodiments, the sense strand comprises 1 to 2 phosphorothioate internucleoside linkages. In some embodiments, the sense strand comprises 2 to 4 phosphorothioate internucleoside linkages. In some embodiments, at least one phosphorothioate internucleoside linkage is between the nucleotides at positions 1 and 2 from the 5′ end of the first nucleotide sequence. In some embodiments, at least one phosphorothioate internucleoside linkage is between the nucleotides at positions 2 and 3 from the 5′ end of the first nucleotide sequence. In some embodiments, the sense strand comprises two phosphorothioate internucleoside linkages between the nucleotides at positions 1 to 3 from the 5′ end of the first nucleotide sequence.

In some embodiments, the sense strand may further comprise at least 1, 2, 3, 4, 5, 6, 7, 8, 9, 10, 11, 12, 13, 14, or 15 or more mesyl phosphoramidate internucleoside linkages. In some embodiments, the sense strand comprises 20, 19, 18, 17, 16, 15, 14, 13, 12, 11, 10, 9, 8, 7, 6, 5, 4, or 3 or fewer mesyl phosphoramidate internucleoside linkages. In some embodiments, the sense strand comprises 2 to 10, 2 to 8, 2 to 6, 1 to 5, 1 to 4, 1 to 3, or 1 to 2 mesyl phosphoramidate internucleoside linkages. In some embodiments, the sense strand comprises 1 to 2 mesyl phosphoramidate internucleoside linkages. In some embodiments, the sense strand comprises 2 to 4 mesyl phosphoramidate internucleoside linkages.

In some embodiments, the sense strand may comprise any of the modified nucleotides disclosed in the sub-section titled “Modified Nucleotides” below. In some embodiments, the sense strand may comprise a 5′-stabilized end cap, and the 5′-stabilized end cap may be selected from those disclosed in the sub-section titled “5′-Stabilized End Cap” below.

### B. SiNA Antisense Strand

Any of the siNA molecules described herein may comprise an antisense strand. The antisense strand may comprise a second nucleotide sequence. The second nucleotide sequence may be 15 to 30, 15 to 25, 15 to 23, 17 to 23, 19 to 23, or 19 to 21 nucleotides in length. In some embodiments, the second nucleotide sequence is 15, 16, 17, 18, 19, 20, 21, 22, 23, 24, 25, 26, 27, 28, 29, or 30 nucleotides in length. In some embodiments, the second nucleotide sequence is at least 19 nucleotides in length. In some embodiments, the second nucleotide sequence is at least 21 nucleotides in length.

In some embodiments, the antisense strand is the same length as the second nucleotide sequence. In some embodiments, the antisense strand is longer than the second nucleotide sequence. In some embodiments, the antisense strand may further comprise 1, 2, 3, 4, or 5 or more nucleotides than the second nucleotide sequence. In some embodiments, the antisense strand is the same length as the sense strand. In some embodiments, the antisense strand is longer than the sense strand. In some embodiments, the antisense strand may further comprise 1, 2, 3, 4, or 5 or more nucleotides than the sense strand. In some embodiments, the antisense strand may further comprise a deoxyribonucleic acid (DNA). In some embodiments, the DNA is thymine (T). In some embodiments, the antisense strand may further comprise a TT sequence. In some embodiments, the antisense strand may further comprise one or more modified nucleotides that are adjacent to the second nucleotide sequence. In some embodiments, the one or more modified nucleotides are independently selected from any of the modified nucleotides disclosed herein (e.g., 2′-fluoro nucleotide, 2′-O-methyl nucleotide, 2′-fluoro nucleotide mimic, 2′-O-methyl nucleotide mimic, or a nucleotide comprising a modified nucleobase).

In some embodiments, the second nucleotide sequence comprises 15, 16, 17, 18, 19, 20, 21, 22, 23, or more modified nucleotides independently selected from a 2′-O-methyl nucleotide and a 2′-fluoro nucleotide. In some embodiments, 70%, 75%, 80%, 85%, 90%, 95% or 100% of the nucleotides in the second nucleotide sequence are modified nucleotides independently selected from a 2′-O-methyl nucleotide and a 2′-fluoro nucleotide. In some embodiments, 100% of the nucleotides in the second nucleotide sequence are modified nucleotides independently selected from a 2′-O-methyl nucleotide and a 2′-fluoro nucleotide.

In some embodiments, between about 15 to 30, 15 to 25, 15 to 24, 15 to 23, 15 to 22, 15 to 21, 17 to 30, 17 to 25, 17 to 24, 17 to 23, 17 to 22, 17 to 21, 18 to 30, 18 to 25, 18 to 24, 18 to 23, 18 to 22, 18 to 21, 19 to 30, 19 to 25, 19 to 24, 19 to 23, 19 to 22, 19 to 21, 20 to 25, 20 to 24, 20 to 23, 21 to 25, 21 to 24, or 21 to 23 modified nucleotides of the second nucleotide sequence are 2′-O-methyl nucleotides. In some embodiments, between about 2 to 20 modified nucleotides of the second nucleotide sequence are 2′-O-methyl nucleotides. In some embodiments, between about 5 to 25 modified nucleotides of the second nucleotide sequence are 2′-O-methyl nucleotides. In some embodiments, between about 10 to 25 modified nucleotides of the second nucleotide sequence are 2′-O-methyl nucleotides. In some embodiments, between about 12 to 25 modified nucleotides of the second nucleotide sequence are 2′-O-methyl nucleotides. In some embodiments, at least 10, 11, 12, 13, 14, 15, 16, 17, 18, 19, 20, 21, or 22 modified nucleotides of the second nucleotide sequence are 2′-O-methyl nucleotides. In some embodiments, at least about 12 modified nucleotides of the second nucleotide sequence are 2′-O-methyl nucleotides. In some embodiments, at least about 13 modified nucleotides of the second nucleotide sequence are 2′-O-methyl nucleotides. In some embodiments, at least about 14 modified nucleotides of the second nucleotide sequence are 2′-O-methyl nucleotides. In some embodiments, at least about 15 modified nucleotides of the second nucleotide sequence are 2′-O-methyl nucleotides. In some embodiments, at least about 16 modified nucleotides of the second nucleotide sequence are 2′-O-methyl nucleotides. In some embodiments, at least about 17 modified nucleotides of the second nucleotide sequence are 2′-O-methyl nucleotides. In some embodiments, at least about 18 modified nucleotides of the second nucleotide sequence are 2′-O-methyl nucleotides. In some embodiments, at least about 19 modified nucleotides of the second nucleotide sequence are 2′-O-methyl nucleotides. In some embodiments, less than or equal to 25, 24, 23, 22, 21, 20, 19, 18, 17, 16, 15, 14, 13, 12, 11, 10, 9, 8, 7, 6, 5, 4, 3, or 2 modified nucleotides of the second nucleotide sequence are 2′-O-methyl nucleotides. In some embodiments, less than or equal to 21 modified nucleotides of the second nucleotide sequence are 2′-O-methyl nucleotides. In some embodiments, less than or equal to 20 modified nucleotides of the second nucleotide sequence are 2′-O-methyl nucleotides. In some embodiments, less than or equal to 19 modified nucleotides of the second nucleotide sequence are 2′-O-methyl nucleotides. In some embodiments, less than or equal to 18 modified nucleotides of the second nucleotide sequence are 2′-O-methyl nucleotides. In some embodiments, less than or equal to 17 modified nucleotides of the second nucleotide sequence are 2′-O-methyl nucleotides. In some embodiments, less than or equal to 16 modified nucleotides of the second nucleotide sequence are 2′-O-methyl nucleotides. In some embodiments, less than or equal to 15 modified nucleotides of the second nucleotide sequence are 2′-O-methyl nucleotides. In some embodiments, less than or equal to 14 modified nucleotides of the second nucleotide sequence are 2′-O-methyl nucleotides. In some embodiments, less than or equal to 13 modified nucleotides of the second nucleotide sequence are 2′-O-methyl nucleotides. In some embodiments, at least one modified nucleotide of the second nucleotide sequence is a 2′-O-methyl pyrimidine. In some embodiments, at least 5, 6, 7, 8, 9, or 10 modified nucleotides of the second nucleotide sequence are 2′-O-methyl pyrimidines. In some embodiments, at least one modified nucleotide of the second nucleotide sequence is a 2′-O-methyl purine. In some embodiments, at least 5, 6, 7, 8, 9, or 10 modified nucleotides of the second nucleotide sequence are 2′-O-methyl purines. In some embodiments, the 2′-O-methyl nucleotide is a 2′-O-methyl nucleotide mimic.

In some embodiments, between 2 to 15 modified nucleotides of the second nucleotide sequence are 2′-fluoro nucleotides. In some embodiments, between 2 to 10 modified nucleotides of the second nucleotide sequence are 2′-fluoro nucleotides. In some embodiments, between 2 to 6 modified nucleotides of the second nucleotide sequence are 2′-fluoro nucleotides. In some embodiments, 1 to 6, 1 to 5, 1 to 4, or 1 to 3 modified nucleotides of the second nucleotide sequence are 2′-fluoro nucleotides. In some embodiments, at least 1, 2, 3, 4, 5, or 6 modified nucleotides of the second nucleotide sequence are 2′-fluoro nucleotides. In some embodiments, at least 1 modified nucleotide of the second nucleotide sequence is a 2′-fluoro nucleotide. In some embodiments, at least 2 modified nucleotides of the second nucleotide sequence are 2′-fluoro nucleotides. In some embodiments, at least 3 modified nucleotides of the second nucleotide sequence are 2′-fluoro nucleotides. In some embodiments, at least 4 modified nucleotides of the second nucleotide sequence are 2′-fluoro nucleotides. In some embodiments, at least 5 modified nucleotides of the second nucleotide sequence are 2′-fluoro nucleotides. In some embodiments, 10, 9, 8, 7, 6, 5, 4, 3 or fewer modified nucleotides of the second nucleotide sequence are 2′-fluoro nucleotides. In some embodiments, 10 or fewer modified nucleotides of the second nucleotide sequence are 2′-fluoro nucleotides. In some embodiments, 7 or fewer modified nucleotides of the second nucleotide sequence are 2′-fluoro nucleotides. In some embodiments, 6 or fewer modified nucleotides of the second nucleotide sequence are 2′-fluoro nucleotides. In some embodiments, 5 or fewer modified nucleotides of the second nucleotide sequence are 2′-fluoro nucleotides. In some embodiments, 4 or fewer modified nucleotides of the second nucleotide sequence are 2′-fluoro nucleotides. In some embodiments, 3 or fewer modified nucleotides of the second nucleotide sequence are 2′-fluoro nucleotides. In some embodiments, 2 or fewer modified nucleotides of the second nucleotide sequence are 2′-fluoro nucleotides. In some embodiments, at least one modified nucleotide of the second nucleotide sequence is a 2′-fluoro pyrimidine. In some embodiments, 1, 2, 3, 4, 5, or 6 modified nucleotides of the second nucleotide sequence are 2′-fluoro pyrimidines. In some embodiments, at least one modified nucleotide of the second nucleotide sequence is a 2′-fluoro purine. In some embodiments, 1, 2, 3, 4, 5, or 6 modified nucleotides of the second nucleotide sequence are 2′-fluoro purines. In some embodiments, the 2′-fluoro nucleotide is a 2′-fluoro nucleotide mimic.

In some embodiments, the 2′-fluoro nucleotide or 2′-O-methyl nucleotide is a 2′-fluoro or 2′-O-methyl nucleotide mimic. In some embodiments, the 2′-fluoro or 2′-O-methyl nucleotide mimic is a nucleotide mimic of Formula (V):

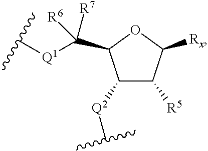

wherein Rₓ is independently a nucleobase, aryl, heteroaryl, or H, Q¹ and Q² are independently S or O, R⁵ is independently -OCD₃, —F, or —OCH₃, and R⁶ and R⁷ are independently H, D, or CD₃. In some embodiments, the nucleobase is selected from thymine, cytosine, guanine, adenine, uracil, and an analogue or derivative thereof.

In some embodiments, the 2′-fluoro or 2′-O-methyl nucleotide mimic is a nucleotide mimic of Formula (16)-Formula (20):

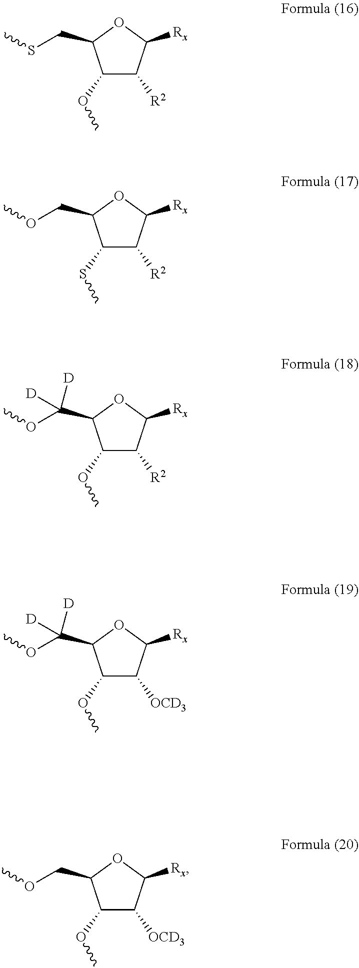

wherein Rₓ is a nucleobase, aryl, heteroaryl, or H and R² is independently F or —OCH₃. In some embodiments, the nucleobase is selected from thymine, cytosine, guanine, adenine, uracil, and an analogue or derivative thereof.

In some embodiments, the antisense strand may comprise 1, 2, 3, 4, or 5 or more modified nucleotide(s) selected from:

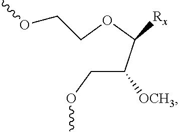

wherein Rx is a nucleobase, aryl, heteroaryl, or H,

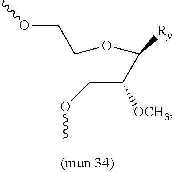

wherein R_{y} is a nucleobase,

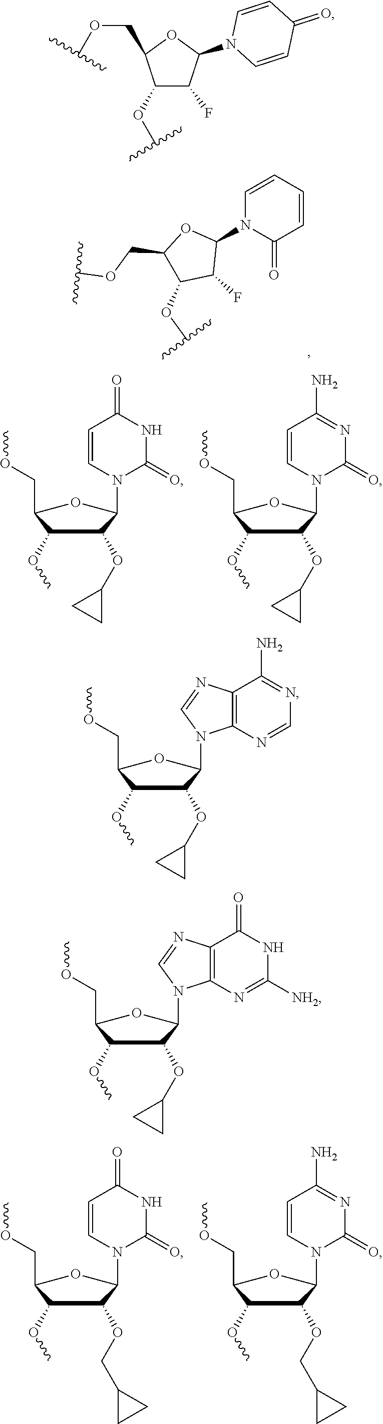

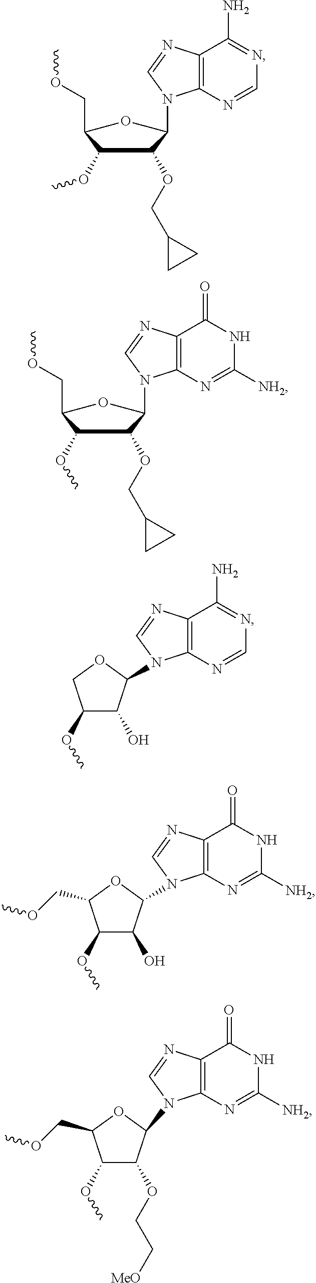

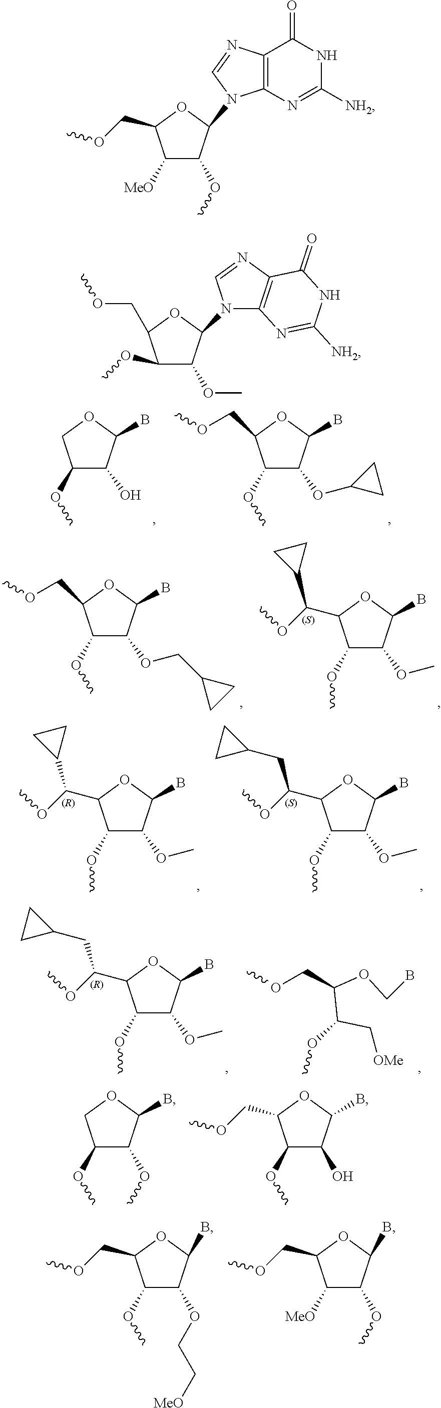

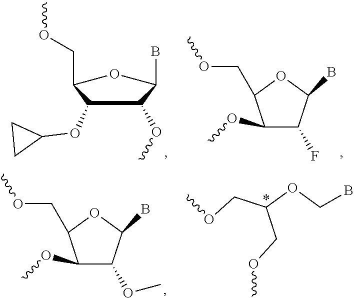

(wherein * represent chiral center), and

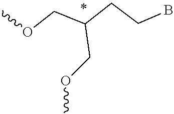

(wherein * represent chiral center), wherein B is a nucleobase, aryl, heteroaryl, or H, and wherein [symbol] or [symbol] represents a phosphodiester linkage, a phosphorothioate linkage, a mesyl phosphoramidate linkage, or H. In some embodiments, the nucleobase is selected from thymine, cytosine, guanine, adenine, uracil, and an analogue or derivative thereof.

In some embodiments, at least 1, 2, 3, 4, 5, 6, 7, 8, or 9 nucleotides at position 2, 5, 6, 8, 10, 14, 16, 17, and/or 18 from the 5′ end of the second nucleotide sequence is a 2′-fluoro nucleotide. In some embodiments, the nucleotide at position 2, 5, 6, 8, 10, 14, 16, 17, and/or 18 from the 5′ end of the second nucleotide sequence is a 2′-fluoro nucleotide. In some embodiments, at least two nucleotides at positions 2, 5, 6, 8, 10, 14, 16, 17, and/or 18 from the 5′ end of the second nucleotide sequence are 2′-fluoro nucleotides. In some embodiments, at least three nucleotides at positions 2, 5, 6, 8, 10, 14, 16, 17, and/or 18 from the 5′ end of the second nucleotide sequence are 2′-fluoro nucleotides. In some embodiments, at least four nucleotides at positions 2, 5, 6, 8, 10, 14, 16, 17, and/or 18 from the 5′ end of the second nucleotide sequence are 2′-fluoro nucleotides. In some embodiments, at least five nucleotides at positions 2, 5, 6, 8, 10, 14, 16, 17, and/or 18 from the 5′ end of the second nucleotide sequence are 2′-fluoro nucleotides. In some embodiments, the nucleotides at positions 2 and/or 14 from the 5′ end of the second nucleotide sequence are 2′-fluoro nucleotides. In some embodiments, the nucleotides at positions 2, 6, and/or 16 from the 5′ end of the second nucleotide sequence are 2′-fluoro nucleotides. In some embodiments, the nucleotides at positions 2, 6, 14, and/or 16 from the 5′ end of the second nucleotide sequence are 2′-fluoro nucleotides. In some embodiments, the nucleotides at positions 2, 6, 10, 14, and/or 18 from the 5′ end of the second nucleotide sequence are 2′-fluoro nucleotides. In some embodiments, the nucleotides at positions 2, 5, 8, 14, and/or 17 from the 5′ end of the second nucleotide sequence are 2′-fluoro nucleotides. In some embodiments, the nucleotide at position 2 from the 5′ end of the second nucleotide sequence is a 2′-fluoro nucleotide. In some embodiments, the nucleotide at position 5 from the 5′ end of the second nucleotide sequence is a 2′-fluoro nucleotide. In some embodiments, the nucleotide at position 6 from the 5′ end of the second nucleotide sequence is a 2′-fluoro nucleotide. In some embodiments, the nucleotide at position 8 from the 5′ end of the second nucleotide sequence is a 2′-fluoro nucleotide. In some embodiments, the nucleotide at position 10 from the 5′ end of the second nucleotide sequence is a 2′-fluoro nucleotide. In some embodiments, the nucleotide at position 14 from the 5′ end of the second nucleotide sequence is a 2′-fluoro nucleotide. In some embodiments, the nucleotide at position 16 from the 5′ end of the second nucleotide sequence is a 2′-fluoro nucleotide. In some embodiments, the nucleotide at position 17 from the 5′ end of the second nucleotide sequence is a 2′-fluoro nucleotide. In some embodiments, the nucleotide at position 18 from the 5′ end of the second nucleotide sequence is a 2′-fluoro nucleotide. In some embodiments, the 2′-fluoro nucleotide is a 2′-fluoro nucleotide mimic.

In some embodiments, the nucleotides in the second nucleotide sequence are arranged in an alternating 1:3 modification pattern, wherein 1 nucleotide is a 2′-fluoro nucleotide and 3 nucleotides are 2′-O-methyl nucleotides, and wherein the alternating 1:3 modification pattern occurs at least 2 times. In some embodiments, the alternating 1:3 modification pattern occurs 2-5 times. In some embodiments, at least two of the alternating 1:3 modification pattern occur consecutively. In some embodiments, at least two of the alternating 1:3 modification pattern occurs nonconsecutively. In some embodiments, at least 1, 2, 3, 4, or 5 alternating 1:3 modification pattern begins at nucleotide position 2, 6, 10, 14, and/or 18 from the 5′ end of the antisense strand. In some embodiments, at least one alternating 1:3 modification pattern begins at nucleotide position 2 from the 5′ end of the antisense strand. In some embodiments, wherein at least one alternating 1:3 modification pattern begins at nucleotide position 6 from the 5′ end of the antisense strand. In some embodiments, at least one alternating 1:3 modification pattern begins at nucleotide position 10 from the 5′ end of the antisense strand. In some embodiments, at least one alternating 1:3 modification pattern begins at nucleotide position 14 from the 5′ end of the antisense strand. In some embodiments, at least one alternating 1:3 modification pattern begins at nucleotide position 18 from the 5′ end of the antisense strand. In some embodiments, the 2′-fluoro nucleotide is a 2′-fluoro nucleotide mimic.

In some embodiments, the nucleotides in the second nucleotide sequence are arranged in an alternating 1:2 modification pattern, wherein 1 nucleotide is a 2′-fluoro nucleotide and 2 nucleotides are 2′-O-methyl nucleotides, and wherein the alternating 1:2 modification pattern occurs at least 2 times. In some embodiments, the alternating 1:2 modification pattern occurs 2-5 times. In some embodiments, at least two of the alternating 1:2 modification pattern occurs consecutively. In some embodiments, at least two of the alternating 1:2 modification pattern occurs nonconsecutively. In some embodiments, at least 1, 2, 3, 4, or 5 alternating 1:2 modification pattern begins at nucleotide position 2, 5, 8, 14, and/or 17 from the 5′ end of the antisense strand. In some embodiments, at least one alternating 1:2 modification pattern begins at nucleotide position 2 from the 5′ end of the antisense strand. In some embodiments, at least one alternating 1:2 modification pattern begins at nucleotide position 5 from the 5′ end of the antisense strand. In some embodiments, at least one alternating 1:2 modification pattern begins at nucleotide position 8 from the 5′ end of the antisense strand. In some embodiments, at least one alternating 1:2 modification pattern begins at nucleotide position 14 from the 5′ end of the antisense strand. In some embodiments, at least one alternating 1:2 modification pattern begins at nucleotide position 17 from the 5′ end of the antisense strand. In some embodiments, the 2′-fluoro nucleotide is a 2′-fluoro nucleotide mimic.

In some embodiments, the second nucleotide sequence comprises, consists of, or consists essentially of ribonucleic acids (RNAs). In some embodiments, the second nucleotide sequence comprises, consists of, or consists essentially of modified RNAs. In some embodiments, the modified RNAs are selected from a 2′-O-methyl RNA and 2′-fluoro RNA. In some embodiments, 15, 16, 17, 18, 19, 20, 21, 22, or 23 modified nucleotides of the second nucleotide sequence are independently selected from 2′-O-methyl RNA and 2′-fluoro RNA. In some embodiments, the 2′-fluoro nucleotide is a 2′-fluoro nucleotide mimic.

In some embodiments, the sense strand may further comprise one or more internucleoside linkages independently selected from a phosphodiester (PO) internucleoside linkage, phosphorothioate (PS) internucleoside linkage, phosphorodithioate internucleoside linkage, and PS-mimic internucleoside linkage. In some embodiments, the PS-mimic internucleoside linkage is a 3′- or 5′-sulfur linkage.

In some embodiments, the antisense strand may further comprise at least 1, 2, 3, 4, 5, 6, 7, 8, 9, 10, 11, 12, 13, 14, or 15 or more phosphorothioate internucleoside linkages. In some embodiments, the antisense strand comprises 20, 19, 18, 17, 16, 15, 14, 13, 12, 11, 10, 9, 8, 7, 6, 5, 4, or 3 or fewer phosphorothioate internucleoside linkages. In some embodiments, the antisense strand comprises 2 to 10, 2 to 8, 2 to 6, 1 to 5, 1 to 4, 1 to 3, or 1 to 2 phosphorothioate internucleoside linkages. In some embodiments, the antisense strand comprises 2 to 10, 2 to 8, 2 to 6, 1 to 5, 1 to 4, 1 to 3, or 1 to 2 phosphorothioate internucleoside linkages. In some embodiments, the antisense strand comprises 2 to 8 phosphorothioate internucleoside linkages. In some embodiments, the antisense strand comprises 3 to 8 phosphorothioate internucleoside linkages. In some embodiments, the antisense strand comprises 4 to 8 phosphorothioate internucleoside linkages. In some embodiments, at least one phosphorothioate internucleoside linkage is between the nucleotides at positions 1 and 2 from the 5′ end of the second nucleotide sequence. In some embodiments, at least one phosphorothioate internucleoside linkage is between the nucleotides at positions 2 and 3 from the 5′ end of the second nucleotide sequence. In some embodiments, at least one phosphorothioate internucleoside linkage is between the nucleotides at positions 1 and 2 from the 3′ end of the second nucleotide sequence. In some embodiments, at least one phosphorothioate internucleoside linkage is between the nucleotides at positions 2 and 3 from the 3′ end of the second nucleotide sequence. In some embodiments, the antisense strand comprises two phosphorothioate internucleoside linkages between the nucleotides at positions 1 to 3 from the 5′ end of the first nucleotide sequence. In some embodiments, the antisense strand comprises two phosphorothioate internucleoside linkages between the nucleotides at positions 1 to 3 from the 3′ end of the first nucleotide sequence. In some embodiments, the antisense strand comprises (a) two phosphorothioate internucleoside linkages between the nucleotides at positions 1 to 3 from the 5′ end of the first nucleotide sequence; and (b) two phosphorothioate internucleoside linkages between the nucleotides at positions 1 to 3 from the 3′ end of the first nucleotide sequence.

In some embodiments, the antisense strand may further comprise at least 1, 2, 3, 4, 5, 6, 7, 8, 9, 10, 11, 12, 13, 14, or 15 or more mesyl phosphoramidate internucleoside linkages. In some embodiments, the antisense strand comprises 20, 19, 18, 17, 16, 15, 14, 13, 12, 11, 10, 9, 8, 7, 6, 5, 4, or 3 or fewer mesyl phosphoramidate internucleoside linkages. In some embodiments, the antisense strand comprises 2 to 10, 2 to 8, 2 to 6, 1 to 5, 1 to 4, 1 to 3, or 1 to 2 mesyl phosphoramidate internucleoside linkages. In some embodiments, the antisense strand comprises 2 to 10, 2 to 8, 2 to 6, 1 to 5, 1 to 4, 1 to 3, or 1 to 2 mesyl phosphoramidate internucleoside linkages. In some embodiments, the antisense strand comprises 2 to 8 mesyl phosphoramidate internucleoside linkages. In some embodiments, the antisense strand comprises 3 to 8 mesyl phosphoramidate internucleoside linkages. In some embodiments, the antisense strand comprises 4 to 8 mesyl phosphoramidate internucleoside linkages.

In some embodiments, at least one end of the ds-siNA is a blunt end. In some embodiments, at least one end of the ds-siNA comprises an overhang, wherein the overhang comprises at least one nucleotide. In some embodiments, both ends of the ds-siNA comprise an overhang, wherein the overhang comprises at least one nucleotide. In some embodiments, the overhang comprises 1 to 5 nucleotides, 1 to 4 nucleotides, 1 to 3 nucleotides, or 1 to 2 nucleotides. In some embodiments, the overhang consists of 1 to 2 nucleotides.

In some embodiments, the antisense strand may comprise any of the modified nucleotides disclosed in the sub-section titled “Modified Nucleotides” below. In some embodiments, the antisense strand may comprise a 5′-stabilized end cap, and the 5′-stabilized end cap may be selected from those disclosed in the sub-section titled “5′-Stabilized End Cap” below.

### Modified Nucleotides

The siNA molecules disclosed herein comprise one or more modified nucleotides. In some embodiments, the sense strands disclosed herein comprise one or more modified nucleotides. In some embodiments, any of the first nucleotide sequences disclosed herein comprise one or more modified nucleotides. In some embodiments, the antisense strands disclosed herein comprise one or more modified nucleotides. In some embodiments, any of the second nucleotide sequences disclosed herein comprise one or more modified nucleotides. In some embodiments, the one or more modified nucleotides is adjacent to the first nucleotide sequence. In some embodiments, at least one modified nucleotide is adjacent to the 5′ end of the first nucleotide sequence. In some embodiments, at least one modified nucleotide is adjacent to the 3′ end of the first nucleotide sequence. In some embodiments, at least one modified nucleotide is adjacent to the 5′ end of the first nucleotide sequence and at least one modified nucleotide is adjacent to the 3′ end of the first nucleotide sequence. In some embodiments, the one or more modified nucleotides is adjacent to the second nucleotide sequence. In some embodiments, at least one modified nucleotide is adjacent to the 5′ end of the second nucleotide sequence. In some embodiments, at least one modified nucleotide is adjacent to the 3′ end of the second nucleotide sequence. In some embodiments, at least one modified nucleotide is adjacent to the 5′ end of the second nucleotide sequence and at least one modified nucleotide is adjacent to the 3′ end of the second nucleotide sequence. In some embodiments, a 2′-O-methyl nucleotide in any of sense strands or first nucleotide sequences disclosed herein is replaced with a modified nucleotide. In some embodiments, a 2′-O-methyl nucleotide in any of antisense strands or second nucleotide sequences disclosed herein is replaced with a modified nucleotide.

In some embodiments, any of the siNA molecules, siNAs, sense strands, first nucleotide sequences, antisense strands, and second nucleotide sequences disclosed herein comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 or more modified nucleotides. In some embodiments, 1%, 2%, 3%, 4%, 5%, 10%, 15%, 20%, 25%, 30%, 35%, 40%, 45%, 50%, 55%, 60%, 70%, 75%, 80%, 85%, 86%, 87%, 88%, 90%, 91%, 92%, 93%, 94%, 95%, 96%, 97%, 98%, 99% or 100% of the nucleotides in the siNA molecule, siNA, sense strand, first nucleotide sequence, antisense strand, or second nucleotide sequence are modified nucleotides.

In some embodiments, a modified nucleotide is selected from the group consisting of 2′-fluoro nucleotide, 2′-O-methyl nucleotide, 2′-fluoro nucleotide mimic, 2′-O-methyl nucleotide mimic, a locked nucleic acid, an unlocked nucleic acid, and a nucleotide comprising a modified nucleobase. In some embodiments, the unlocked nucleic acid is a 2′,3′-unlocked nucleic acid. In some embodiments, the unlocked nucleic acid is a 3′,4′-unlocked nucleic acid (e.g., mun34) in which the furanose ring lacks a bond between the 3′ and 4; carbons.

For example, the siNA of the present disclosure may comprise one or more modified nucleotide(s) selected from:

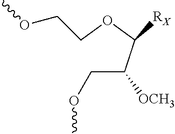

wherein Rx is a nucleobase, aryl, heteroaryl, or H,

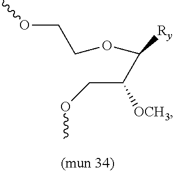

wherein R_{y} is a nucleobase,

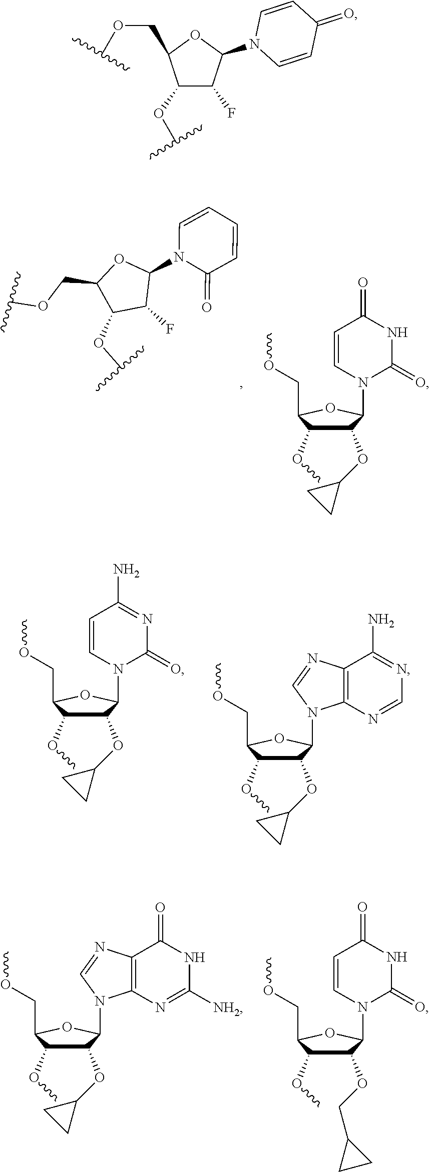

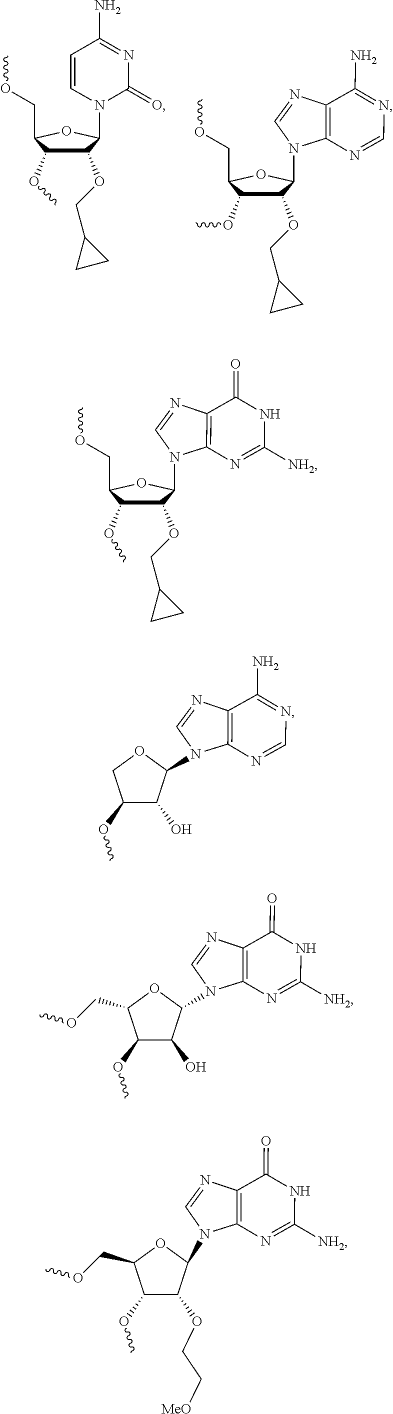

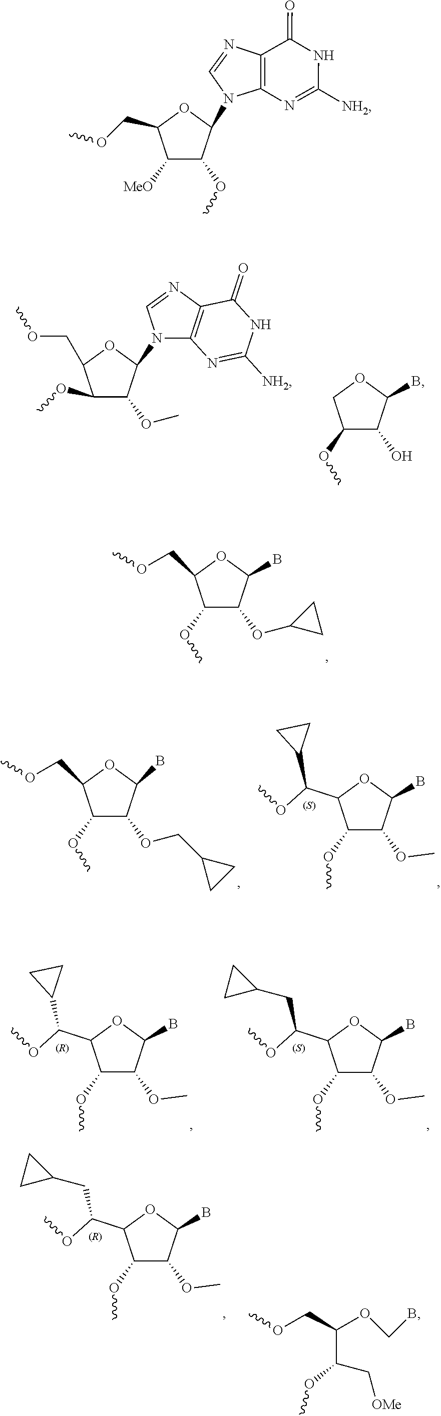

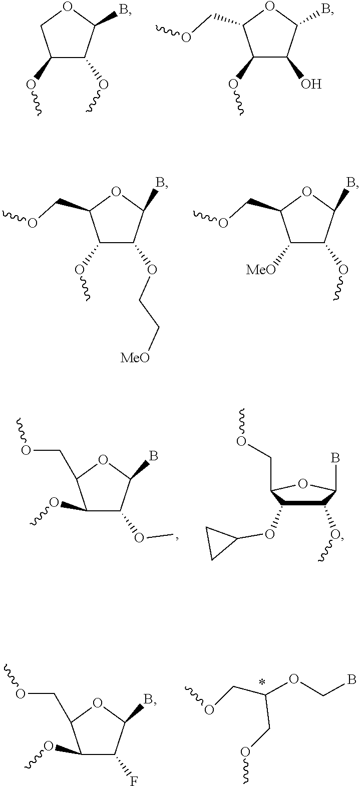

(wherein * represent chiral center), and

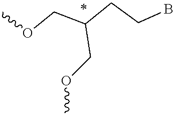

(wherein * represent chiral center), wherein B is a nucleobase, aryl, heteroaryl, or H, and wherein [symbol] or [symbol] represents a phosphodiester linkage, a phosphorothioate linkage, a mesyl phosphoramidate linkage, or H. In some embodiments, both the sense strand and the antisense strand may each independently comprise 1, 2, 3, 4, or 5 or more of the foregoing modified nucleotides. In some embodiments, one or more (e.g., 1, 2, 3, 4, 5, or more) of the foregoing modified nucleotides may independently be present in the sense strand, first nucleotide sequence, antisense strand, and/or second nucleotide sequence of any of the siNA disclosed herein. In some embodiments, the nucleobase is selected from thymine, cytosine, guanine, adenine, uracil, and an analogue or derivative thereof.

Additionally or alternatively, any of the siNAs disclosed herein may comprise other modified nucleotides, such as 2′-fluoro or 2′-O-methyl nucleotide mimics. For example, the disclosed siNA may comprise at least 1, 2, 3, 4, 5, 6, 7, 8, 9, or 10 or more 2′-fluoro or 2′-O-methyl nucleotide mimics. In some embodiments, any of the sense strands disclosed herein comprise at least 1, 2, 3, 4, 5, 6, 7, 8, 9, or 10 or more 2′-fluoro or 2′-O-methyl nucleotide mimics. In some embodiments, any of the first nucleotide sequences disclosed herein comprise at least 1, 2, 3, 4, 5, 6, 7, 8, 9, or 10 or more 2′-fluoro or 2′-O-methyl nucleotide mimics. In some embodiments, any of the antisense strands disclosed herein comprise at least 1, 2, 3, 4, 5, 6, 7, 8, 9, or 10 or more 2′-fluoro or 2′-O-methyl nucleotide mimics. In some embodiments, any of the second nucleotide sequences disclosed herein comprise at least 1, 2, 3, 4, 5, 6, 7, 8, 9, or 10 or more 2′-fluoro or 2′-O-methyl nucleotide mimics. In some embodiments, the 2′-fluoro or 2′-O-methyl nucleotide mimic is a nucleotide mimic of Formula (16)-Formula (20):

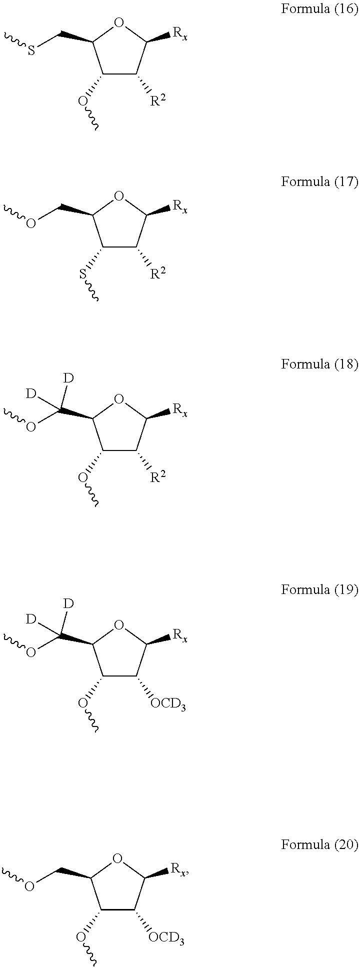

wherein Rₓ is a nucleobase, aryl, heteroaryl, or H and R² is independently F or —OCH₃. In some embodiments, the nucleobase is selected from thymine, cytosine, guanine, adenine, uracil, and an analogue or derivative thereof.

In some embodiments, the siNA molecules disclosed herein comprise at least one 2′-fluoro nucleotide, at least one 2′-O-methyl nucleotide, and at least one 2′-fluoro or 2′-O-methyl nucleotide mimic. In some embodiments, the at least one 2′-fluoro or 2′-O-methyl nucleotide mimic is adjacent to the first nucleotide sequence. In some embodiments, the at least one 2′-fluoro or 2′-O-methyl nucleotide mimic is adjacent to the 5′ end of first nucleotide sequence. In some embodiments, the at least one 2′-fluoro or 2′-O-methyl nucleotide mimic is adjacent to the 3′ end of first nucleotide sequence. In some embodiments, the at least one 2′-fluoro or 2′-O-methyl nucleotide mimic is adjacent to the second nucleotide sequence. In some embodiments, the at least one 2′-fluoro or 2′-O-methyl nucleotide mimic is adjacent to the 5′ end of second nucleotide sequence. In some embodiments, the at least one 2′-fluoro or 2′-O-methyl nucleotide mimic is adjacent to the 3′ end of second nucleotide sequence. In some embodiments, the first nucleotide sequence does not comprise a 2′-fluoro nucleotide mimic. In some embodiments, the first nucleotide sequence does not comprise a 2′-O-methyl nucleotide mimic. In some embodiments, the second nucleotide sequence does not comprise a 2′-fluoro nucleotide mimic. In some embodiments, the second nucleotide sequence does not comprise a 2′-O-methyl nucleotide mimic.

### Conjugated Moiety

Further disclosed herein are siNA molecules comprising a conjugated moiety. In some embodiments, the conjugated moiety is selected from galactosamine, peptides, proteins, sterols, lipids, phospholipids, biotin, phenoxazines, active drug substance, cholesterols, phenanthridine, anthraquinone, acridine, fluoresceins, rhodamines, coumarins, and dyes. In some embodiments, the conjugated moiety is attached to the 3′ end of the sense strand or first nucleotide sequence. In some embodiments, the conjugated moiety is attached to the 3′ end of the sense strand or first nucleotide sequence via 1, 2, 3, 4, or 5 or more linkers. In some embodiments, the conjugated moiety is attached to the 5′ end of the sense strand or first nucleotide sequence. In some embodiments, the conjugated moiety is attached to the 5′ end of the sense strand or first nucleotide sequence via 1, 2, 3, 4, or 5 or more linkers. In some embodiments, the conjugated moiety is attached to the 3′ end of the antisense strand or second nucleotide sequence. In some embodiments, the conjugated moiety is attached to the 3′ end of the antisense strand or second nucleotide sequence via 1, 2, 3, 4, or 5 or more linkers. In some embodiments, the conjugated moiety is attached to the 5′ end of the antisense strand or second nucleotide sequence. In some embodiments, the conjugated moiety is attached to the 5′ end of the antisense strand or second nucleotide sequence via 1, 2, 3, 4, or 5 or more linkers. In some embodiments, the one or more linkers are independently selected from the group consisting of a phosphodiester linker, phosphorothioate linker, phosphorodithioate linker, and mesyl phosphoramidate linker.

In some embodiments, the conjugated moiety is galactosamine. In some embodiments, any of the siNAs disclosed herein are attached to a conjugated moiety that is galactosamine. In some embodiments, the galactosamine is N-acetylgalactosamine (GalNAc). In some embodiments, any of the siNA molecules disclosed herein comprise GalNAc. In some embodiments, the GalNAc is of Formula (VI):

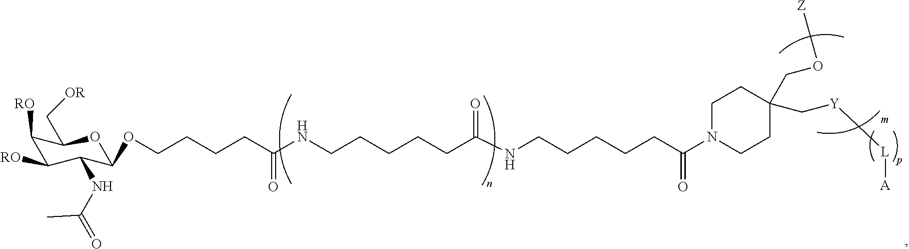

wherein m is 1, 2, 3, 4, or 5; each n is independently 1 or 2; p is 0 or 1; each R is independently H or a first protecting group; each Y is independently selected from —O—P(═O)(SH)—, —O—P(═O)(O)—, —O—P(═O)(OH)—, —O—P(S)S—, and —O—; Z is H or a second protecting group; either L is a linker or L and Y in combination are a linker; and A is H, OH, a third protecting group, an activated group, or an oligonucleotide. In some embodiments, the first protecting group is acetyl. In some embodiments, the second protecting group is trimethoxytrityl (TMT). In some embodiments, the activated group is a phosphoramidite group. In some embodiments, the phosphoramidite group is a cyanoethoxy N,N-diisopropylphosphoramidite group. In some embodiments, the linker is a C₆—NH₂ group. In some embodiments, A is a short interfering nucleic acid (siNA) or siNA molecule. In some embodiments, m is 3. In some embodiments, R is H, Z is H, and n is 1. In some embodiments, R is H, Z is H, and n is 2.

In some embodiments, the GalNAc is Formula (VII):

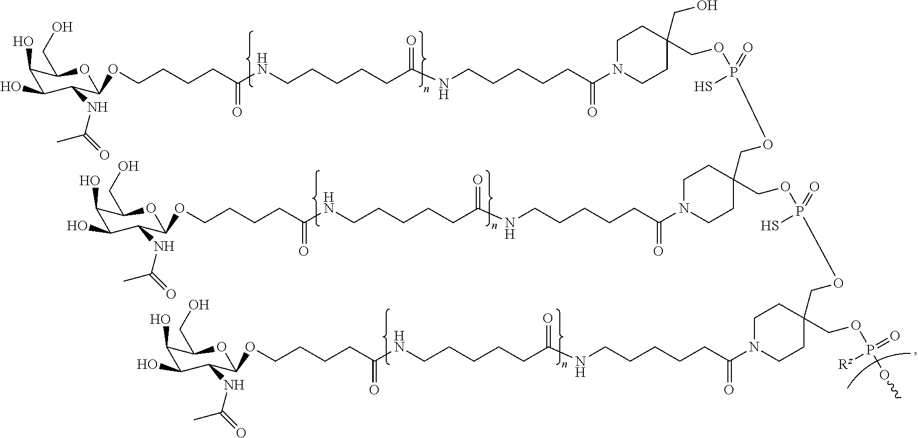

wherein R^{z} is OH or SH; and each n is independently 1 or 2. In some embodiments, the targeting ligand may be a GalNAc targeting ligand may comprise 1, 2, 3, 4, 5 or 6 GalNAc units. In some embodiments, the targeting ligand may be a GalNAc selected from GalNAc2, GalNAc3, GalNAc4 (the GalNAc of Formula VII, wherein n=1 and R^{z}=OH), GalNAc5, and GalNAc6.

In some embodiments, the GalNAc may be GalNAc amidite (i.e., compound 40-9, see Example 22), GalNAc4 CPG GalNAc phophoramidite, or GalNAc4-ps-GalNAc4-ps-GalNAc4. These GalNAc moieties are shown below:

GalNAc 4 moieties
GalNac4 phosphoramidite

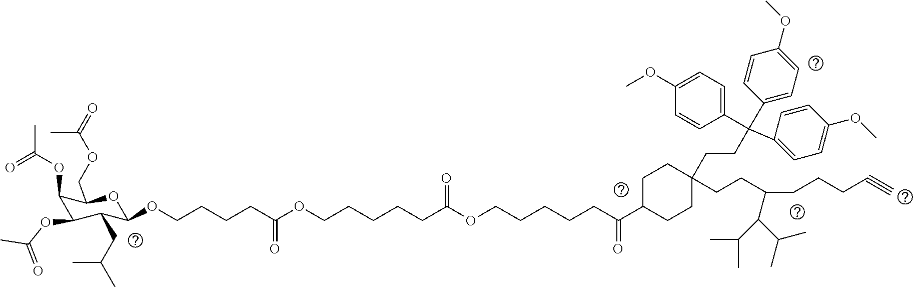

GalNAc4 CPG

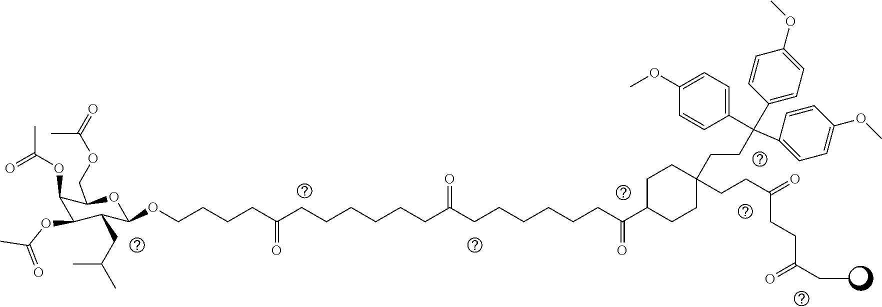

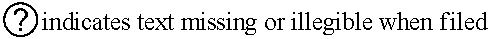

GalNAc3, GalNAc4, GalNAc5 and GalNAc6 may be conjugated to an siNA disclosed herein during synthesis with 1 2, or 3 moieties. Further GalNAc moieties, such as GalNAc1 and GalNAc2, can be used to form 5′ and 3′-GalNAc using post synthesis conjugation.

GalNAc Phosphoramidites
GalNAc building blocks
GalNAc-3 phosphoramidite

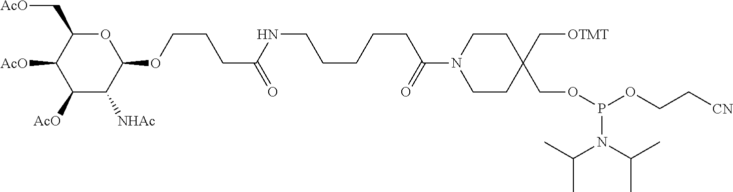

GalNAc-4 phosphoramidite

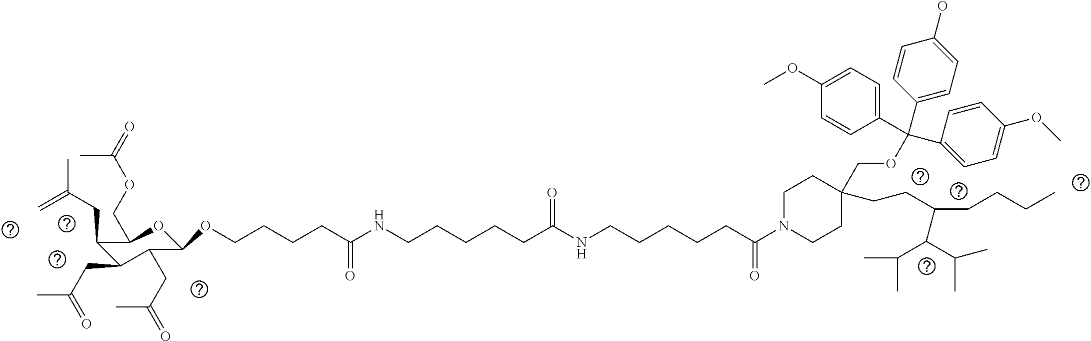

GalNAc-5 phosphoramidite

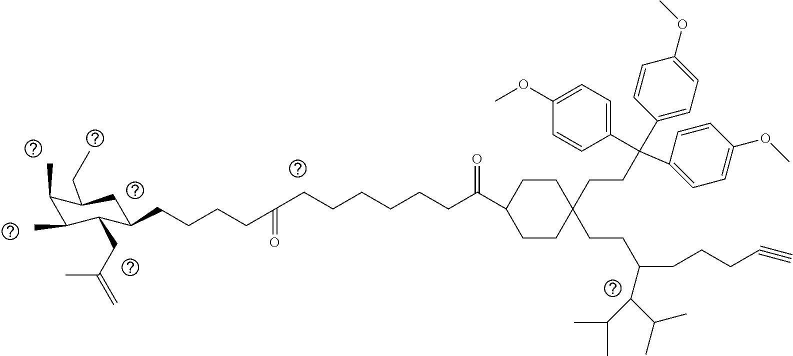

GalNAc-6 phosphoramidite

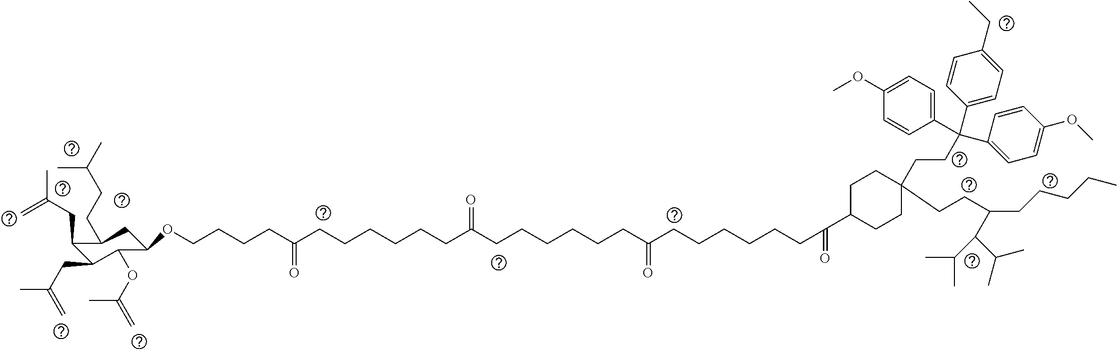

After Attachment to Oligos (Nomenclature)

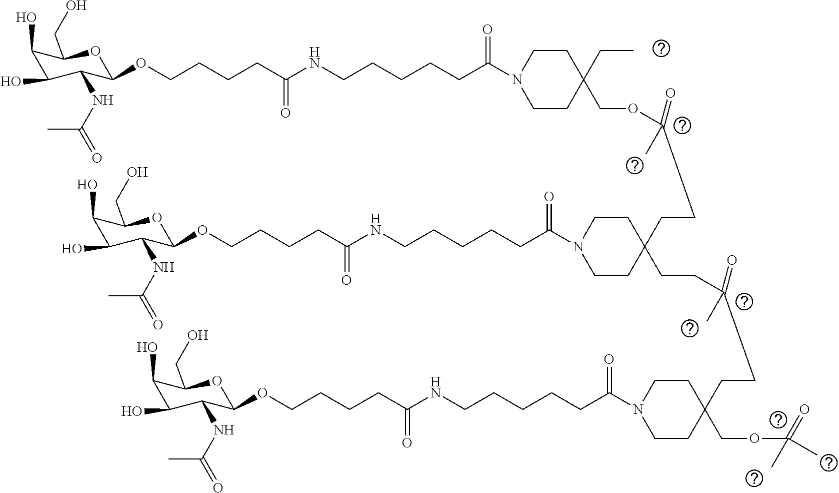

(GalNAc3-(PS)2-p)

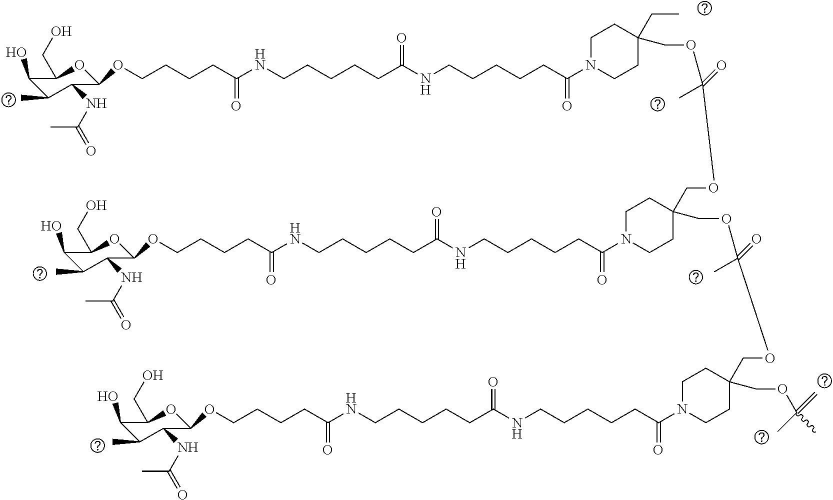

(GalNAc4-(PS)2-p)

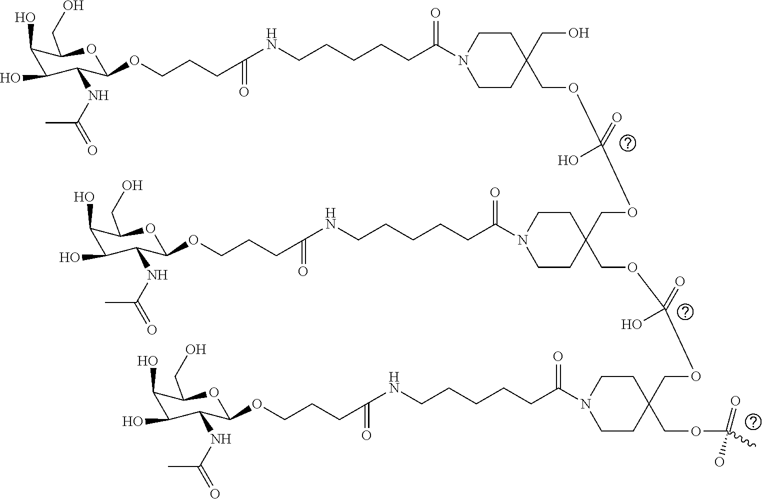

(GalNAc5-(PS)2-p)

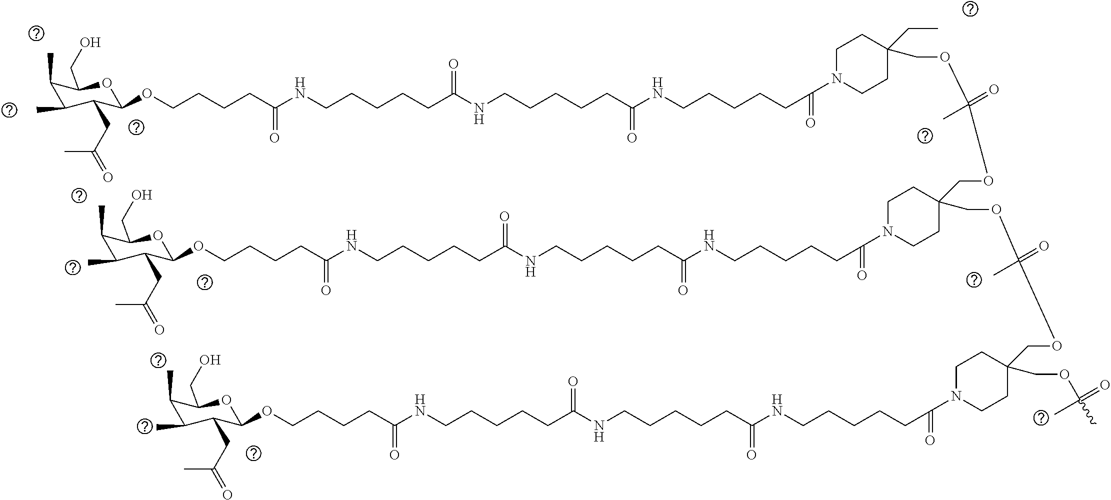

(GalNAc6-(PS)2-p)

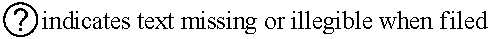

In some embodiments, the galactosamine is attached to the 3′ end of the sense strand or first nucleotide sequence. In some embodiments, the galactosamine is attached to the 3′ end of the sense strand or first nucleotide sequence via 1, 2, 3, 4, or 5 or more linkers. In some embodiments, the galactosamine is attached to the 5′ end of the sense strand or first nucleotide sequence. In some embodiments, the galactosamine is attached to the 5′ end of the sense strand or first nucleotide sequence via 1, 2, 3, 4, or 5 or more linkers. In some embodiments, the galactosamine is attached to the 3′ end of the antisense strand or second nucleotide sequence. In some embodiments, the galactosamine is attached to the 3′ end of the antisense strand or second nucleotide sequence via 1, 2, 3, 4, or 5 or more linkers. In some embodiments, the galactosamine is attached to the 5′ end of the antisense strand or second nucleotide sequence. In some embodiments, the galactosamine is attached to the 5′ end of the antisense strand or second nucleotide sequence via 1, 2, 3, 4, or 5 or more linkers. In some embodiments, the one or more linkers are independently selected from the group consisting of a phosphodiester (p or po) linker, phosphorothioate (ps) linker, mesyl phosphoramidate linker (Ms), phosphoramidite (HEG) linker, triethylene glycol (TEG) linker, and/or phosphorodithioate linker. In some embodiments, the one or more linkers are independently selected from the group consisting of p-(PS)2, (PS)2-p-TEG-p, (PS)2-p-HEG-p, and (PS)2-p-(HEG-p) 2.

In some embodiments, the conjugated moiety is a lipid moiety. In some embodiments, any of the siNAs disclosed herein are attached to a conjugated moiety that is a lipid moiety. Examples of lipid moieties include, but are not limited to, a cholesterol moiety, a thioether, e.g., hexyl-S-tritylthiol, a thiocholesterol, an aliphatic chain, e.g., dodecandiol or undecyl residues a phospholipid, e.g., di-hexadecyl-rac-glycerol or triethylammonium 1-di-O-hexadecyl-rac-glycero-S—H-phosphonate, a polyamine or a polyethylene glycol chain, adamantane acetic acid, a palmityl moiety, or an octadecylamine or hexylamino-carbonyl-oxycholesterol moiety.

In some embodiments, the conjugated moiety is an active drug substance. In some embodiments, any of the siNAs disclosed herein are attached to a conjugated moiety that is an active drug substance. Examples of active drug substances include, but are not limited to, aspirin, warfarin, phenylbutazone, ibuprofen, suprofen, fenbufen, ketoprofen, (5)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic.

### Phosphorylation Blocker

Any of the disclosed siNA molecules may comprise a phosphorylation blocker. In some embodiments, a 2′-O-methyl nucleotide in any of sense strands or first nucleotide sequences disclosed herein is replaced with a nucleotide containing a phosphorylation blocker. In some embodiments, a 2′-O-methyl nucleotide in any of antisense strands or second nucleotide sequences disclosed herein is replaced with a nucleotide containing a phosphorylation blocker. In some embodiments, a 2′-O-methyl nucleotide in any of sense strands or first nucleotide sequences disclosed herein is further modified to contain a phosphorylation blocker. In some embodiments, a 2′-O-methyl nucleotide in any of antisense strands or second nucleotide sequences disclosed herein is further modified to contain a phosphorylation blocker.

In some embodiments, any of the siNA molecules disclosed herein comprise a phosphorylation blocker of Formula (IV):

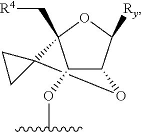

wherein R_{y} is a nucleobase, R⁴ is —O—R³⁰ or —NR³¹R³², R³⁰ is C₁-C₈ substituted or unsubstituted alkyl; and R³¹ and R³² together with the nitrogen to which they are attached form a substituted or unsubstituted heterocyclic ring. In some embodiments, the nucleobase is selected from thymine, cytosine, guanine, adenine, uracil, and an analogue or derivative thereof.

In some embodiments, any of the siNA molecules disclosed herein comprise a phosphorylation blocker of Formula (IV):

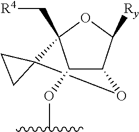

Formula (IV), wherein R_{y} is a nucleobase, and R⁴ is —OCH₃ or —N(CH₂CH₂)₂O. In some embodiments, the nucleobase is selected from thymine, cytosine, guanine, adenine, uracil, and an analogue or derivative thereof.

In some embodiments, a siNA molecule comprises (a) a phosphorylation blocker of Formula (IV):

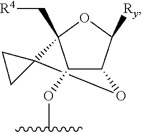

wherein R_{y} is a nucleobase, R⁴ is —O—R³⁰ or —NR³¹R³², R³⁰ is C₁-C₈ substituted or unsubstituted alkyl; and R³¹ and R³² together with the nitrogen to which they are attached form a substituted or unsubstituted heterocyclic ring; and (b) a short interfering nucleic acid (siNA), wherein the phosphorylation blocker is conjugated to the siNA. In some embodiments, the nucleobase is selected from thymine, cytosine, guanine, adenine, uracil, and an analogue or derivative thereof.

In some embodiments, a siNA molecule comprises (a) a phosphorylation blocker of Formula (IV):

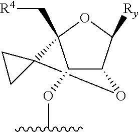

Formula (IV), wherein R^{y} is a nucleobase, and R⁴ is —OCH₃ or —N(CH₂CH₂)₂O; and (b) a short interfering nucleic acid (siNA), wherein the phosphorylation blocker is conjugated to the siNA.

In some embodiments, the phosphorylation blocker is attached to the 3′ end of the sense strand or first nucleotide sequence. In some embodiments, the phosphorylation blocker is attached to the 3′ end of the sense strand or first nucleotide sequence via 1, 2, 3, 4, or 5 or more linkers. In some embodiments, the phosphorylation blocker is attached to the 5′ end of the sense strand or first nucleotide sequence. In some embodiments, the phosphorylation blocker is attached to the 5′ end of the sense strand or first nucleotide sequence via 1, 2, 3, 4, or 5 or more linkers. In some embodiments, the phosphorylation blocker is attached to the 3′ end of the antisense strand or second nucleotide sequence. In some embodiments, the phosphorylation blocker is attached to the 3′ end of the antisense strand or second nucleotide sequence via 1, 2, 3, 4, or 5 or more linkers. In some embodiments, the phosphorylation blocker is attached to the 5′ end of the antisense strand or second nucleotide sequence. In some embodiments, the phosphorylation blocker is attached to the 5′ end of the antisense strand or second nucleotide sequence via 1, 2, 3, 4, or 5 or more linkers. In some embodiments, the one or more linkers are independently selected from the group consisting of a phosphodiester linker, phosphorothioate linker, mesyl phosphoramidate linker and phosphorodithioate linker.

### 5′-Stabilized End Cap

Further disclosed herein are siNA molecules comprising a 5′-stabilized end cap. As used herein the terms “5′-stabilized end cap” and “5′ end cap” are used interchangeably. In some embodiments, a 2′-O-methyl nucleotide in any of sense strands or first nucleotide sequences disclosed herein is replaced with a nucleotide containing a 5′-stabilized end cap. In some embodiments, a 2′-O-methyl nucleotide in any of antisense strands or second nucleotide sequences disclosed herein is replaced with a nucleotide containing a 5′-stabilized end cap. In some embodiments, a 2′-O-methyl nucleotide in any of sense strands or first nucleotide sequences disclosed herein is further modified to contain a 5′-stabilized end cap. In some embodiments, a 2′-O-methyl nucleotide in any of antisense strands or second nucleotide sequences disclosed herein is further modified to contain a 5′-stabilized end cap.

In some embodiments, the 5′-stabilized end cap is a 5′ phosphate mimic. In some embodiments, the 5′-stabilized end cap is a modified 5′ phosphate mimic. In some embodiments, the modified 5′ phosphate is a chemically modified 5′ phosphate. In some embodiments, the 5′-stabilized end cap is a 5′-vinyl phosphonate. In some embodiments, the 5′-vinyl phosphonate is a 5′-(E)-vinyl phosphonate or 5′-(Z)-vinyl phosphonate. In some embodiments, the 5′-vinyl phosphonate is a deuterated vinyl phosphonate. In some embodiments, the deuterated vinyl phosphonate is a mono-deuterated vinyl phosphonate. In some embodiments, the deuterated vinyl phosphonate is a di-deuterated vinyl phosphonate. In some embodiments, the 5′-stabilized end cap is a phosphate mimic. Examples of phosphate mimics are disclosed in Parmar et al., J Med Chem, 201861 (3): 734-744, International Publication Nos. WO2018/045317 and WO2018/044350, and U.S. Pat. No. 10,087,210, each of which is incorporated by reference in its entirety.

In some embodiments, the 5′-stabilized end cap is not a phosphate mimic, per se, but rather includes a novel modified base. Examples of such 5′-stabilized end caps include, but are not limited to

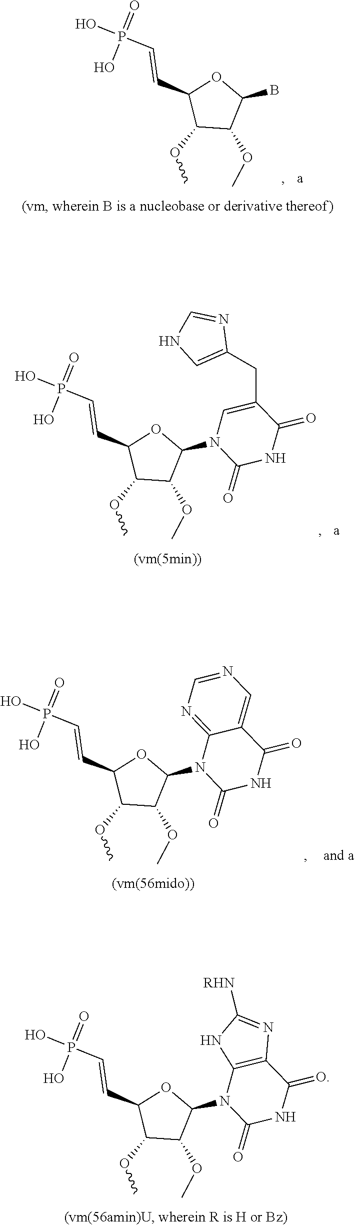

In some embodiments, any of the siNA molecules disclosed herein may comprise a 5′ vinyl phosphonate moiety comprising a structure of:

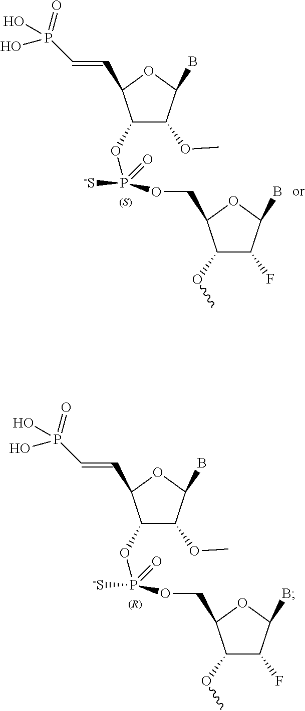

wherein B is a nucleobase, aryl, heteroaryl, or H; wherein [symbol] represents a phosphodiester linkage, a phosphorothioate linkage, or a mesyl phosphoramidate linkage.

In some embodiments, the disclosed siNA may comprise a nucleotide phosphate mimic selected from:

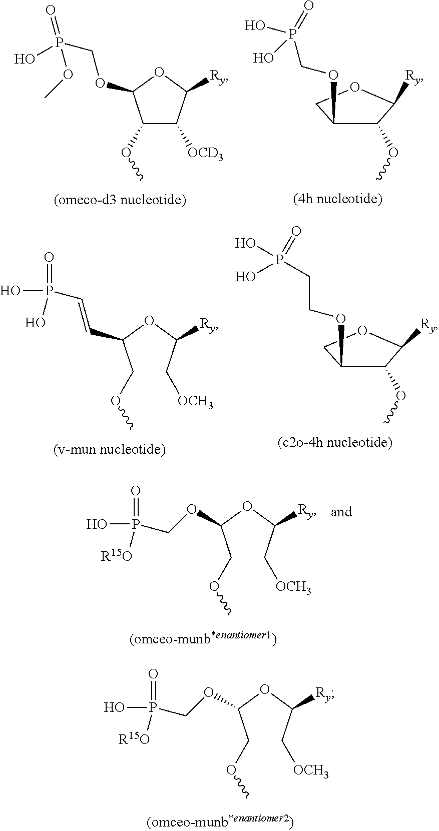

wherein R_{y} is a nucleobase and R¹⁵ is H or CH₃. In some embodiments, the nucleobase is selected from thymine, cytosine, guanine, adenine, uracil, and an analogue or derivative thereof. In some embodiments, the nucleobase is uracil. In some embodiments, the nucleobase is adenine. In some embodiments, the nucleobase is guanine. In some embodiments, the nucleobase is cytosine. In some embodiments, the nucleobase is thymine.

For the purposes of the omeco-d3 nucleotide, 4 h nucleotide, v-mun nucleotide, c20-4h nucleotide, omeco-munb nucleotide, and d2vm nucleotide, uridine can be a particularly suitable nucleobase. In some embodiments, one of these nucleotide phosphate mimics (e.g., omeco-d3 nucleotide, 4 h nucleotide, v-mun nucleotide, c20-4 h nucleotide, omeco-munb nucleotide, or d2vm nucleotide) are located at the 5′ end of the antisense strand; however, these nucleotide phosphate mimics may also be incorporated at the 5′ end of the sense strand, the 3′ end of the antisense strand, or the 3′ end of the sense strand.

Additionally or alternatively, the siNA molecules disclosed herein may comprise in the sense strand, the antisense strand, or both a 5′-stabilized end cap of Formula (Ia):

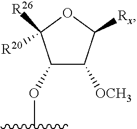

wherein Rₓ is H, a nucleobase, aryl, or heteroaryl; R²⁶ is

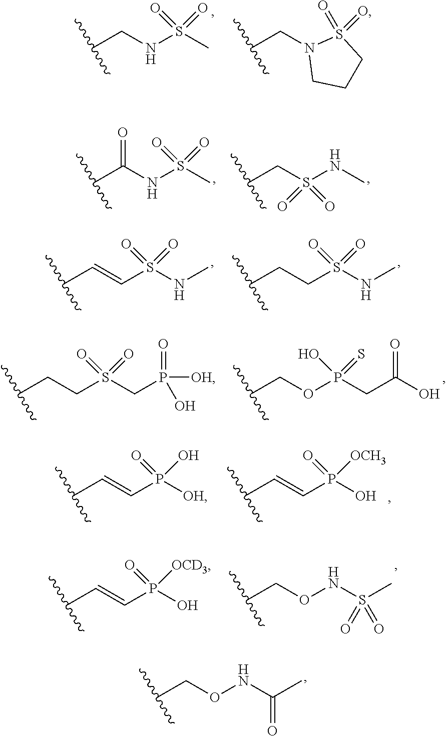

—CH=CD-Z, -CD-CH—Z, -CD-CD-Z, —(CR²¹R²²)ₙ—Z, or —(C₂-C₆ alkenylene)-Z and R²⁰ is H; or R²⁶ and R²⁰ together form a 3- to 7-membered carbocyclic ring substituted with —(CR²¹R²²)ₙ—Z or —(C₂-C₆ alkenylene)-Z; n is 1, 2, 3, or 4; Z is —ONR²³R²⁴, —OP(O)OH(CH₂)ₘCO₂R²³, —OP(S)OH(CH₂)ₘCO₂R²³, —P(O)(OH)₂, —P(O)(OH)(OCH₃), —P(O)(OH)(OCD₃), —SO₂ (CH₂)ₘP(O)(OH)₂, —SO₂NR²³R²⁵, —NR²³R²⁴, —NR²³SO₂R²⁴; either R²¹ and R²² are independently hydrogen or C₁-C₆ alkyl, or R²¹ and R²² together form an oxo group; R²³ is hydrogen or C₁-C₆ alkyl; R²⁴ is —SO₂R²⁵ or —C(O)R²⁵; or R²³ and R²⁴ together with the nitrogen to which they are attached form a substituted or unsubstituted heterocyclic ring; R²⁵ is C₁-C₆ alkyl; and m is 1, 2, 3, or 4. In some embodiments, R¹ is an aryl. In some embodiments, the aryl is a phenyl ring.

Additionally or alternatively, the siNA molecules disclosed herein may comprise in the sense strand, the antisense strand, or both a 5′-stabilized end cap of Formula (Ib):

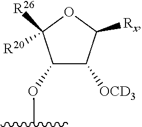

wherein Rₓ is H, a nucleobase, aryl, or heteroaryl; R²⁶ is

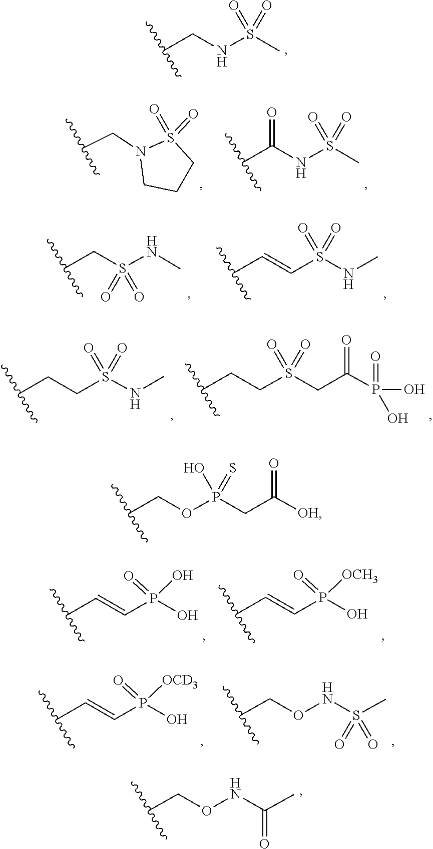

—CH═CD-Z, -CD═CH—Z, -CD=CD-Z, —(CR²¹R²²)ₙ—Z, or —(C₂-C₆ alkenylene)-Z and R²⁰ is H; or R²⁶ and R²⁰ together form a 3- to 7-membered carbocyclic ring substituted with —(CR²¹R²²) n-Z or —(C₂-C₆ alkenylene)-Z; n is 1, 2, 3, or 4; Z is —ONR²³R²⁴, —OP(O)OH(CH₂)ₘCO₂R²³, —OP(S)OH(CH₂)ₘCO₂R²³, —P(O)(OH)₂, —P(O)(OH)(OCH₃), —P(O)(OH)(OCD₃), —SO₂ (CH₂)ₘP(O)(OH)₂, —SO₂NR²³R²⁵, —NR²³R²⁴, —NR²³SO₂R²⁴; either R²¹ and R²² are independently hydrogen or C₁-C₆ alkyl, or R²¹ and R²² together form an oxo group; R²³ is hydrogen or C₁-C₆ alkyl; R²⁴ is —SO₂R²⁵ or —C(O)R²⁵; or R²³ and R²⁴ together with the nitrogen to which they are attached form a substituted or unsubstituted heterocyclic ring; R²⁵ is C₁-C₆ alkyl; and m is 1, 2, 3, or 4. In some embodiments, R¹ is an aryl. In some embodiments, the aryl is a phenyl ring.

Additionally or alternatively, the siNA molecules disclosed herein may comprise in the sense strand, the antisense strand, or both a 5′-stabilized end cap of Formula (Ic):

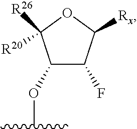

wherein Rₓ is a nucleobase, aryl, heteroaryl, or H,
R²⁶ is

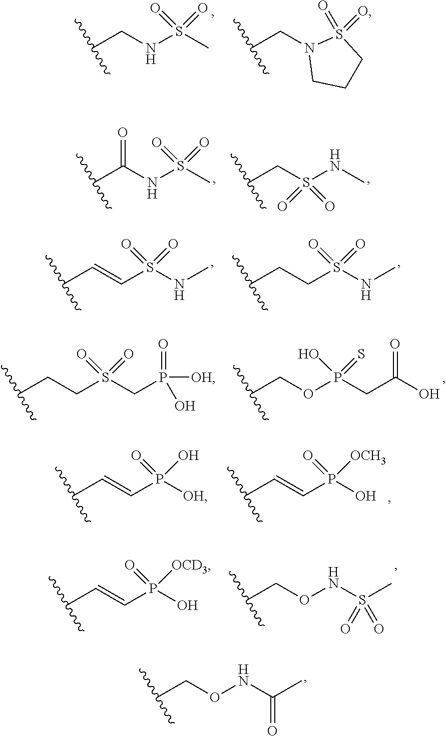

—CH=CD-Z, -CD=CH—Z, -CD-CD-Z, —(CR²¹R²²)ₙ—Z, or —(C₂-C₆ alkenylene)-Z and R²⁰ is hydrogen; or R²⁶ and R²⁰ together form a 3- to 7-membered carbocyclic ring substituted with —(CR²¹R²²)ₙ—Z or —(C₂-C₆ alkenylene)-Z; n is 1, 2, 3, or 4; Z is —ONR²³R²⁴, —OP(O)OH(CH₂)ₘCO₂R²³, —OP(S)OH(CH₂)ₘCO₂R²³, —P(O)(OH)₂, —P(O)(OH)(OCH₃), —P(O)(OH)(OCD₃), —SO₂ (CH₂)ₘP(O)(OH)₂, —SO₂NR²³R²⁵, —NR²³R²⁴, or —NR²³SO₂R²⁴; R²¹ and R²² either are independently hydrogen or C₁-C₆ alkyl, or R²¹ and R²² together form an oxo group; R²³ is hydrogen or C₁-C₆ alkyl; R²⁴ is —SO₂R²⁵ or —C(O)R²⁵; or R²³ and R²⁴ together with the nitrogen to which they are attached form a substituted or unsubstituted heterocyclic ring; R²⁵ is C₁-C₆ alkyl; and m is 1, 2, 3, or 4. In some embodiments, R¹ is an aryl. In some embodiments, the aryl is a phenyl.

Additionally or alternatively, the siNA molecules disclosed herein may comprise in the sense strand, the antisense strand, or both a 5′-stabilized end cap of Formula (IIa):

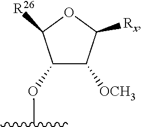

wherein Rₓ is a nucleobase, aryl, heteroaryl, or H, R²⁶ is

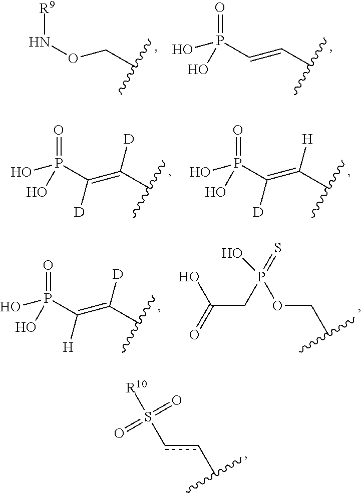

—CH₂SO₂NHCH₃, or

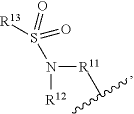

R⁹ is —SO₂CH₃ or —COCH₃, [symbol] is a double or single bond, R¹⁰=—CH₂PO₃H or —NHCH₃, R¹¹ is —CH₂— or —CO—, and R¹² is H and R¹³ is CH₃ or R¹² and R¹³ together form —CH₂CH₂CH₂—. In some embodiments, R¹ is an aryl. In some embodiments, the aryl is a phenyl ring.

Additionally or alternatively, the siNA molecules disclosed herein may comprise in the sense strand, the antisense strand, or both a 5′-stabilized end cap of Formula (IIb):

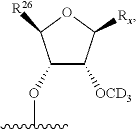

wherein Rₓ is a nucleobase, aryl, heteroaryl, or H, R²⁶ is

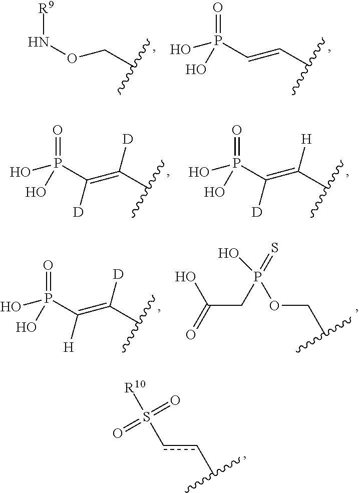

—CH₂SO₂NHCH₃, or

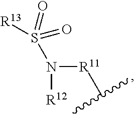

R⁹ is —SO₂CH₃ or —COCH₃, [symbol] is a double or single bond, R¹⁰=—CH₂PO₃H or —NHCH₃, R¹¹ is —CH₂— or —CO—, and R¹² is H and R¹³ is CH₃ or R¹² and R¹³ together form —CH₂CH₂CH₂—. In some embodiments, R¹ is an aryl. In some embodiments, the aryl is a phenyl ring.

Additionally or alternatively, the siNA molecules disclosed herein may comprise in the sense strand, the antisense strand, or both a 5′-stabilized end cap of Formula (III):

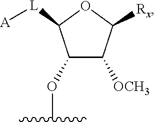

wherein Rₓ is a nucleobase, aryl, heteroaryl, or H, L is —CH₂—, —CH═CH—, —CO—, or —CH₂CH₂—, and A is —ONHCOCH₃, —ONHSO₂CH₃, —PO₃H, —OP(SOH)CH₂CO₂H, —SO₂CH₂PO₃H, —SO₂NHCH₃, —NHSO₂CH₃, or —N(SO₂CH₂CH₂CH₂). In some embodiments, R¹ is an aryl. In some embodiments, the aryl is a phenyl ring.

Additionally or alternatively, the siNA molecules disclosed herein may comprise a 5′-stabilized end cap selected from the group consisting of Formula (1) to Formula (16), Formula (9X) to Formula (12X), Formula (16X), Formula (9Y) to Formula (12Y), Formula (16Y), Formula (21) to Formula (36), Formula 36X, Formula (41) to (56), Formula (49X) to (52X), Formula (49Y) to (52Y), Formula 56X, Formula 56Y, Formula (61) and Formula (62):

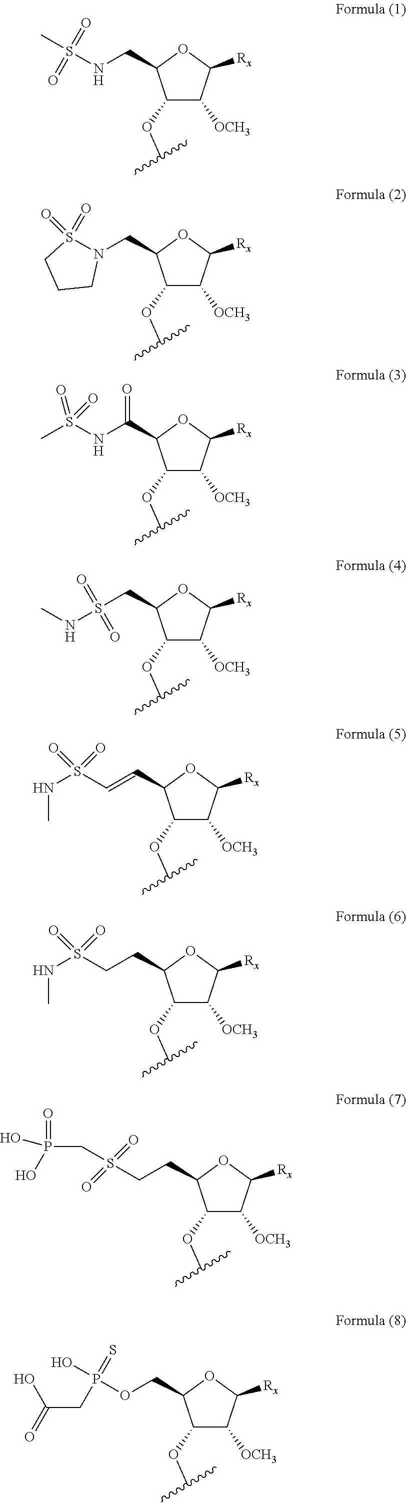

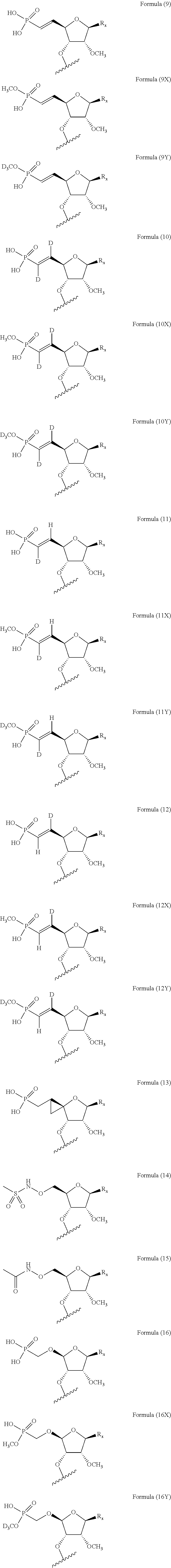

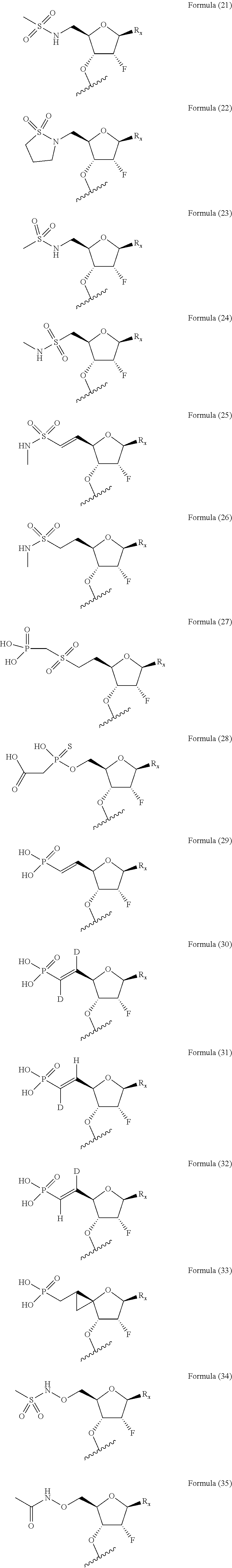

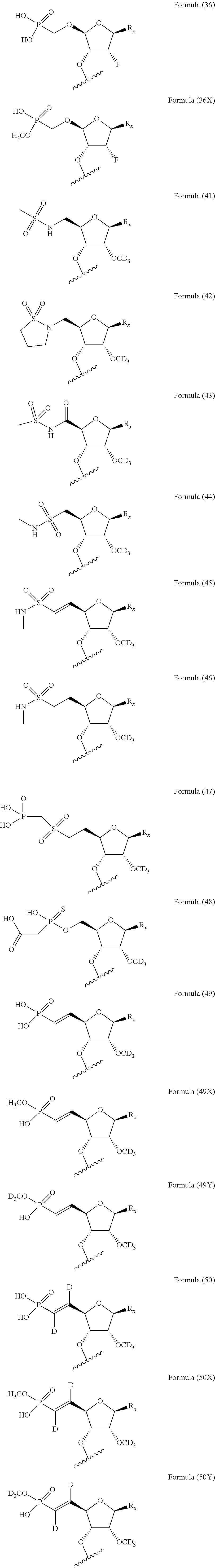

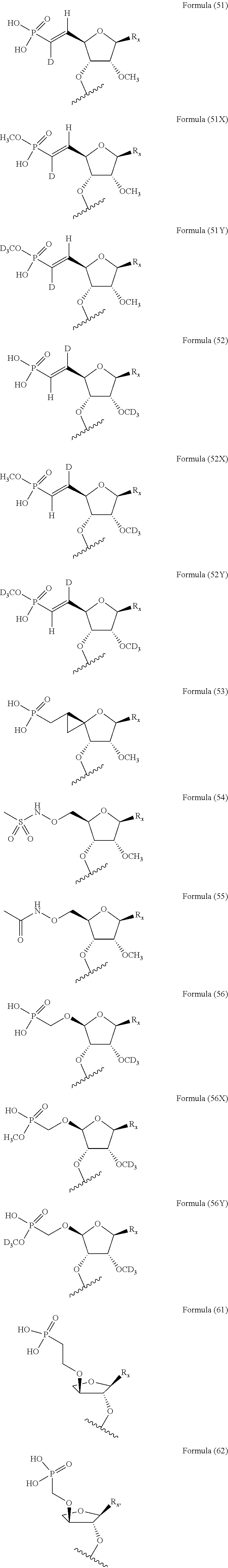

wherein Rₓ is a nucleobase, aryl, heteroaryl, or H.

In some embodiments, any of the siNA molecules disclosed herein comprise a 5′-stabilized end cap selected from the group consisting of Formula (50), Formula (50X), Formula (50Y), Formula (56), Formula (56X), Formula (56Y), Formula (61), and Formula (62):

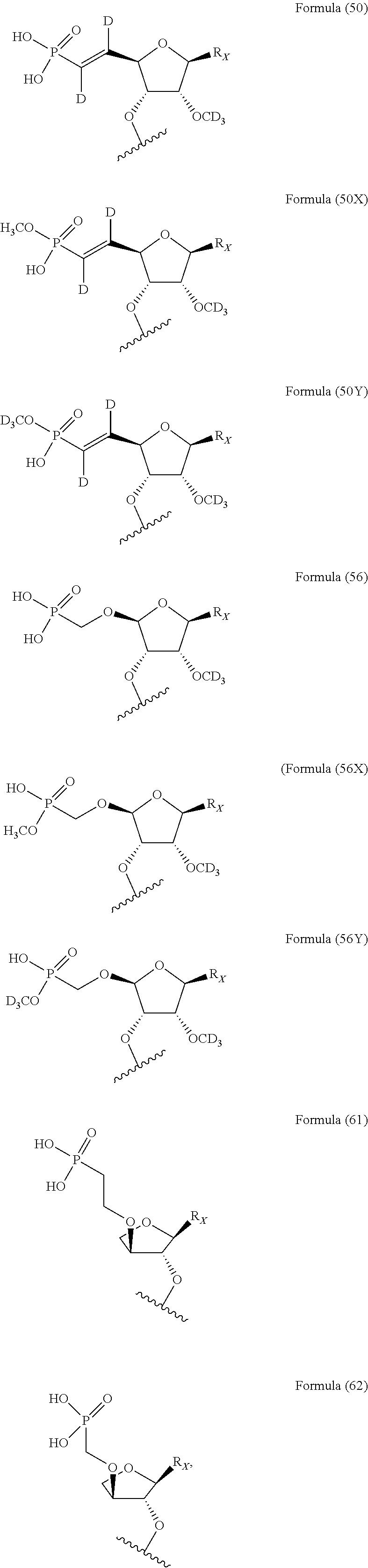

wherein Rₓ is a nucleobase, aryl, heteroaryl, or H.

In some embodiments, any of the siNA molecules disclosed herein comprise a 5′-stabilized end cap selected from the group consisting of Formula (71) to Formula (86), Formula (79X) to Formula (82X), Formula (79Y) to (82Y), Formula 86X, Formula 86X′, Formula 86Y, and Formula 86Y′:

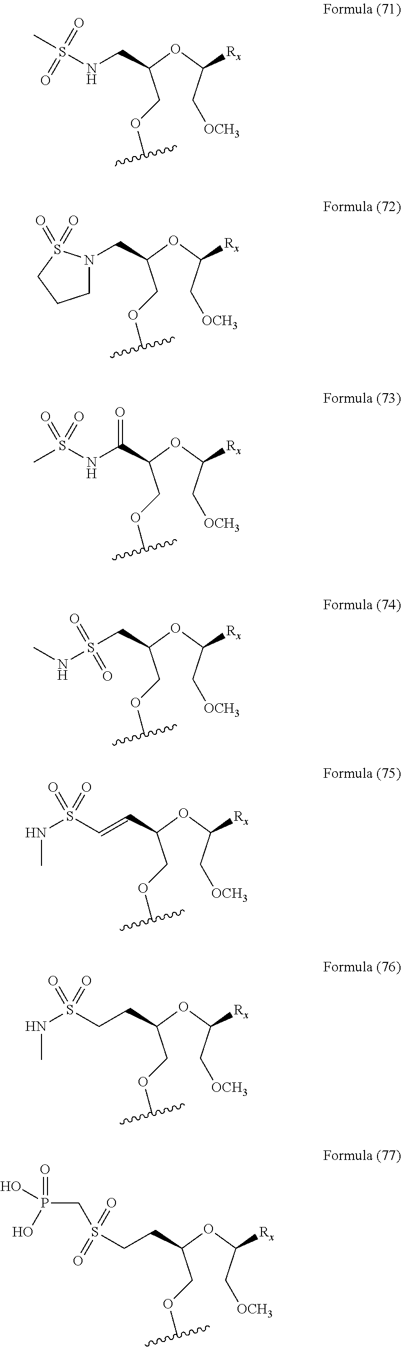

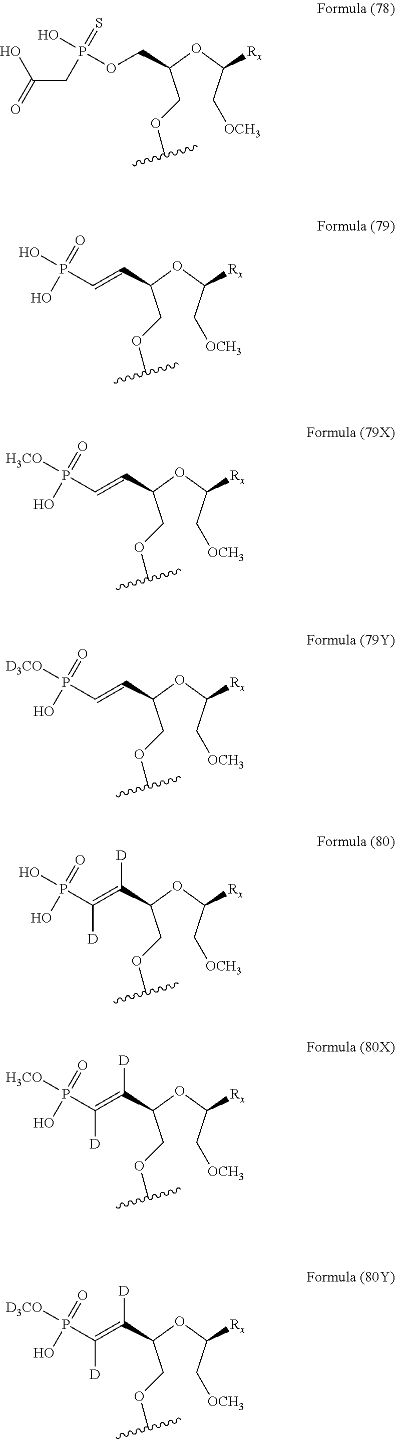

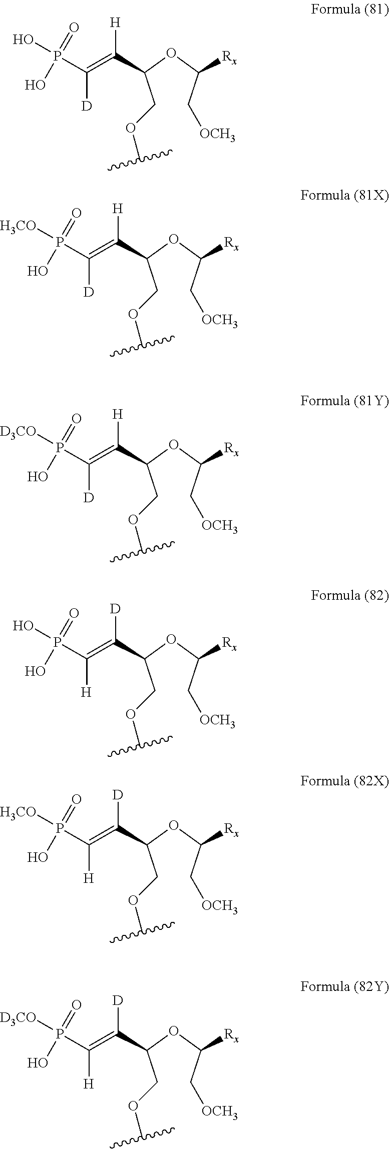

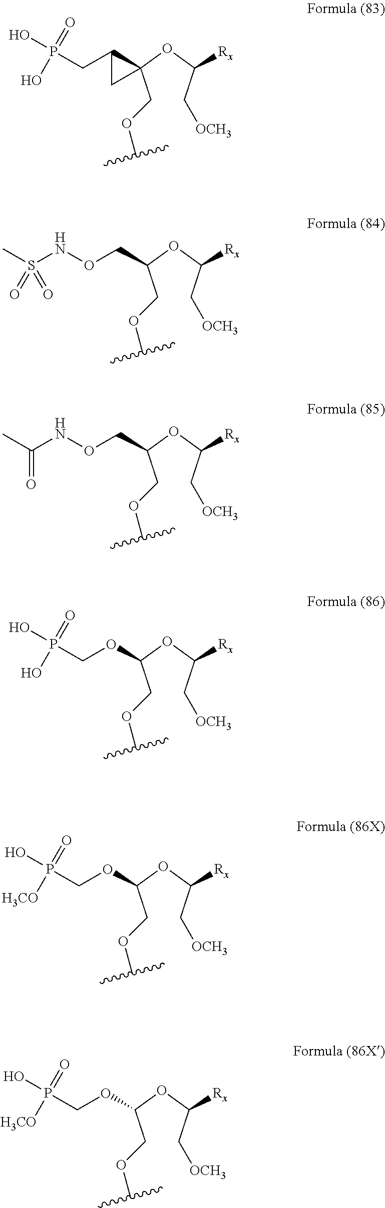

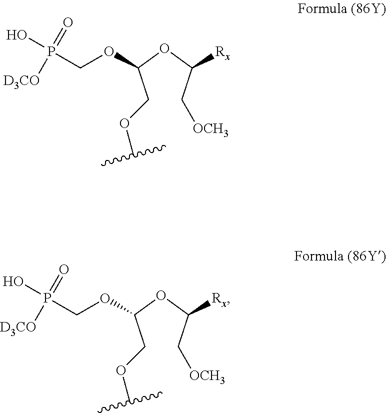

wherein Rₓ is a nucleobase, aryl, heteroaryl, or H.

In some embodiments, any of the siNA molecules disclosed herein comprise a 5′-stabilized end cap selected from the group consisting of Formula (78), Formula (79), Formula (79X), Formula (79Y), Formula (86), Formula (86X), and Formula (86X′):

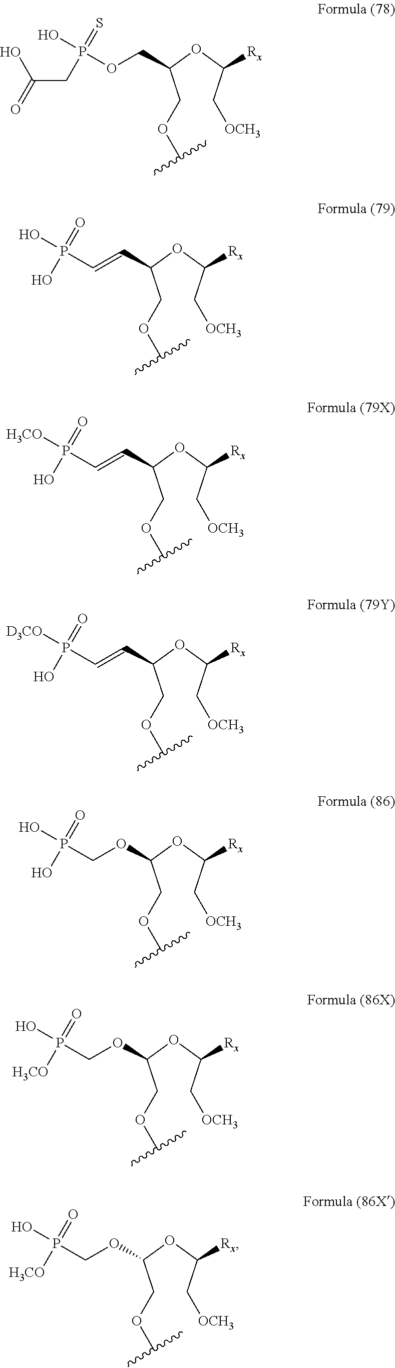

wherein Rₓ is a nucleobase, aryl, heteroaryl, or H.

In some embodiments, any of the siNA molecules disclosed herein comprise a 5′-stabilized end cap selected from the group consisting of Formulas (1A)-(15A), Formulas (1A-1)-(7A-1), Formulas (1A-2)-(7A-2), Formulas (1A-3)-(7A-3), Formulas (1A-4)-(7A-4), Formulas (9B)-(12B), Formulas (9AX)-(12AX), Formulas (9AY)-(12AY), Formulas (9BX)-(12BX), and Formulas (9BY)-(12BY):

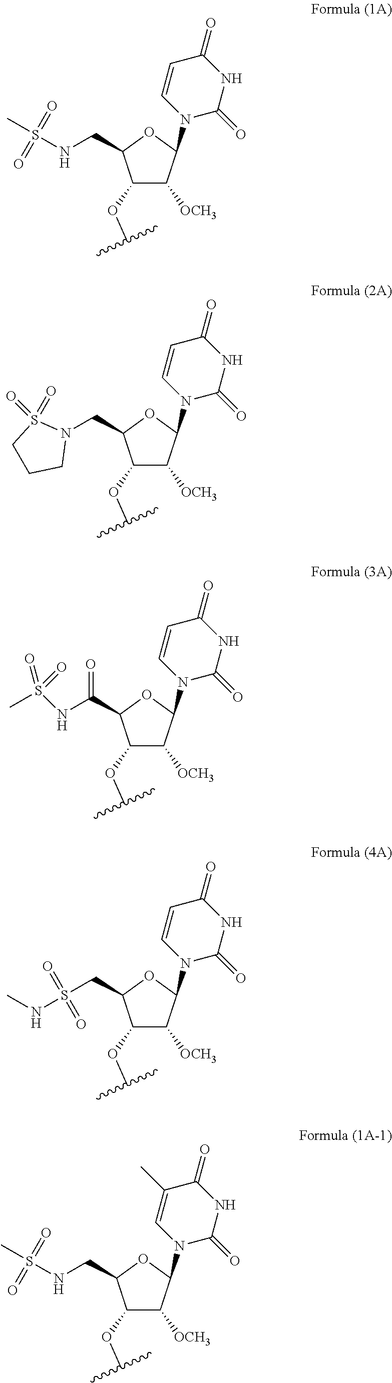 [SMILES: *O[C@H]1[C@@H](OC)[C@H](n2ccc(=O)[nH]c2=O)O[C@@H]1CNS(C)(=O)=O ; *O[C@H]1[C@@H](OC)[C@H](n2ccc(=O)[nH]c2=O)O[C@@H]1CN1CCCS1(=O)=O ; *O[C@H]1[C@@H](OC)[C@H](n2ccc(=O)[nH]c2=O)O[C@@H]1C(=O)NS(C)(=O)=O ; *O[C@H]1[C@@H](OC)[C@H](n2ccc(=O)[nH]c2=O)O[C@@H]1CS(=O)(=O)NC ; *O[C@H]1[C@@H](OC)[C@H](n2cc(C)c(=O)[nH]c2=O)O[C@@H]1CNS(C)(=O)=O]

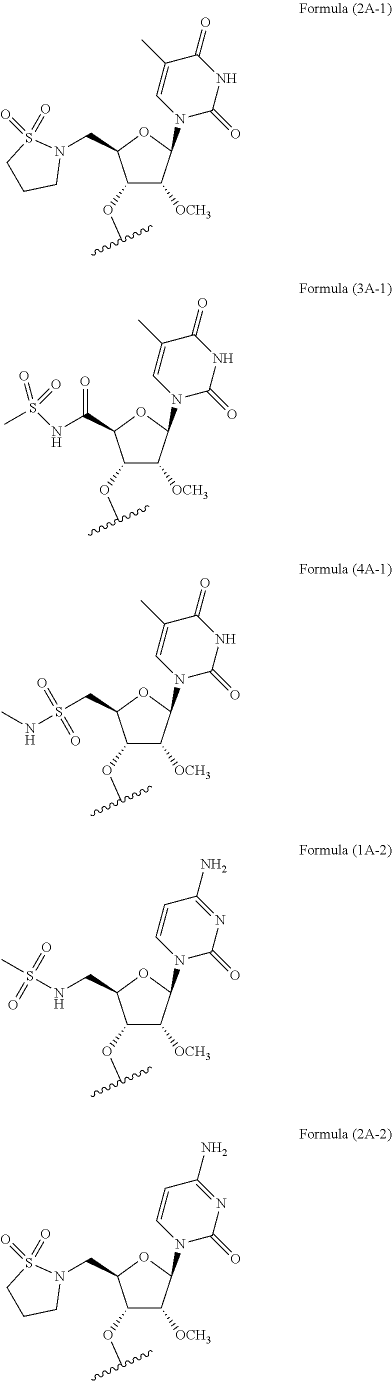 [SMILES: *O[C@H]1[C@@H](OC)[C@H](n2cc(C)c(=O)[nH]c2=O)O[C@@H]1CN1CCCS1(=O)=O ; *O[C@H]1[C@@H](OC)[C@H](n2cc(C)c(=O)[nH]c2=O)O[C@@H]1C(=O)NS(C)(=O)=O ; *O[C@H]1[C@@H](OC)[C@H](n2cc(C)c(=O)[nH]c2=O)O[C@@H]1CS(=O)(=O)NC ; *O[C@H]1[C@@H](OC)[C@H](n2ccc(N)nc2=O)O[C@@H]1CNS(C)(=O)=O ; *O[C@H]1[C@@H](OC)[C@H](n2ccc(N)nc2=O)O[C@@H]1CN1CCCS1(=O)=O]

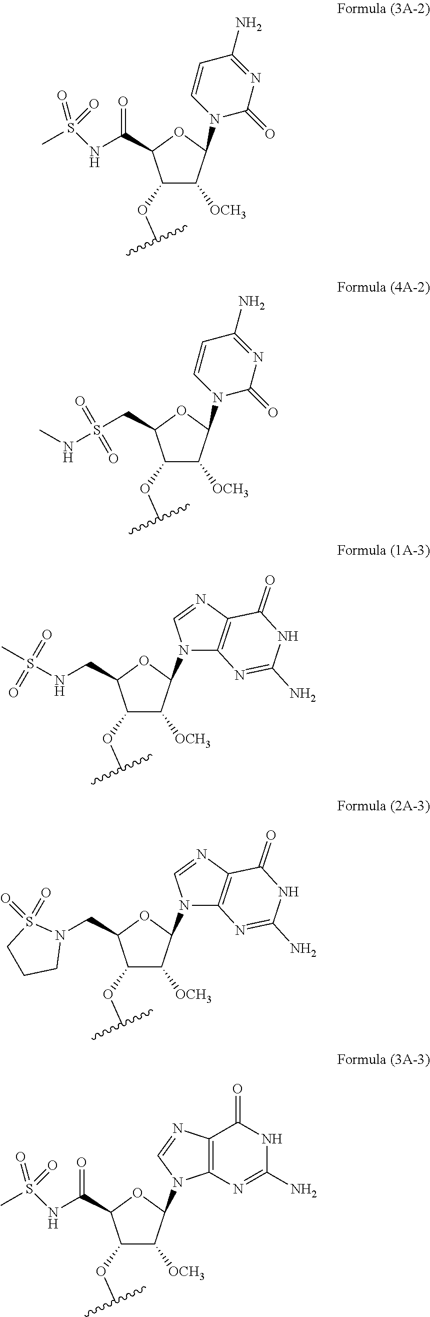 [SMILES: *O[C@H]1[C@@H](OC)[C@H](n2ccc(N)nc2=O)O[C@@H]1C(=O)NS(C)(=O)=O ; *O[C@H]1[C@@H](OC)[C@H](n2ccc(N)nc2=O)O[C@@H]1CS(=O)(=O)NC ; *O[C@H]1[C@@H](OC)[C@H](n2cnc3c(=O)[nH]c(N)nc32)O[C@@H]1CNS(C)(=O)=O ; *O[C@H]1[C@@H](OC)[C@H](n2cnc3c(=O)[nH]c(N)nc32)O[C@@H]1CN1CCCS1(=O)=O ; *O[C@H]1[C@@H](OC)[C@H](n2cnc3c(=O)[nH]c(N)nc32)O[C@@H]1C(=O)NS(C)(=O)=O]

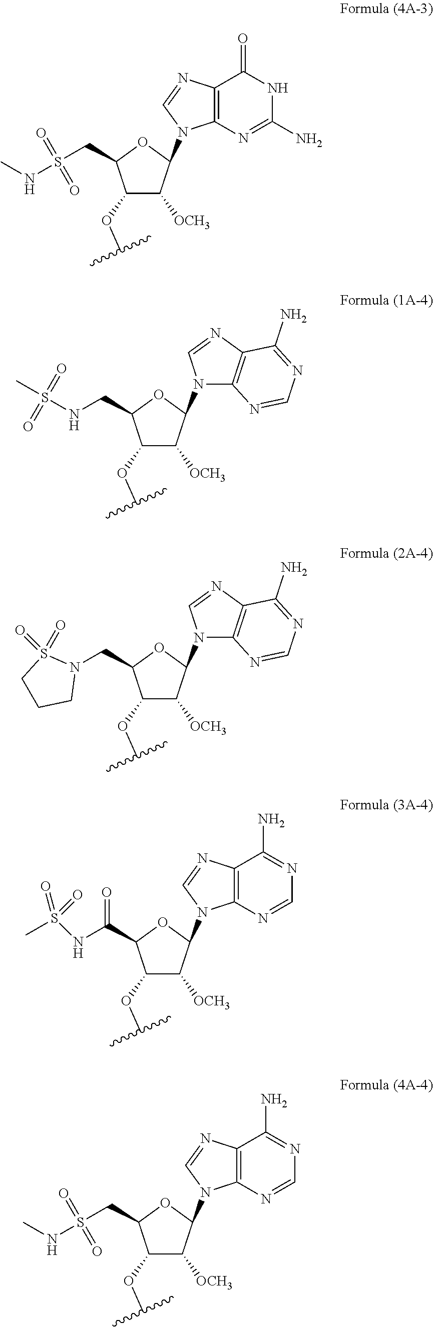 [SMILES: *O[C@H]1[C@@H](OC)[C@H](n2cnc3c(=O)[nH]c(N)nc32)O[C@@H]1CS(=O)(=O)NC ; *O[C@H]1[C@@H](OC)[C@H](n2cnc3c(N)ncnc32)O[C@@H]1CNS(C)(=O)=O ; *O[C@H]1[C@@H](OC)[C@H](n2cnc3c(N)ncnc32)O[C@@H]1CN1CCCS1(=O)=O ; *O[C@H]1[C@@H](OC)[C@H](n2cnc3c(N)ncnc32)O[C@@H]1C(=O)NS(C)(=O)=O ; *O[C@H]1[C@@H](OC)[C@H](n2cnc3c(N)ncnc32)O[C@@H]1CS(=O)(=O)NC]

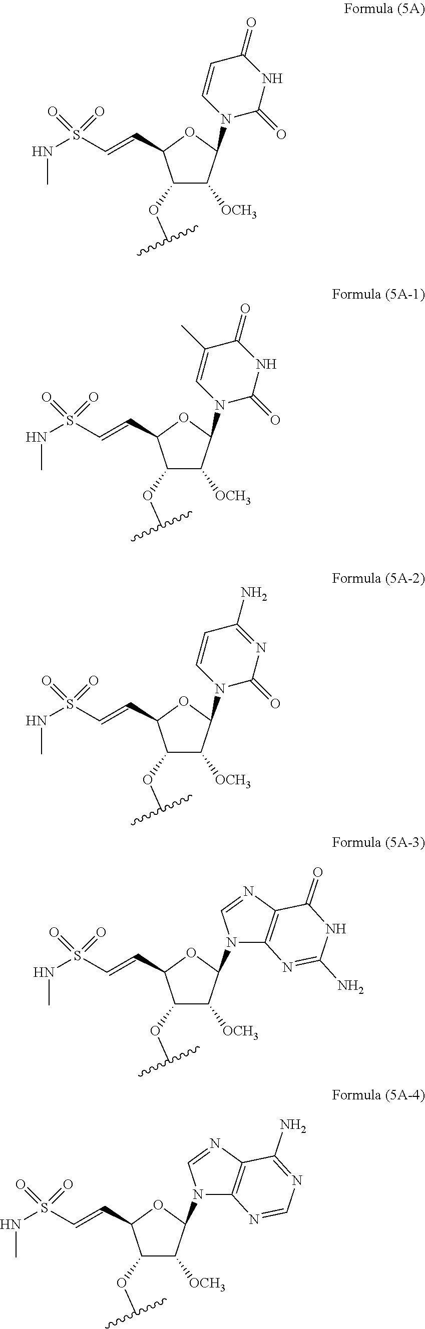 [SMILES: *O[C@H]1[C@@H](OC)[C@H](n2ccc(=O)[nH]c2=O)O[C@@H]1/C=C/S(=O)(=O)NC ; *O[C@H]1[C@@H](OC)[C@H](n2cc(C)c(=O)[nH]c2=O)O[C@@H]1/C=C/S(=O)(=O)NC ; *O[C@H]1[C@@H](OC)[C@H](n2ccc(N)nc2=O)O[C@@H]1/C=C/S(=O)(=O)NC ; *O[C@H]1[C@@H](OC)[C@H](n2cnc3c(=O)[nH]c(N)nc32)O[C@@H]1/C=C/S(=O)(=O)NC ; *O[C@H]1[C@@H](OC)[C@H](n2cnc3c(N)ncnc32)O[C@@H]1/C=C/S(=O)(=O)NC]

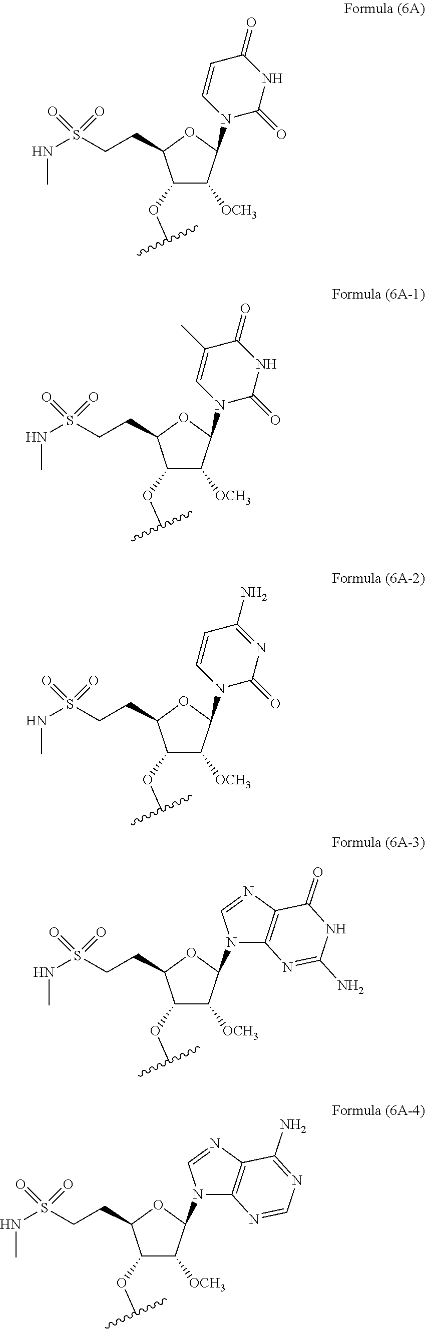 [SMILES: *O[C@H]1[C@@H](OC)[C@H](n2ccc(=O)[nH]c2=O)O[C@@H]1CCS(=O)(=O)NC ; *O[C@H]1[C@@H](OC)[C@H](n2cc(C)c(=O)[nH]c2=O)O[C@@H]1CCS(=O)(=O)NC ; *O[C@H]1[C@@H](OC)[C@H](n2ccc(N)nc2=O)O[C@@H]1CCS(=O)(=O)NC ; *O[C@H]1[C@@H](OC)[C@H](n2cnc3c(=O)[nH]c(N)nc32)O[C@@H]1CCS(=O)(=O)NC ; *O[C@H]1[C@@H](OC)[C@H](n2cnc3c(N)ncnc32)O[C@@H]1CCS(=O)(=O)NC]

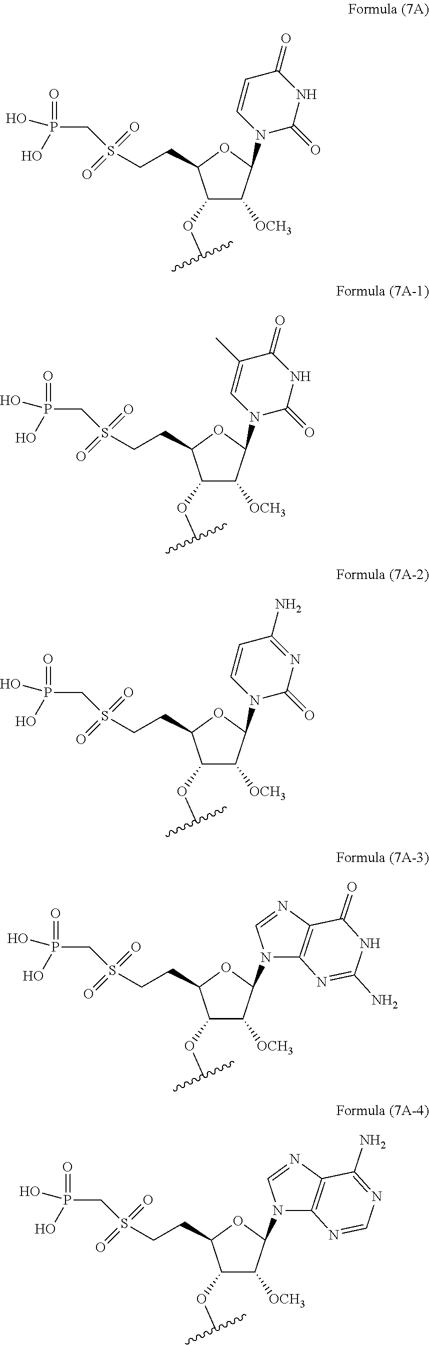 [SMILES: *O[C@H]1[C@@H](OC)[C@H](n2ccc(=O)[nH]c2=O)O[C@@H]1CCS(=O)(=O)CP(=O)(O)O ; *O[C@H]1[C@@H](OC)[C@H](n2cc(C)c(=O)[nH]c2=O)O[C@@H]1CCS(=O)(=O)CP(=O)(O)O ; *O[C@H]1[C@@H](OC)[C@H](n2ccc(N)nc2=O)O[C@@H]1CCS(=O)(=O)CP(=O)(O)O ; *O[C@H]1[C@@H](OC)[C@H](n2cnc3c(=O)[nH]c(N)nc32)O[C@@H]1CCS(=O)(=O)CP(=O)(O)O ; *O[C@H]1[C@@H](OC)[C@H](n2cnc3c(N)ncnc32)O[C@@H]1CCS(=O)(=O)CP(=O)(O)O]

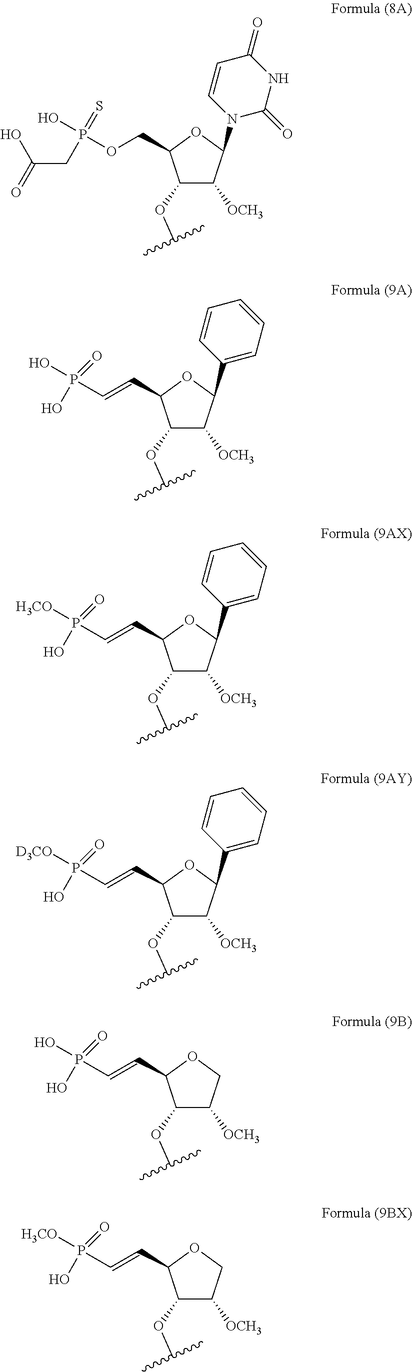

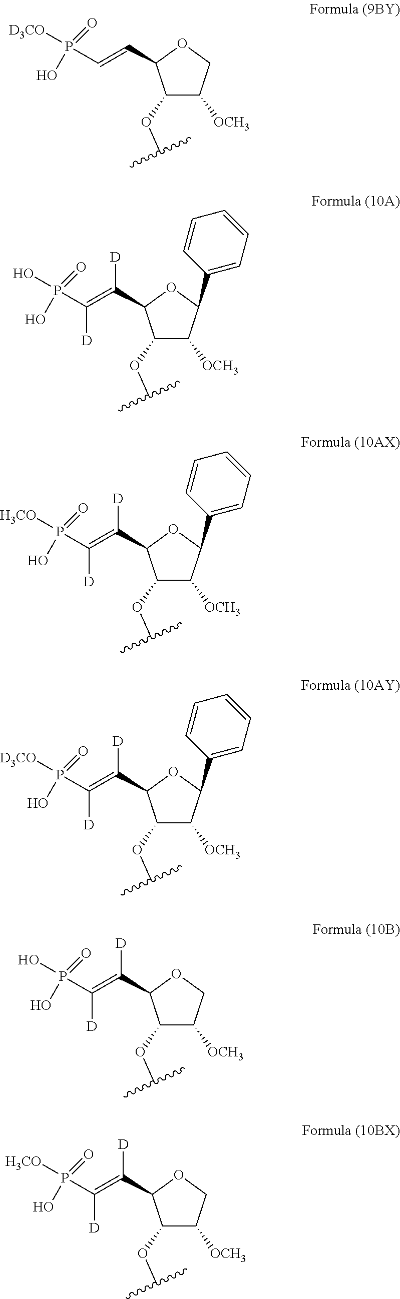

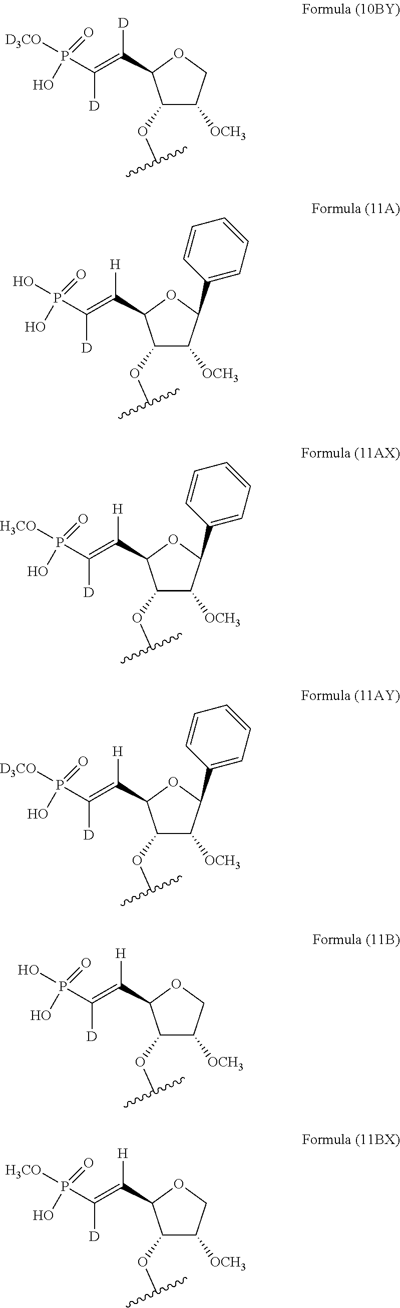

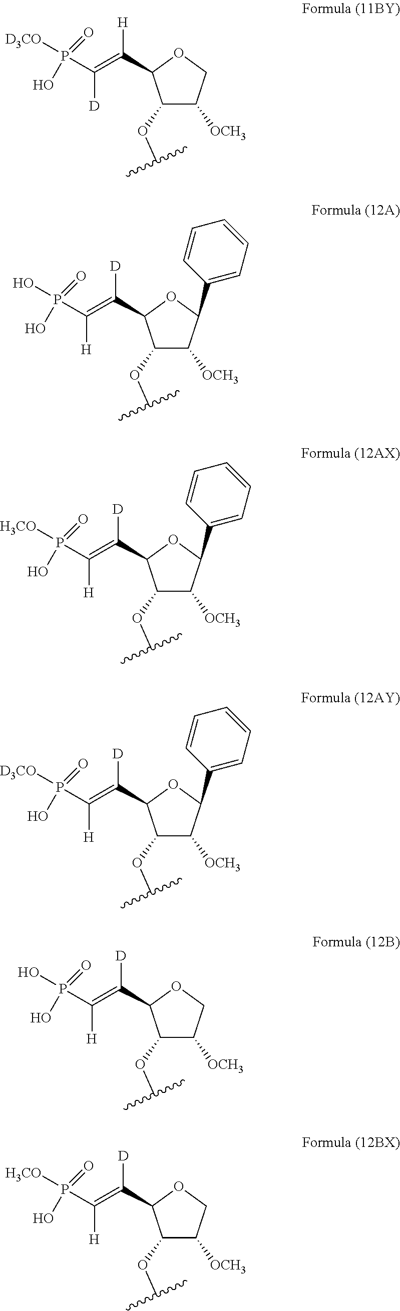

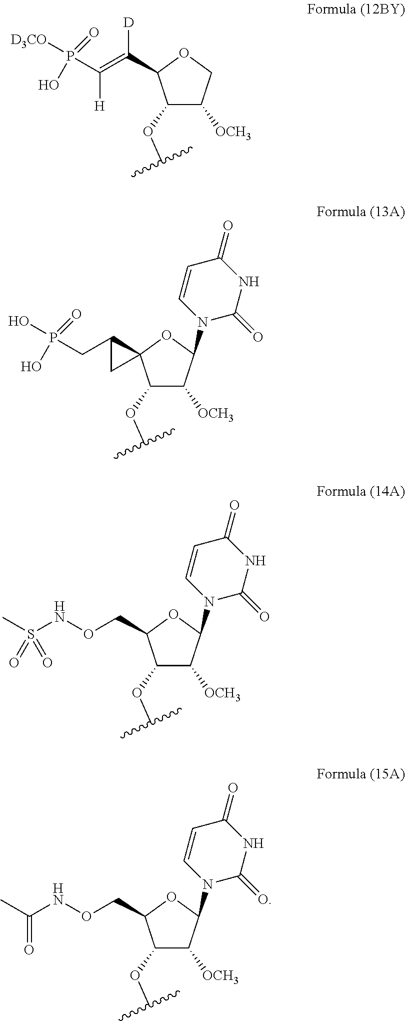

In some embodiments, any of the siNA molecules disclosed herein comprise a 5′-stabilized end cap selected from the group consisting of Formulas (21A)-(35A), Formulas (29B)-(32B), Formulas (29AX)-(32AX), Formulas (29AY)-(32AY), Formulas (29BX)-(32BX), and Formulas (29BY)-(32BY):

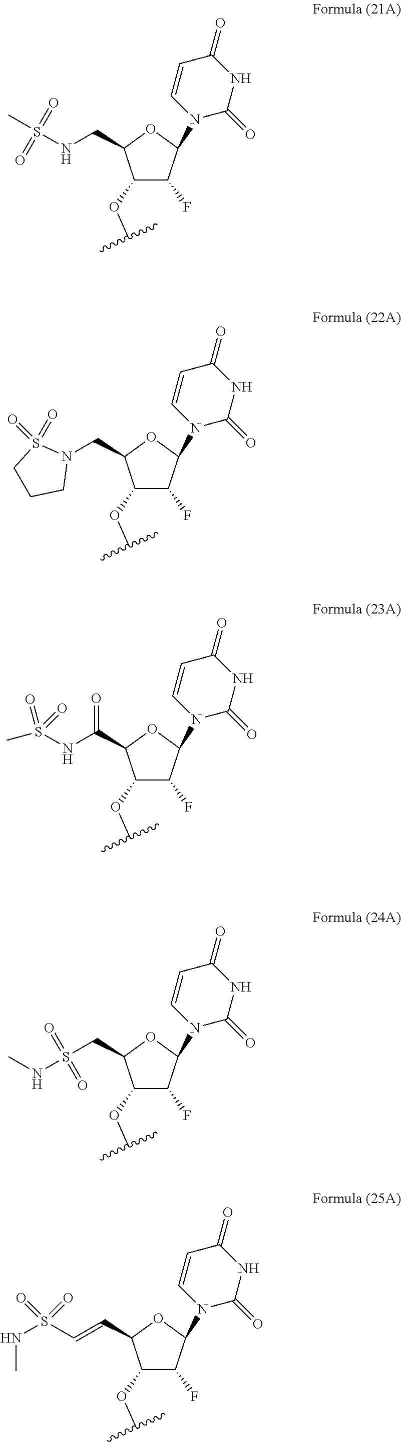 [SMILES: *O[C@H]1[C@@H](F)[C@H](n2ccc(=O)[nH]c2=O)O[C@@H]1CNS(C)(=O)=O ; *O[C@H]1[C@@H](F)[C@H](n2ccc(=O)[nH]c2=O)O[C@@H]1CN1CCCS1(=O)=O ; *O[C@H]1[C@@H](F)[C@H](n2ccc(=O)[nH]c2=O)O[C@@H]1C(=O)NS(C)(=O)=O ; *O[C@H]1[C@@H](F)[C@H](n2ccc(=O)[nH]c2=O)O[C@@H]1CS(=O)(=O)NC ; *O[C@H]1[C@@H](F)[C@H](n2ccc(=O)[nH]c2=O)O[C@@H]1/C=C/S(=O)(=O)NC]

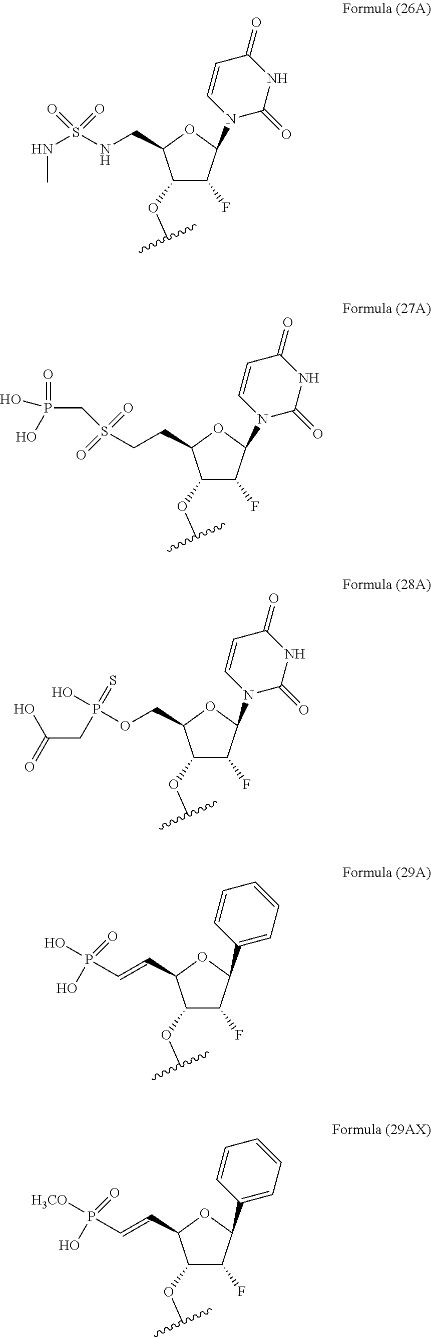 [SMILES: *O[C@H]1[C@@H](F)[C@H](n2ccc(=O)[nH]c2=O)O[C@@H]1CNS(=O)(=O)NC ; *O[C@H]1[C@@H](F)[C@H](n2ccc(=O)[nH]c2=O)O[C@@H]1CCS(=O)(=O)CP(=O)(O)O ; *O[C@H]1[C@@H](F)[C@H](n2ccc(=O)[nH]c2=O)O[C@@H]1COP(O)(=S)CC(=O)O ; *O[C@H]1[C@@H](F)[C@H](c2ccccc2)O[C@@H]1/C=C/P(=O)(O)O ; *O[C@H]1[C@@H](F)[C@H](c2ccccc2)O[C@@H]1/C=C/P(=O)(O)OC]

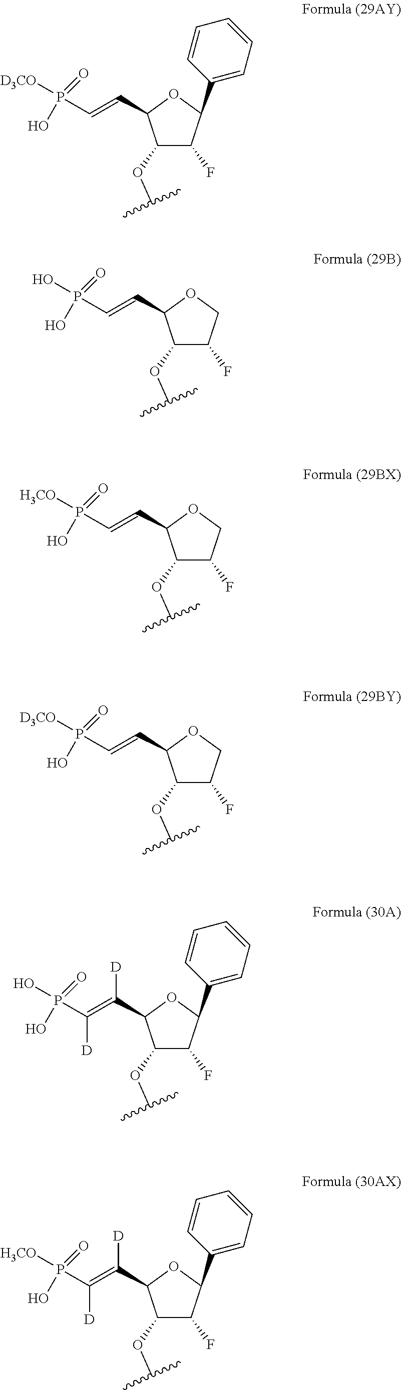

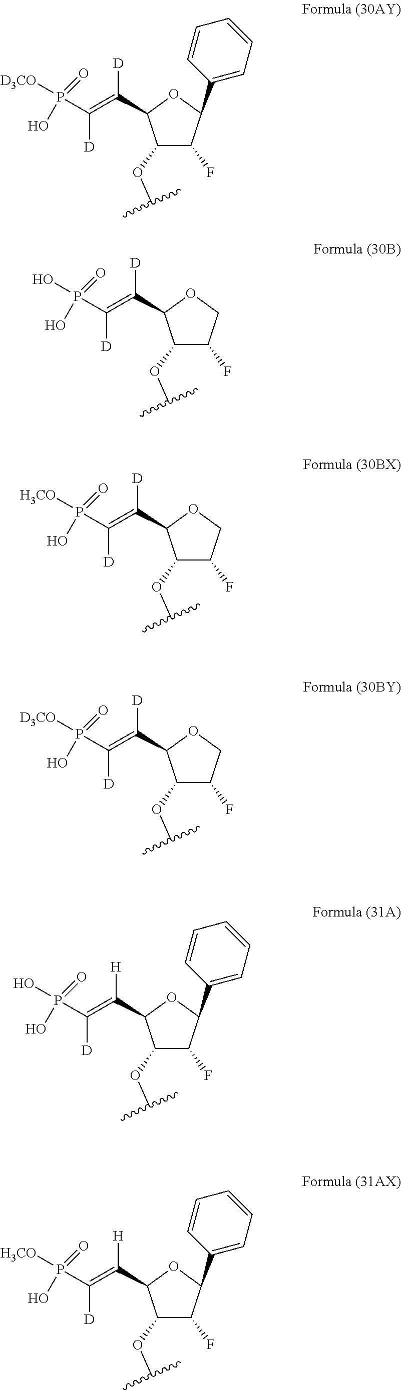

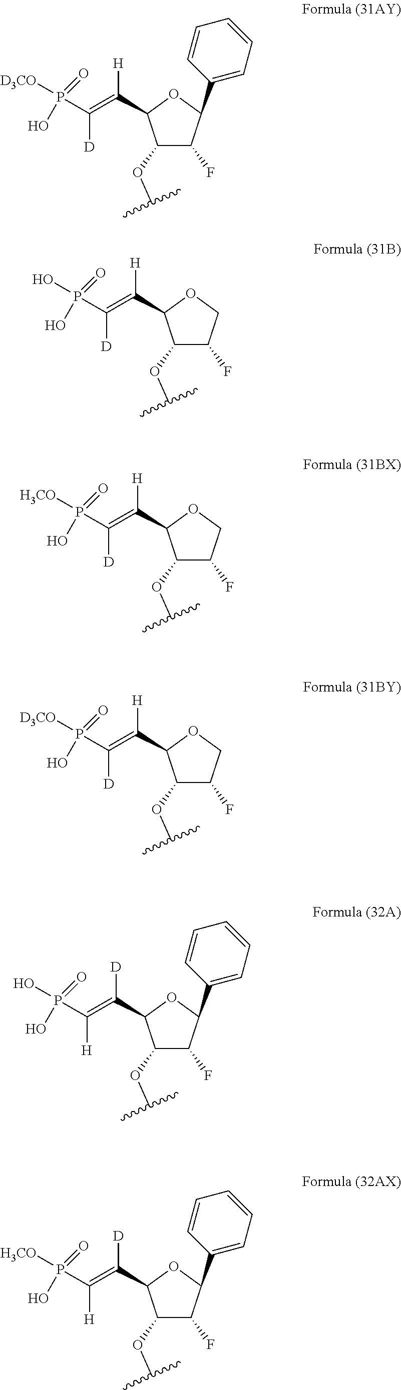

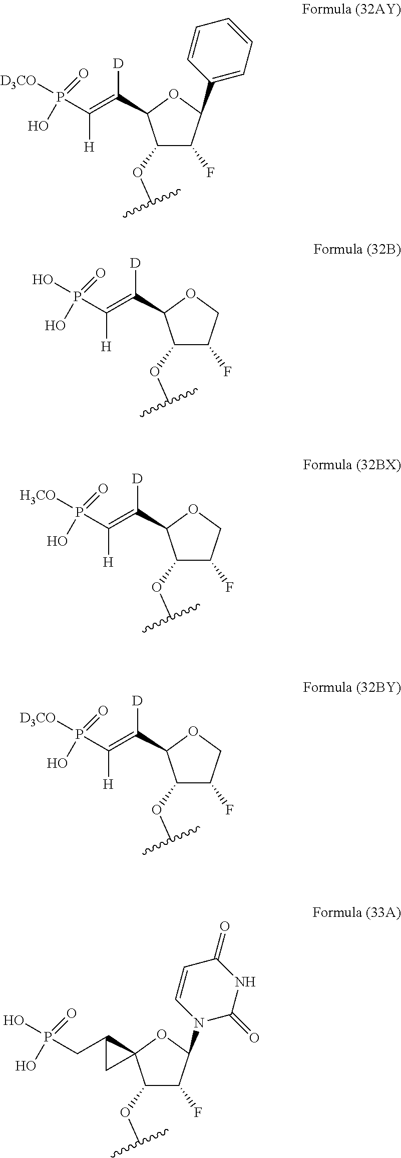

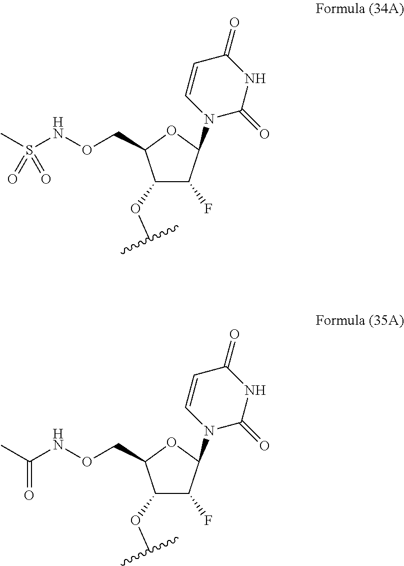 [SMILES: *O[C@H]1[C@@H](F)[C@H](n2ccc(=O)[nH]c2=O)O[C@@H]1CONS(C)(=O)=O ; *O[C@H]1[C@@H](F)[C@H](n2ccc(=O)[nH]c2=O)O[C@@H]1CONC(C)=O]

In some embodiments, any of the siNA molecules disclosed herein comprise a 5′-stabilized end cap selected from the group consisting of Formulas (71A)-(86A), Formulas (79XA)-(82XA), Formulas (79YA)-(82YA); Formula (86XA), Formula (86X′A), Formula (86Y), and Formula (86Y′):

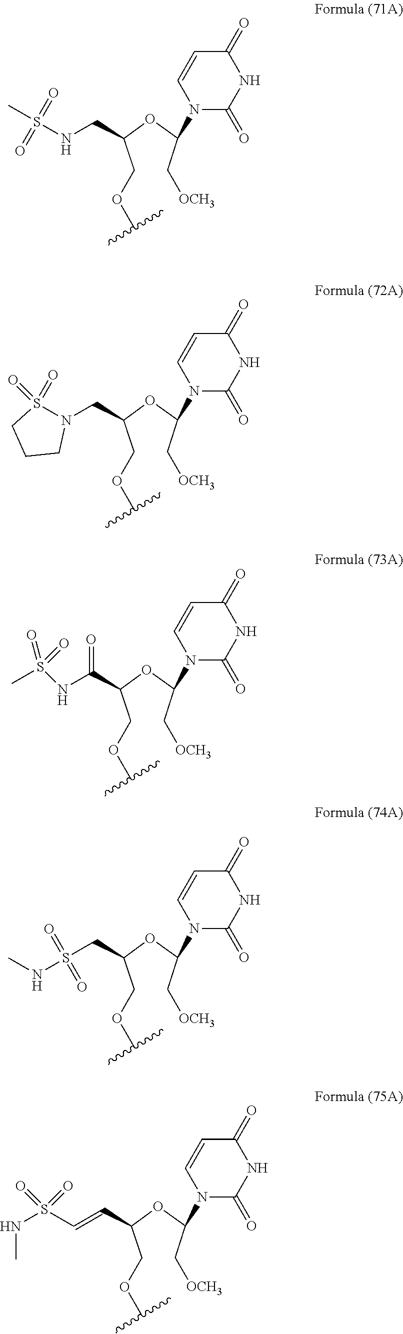 [SMILES: *OC[C@@H](CNS(C)(=O)=O)O[C@H](COC)n1ccc(=O)[nH]c1=O ; *OC[C@@H](CN1CCCS1(=O)=O)O[C@H](COC)n1ccc(=O)[nH]c1=O ; *OC[C@H](O[C@H](COC)n1ccc(=O)[nH]c1=O)C(=O)NS(C)(=O)=O ; *OC[C@@H](CS(=O)(=O)NC)O[C@H](COC)n1ccc(=O)[nH]c1=O ; *OC[C@@H](/C=C/S(=O)(=O)NC)O[C@H](COC)n1ccc(=O)[nH]c1=O]

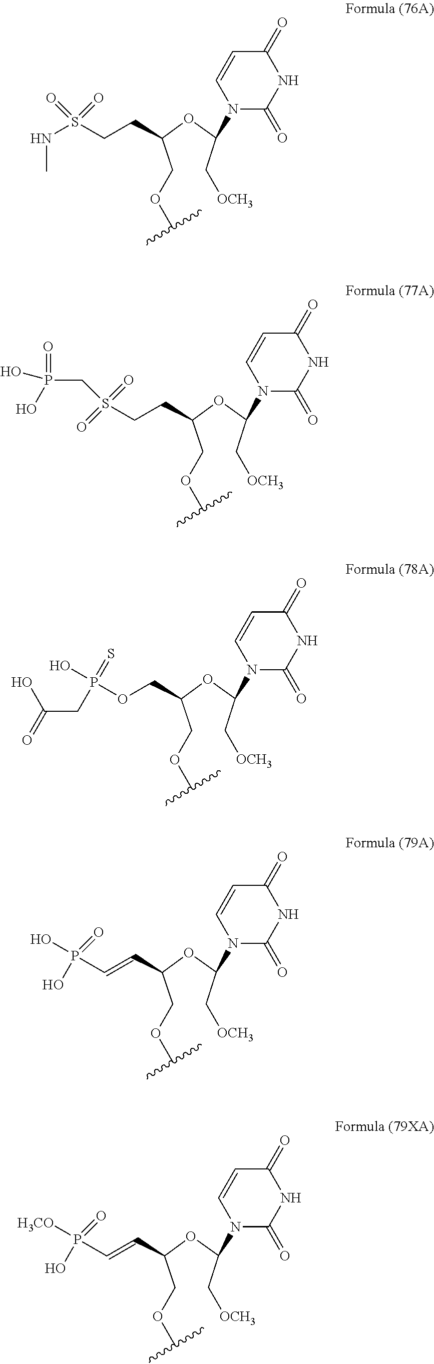 [SMILES: *OC[C@@H](CCS(=O)(=O)NC)O[C@H](COC)n1ccc(=O)[nH]c1=O ; *OC[C@@H](CCS(=O)(=O)CP(=O)(O)O)O[C@H](COC)n1ccc(=O)[nH]c1=O ; *OC[C@@H](COP(O)(=S)CC(=O)O)O[C@H](COC)n1ccc(=O)[nH]c1=O ; *OC[C@@H](/C=C/P(=O)(O)O)O[C@H](COC)n1ccc(=O)[nH]c1=O ; *OC[C@@H](/C=C/P(=O)(O)OC)O[C@H](COC)n1ccc(=O)[nH]c1=O]

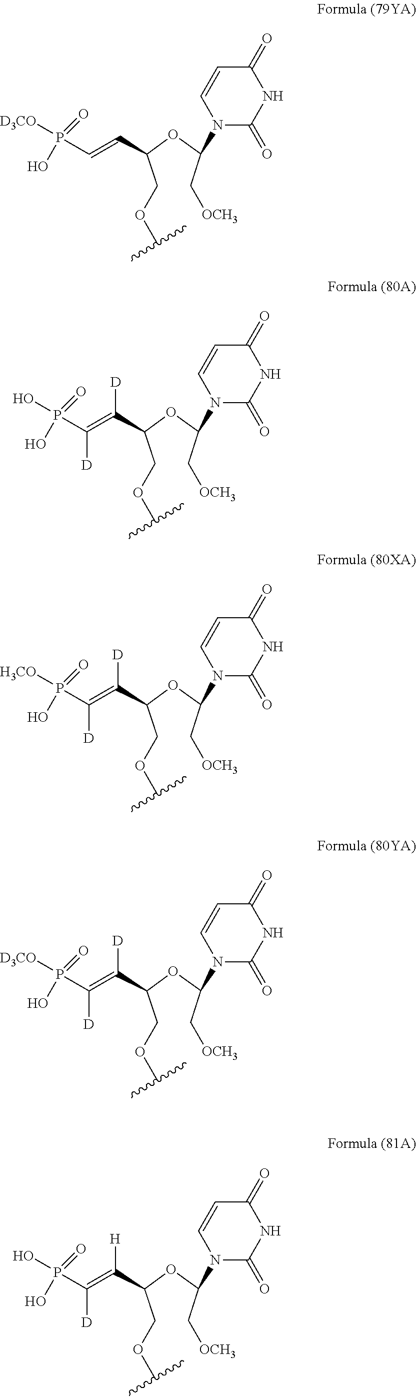

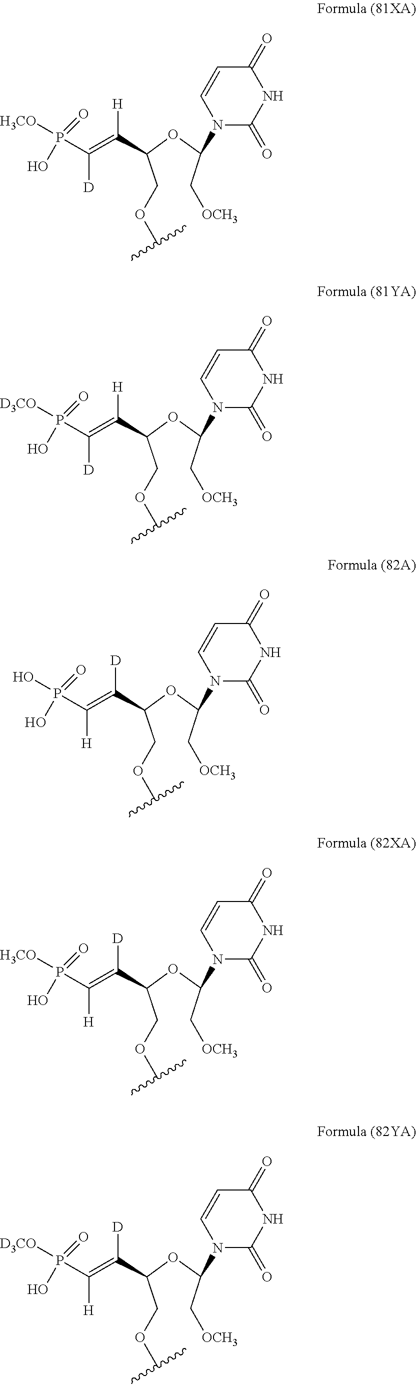

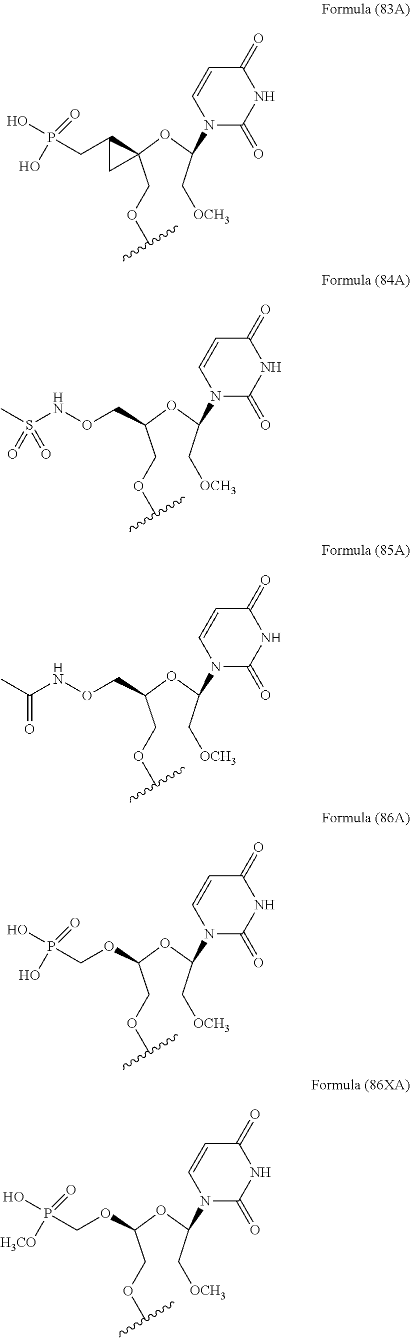

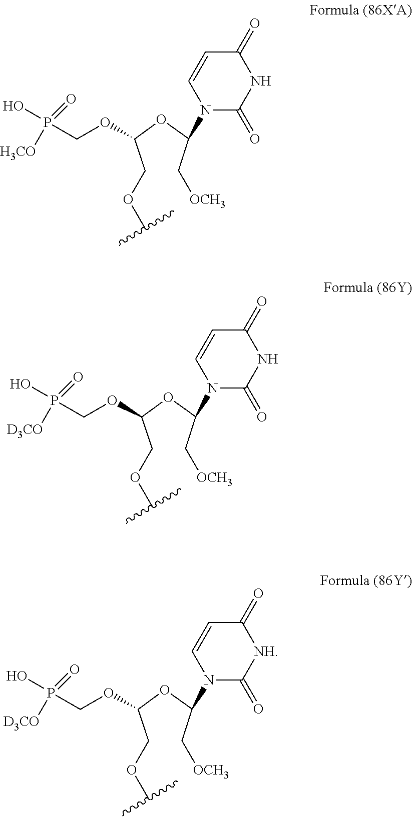

In some embodiments, any of the siNA molecules disclosed herein comprise a 5′-stabilized end cap selected from the group consisting of Formula (78A), Formula (79A), Formula (79XA), Formula (79YA), Formula (86A), Formula (86XA), and Formula (86X′A):

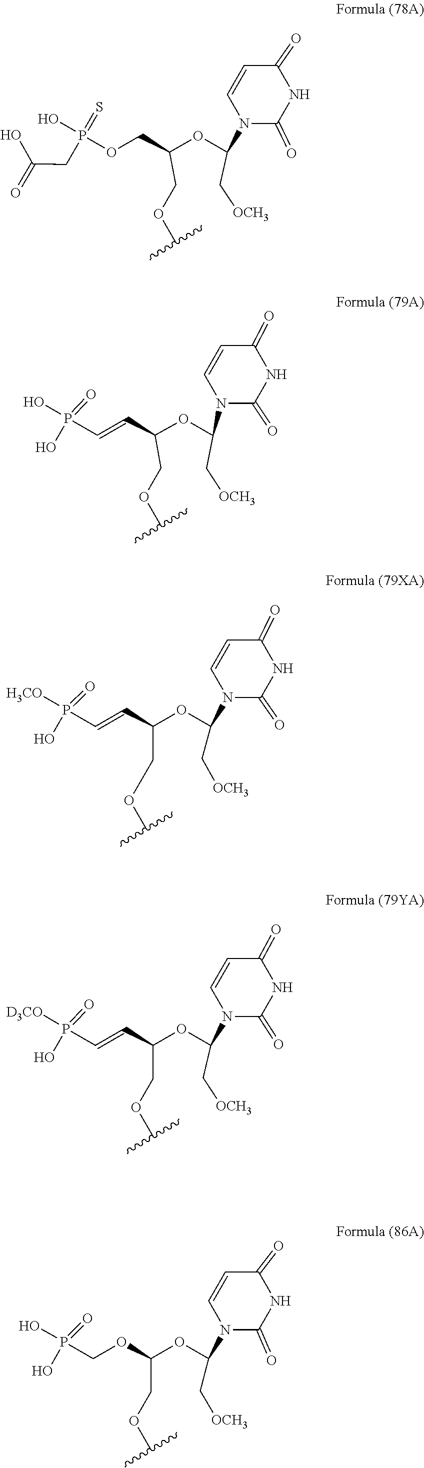

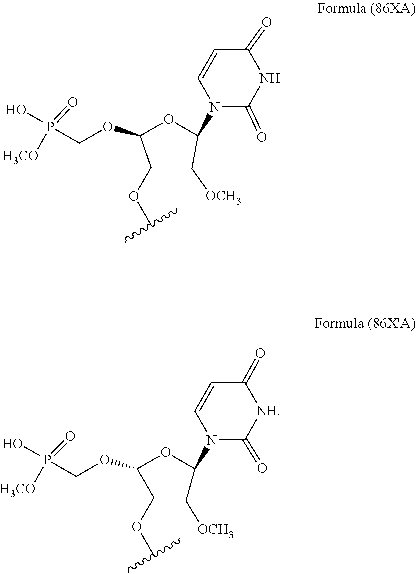 [SMILES: *OC[C@@H](OCP(=O)(O)OC)O[C@H](COC)n1ccc(=O)[nH]c1=O ; *OC[C@H](OCP(=O)(O)OC)O[C@H](COC)n1ccc(=O)[nH]c1=O]

In some embodiments, any of the siNA molecules disclosed herein may comprise a 5′ stabilized end cap with the structure:

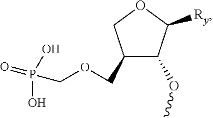

wherein R_{y} is a nucleobase.

In some embodiments, any of the siNA molecules disclosed herein may comprise a 5′ stabilized end cap with the structure:

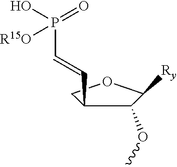

wherein R_{y} is a nucleobase and R¹⁵ is H or CH₃.

In some embodiments, the 5′-stabilized end cap is attached to the 5′ end of the antisense strand. In some embodiments, the 5′-stabilized end cap is attached to the 5′ end of the antisense strand via 1, 2, 3, 4, or 5 or more linkers. In some embodiments, the one or more linkers are independently selected from the group consisting of a phosphodiester (p or po) linker, phosphorothioate (ps) linker, mesyl phosphoramidate (Ms) linker, phosphoramidite (HEG) linker, triethylene glycol (TEG) linker, and/or phosphorodithioate linker. In some embodiments, the one or more linkers are independently selected from the group consisting of p-(PS)2, (PS)2-p-TEG-p, (PS)2-p-HEG-p, and (PS)2-p-(HEG-p)2.

As indicated above, the present disclosure provides compositions comprising any of the siNA molecules, sense strands, antisense strands, first nucleotide sequences, or second nucleotide sequences described herein. The disclosed siNA and compositions thereof can be used in the treatment of various diseases and conditions associated with ANGPTL3 and/or ANGPTL8.

### Internucleoside Linkages

In some embodiments, any of the siNAs, sense strands, first nucleotide sequences, antisense strands, and/or second nucleotide sequences disclosed herein comprise 1, 2, 3, 4, 5, 6, 7, 8, 9, 10, 11, 12, 13, 14, 15, 16, 17, 18, 19, 20, 21, 22, 23 or more internucleoside linkages. In some embodiments, 1, 2, 3, 4, 5, 6, 7, 8, 9, 10 or more internucleoside linkages are independently selected from the group consisting of a phosphodiester (p or po), phosphorothioate (ps), mesyl phosphoramidate (Ms), or phosphorodithioate.

In some embodiments, any of the siNAs, sense strands, first nucleotide sequences, antisense strands, and/or second nucleotide sequences disclosed herein further comprise 1, 2, 3, 4 or more linkages or other linkers that attach a conjugated moiety, phosphorylation blocker, and/or 5′ end cap to the siNA, sense strand, first nucleotide sequence, antisense strand, and/or second nucleotide sequences. In some embodiments, the 1, 2, 3, 4 or more linkers are independently selected from the group consisting of a phosphodiester (p or po) linker, phosphorothioate (ps) linker, mesyl phosphoramidate (Ms), phosphoramidite (HEG) linker, triethylene glycol (TEG) linker, and/or phosphorodithioate linker. In some embodiments, the one or more linkers are independently selected from the group consisting of p-(PS)2, (PS)2-p-TEG-p, (PS)2-p-HEG-p, and (PS)2-p-(HEG-p)2.

Examples of siNA Targeting ANGPTL3 or ANGPTL8

The tables below provide examples of siNA that target ANGPTL3 or ANGPTL8.

TABLE 1
ANGPTL8 siNA SS and AS Unmodified Sequences (5′ to 3′)

| | SEQ | | SEQ | |
|---|---|---|---|---|
| | ID | | ID | |
| Identifier | NO: | SS 5′->3′ | NO: | AS 5′->3′ |
| dsNA-001 | 1 | ACCCTCAGTCATGCCAGTGCC | 317 | GGCACTGGCATGACTGAGGGTCT |
| dsNA-002 | 2 | AGTCATGCCAGTGCCTGCTCT | 318 | AGAGCAGGCACTGGCATGACTGA |
| dsNA-003 | 3 | TGCCTGCTCTGTGCCTGCTCT | 319 | AGAGCAGGCACAGAGCAGGCACT |
| dsNA-004 | 4 | CTCTGTGCCTGCTCTGGGCCC | 320 | GGGCCCAGAGCAGGCACAGAGCA |
| dsNA-005 | 5 | GGCCCTGGCAATGGTGACCCG | 321 | CGGGTCACCATTGCCAGGGCCCA |
| dsNA-006 | 6 | CCAGAACTGGCACAGCATGAG | 322 | CTCATGCTGTGCCAGTTCTGGGC |
| dsNA-007 | 7 | CTGGCACAGCATGAGGAGCTG | 323 | CAGCTCCTCATGCTGTGCCAGTT |
| dsNA-008 | 8 | GCACAGCATGAGGAGCTGACC | 324 | GGTCAGCTCCTCATGCTGTGCCA |
| dsNA-009 | 9 | CTGACCCTGCTCTTCCATGGG | 325 | CCCATGGAAGAGCAGGGTCAGCT |
| dsNA-010 | 10 | ACCCTGCTCTTCCATGGGACC | 326 | GGTCCCATGGAAGAGCAGGGTCA |
| dsNA-011 | 11 | CTGCTCTTCCATGGGACCCTG | 327 | CAGGGTCCCATGGAAGAGCAGGG |
| dsNA-012 | 12 | CTCTTCCATGGGACCCTGCAG | 328 | CTGCAGGGTCCCATGGAAGAGCA |
| dsNA-013 | 13 | CATGGGACCCTGCAGCTGGGC | 329 | GCCCAGCTGCAGGGTCCCATGGA |
| dsNA-014 | 14 | AGCTGGGCCAGGCCCTCAACG | 330 | CGTTGAGGGCCTGGCCCAGCTGC |
| dsNA-015 | 15 | TGGGCCAGGCCCTCAACGGTG | 331 | CACCGTTGAGGGCCTGGCCCAGC |
| dsNA-016 | 16 | GCCAGGCCCTCAACGGTGTGT | 332 | ACACACCGTTGAGGGCCTGGCCC |
| dsNA-017 | 17 | GGCCCTCAACGGTGTGTACAG | 333 | CTGTACACACCGTTGAGGGCCTG |
| dsNA-018 | 18 | TGTGTACAGGACCACGGAGGG | 334 | CCCTCCGTGGTCCTGTACACACC |
| dsNA-019 | 19 | GTACAGGACCACGGAGGGACG | 335 | CGTCCCTCCGTGGTCCTGTACAC |
| dsNA-020 | 20 | AGGACCACGGAGGGACGGCTG | 336 | CAGCCGTCCCTCCGTGGTCCTGT |
| dsNA-021 | 21 | CCACGGAGGGACGGCTGACAA | 337 | TTGTCAGCCGTCCCTCCGTGGTC |
| dsNA-022 | 22 | CGGAGGGACGGCTGACAAAGG | 338 | CCTTTGTCAGCCGTCCCTCCGTG |
| dsNA-023 | 23 | AGGGACGGCTGACAAAGGCCA | 339 | TGGCCTTTGTCAGCCGTCCCTCC |
| dsNA-024 | 24 | CGGCTGACAAAGGCCAGGAAC | 340 | GTTCCTGGCCTTTGTCAGCCGTC |
| dsNA-025 | 25 | TGACAAAGGCCAGGAACAGCC | 341 | GGCTGTTCCTGGCCTTTGTCAGC |
| dsNA-026 | 26 | CAAAGGCCAGGAACAGCCTGG | 342 | CCAGGCTGTTCCTGGCCTTTGTC |
| dsNA-027 | 27 | AGGCCAGGAACAGCCTGGGTC | 343 | GACCCAGGCTGTTCCTGGCCTTT |
| dsNA-028 | 28 | CAGGAACAGCCTGGGTCTCTA | 344 | TAGAGACCCAGGCTGTTCCTGGC |
| dsNA-029 | 29 | GAACAGCCTGGGTCTCTATGG | 345 | CCATAGAGACCCAGGCTGTTCCT |
| dsNA-030 | 30 | AGCCTGGGTCTCTATGGCCGC | 346 | GCGGCCATAGAGACCCAGGCTGT |
| dsNA-031 | 31 | CTGGGTCTCTATGGCCGCACA | 347 | TGTGCGGCCATAGAGACCCAGGC |
| dsNA-032 | 32 | GAACTCCTGGGGCAGGAGGTC | 348 | GACCTCCTGCCCCAGGAGTTCTA |
| dsNA-033 | 33 | CCTGGGGCAGGAGGTCAGCCG | 349 | CGGCTGACCTCCTGCCCCAGGAG |
| dsNA-034 | 34 | GGAGGTCAGCCGGGGCCGGGA | 350 | TCCCGGCCCCGGCTGACCTCCTG |
| dsNA-035 | 35 | GGTCAGCCGGGGCCGGGATGC | 351 | GCATCCCGGCCCCGGCTGACCTC |
| dsNA-036 | 36 | CAGCCGGGGCCGGGATGCAGC | 352 | GCTGCATCCCGGCCCCGGCTGAC |
| dsNA-037 | 37 | GCCGGGATGCAGCCCAGGAAC | 353 | GTTCCTGGGCTGCATCCCGGCCC |
| dsNA-038 | 38 | GGGATGCAGCCCAGGAACTTC | 354 | GAAGTTCCTGGGCTGCATCCCGG |
| dsNA-039 | 39 | ATGCAGCCCAGGAACTTCGGG | 355 | CCCGAAGTTCCTGGGCTGCATCC |
| dsNA-040 | 40 | CAGCCCAGGAACTTCGGGCAA | 356 | TTGCCCGAAGTTCCTGGGCTGCA |
| dsNA-041 | 41 | CCCAGGAACTTCGGGCAAGCC | 357 | GGCTTGCCCGAAGTTCCTGGGCT |
| dsNA-042 | 42 | AGGAACTTCGGGCAAGCCTGT | 358 | ACAGGCTTGCCCGAAGTTCCTGG |
| dsNA-043 | 43 | AACTTCGGGCAAGCCTGTTGG | 359 | CCAACAGGCTTGCCCGAAGTTCC |
| dsNA-044 | 44 | GGGCAAGCCTGTTGGAGACTC | 360 | GAGTCTCCAACAGGCTTGCCCGA |
| dsNA-045 | 45 | CAAGCCTGTTGGAGACTCAGA | 361 | TCTGAGTCTCCAACAGGCTTGCC |
| dsNA-046 | 46 | GCCTGTTGGAGACTCAGATGG | 362 | CCATCTGAGTCTCCAACAGGCTT |
| dsNA-047 | 47 | TGTTGGAGACTCAGATGGAGG | 363 | CCTCCATCTGAGTCTCCAACAGG |
| dsNA-048 | 48 | GAGACTCAGATGGAGGAGGAT | 364 | ATCCTCCTCCATCTGAGTCTCCA |
| dsNA-049 | 49 | CTCAGATGGAGGAGGATATTC | 365 | GAATATCCTCCTCCATCTGAGTC |
| dsNA-050 | 50 | GGAGGAGGATATTCTGCAGCT | 366 | AGCTGCAGAATATCCTCCTCCAT |
| dsNA-051 | 51 | GGATATTCTGCAGCTGCAGGC | 367 | GCCTGCAGCTGCAGAATATCCTC |
| dsNA-052 | 52 | GGCCCAGGCACAGAAGGTGCT | 368 | AGCACCTTCTGTGCCTGGGCCAC |
| dsNA-053 | 53 | CCAGGCACAGAAGGTGCTACG | 369 | CGTAGCACCTTCTGTGCCTGGGC |
| dsNA-054 | 54 | GGCACAGAAGGTGCTACGGGA | 370 | TCCCGTAGCACCTTCTGTGCCTG |
| dsNA-055 | 55 | ACAGAAGGTGCTACGGGACAG | 371 | CTGTCCCGTAGCACCTTCTGTGC |
| dsNA-056 | 56 | GAAGGTGCTACGGGACAGCGT | 372 | ACGCTGTCCCGTAGCACCTTCTG |
| dsNA-057 | 57 | GGTGCTACGGGACAGCGTGCA | 373 | TGCACGCTGTCCCGTAGCACCTT |
| dsNA-058 | 58 | CAGCGTGCAGCGGCTAGAAGT | 374 | ACTTCTAGCCGCTGCACGCTGTC |
| dsNA-059 | 59 | CGTGCAGCGGCTAGAAGTCCA | 375 | TGGACTTCTAGCCGCTGCACGCT |
| dsNA-060 | 60 | GCAGCGGCTAGAAGTCCAGCT | 376 | AGCTGGACTTCTAGCCGCTGCAC |
| dsNA-061 | 61 | GCGGCTAGAAGTCCAGCTGAG | 377 | CTCAGCTGGACTTCTAGCCGCTG |
| dsNA-062 | 62 | CTAGAAGTCCAGCTGAGGAGC | 378 | GCTCCTCAGCTGGACTTCTAGCC |
| dsNA-063 | 63 | AAGTCCAGCTGAGGAGCGCCT | 379 | AGGCGCTCCTCAGCTGGACTTCT |
| dsNA-064 | 64 | TCCAGCTGAGGAGCGCCTGGC | 380 | GCCAGGCGCTCCTCAGCTGGACT |
| dsNA-065 | 65 | CTGAGGAGCGCCTGGCTGGGC | 381 | GCCCAGCCAGGCGCTCCTCAGCT |
| dsNA-066 | 66 | AGGAGCGCCTGGCTGGGCCCT | 382 | AGGGCCCAGCCAGGCGCTCCTCA |
| dsNA-067 | 67 | GCCCTGCCTACCGAGAATTTG | 383 | CAAATTCTCGGTAGGCAGGGCCC |
| dsNA-068 | 68 | TGCCTACCGAGAATTTGAGGT | 384 | ACCTCAAATTCTCGGTAGGCAGG |
| dsNA-069 | 69 | CTACCGAGAATTTGAGGTCTT | 385 | AAGACCTCAAATTCTCGGTAGGC |
| dsNA-070 | 70 | CGAGAATTTGAGGTCTTAAAG | 386 | CTTTAAGACCTCAAATTCTCGGT |
| dsNA-071 | 71 | GAATTTGAGGTCTTAAAGGCT | 387 | AGCCTTTAAGACCTCAAATTCTC |
| dsNA-072 | 72 | TTGAGGTCTTAAAGGCTCACG | 388 | CGTGAGCCTTTAAGACCTCAAAT |
| dsNA-073 | 73 | CTTAAAGGCTCACGCTGACAA | 389 | TTGTCAGCGTGAGCCTTTAAGAC |
| dsNA-074 | 74 | AAAGGCTCACGCTGACAAGCA | 390 | TGCTTGTCAGCGTGAGCCTTTAA |
| dsNA-075 | 75 | GGCTCACGCTGACAAGCAGAG | 391 | CTCTGCTTGTCAGCGTGAGCCTT |
| dsNA-076 | 76 | GCTGACAAGCAGAGCCACATC | 392 | GATGTGGCTCTGCTTGTCAGCGT |
| dsNA-077 | 77 | ACAAGCAGAGCCACATCCTAT | 393 | ATAGGATGTGGCTCTGCTTGTCA |
| dsNA-078 | 78 | AGCAGAGCCACATCCTATGGG | 394 | CCCATAGGATGTGGCTCTGCTTG |
| dsNA-079 | 79 | AGAGCCACATCCTATGGGCCC | 395 | GGGCCCATAGGATGTGGCTCTGC |
| dsNA-080 | 80 | ACATCCTATGGGCCCTCACAG | 396 | CTGTGAGGGCCCATAGGATGTGG |
| dsNA-081 | 81 | CTATGGGCCCTCACAGGCCAC | 397 | GTGGCCTGTGAGGGCCCATAGGA |
| dsNA-082 | 82 | TGGGCCCTCACAGGCCACGTG | 398 | CACGTGGCCTGTGAGGGCCCATA |
| dsNA-083 | 83 | CCCTCACAGGCCACGTGCAGC | 399 | GCTGCACGTGGCCTGTGAGGGCC |
| dsNA-084 | 84 | CACAGGCCACGTGCAGCGGCA | 400 | TGCCGCTGCACGTGGCCTGTGAG |
| dsNA-085 | 85 | AGGCCACGTGCAGCGGCAGAG | 401 | CTCTGCCGCTGCACGTGGCCTGT |
| dsNA-086 | 86 | CACGTGCAGCGGCAGAGGCGG | 402 | CCGCCTCTGCCGCTGCACGTGGC |
| dsNA-087 | 87 | GTGCAGCGGCAGAGGCGGGAG | 403 | CTCCCGCCTCTGCCGCTGCACGT |
| dsNA-088 | 88 | CAGCGGCAGAGGCGGGAGATG | 404 | CATCTCCCGCCTCTGCCGCTGCA |
| dsNA-089 | 89 | GGCAGAGGCGGGAGATGGTGG | 405 | CCACCATCTCCCGCCTCTGCCGC |
| dsNA-090 | 90 | GCGGGAGATGGTGGCACAGCA | 406 | TGCTGTGCCACCATCTCCCGCCT |
| dsNA-091 | 91 | GAGATGGTGGCACAGCAGCAT | 407 | ATGCTGCTGTGCCACCATCTCCC |
| dsNA-092 | 92 | TGGTGGCACAGCAGCATCGGC | 408 | GCCGATGCTGCTGTGCCACCATC |
| dsNA-093 | 93 | TGGCACAGCAGCATCGGCTGC | 409 | GCAGCCGATGCTGCTGTGCCACC |
| dsNA-094 | 94 | CACAGCAGCATCGGCTGCGAC | 410 | GTCGCAGCCGATGCTGCTGTGCC |
| dsNA-095 | 95 | CAGCATCGGCTGCGACAGATC | 411 | GATCTGTCGCAGCCGATGCTGCT |
| dsNA-096 | 96 | CATCGGCTGCGACAGATCCAG | 412 | CTGGATCTGTCGCAGCCGATGCT |
| dsNA-097 | 97 | CGGCTGCGACAGATCCAGGAG | 413 | CTCCTGGATCTGTCGCAGCCGAT |
| dsNA-098 | 98 | GACAGATCCAGGAGAGACTCC | 414 | GGAGTCTCTCCTGGATCTGTCGC |
| dsNA-099 | 99 | AGATCCAGGAGAGACTCCACA | 415 | TGTGGAGTCTCTCCTGGATCTGT |
| dsNA-100 | 100 | AGCGGCGCTCCCAGCCTGAAT | 416 | ATTCAGGCTGGGAGCGCCGCTGT |
| dsNA-101 | 101 | GGCGCTCCCAGCCTGAATCTG | 417 | CAGATTCAGGCTGGGAGCGCCGC |
| dsNA-102 | 102 | GCTCCCAGCCTGAATCTGCCT | 418 | AGGCAGATTCAGGCTGGGAGCGC |
| dsNA-103 | 103 | AGCCTGAATCTGCCTGGATGG | 419 | CCATCCAGGCAGATTCAGGCTGG |
| dsNA-104 | 104 | CTGAATCTGCCTGGATGGAAC | 420 | GTTCCATCCAGGCAGATTCAGGC |
| dsNA-105 | 105 | ATCTGCCTGGATGGAACTGAG | 421 | CTCAGTTCCATCCAGGCAGATTC |
| dsNA-106 | 106 | GCCTGGATGGAACTGAGGACC | 422 | GGTCCTCAGTTCCATCCAGGCAG |
| dsNA-107 | 107 | GGATGGAACTGAGGACCAATC | 423 | GATTGGTCCTCAGTTCCATCCAG |
| dsNA-108 | 108 | GAACTGAGGACCAATCATGCT | 424 | AGCATGATTGGTCCTCAGTTCCA |
| dsNA-109 | 109 | GAGGACCAATCATGCTGCAAG | 425 | CTTGCAGCATGATTGGTCCTCAG |
| dsNA-110 | 110 | ACCAATCATGCTGCAAGGAAC | 426 | GTTCCTTGCAGCATGATTGGTCC |
| dsNA-111 | 111 | TCATGCTGCAAGGAACACTTC | 427 | GAAGTGTTCCTTGCAGCATGATT |
| dsNA-112 | 112 | CTGCAAGGAACACTTCCACGC | 428 | GCGTGGAAGTGTTCCTTGCAGCA |
| dsNA-113 | 113 | AAGGAACACTTCCACGCCCCG | 429 | CGGGGCGTGGAAGTGTTCCTTGC |
| dsNA-114 | 114 | AACACTTCCACGCCCCGTGAG | 430 | CTCACGGGGCGTGGAAGTGTTCC |
| dsNA-115 | 115 | ACTTCCACGCCCCGTGAGGCC | 431 | GGCCTCACGGGGCGTGGAAGTGT |
| dsNA-116 | 116 | TCCACGCCCCGTGAGGCCCCT | 432 | AGGGGCCTCACGGGGCGTGGAAG |
| dsNA-117 | 117 | GGAGGAGCTGCCTGTTCACTG | 433 | CAGTGAACAGGCAGCTCCTCCCT |
| dsNA-118 | 118 | GGAGCTGCCTGTTCACTGGGA | 434 | TCCCAGTGAACAGGCAGCTCCTC |
| dsNA-119 | 119 | GCTGCCTGTTCACTGGGATCA | 435 | TGATCCCAGTGAACAGGCAGCTC |
| dsNA-120 | 120 | GCCTGTTCACTGGGATCAGCC | 436 | GGCTGATCCCAGTGAACAGGCAG |
| dsNA-121 | 121 | GTTCACTGGGATCAGCCAGGG | 437 | CCCTGGCTGATCCCAGTGAACAG |
| dsNA-122 | 122 | ACTGGGATCAGCCAGGGCGCC | 438 | GGCGCCCTGGCTGATCCCAGTGA |
| dsNA-123 | 123 | GGGATCAGCCAGGGCGCCGGG | 439 | CCCGGCGCCCTGGCTGATCCCAG |
| dsNA-124 | 124 | TCAGCCAGGGCGCCGGGCCCC | 440 | GGGGCCCGGCGCCCTGGCTGATC |
| dsNA-125 | 125 | GGGCGCCGGGCCCCACTTCTG | 441 | CAGAAGTGGGGCCCGGCGCCCTG |
| dsNA-126 | 126 | GCCGGGCCCCACTTCTGAGCA | 442 | TGCTCAGAAGTGGGGCCCGGCGC |
| dsNA-127 | 127 | GGGCCCCACTTCTGAGCACAG | 443 | CTGTGCTCAGAAGTGGGGCCCGG |
| dsNA-128 | 128 | CCCCACTTCTGAGCACAGAGC | 444 | GCTCTGTGCTCAGAAGTGGGGCC |
| dsNA-129 | 129 | TTCTGAGCACAGAGCAGAGAC | 445 | GTCTCTGCTCTGTGCTCAGAAGT |
| dsNA-130 | 130 | TGAGCACAGAGCAGAGACAGA | 446 | TCTGTCTCTGCTCTGTGCTCAGA |
| dsNA-131 | 131 | GCACAGAGCAGAGACAGACGC | 447 | GCGTCTGTCTCTGCTCTGTGCTC |
| dsNA-132 | 132 | CAGAGCAGAGACAGACGCAGG | 448 | CCTGCGTCTGTCTCTGCTCTGTG |
| dsNA-133 | 133 | AGCAGAGACAGACGCAGGCGG | 449 | CCGCCTGCGTCTGTCTCTGCTCT |
| dsNA-134 | 134 | AGACAGACGCAGGCGGGGACA | 450 | TGTCCCCGCCTGCGTCTGTCTCT |
| dsNA-135 | 135 | CAGACGCAGGCGGGGACAAAG | 451 | CTTTGTCCCCGCCTGCGTCTGTC |
| dsNA-136 | 136 | ACGCAGGCGGGGACAAAGGCA | 452 | TGCCTTTGTCCCCGCCTGCGTCT |
| dsNA-137 | 137 | AGGCGGGGACAAAGGCAGAGG | 453 | CCTCTGCCTTTGTCCCCGCCTGC |
| dsNA-138 | 138 | CAAAGGCAGAGGATGTAGCCC | 454 | GGGCTACATCCTCTGCCTTTGTC |
| dsNA-139 | 139 | AGGCAGAGGATGTAGCCCCAT | 455 | ATGGGGCTACATCCTCTGCCTTT |
| dsNA-140 | 140 | CAGAGGATGTAGCCCCATTGG | 456 | CCAATGGGGCTACATCCTCTGCC |
| dsNA-141 | 141 | AGGATGTAGCCCCATTGGGGA | 457 | TCCCCAATGGGGCTACATCCTCT |
| dsNA-142 | 142 | ATGTAGCCCCATTGGGGAGGG | 458 | CCCTCCCCAATGGGGCTACATCC |
| dsNA-143 | 143 | GCCCCATTGGGGAGGGGTGGA | 459 | TCCACCCCTCCCCAATGGGGCTA |
| dsNA-144 | 144 | TTGGGGAGGGGTGGAGGAAGG | 460 | CCTTCCTCCACCCCTCCCCAATG |
| dsNA-145 | 145 | CCTCATTAAAGCAGAGTCGTG | 461 | CACGACTCTGCTTTAATGAGGGG |
| dsNA-146 | 146 | CATTAAAGCAGAGTCGTGGCA | 462 | TGCCACGACTCTGCTTTAATGAG |
| dsNA-147 | 147 | TAAAGCAGAGTCGTGGCATCT | 463 | AGATGCCACGACTCTGCTTTAAT |
| dsNA-148 | 148 | GGAGGAGGATATTCTGCAGCT | 464 | AGCTGCAGAATATCCTCCTCCAT |
| dsNA-149 | 149 | CGAGAATTTGAGGTCTTAAAT | 465 | ATTTAAGACCTCAAATTCTCGGT |
| dsNA-150 | 150 | GAATTTGAGGTCTTAAAGGCT | 466 | AGCCTTTAAGACCTCAAATTCTC |
| dsNA-151 | 151 | ACAAGCAGAGCCACATCCTAT | 467 | ATAGGATGTGGCTCTGCTTGTCA |
| dsNA-152 | 152 | CTGAATCTGCCTGGATGGAAT | 468 | ATTCCATCCAGGCAGATTCAGGC |
| dsNA-153 | 153 | CATTAAAGCAGAGTCGTGGCT | 469 | AGCCACGACTCTGCTTTAATGAG |
| dsNA-154 | 154 | CAGGAACAGCCTGGGTCTCTT | 470 | AAGAGACCCAGGCTGTTCCTGGC |
| dsNA-155 | 155 | CTACCGAGAATTTGAGGTCTT | 471 | AAGACCTCAAATTCTCGGTAGGC |
| dsNA-156 | 156 | GCTGACAAGCAGAGCCACATT | 472 | AATGTGGCTCTGCTTGTCAGCGT |
| dsNA-157 | 157 | GAGATGGTGGCACAGCAGCAT | 473 | ATGCTGCTGTGCCACCATCTCCC |
| dsNA-158 | 158 | CCTCATTAAAGCAGAGTCGTT | 474 | AACGACTCTGCTTTAATGAGGGG |

TABLE 2
ANGPTL8 siNA SS and AS Sequences with Nucleotide Modifications

| | SEQ | | SEQ | |
|---|---|---|---|---|
| | ID | | ID | |
| Identifier | NO: | SS 5′->3′ | NO: | AS 5′->3′ |
| dsNA-001 | 159 | mApsmCpsmCmCmUmCfAmGfUfCfA | 475 | vmApsfGpsmCmAmCfUmGmGmCmAmUm |
| | | mUmGmCmCmAmGmUmGmCpsmU | | GmAfCmUfGmAmGmGmGmUpsmCpsmU |
| dsNA-002 | 160 | mApsmGpsmUmCmAmUfGmCfCfAfG | 476 | vmApsfGpsmAmGmCfAmGmGmCmAmCm |
| | | mUmGmCmCmUmGmCmUmCpsmU | | UmGfGmCfAmUmGmAmCmUpsmGpsmA |
| dsNA-003 | 161 | mUpsmGpsmCmCmUmGfCmUfCfUfG | 477 | vmApsfGpsmAmGmCfAmGmGmCmAmCm |
| | | mUmGmCmCmUmGmCmUmCpsmU | | AmGfAmGfCmAmGmGmCmApsmCpsmU |
| dsNA-004 | 162 | mCpsmUpsmCmUmGmUfGmCfCfUfG | 478 | vmApsfGpsmGmCmCfCmAmGmAmGmCm |
| | | mCmUmCmUmGmGmGmCmCpsmU | | AmGfGmCfAmCmAmGmAmGpsmCpsmA |
| dsNA-005 | 163 | mGpsmGpsmCmCmCmUfGmGfCfAfA | 479 | vmApsfGpsmGmGmUfCmAmCmCmAmUm |
| | | mUmGmGmUmGmAmCmCmCpsmU | | UmGfCmCfAmGmGmGmCmCpsmCpsmA |
| dsNA-006 | 164 | mCpsmCpsmAmGmAmAfCmUfGfGfC | 480 | vmApsfUpsmCmAmUfGmCmUmGmUmGm |
| | | mAmCmAmGmCmAmUmGmApsmU | | CmCfAmGfUmUmCmUmGmGpsmGpsmC |
| dsNA-007 | 165 | mCpsmUpsmGmGmCmAfCmAfGfCfA | 481 | vmApsfApsmGmCmUfCmCmUmCmAmUm |
| | | mUmGmAmGmGmAmGmCmUpsmU | | GmCfUmGfUmGmCmCmAmGpsmUpsmU |
| dsNA-008 | 166 | mGpsmCpsmAmCmAmGfCmAfUfGfA | 482 | vmApsfGpsmUmCmAfGmCmUmCmCmUm |
| | | mGmGmAmGmCmUmGmAmCpsmU | | CmAfUmGfCmUmGmUmGmCpsmCpsmA |
| dsNA-009 | 167 | mCpsmUpsmGmAmCmCfCmUfGfCfU | 483 | vmApsfCpsmCmAmUfGmGmAmAmGmAm |
| | | mCmUmUmCmCmAmUmGmGpsmU | | GmCfAmGfGmGmUmCmAmGpsmCpsmU |
| dsNA-010 | 168 | mApsmCpsmCmCmUmGfCmUfCfUfU | 484 | vmApsfGpsmUmCmCfCmAmUmGmGmAm |
| | | mCmCmAmUmGmGmGmAmCpsmU | | AmGfAmGfCmAmGmGmGmUpsmCpsmA |
| dsNA-011 | 169 | mCpsmUpsmGmCmUmCfUmUfCfCfA | 485 | vmApsfApsmGmGmGfUmCmCmCmAmUm |
| | | mUmGmGmGmAmCmCmCmUpsmU | | GmGfAmAfGmAmGmCmAmGpsmGpsmG |
| dsNA-012 | 170 | mCpsmUpsmCmUmUmCfCmAfUfGfG | 486 | vmApsfUpsmGmCmAfGmGmGmUmCmCm |
| | | mGmAmCmCmCmUmGmCmApsmU | | CmAfUmGfGmAmAmGmAmGpsmCpsmA |
| dsNA-013 | 171 | mCpsmApsmUmGmGmGfAmCfCfCfU | 487 | vmApsfCpsmCmCmAfGmCmUmGmCmAm |
| | | mGmCmAmGmCmUmGmGmGpsmU | | GmGfGmUfCmCmCmAmUmGpsmGpsmA |
| dsNA-014 | 172 | mApsmGpsmCmUmGmGfGmCfCfAfG | 488 | vmApsfGpsmUmUmGfAmGmGmGmCmCm |
| | | mGmCmCmCmUmCmAmAmCpsmU | | UmGfGmCfCmCmAmGmCmUpsmGpsmC |
| dsNA-015 | 173 | mUpsmGpsmGmGmCmCfAmGfGfCfC | 489 | vmApsfApsmCmCmGfUmUmGmAmGmGm |
| | | mCmUmCmAmAmCmGmGmUpsmU | | GmCfCmUfGmGmCmCmCmApsmGpsmC |
| dsNA-016 | 174 | mGpsmCpsmCmAmGmGfCmCfCfUfC | 490 | vmApsfCpsmAmCmAfCmCmGmUmUmGm |
| | | mAmAmCmGmGmUmGmUmGpsmU | | AmGfGmGfCmCmUmGmGmCpsmCpsmC |
| dsNA-017 | 175 | mGpsmGpsmCmCmCmUfCmAfAfCfG | 491 | vmApsfUpsmGmUmAfCmAmCmAmCmCm |
| | | mGmUmGmUmGmUmAmCmApsmU | | GmUfUmGfAmGmGmGmCmCpsmUpsmG |
| dsNA-018 | 176 | mUpsmGpsmUmGmUmAfCmAfGfGfA | 492 | vmApsfCpsmCmUmCfCmGmUmGmGmUm |
| | | mCmCmAmCmGmGmAmGmGpsmU | | CmCfUmGfUmAmCmAmCmApsmCpsmC |
| dsNA-019 | 177 | mGpsmUpsmAmCmAmGfGmAfCfCfA | 493 | vmApsfGpsmUmCmCfCmUmCmCmGmUm |
| | | mCmGmGmAmGmGmGmAmCpsmU | | GmGfUmCfCmUmGmUmAmCpsmApsmC |
| dsNA-020 | 178 | mApsmGpsmGmAmCmCfAmCfGfGfA | 494 | vmApsfApsmGmCmCfGmUmCmCmCmUmC |
| | | mGmGmGmAmCmGmGmCmUpsmU | | mCfGmUfGmGmUmCmCmUpsmGpsmU |
| dsNA-021 | 179 | mCpsmCpsmAmCmGmGfAmGfGfGfA | 495 | vmApsfUpsmGmUmCfAmGmCmCmGmUm |
| | | mCmGmGmCmUmGmAmCmApsmU | | CmCfCmUfCmCmGmUmGmGpsmUpsmC |
| dsNA-022 | 180 | mCpsmGpsmGmAmGmGfGmAfCfGfG | 496 | vmApsfCpsmUmUmUfGmUmCmAmGmCm |
| | | mCmUmGmAmCmAmAmAmGpsmU | | CmGfUmCfCmCmUmCmCmGpsmUpsmG |
| dsNA-023 | 181 | mApsmGpsmGmGmAmCfGmGfCfUfG | 497 | vmApsfGpsmGmCmCfUmUmUmGmUmCm |
| | | mAmCmAmAmAmGmGmCmCpsmU | | AmGfCmCfGmUmCmCmCmUpsmCpsmC |
| dsNA-024 | 182 | mCpsmGpsmGmCmUmGfAmCfAfAfA | 498 | vmApsfUpsmUmCmCfUmGmGmCmCmUm |
| | | mGmGmCmCmAmGmGmAmApsmU | | UmUfGmUfCmAmGmCmCmGpsmUpsmC |
| dsNA-025 | 183 | mUpsmGpsmAmCmAmAfAmGfGfCfC | 499 | vmApsfGpsmCmUmGfUmUmCmCmUmGm |
| | | mAmGmGmAmAmCmAmGmCpsmU | | GmCfCmUfUmUmGmUmCmApsmGpsmC |
| dsNA-026 | 184 | mCpsmApsmAmAmGmGfCmCfAfGfG | 500 | vmApsfCpsmAmGmGfCmUmGmUmUmCm |
| | | mAmAmCmAmGmCmCmUmGpsmU | | CmUfGmGfCmCmUmUmUmGpsmUpsmC |
| dsNA-027 | 185 | mApsmGpsmGmCmCmAfGmGfAfAfC | 501 | vmApsfApsmCmCmCfAmGmGmCmUmGm |
| | | mAmGmCmCmUmGmGmGmUpsmU | | UmUfCmCfUmGmGmCmCmUpsmUpsmU |
| dsNA-028 | 186 | mCpsmApsmGmGmAmAfCmAfGfCfC | 502 | vmApsfApsmGmAmGfAmCmCmCmAmGm |
| | | mUmGmGmGmUmCmUmCmUpsmU | | GmCfUmGfUmUmCmCmUmGpsmGpsmC |
| dsNA-029 | 187 | mGpsmApsmAmCmAmGfCmCfUfGfG | 503 | vmApsfCpsmAmUmAfGmAmGmAmCmCm |
| | | mGmUmCmUmCmUmAmUmGpsmU | | CmAfGmGfCmUmGmUmUmCpsmCpsmU |
| dsNA-030 | 188 | mApsmGpsmCmCmUmGfGmGfUfCfU | 504 | vmApsfCpsmGmGmCfCmAmUmAmGmAm |
| | | mCmUmAmUmGmGmCmCmGpsmU | | GmAfCmCfCmAmGmGmCmUpsmGpsmU |
| dsNA-031 | 189 | mCpsmUpsmGmGmGmUfCmUfCfUfA | 505 | vmApsfGpsmUmGmCfGmGmCmCmAmUm |
| | | mUmGmGmCmCmGmCmAmCpsmU | | AmGfAmGfAmCmCmCmAmGpsmGpsmC |
| dsNA-032 | 190 | mGpsmApsmAmCmUmCfCmUfGfGfG | 506 | vmApsfApsmCmCmUfCmCmUmGmCmCmC |
| | | mGmCmAmGmGmAmGmGmUpsmU | | mCfAmGfGmAmGmUmUmCpsmUpsmA |
| dsNA-033 | 191 | mCpsmCpsmUmGmGmGfGmCfAfGfG | 507 | vmApsfGpsmGmCmUfGmAmCmCmUmCm |
| | | mAmGmGmUmCmAmGmCmCpsmU | | CmUfGmCfCmCmCmAmGmGpsmApsmG |
| dsNA-034 | 192 | mGpsmGpsmAmGmGmUfCmAfGfCfC | 508 | vmApsfCpsmCmCmGfGmCmCmCmCmGmG |
| | | mGmGmGmGmCmCmGmGmGpsmU | | mCfUmGfAmCmCmUmCmCpsmUpsmG |
| dsNA-035 | 193 | mGpsmGpsmUmCmAmGfCmCfGfGfG | 509 | vmApsfCpsmAmUmCfCmCmGmGmCmCmC |
| | | mGmCmCmGmGmGmAmUmGpsmU | | mCfGmGfCmUmGmAmCmCpsmUpsmC |
| dsNA-036 | 194 | mCpsmApsmGmCmCmGfGmGfGfCfC | 510 | vmApsfCpsmUmGmCfAmUmCmCmCmGm |
| | | mGmGmGmAmUmGmCmAmGpsmU | | GmCfCmCfCmGmGmCmUmGpsmApsmC |
| dsNA-037 | 195 | mGpsmCpsmCmGmGmGfAmUfGfCfA | 511 | vmApsfUpsmUmCmCfUmGmGmGmCmUm |
| | | mGmCmCmCmAmGmGmAmApsmU | | GmCfAmUfCmCmCmGmGmCpsmCpsmC |
| dsNA-038 | 196 | mGpsmGpsmGmAmUmGfCmAfGfCfC | 512 | vmApsfApsmAmGmUfUmCmCmUmGmGm |
| | | mCmAmGmGmAmAmCmUmUpsmU | | GmCfUmGfCmAmUmCmCmCpsmGpsmG |
| dsNA-039 | 197 | mApsmUpsmGmCmAmGfCmCfCfAfG | 513 | vmApsfCpsmCmGmAfAmGmUmUmCmCm |
| | | mGmAmAmCmUmUmCmGmGpsmU | | UmGfGmGfCmUmGmCmAmUpsmCpsmC |
| dsNA-040 | 198 | mCpsmApsmGmCmCmCfAmGfGfAfA | 514 | vmApsfUpsmGmCmCfCmGmAmAmGmUm |
| | | mCmUmUmCmGmGmGmCmApsmU | | UmCfCmUfGmGmGmCmUmGpsmCpsmA |
| dsNA-041 | 199 | mCpsmCpsmCmAmGmGfAmAfCfUfU | 515 | vmApsfGpsmCmUmUfGmCmCmCmGmAm |
| | | mCmGmGmGmCmAmAmGmCpsmU | | AmGfUmUfCmCmUmGmGmGpsmCpsmU |
| dsNA-042 | 200 | mApsmGpsmGmAmAmCfUmUfCfGfG | 516 | vmApsfCpsmAmGmGfCmUmUmGmCmCm |
| | | mGmCmAmAmGmCmCmUmGpsmU | | CmGfAmAfGmUmUmCmCmUpsmGpsmG |
| dsNA-043 | 201 | mApsmApsmCmUmUmCfGmGfGfCfA | 517 | vmApsfCpsmAmAmCfAmGmGmCmUmUm |
| | | mAmGmCmCmUmGmUmUmGpsmU | | GmCfCmCfGmAmAmGmUmUpsmCpsmC |
| dsNA-044 | 202 | mGpsmGpsmGmCmAmAfGmCfCfUfG | 518 | vmApsfApsmGmUmCfUmCmCmAmAmCm |
| | | mUmUmGmGmAmGmAmCmUpsmU | | AmGfGmCfUmUmGmCmCmCpsmGpsmA |
| dsNA-045 | 203 | mCpsmApsmAmGmCmCfUmGfUfUfG | 519 | vmApsfCpsmUmGmAfGmUmCmUmCmCm |
| | | mGmAmGmAmCmUmCmAmGpsmU | | AmAfCmAfGmGmCmUmUmGpsmCpsmC |
| dsNA-046 | 204 | mGpsmCpsmCmUmGmUfUmGfGfAfG | 520 | vmApsfCpsmAmUmCfUmGmAmGmUmCm |
| | | mAmCmUmCmAmGmAmUmGpsmU | | UmCfCmAfAmCmAmGmGmCpsmUpsmU |
| dsNA-047 | 205 | mUpsmGpsmUmUmGmGfAmGfAfCfU | 521 | vmApsfCpsmUmCmCfAmUmCmUmGmAm |
| | | mCmAmGmAmUmGmGmAmGpsmU | | GmUfCmUfCmCmAmAmCmApsmGpsmG |
| dsNA-048 | 206 | mGpsmApsmGmAmCmUfCmAfGfAfU | 522 | vmApsfUpsmCmCmUfCmCmUmCmCmAmU |
| | | mGmGmAmGmGmAmGmGmApsmU | | mCfUmGfAmGmUmCmUmCpsmCpsmA |
| dsNA-049 | 207 | mCpsmUpsmCmAmGmAfUmGfGfAfG | 523 | vmApsfApsmAmUmAfUmCmCmUmCmCm |
| | | mGmAmGmGmAmUmAmUmUpsmU | | UmCfCmAfUmCmUmGmAmGpsmUpsmC |
| dsNA-050 | 208 | mGpsmGpsmAmGmGmAfGmGfAfUfA | 524 | vmApsfGpsmCmUmGfCmAmGmAmAmUm |
| | | mUmUmCmUmGmCmAmGmCpsmU | | AmUfCmCfUmCmCmUmCmCpsmApsmU |
| dsNA-051 | 209 | mGpsmGpsmAmUmAmUfUmCfUfGfC | 525 | vmApsfCpsmCmUmGfCmAmGmCmUmGm |
| | | mAmGmCmUmGmCmAmGmGpsmU | | CmAfGmAfAmUmAmUmCmCpsmUpsmC |
| dsNA-052 | 210 | mGpsmGpsmCmCmCmAfGmGfCfAfC | 526 | vmApsfGpsmCmAmCfCmUmUmCmUmGm |
| | | mAmGmAmAmGmGmUmGmCpsmU | | UmGfCmCfUmGmGmGmCmCpsmApsmC |
| dsNA-053 | 211 | mCpsmCpsmAmGmGmCfAmCfAfGfA | 527 | vmApsfGpsmUmAmGfCmAmCmCmUmUm |
| | | mAmGmGmUmGmCmUmAmCpsmU | | CmUfGmUfGmCmCmUmGmGpsmGpsmC |
| dsNA-054 | 212 | mGpsmGpsmCmAmCmAfGmAfAfGfG | 528 | vmApsfCpsmCmCmGfUmAmGmCmAmCmC |
| | | mUmGmCmUmAmCmGmGmGpsmU | | mUfUmCfUmGmUmGmCmCpsmUpsmG |
| dsNA-055 | 213 | mApsmCpsmAmGmAmAfGmGfUfGfC | 529 | vmApsfUpsmGmUmCfCmCmGmUmAmGm |
| | | mUmAmCmGmGmGmAmCmApsmU | | CmAfCmCfUmUmCmUmGmUpsmGpsmC |
| dsNA-056 | 214 | mGpsmApsmAmGmGmUfGmCfUfAfC | 530 | vmApsfCpsmGmCmUfGmUmCmCmCmGm |
| | | mGmGmGmAmCmAmGmCmGpsmU | | UmAfGmCfAmCmCmUmUmCpsmUpsmG |
| dsNA-057 | 215 | mGpsmGpsmUmGmCmUfAmCfGfGfG | 531 | vmApsfGpsmCmAmCfGmCmUmGmUmCm |
| | | mAmCmAmGmCmGmUmGmCpsmU | | CmCfGmUfAmGmCmAmCmCpsmUpsmU |
| dsNA-058 | 216 | mCpsmApsmGmCmGmUfGmCfAfGfC | 532 | vmApsfCpsmUmUmCfUmAmGmCmCmGm |
| | | mGmGmCmUmAmGmAmAmGpsmU | | CmUfGmCfAmCmGmCmUmGpsmUpsmC |
| dsNA-059 | 217 | mCpsmGpsmUmGmCmAfGmCfGfGfC | 533 | vmApsfGpsmGmAmCfUmUmCmUmAmGm |
| | | mUmAmGmAmAmGmUmCmCpsmU | | CmCfGmCfUmGmCmAmCmGpsmCpsmU |
| dsNA-060 | 218 | mGpsmCpsmAmGmCmGfGmCfUfAfG | 534 | vmApsfGpsmCmUmGfGmAmCmUmUmCm |
| | | mAmAmGmUmCmCmAmGmCpsmU | | UmAfGmCfCmGmCmUmGmCpsmApsmC |
| dsNA-061 | 219 | mGpsmCpsmGmGmCmUfAmGfAfAfG | 535 | vmApsfUpsmCmAmGfCmUmGmGmAmCm |
| | | mUmCmCmAmGmCmUmGmApsmU | | UmUfCmUfAmGmCmCmGmCpsmUpsmG |
| dsNA-062 | 220 | mCpsmUpsmAmGmAmAfGmUfCfCfA | 536 | vmApsfCpsmUmCmCfUmCmAmGmCmUm |
| | | mGmCmUmGmAmGmGmAmGpsmU | | GmGfAmCfUmUmCmUmAmGpsmCpsmC |
| dsNA-063 | 221 | mApsmApsmGmUmCmCfAmGfCfUfG | 537 | vmApsfGpsmGmCmGfCmUmCmCmUmCm |
| | | mAmGmGmAmGmCmGmCmCpsmU | | AmGfCmUfGmGmAmCmUmUpsmCpsmU |
| dsNA-064 | 222 | mUpsmCpsmCmAmGmCfUmGfAfGfG | 538 | vmApsfCpsmCmAmGfGmCmGmCmUmCmC |
| | | mAmGmCmGmCmCmUmGmGpsmU | | mUfCmAfGmCmUmGmGmApsmCpsmU |
| dsNA-065 | 223 | mCpsmUpsmGmAmGmGfAmGfCfGfC | 539 | vmApsfCpsmCmCmAfGmCmCmAmGmGmC |
| | | mCmUmGmGmCmUmGmGmGpsmU | | mGfCmUfCmCmUmCmAmGpsmCpsmU |
| dsNA-066 | 224 | mApsmGpsmGmAmGmCfGmCfCfUfG | 540 | vmApsfGpsmGmGmCfCmCmAmGmCmCm |
| | | mGmCmUmGmGmGmCmCmCpsmU | | AmGfGmCfGmCmUmCmCmUpsmCpsmA |
| dsNA-067 | 225 | mGpsmCpsmCmCmUmGfCmCfUfAfC | 541 | vmApsfApsmAmAmUfUmCmUmCmGmGm |
| | | mCmGmAmGmAmAmUmUmUpsmU | | UmAfGmGfCmAmGmGmGmCpsmCpsmC |
| dsNA-068 | 226 | mUpsmGpsmCmCmUmAfCmCfGfAfG | 542 | vmApsfCpsmCmUmCfAmAmAmUmUmCm |
| | | mAmAmUmUmUmGmAmGmGpsmU | | UmCfGmGfUmAmGmGmCmApsmGpsmG |
| dsNA-069 | 227 | mCpsmUpsmAmCmCmGfAmGfAfAfU | 543 | vmApsfApsmGmAmCfCmUmCmAmAmAm |
| | | mUmUmGmAmGmGmUmCmUpsmU | | UmUfCmUfCmGmGmUmAmGpsmGpsmC |
| dsNA-070 | 228 | mCpsmGpsmAmGmAmAfUmUfUfGfA | 544 | vmApsfUpsmUmUmAfAmGmAmCmCmUm |
| | | mGmGmUmCmUmUmAmAmApsmU | | CmAfAmAfUmUmCmUmCmGpsmGpsmU |
| dsNA-071 | 229 | mGpsmApsmAmUmUmUfGmAfGfGfU | 545 | vmApsfGpsmCmCmUfUmUmAmAmGmAm |
| | | mCmUmUmAmAmAmGmGmCpsmU | | CmCfUmCfAmAmAmUmUmCpsmUpsmC |
| dsNA-072 | 230 | mUpsmUpsmGmAmGmGfUmCfUfUfA | 546 | vmApsfGpsmUmGmAfGmCmCmUmUmUm |
| | | mAmAmGmGmCmUmCmAmCpsmU | | AmAfGmAfCmCmUmCmAmApsmApsmU |
| dsNA-073 | 231 | mCpsmUpsmUmAmAmAfGmGfCfUfC | 547 | vmApsfUpsmGmUmCfAmGmCmGmUmGm |
| | | mAmCmGmCmUmGmAmCmApsmU | | AmGfCmCfUmUmUmAmAmGpsmApsmC |
| dsNA-074 | 232 | mApsmApsmAmGmGmCfUmCfAfCfG | 548 | vmApsfGpsmCmUmUfGmUmCmAmGmCm |
| | | mCmUmGmAmCmAmAmGmCpsmU | | GmUfGmAfGmCmCmUmUmUpsmApsmA |
| dsNA-075 | 233 | mGpsmGpsmCmUmCmAfCmGfCfUfG | 549 | vmApsfUpsmCmUmGfCmUmUmGmUmCm |
| | | mAmCmAmAmGmCmAmGmApsmU | | AmGfCmGfUmGmAmGmCmCpsmUpsmU |
| dsNA-076 | 234 | mGpsmCpsmUmGmAmCfAmAfGfCfA | 550 | vmApsfApsmUmGmUfGmGmCmUmCmUm |
| | | mGmAmGmCmCmAmCmAmUpsmU | | GmCfUmUfGmUmCmAmGmCpsmGpsmU |
| dsNA-077 | 235 | mApsmCpsmAmAmGmCfAmGfAfGfC | 551 | vmApsfUpsmAmGmGfAmUmGmUmGmGm |
| | | mCmAmCmAmUmCmCmUmApsmU | | CmUfCmUfGmCmUmUmGmUpsmCpsmA |
| dsNA-078 | 236 | mApsmGpsmCmAmGmAfGmCfCfAfC | 552 | vmApsfCpsmCmAmUfAmGmGmAmUmGm |
| | | mAmUmCmCmUmAmUmGmGpsmU | | UmGfGmCfUmCmUmGmCmUpsmUpsmG |
| dsNA-079 | 237 | mApsmGpsmAmGmCmCfAmCfAfUfC | 553 | vmApsfGpsmGmCmCfCmAmUmAmGmGm |
| | | mCmUmAmUmGmGmGmCmCpsmU | | AmUfGmUfGmGmCmUmCmUpsmGpsmC |
| dsNA-080 | 238 | mApsmCpsmAmUmCmCfUmAfUfGfG | 554 | vmApsfUpsmGmUmGfAmGmGmGmCmCm |
| | | mGmCmCmCmUmCmAmCmApsmU | | CmAfUmAfGmGmAmUmGmUpsmGpsmG |
| dsNA-081 | 239 | mCpsmUpsmAmUmGmGfGmCfCfCfU | 555 | vmApsfUpsmGmGmCfCmUmGmUmGmAm |
| | | mCmAmCmAmGmGmCmCmApsmU | | GmGfGmCfCmCmAmUmAmGpsmGpsmA |
| dsNA-082 | 240 | mUpsmGpsmGmGmCmCfCmUfCfAfC | 556 | vmApsfApsmCmGmUfGmGmCmCmUmGm |
| | | mAmGmGmCmCmAmCmGmUpsmU | | UmGfAmGfGmGmCmCmCmApsmUpsmA |
| dsNA-083 | 241 | mCpsmCpsmCmUmCmAfCmAfGfGfC | 557 | vmApsfCpsmUmGmCfAmCmGmUmGmGm |
| | | mCmAmCmGmUmGmCmAmGpsmU | | CmCfUmGfUmGmAmGmGmGpsmCpsmC |
| dsNA-084 | 242 | mCpsmApsmCmAmGmGfCmCfAfCfG | 558 | vmApsfGpsmCmCmGfCmUmGmCmAmCm |
| | | mUmGmCmAmGmCmGmGmCpsmU | | GmUfGmGfCmCmUmGmUmGpsmApsmG |
| dsNA-085 | 243 | mApsmGpsmGmCmCmAfCmGfUfGfC | 559 | vmApsfUpsmCmUmGfCmCmGmCmUmGm |
| | | mAmGmCmGmGmCmAmGmApsmU | | CmAfCmGfUmGmGmCmCmUpsmGpsmU |
| dsNA-086 | 244 | mCpsmApsmCmGmUmGfCmAfGfCfG | 560 | vmApsfCpsmGmCmCfUmCmUmGmCmCmG |
| | | mGmCmAmGmAmGmGmCmGpsmU | | mCfUmGfCmAmCmGmUmGpsmGpsmC |
| dsNA-087 | 245 | mGpsmUpsmGmCmAmGfCmGfGfCfA | 561 | vmApsfUpsmCmCmCfGmCmCmUmCmUmG |
| | | mGmAmGmGmCmGmGmGmApsmU | | mCfCmGfCmUmGmCmAmCpsmGpsmU |
| dsNA-088 | 246 | mCpsmApsmGmCmGmGfCmAfGfAfG | 562 | vmApsfApsmUmCmUfCmCmCmGmCmCmU |
| | | mGmCmGmGmGmAmGmAmUpsmU | | mCfUmGfCmCmGmCmUmGpsmCpsmA |
| dsNA-089 | 247 | mGpsmGpsmCmAmGmAfGmGfCfGfG | 563 | vmApsfCpsmAmCmCfAmUmCmUmCmCmC |
| | | mGmAmGmAmUmGmGmUmGpsmU | | mGfCmCfUmCmUmGmCmCpsmGpsmC |
| dsNA-090 | 248 | mGpsmCpsmGmGmGmAfGmAfUfGfG | 564 | vmApsfGpsmCmUmGfUmGmCmCmAmCm |
| | | mUmGmGmCmAmCmAmGmCpsmU | | CmAfUmCfUmCmCmCmGmCpsmCpsmU |
| dsNA-091 | 249 | mGpsmApsmGmAmUmGfGmUfGfGfC | 565 | vmApsfUpsmGmCmUfGmCmUmGmUmGm |
| | | mAmCmAmGmCmAmGmCmApsmU | | CmCfAmCfCmAmUmCmUmCpsmCpsmC |
| dsNA-092 | 250 | mUpsmGpsmGmUmGmGfCmAfCfAfG | 566 | vmApsfCpsmCmGmAfUmGmCmUmGmCm |
| | | mCmAmGmCmAmUmCmGmGpsmU | | UmGfUmGfCmCmAmCmCmApsmUpsmC |
| dsNA-093 | 251 | mUpsmGpsmGmCmAmCfAmGfCfAfG | 567 | vmApsfCpsmAmGmCfCmGmAmUmGmCm |
| | | mCmAmUmCmGmGmCmUmGpsmU | | UmGfCmUfGmUmGmCmCmApsmCpsmC |
| dsNA-094 | 252 | mCpsmApsmCmAmGmCfAmGfCfAfU | 568 | vmApsfUpsmCmGmCfAmGmCmCmGmAm |
| | | mCmGmGmCmUmGmCmGmApsmU | | UmGfCmUfGmCmUmGmUmGpsmCpsmC |
| dsNA-095 | 253 | mCpsmApsmGmCmAmUfCmGfGfCfU | 569 | vmApsfApsmUmCmUfGmUmCmGmCmAm |
| | | mGmCmGmAmCmAmGmAmUpsmU | | GmCfCmGfAmUmGmCmUmGpsmCpsmU |
| dsNA-096 | 254 | mCpsmApsmUmCmGmGfCmUfGfCfG | 570 | vmApsfUpsmGmGmAfUmCmUmGmUmCm |
| | | mAmCmAmGmAmUmCmCmApsmU | | GmCfAmGfCmCmGmAmUmGpsmCpsmU |
| dsNA-097 | 255 | mCpsmGpsmGmCmUmGfCmGfAfCfA | 571 | vmApsfUpsmCmCmUfGmGmAmUmCmUm |
| | | mGmAmUmCmCmAmGmGmApsmU | | GmUfCmGfCmAmGmCmCmGpsmApsmU |
| dsNA-098 | 256 | mGpsmApsmCmAmGmAfUmCfCfAfG | 572 | vmApsfGpsmAmGmUfCmUmCmUmCmCm |
| | | mGmAmGmAmGmAmCmUmCpsmU | | UmGfGmAfUmCmUmGmUmCpsmGpsmC |
| dsNA-099 | 257 | mApsmGpsmAmUmCmCfAmGfGfAfG | 573 | vmApsfGpsmUmGmGfAmGmUmCmUmCm |
| | | mAmGmAmCmUmCmCmAmCpsmU | | UmCfCmUfGmGmAmUmCmUpsmGpsmU |
| dsNA-100 | 258 | mApsmGpsmCmGmGmCfGmCfUfCfC | 574 | vmApsfUpsmUmCmAfGmGmCmUmGmGm |
| | | mCmAmGmCmCmUmGmAmApsmU | | GmAfGmCfGmCmCmGmCmUpsmGpsmU |
| dsNA-101 | 259 | mGpsmGpsmCmGmCmUfCmCfCfAfG | 575 | vmApsfApsmGmAmUfUmCmAmGmGmCm |
| | | mCmCmUmGmAmAmUmCmUpsmU | | UmGfGmGfAmGmCmGmCmCpsmGpsmC |
| dsNA-102 | 260 | mGpsmCpsmUmCmCmCfAmGfCfCfU | 576 | vmApsfGpsmGmCmAfGmAmUmUmCmAm |
| | | mGmAmAmUmCmUmGmCmCpsmU | | GmGfCmUfGmGmGmAmGmCpsmGpsmC |
| dsNA-103 | 261 | mApsmGpsmCmCmUmGfAmAfUfCfU | 577 | vmApsfCpsmAmUmCfCmAmGmGmCmAm |
| | | mGmCmCmUmGmGmAmUmGpsmU | | GmAfUmUfCmAmGmGmCmUpsmGpsmG |
| dsNA-104 | 262 | mCpsmUpsmGmAmAmUfCmUfGfCfC | 578 | vmApsfUpsmUmCmCfAmUmCmCmAmGm |
| | | mUmGmGmAmUmGmGmAmApsmU | | GmCfAmGfAmUmUmCmAmGpsmGpsmC |
| dsNA-105 | 263 | mApsmUpsmCmUmGmCfCmUfGfGfA | 579 | vmApsfUpsmCmAmGfUmUmCmCmAmUm |
| | | mUmGmGmAmAmCmUmGmApsmU | | CmCfAmGfGmCmAmGmAmUpsmUpsmC |
| dsNA-106 | 264 | mGpsmCpsmCmUmGmGfAmUfGfGfA | 580 | vmApsfGpsmUmCmCfUmCmAmGmUmUm |
| | | mAmCmUmGmAmGmGmAmCpsmU | | CmCfAmUfCmCmAmGmGmCpsmApsmG |
| dsNA-107 | 265 | mGpsmGpsmAmUmGmGfAmAfCfUfG | 581 | vmApsfApsmUmUmGfGmUmCmCmUmCm |
| | | mAmGmGmAmCmCmAmAmUpsmU | | AmGfUmUfCmCmAmUmCmCpsmApsmG |
| dsNA-108 | 266 | mGpsmApsmAmCmUmGfAmGfGfAfC | 582 | vmApsfGpsmCmAmUfGmAmUmUmGmGm |
| | | mCmAmAmUmCmAmUmGmCpsmU | | UmCfCmUfCmAmGmUmUmCpsmCpsmA |
| dsNA-109 | 267 | mGpsmApsmGmGmAmCfCmAfAfUfC | 583 | vmApsfUpsmUmGmCfAmGmCmAmUmGm |
| | | mAmUmGmCmUmGmCmAmApsmU | | AmUfUmGfGmUmCmCmUmCpsmApsmG |
| dsNA-110 | 268 | mApsmCpsmCmAmAmUfCmAfUfGfC | 584 | vmApsfUpsmUmCmCfUmUmGmCmAmGm |
| | | mUmGmCmAmAmGmGmAmApsmU | | CmAfUmGfAmUmUmGmGmUpsmCpsmC |
| dsNA-111 | 269 | mUpsmCpsmAmUmGmCfUmGfCfAfA | 585 | vmApsfApsmAmGmUfGmUmUmCmCmUm |
| | | mGmGmAmAmCmAmCmUmUpsmU | | UmGfCmAfGmCmAmUmGmApsmUpsmU |
| dsNA-112 | 270 | mCpsmUpsmGmCmAmAfGmGfAfAfC | 586 | vmApsfCpsmGmUmGfGmAmAmGmUmGm |
| | | mAmCmUmUmCmCmAmCmGpsmU | | UmUfCmCfUmUmGmCmAmGpsmCpsmA |
| dsNA-113 | 271 | mApsmApsmGmGmAmAfCmAfCfUfU | 587 | vmApsfGpsmGmGmGfCmGmUmGmGmAm |
| | | mCmCmAmCmGmCmCmCmCpsmU | | AmGfUmGfUmUmCmCmUmUpsmGpsmC |
| dsNA-114 | 272 | mApsmApsmCmAmCmUfUmCfCfAfC | 588 | vmApsfUpsmCmAmCfGmGmGmGmCmGm |
| | | mGmCmCmCmCmGmUmGmApsmU | | UmGfGmAfAmGmUmGmUmUpsmCpsmC |
| dsNA-115 | 273 | mApsmCpsmUmUmCmCfAmCfGfCfC | 589 | vmApsfGpsmCmCmUfCmAmCmGmGmGm |
| | | mCmCmGmUmGmAmGmGmCpsmU | | GmCfGmUfGmGmAmAmGmUpsmGpsmU |
| dsNA-116 | 274 | mUpsmCpsmCmAmCmGfCmCfCfCfGm | 590 | vmApsfGpsmGmGmGfCmCmUmCmAmCm |
| | | UmGmAmGmGmCmCmCmCpsmU | | GmGfGmGfCmGmUmGmGmApsmApsmG |
| dsNA-117 | 275 | mGpsmGpsmAmGmGmAfGmCfUfGfC | 591 | vmApsfApsmGmUmGfAmAmCmAmGmGm |
| | | mCmUmGmUmUmCmAmCmUpsmU | | CmAfGmCfUmCmCmUmCmCpsmCpsmU |
| dsNA-118 | 276 | mGpsmGpsmAmGmCmUfGmCfCfUfG | 592 | vmApsfCpsmCmCmAfGmUmGmAmAmCm |
| | | mUmUmCmAmCmUmGmGmGpsmU | | AmGfGmCfAmGmCmUmCmCpsmUpsmC |
| dsNA-119 | 277 | mGpsmCpsmUmGmCmCfUmGfUfUfC | 593 | vmApsfGpsmAmUmCfCmCmAmGmUmGm |
| | | mAmCmUmGmGmGmAmUmCpsmU | | AmAfCmAfGmGmCmAmGmCpsmUpsmC |
| dsNA-120 | 278 | mGpsmCpsmCmUmGmUfUmCfAfCfU | 594 | vmApsfGpsmCmUmGfAmUmCmCmCmAm |
| | | mGmGmGmAmUmCmAmGmCpsmU | | GmUfGmAfAmCmAmGmGmCpsmApsmG |
| dsNA-121 | 279 | mGpsmUpsmUmCmAmCfUmGfGfGfA | 595 | vmApsfCpsmCmUmGfGmCmUmGmAmUm |
| | | mUmCmAmGmCmCmAmGmGpsmU | | CmCfCmAfGmUmGmAmAmCpsmApsmG |
| dsNA-122 | 280 | mApsmCpsmUmGmGmGfAmUfCfAfG | 596 | vmApsfGpsmCmGmCfCmCmUmGmGmCm |
| | | mCmCmAmGmGmGmCmGmCpsmU | | UmGfAmUfCmCmCmAmGmUpsmGpsmA |
| dsNA-123 | 281 | mGpsmGpsmGmAmUmCfAmGfCfCfA | 597 | vmApsfCpsmCmGmGfCmGmCmCmCmUmG |
| | | mGmGmGmCmGmCmCmGmGpsmU | | mGfCmUfGmAmUmCmCmCpsmApsmG |
| dsNA-124 | 282 | mUpsmCpsmAmGmCmCfAmGfGfGfC | 598 | vmApsfGpsmGmGmCfCmCmGmGmCmGm |
| | | mGmCmCmGmGmGmCmCmCpsmU | | CmCfCmUfGmGmCmUmGmApsmUpsmC |
| dsNA-125 | 283 | mGpsmGpsmGmCmGmCfCmGfGfGfC | 599 | vmApsfApsmGmAmAfGmUmGmGmGmGm |
| | | mCmCmCmAmCmUmUmCmUpsmU | | CmCfCmGfGmCmGmCmCmCpsmUpsmG |
| dsNA-126 | 284 | mGpsmCpsmCmGmGmGfCmCfCfCfA | 600 | vmApsfGpsmCmUmCfAmGmAmAmGmUm |
| | | mCmUmUmCmUmGmAmGmCpsmU | | GmGfGmGfCmCmCmGmGmCpsmGpsmC |
| dsNA-127 | 285 | mGpsmGpsmGmCmCmCfCmAfCfUfU | 601 | vmApsfUpsmGmUmGfCmUmCmAmGmAm |
| | | mCmUmGmAmGmCmAmCmApsmU | | AmGfUmGfGmGmGmCmCmCpsmGpsmG |
| dsNA-128 | 286 | mCpsmCpsmCmCmAmCfUmUfCfUfG | 602 | vmApsfCpsmUmCmUfGmUmGmCmUmCm |
| | | mAmGmCmAmCmAmGmAmGpsmU | | AmGfAmAfGmUmGmGmGmGpsmCpsmC |
| dsNA-129 | 287 | mUpsmUpsmCmUmGmAfGmCfAfCfA | 603 | vmApsfUpsmCmUmCfUmGmCmUmCmUm |
| | | mGmAmGmCmAmGmAmGmApsmU | | GmUfGmCfUmCmAmGmAmApsmGpsmU |
| dsNA-130 | 288 | mUpsmGpsmAmGmCmAfCmAfGfAfG | 604 | vmApsfCpsmUmGmUfCmUmCmUmGmCm |
| | | mCmAmGmAmGmAmCmAmGpsmU | | UmCfUmGfUmGmCmUmCmApsmGpsmA |
| dsNA-131 | 289 | mGpsmCpsmAmCmAmGfAmGfCfAfG | 605 | vmApsfCpsmGmUmCfUmGmUmCmUmCm |
| | | mAmGmAmCmAmGmAmCmGpsmU | | UmGfCmUfCmUmGmUmGmCpsmUpsmC |
| dsNA-132 | 290 | mCpsmApsmGmAmGmCfAmGfAfGfA | 606 | vmApsfCpsmUmGmCfGmUmCmUmGmUm |
| | | mCmAmGmAmCmGmCmAmGpsmU | | CmUfCmUfGmCmUmCmUmGpsmUpsmG |
| dsNA-133 | 291 | mApsmGpsmCmAmGmAfGmAfCfAfG | 607 | vmApsfCpsmGmCmCfUmGmCmGmUmCm |
| | | mAmCmGmCmAmGmGmCmGpsmU | | UmGfUmCfUmCmUmGmCmUpsmCpsmU |
| dsNA-134 | 292 | mApsmGpsmAmCmAmGfAmCfGfCfA | 608 | vmApsfGpsmUmCmCfCmCmGmCmCmUmG |
| | | mGmGmCmGmGmGmGmAmCpsmU | | mCfGmUfCmUmGmUmCmUpsmCpsmU |
| dsNA-135 | 293 | mCpsmApsmGmAmCmGfCmAfGfGfC | 609 | vmApsfUpsmUmUmGfUmCmCmCmCmGm |
| | | mGmGmGmGmAmCmAmAmApsmU | | CmCfUmGfCmGmUmCmUmGpsmUpsmC |
| dsNA-136 | 294 | mApsmCpsmGmCmAmGfGmCfGfGfG | 610 | vmApsfGpsmCmCmUfUmUmGmUmCmCm |
| | | mGmAmCmAmAmAmGmGmCpsmU | | CmCfGmCfCmUmGmCmGmUpsmCpsmU |
| dsNA-137 | 295 | mApsmGpsmGmCmGmGfGmGfAfCfA | 611 | vmApsfCpsmUmCmUfGmCmCmUmUmUm |
| | | mAmAmGmGmCmAmGmAmGpsmU | | GmUfCmCfCmCmGmCmCmUpsmGpsmC |
| dsNA-138 | 296 | mCpsmApsmAmAmGmGfCmAfGfAfG | 612 | vmApsfGpsmGmCmUfAmCmAmUmCmCm |
| | | mGmAmUmGmUmAmGmCmCpsmU | | UmCfUmGfCmCmUmUmUmGpsmUpsmC |
| dsNA-139 | 297 | mApsmGpsmGmCmAmGfAmGfGfAfU | 613 | vmApsfUpsmGmGmGfGmCmUmAmCmAm |
| | | mGmUmAmGmCmCmCmCmApsmU | | UmCfCmUfCmUmGmCmCmUpsmUpsmU |
| dsNA-140 | 298 | mCpsmApsmGmAmGmGfAmUfGfUfA | 614 | vmApsfCpsmAmAmUfGmGmGmGmCmUm |
| | | mGmCmCmCmCmAmUmUmGpsmU | | AmCfAmUfCmCmUmCmUmGpsmCpsmC |
| dsNA-141 | 299 | mApsmGpsmGmAmUmGfUmAfGfCfC | 615 | vmApsfCpsmCmCmCfAmAmUmGmGmGm |
| | | mCmCmAmUmUmGmGmGmGpsmU | | GmCfUmAfCmAmUmCmCmUpsmCpsmU |
| dsNA-142 | 300 | mApsmUpsmGmUmAmGfCmCfCfCfA | 616 | vmApsfCpsmCmUmCfCmCmCmAmAmUmG |
| | | mUmUmGmGmGmGmAmGmGpsmU | | mGfGmGfCmUmAmCmAmUpsmCpsmC |
| dsNA-143 | 301 | mGpsmCpsmCmCmCmAfUmUfGfGfG | 617 | vmApsfCpsmCmAmCfCmCmCmUmCmCmC |
| | | mGmAmGmGmGmGmUmGmGpsmU | | mCfAmAfUmGmGmGmGmCpsmUpsmA |
| dsNA-144 | 302 | mUpsmUpsmGmGmGmGfAmGfGfGfG | 618 | vmApsfCpsmUmUmCfCmUmCmCmAmCmC |
| | | mUmGmGmAmGmGmAmAmGpsmU | | mCfCmUfCmCmCmCmAmApsmUpsmG |
| dsNA-145 | 303 | mCpsmCpsmUmCmAmUfUmAfAfAfG | 619 | vmApsfApsmCmGmAfCmUmCmUmGmCm |
| | | mCmAmGmAmGmUmCmGmUpsmU | | UmUfUmAfAmUmGmAmGmGpsmGpsmG |
| dsNA-146 | 304 | mCpsmApsmUmUmAmAfAmGfCfAfG | 620 | vmApsfGpsmCmCmAfCmGmAmCmUmCm |
| | | mAmGmUmCmGmUmGmGmCpsmU | | UmGfCmUfUmUmAmAmUmGpsmApsmG |
| dsNA-147 | 305 | mUpsmApsmAmAmGmCfAmGfAfGfU | 621 | vmApsfGpsmAmUmGfCmCmAmCmGmAm |
| | | mCmGmUmGmGmCmAmUmCpsmU | | CmUfCmUfGmCmUmUmUmApsmApsmU |
| dsNA-148 | 306 | mGpsmGpsmAmGmGmAfGmGfAfUfA | 622 | vmApsfGpsmCmUmGfCmAmGmAmAmUm |
| | | mUmUmCmUmGmCmAmGmCmUp(ps) | | AmUfCmCfUmCmCmUmCmCpsmApsmU |
| | | 2GalNAc4 | | |
| dsNA-149 | 307 | mCpsmGpsmAmGmAmAfUmUfUfGfA | 623 | vmApsfUpsmUmUmAfAmGmAmCmCmUm |
| | | mGmGmUmCmUmUmAmAmAmU | | CmAfAmAfUmUmCmUmCmGpsmGpsmU |
| | | p(ps)2GalNAc4 | | |
| dsNA-150 | 308 | mGpsmApsmAmUmUmUfGmAfGfGfU | 624 | vmApsfGpsmCmCmUfUmUmAmAmGmAm |
| | | mCmUmUmAmAmAmGmGmCmU | | CmCfUmCfAmAmAmUmUmCpsmUpsmC |
| | | p(ps)2GalNAc4 | | |
| dsNA-151 | 309 | mApsmCpsmAmAmGmCfAmGfAfGfC | 625 | vmApsfUpsmAmGmGfAmUmGmUmGmGm |
| | | mCmAmCmAmUmCmCmUmAmU | | CmUfCmUfGmCmUmUmGmUpsmCpsmA |
| | | p(ps)2GalNAc4 | | |
| dsNA-152 | 310 | mCpsmUpsmGmAmAmUfCmUfGfCfC | 626 | vmApsfUpsmUmCmCfAmUmCmCmAmGm |
| | | mUmGmGmAmUmGmGmAmAmU | | GmCfAmGfAmUmUmCmAmGpsmGpsmC |
| | | p(ps)2GalNAc4 | | |
| dsNA-153 | 311 | mCpsmApsmUmUmAmAfAmGfCfAfG | 627 | vmApsfGpsmCmCmAfCmGmAmCmUmCm |
| | | mAmGmUmCmGmUmGmGmCmU | | UmGfCmUfUmUmAmAmUmGpsmApsmG |
| | | p(ps)2GalNAc4 | | |
| dsNA-154 | 312 | mCpsmApsmGmGmAmAfCmAfGfCfC | 628 | vmApsfApsmGmAmGfAmCmCmCmAmGm |
| | | mUmGmGmGmUmCmUmCmUmU | | GmCfUmGfUmUmCmCmUmGpsmGpsmC |
| | | p(ps)2GalNAc4 | | |
| dsNA-155 | 313 | mCpsmUpsmAmCmCmGfAmGfAfAfU | 629 | vmApsfApsmGmAmCfCmUmCmAmAmAm |
| | | mUmUmGmAmGmGmUmCmUmU | | UmUfCmUfCmGmGmUmAmGpsmGpsmC |
| | | p(ps)2GalNAc4 | | |
| dsNA-156 | 314 | mGpsmCpsmUmGmAmCfAmAfGfCfA | 630 | vmApsfApsmUmGmUfGmGmCmUmCmUm |
| | | mGmAmGmCmCmAmCmAmUmU | | GmCfUmUfGmUmCmAmGmCpsmGpsmU |
| | | p(ps)2GalNAc4 | | |
| dsNA-157 | 315 | mGpsmApsmGmAmUmGfGmUfGfGfC | 631 | vmApsfUpsmGmCmUfGmCmUmGmUmGm |
| | | mAmCmAmGmCmAmGmCmAmU | | CmCfAmCfCmAmUmCmUmCpsmCpsmC |
| | | p(ps)2GalNAc4 | | |
| dsNA-158 | 316 | mCpsmCpsmUmCmAmUfUmAfAfAfG | 632 | vmApsfApsmCmGmAfCmUmCmUmGmCm |
| | | mCmAmGmAmGmUmCmGmUmU | | UmUfUmAfAmUmGmAmGmGpsmGpsmG |
| | | p(ps)2GalNAc4 | | |

Abbreviations: f is a 2′-fluoronucleotide,
m is a 2′-O-methylnucleotide,
ps is a phosphorothioate internucleotide linkage,
v is 5′-vinylphosphonate,
and p(ps)2GalNAc4 is

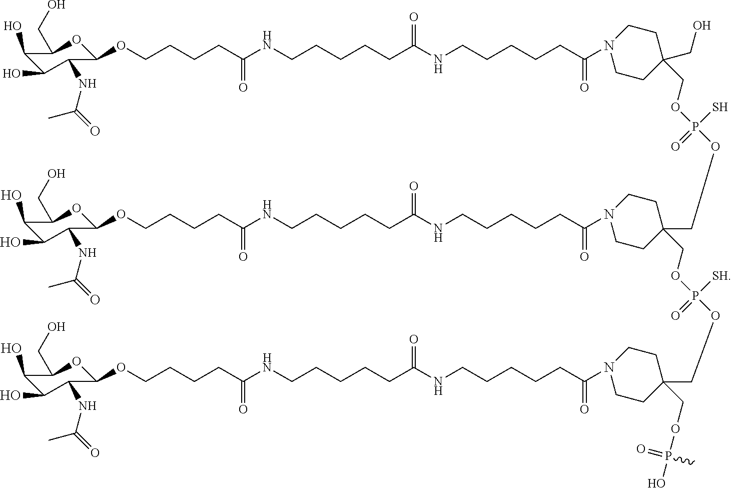

TABLE 3
ANGPTL3 siNA SS and AS Unmodified Sequences (5′ to 3′)

| | SEQ ID | | SEQ ID | |
|---|---|---|---|---|
| Identifier | NO: | SS 5′->3′ | NO: | AS 5′->3′ |
| dsNA-159 | 633 | CAAAAUCAAGAUUUGCUAUGU | 879 | ACAUAGCAAAUCUUGAUUUUGGC |
| dsNA-160 | 634 | AAAAUCAAGAUUUGCUAUGUU | 880 | AACAUAGCAAAUCUUGAUUUUGG |
| dsNA-161 | 635 | UGGUCUUAAAGACUUUGUCCA | 881 | UGGACAAAGUCUUUAAGACCAUG |
| dsNA-162 | 636 | GUCUUAAAGACUUUGUCCAUA | 882 | UAUGGACAAAGUCUUUAAGACCA |
| dsNA-163 | 637 | UCUUAAAGACUUUGUCCAUAA | 883 | UUAUGGACAAAGUCUUUAAGACC |
| dsNA-164 | 638 | CUUAAAGACUUUGUCCAUAAA | 884 | UUUAUGGACAAAGUCUUUAAGAC |
| dsNA-165 | 639 | UUAAAGACUUUGUCCAUAAGA | 885 | UCUUAUGGACAAAGUCUUUAAGA |
| dsNA-166 | 640 | UAAAGACUUUGUCCAUAAGAA | 886 | UUCUUAUGGACAAAGUCUUUAAG |
| dsNA-167 | 641 | AAAGACUUUGUCCAUAAGACA | 887 | UGUCUUAUGGACAAAGUCUUUAA |
| dsNA-168 | 642 | CAACAUAUUUGAUCAGUCUUU | 888 | AAAGACUGAUCAAAUAUGUUGAG |
| dsNA-169 | 643 | ACCAGUGAAAUCAAAGAAGAA | 889 | UUCUUCUUUGAUUUCACUGGUUU |
| dsNA-170 | 644 | GGUAAAGAAUAUGUCACUUGA | 890 | UCAAGUGACAUAUUCUUUACCUC |
| dsNA-171 | 645 | GUAAAGAAUAUGUCACUUGAA | 891 | UUCAAGUGACAUAUUCUUUACCU |
| dsNA-172 | 646 | UAAAGAAUAUGUCACUUGAAA | 892 | UUUCAAGUGACAUAUUCUUUACC |
| dsNA-173 | 647 | AAAGAAUAUGUCACUUGAACU | 893 | AGUUCAAGUGACAUAUUCUUUAC |
| dsNA-174 | 648 | AAGAAUAUGUCACUUGAACUA | 894 | UAGUUCAAGUGACAUAUUCUUUA |
| dsNA-175 | 649 | GAAUAUGUCACUUGAACUCAA | 895 | UUGAGUUCAAGUGACAUAUUCUU |
| dsNA-176 | 650 | AAUAUGUCACUUGAACUCAAA | 896 | UUUGAGUUCAAGUGACAUAUUCU |
| dsNA-177 | 651 | AUAUGUCACUUGAACUCAACU | 897 | AGUUGAGUUCAAGUGACAUAUUC |
| dsNA-178 | 652 | UAUGUCACUUGAACUCAACUA | 898 | UAGUUGAGUUCAAGUGACAUAUU |
| dsNA-179 | 653 | UGUCACUUGAACUCAACUCAA | 899 | UUGAGUUGAGUUCAAGUGACAUA |
| dsNA-180 | 654 | GUCACUUGAACUCAACUCAAA | 900 | UUUGAGUUGAGUUCAAGUGACAU |
| dsNA-181 | 655 | UCACUUGAACUCAACUCAAAA | 901 | UUUUGAGUUGAGUUCAAGUGACA |
| dsNA-182 | 656 | AGAAAUAGAAAAUCAGCUCAA | 902 | UUGAGCUGAUUUUCUAUUUCUUU |
| dsNA-183 | 657 | GAAAUAGAAAAUCAGCUCAGA | 903 | UCUGAGCUGAUUUUCUAUUUCUU |
| dsNA-184 | 658 | AAUAGAAAAUCAGCUCAGAAA | 904 | UUUCUGAGCUGAUUUUCUAUUUC |
| dsNA-185 | 659 | CCAAGAGCACCAAGAACUACU | 905 | AGUAGUUCUUGGUGCUCUUGGCU |
| dsNA-186 | 660 | AAGAGCACCAAGAACUACUCA | 906 | UGAGUAGUUCUUGGUGCUCUUGG |
| dsNA-187 | 661 | AGCACCAAGAACUACUCCCUU | 907 | AAGGGAGUAGUUCUUGGUGCUCU |
| dsNA-188 | 662 | AGGUGAACAUACAAGUGGCAU | 908 | AUGCCACUUGUAUGUUCACCUCU |
| dsNA-189 | 663 | GUGAACAUACAAGUGGCAUGU | 909 | ACAUGCCACUUGUAUGUUCACCU |
| dsNA-190 | 664 | UGAACAUACAAGUGGCAUGUA | 910 | UACAUGCCACUUGUAUGUUCACC |
| dsNA-191 | 665 | GAACAUACAAGUGGCAUGUAU | 911 | AUACAUGCCACUUGUAUGUUCAC |
| dsNA-192 | 666 | GGUUUUGGGAGGCUUGAUGGA | 912 | UCCAUCAAGCCUCCCAAAACCAU |
| dsNA-193 | 667 | UGGGAGGCUUGAUGGAGAAUU | 913 | AAUUCUCCAUCAAGCCUCCCAAA |
| dsNA-194 | 668 | UGUGGAGAAAACAACCUAAAU | 914 | AUUUAGGUUGUUUUCUCCACACU |
| dsNA-195 | 669 | GUGGAGAAAACAACCUAAAUA | 915 | UAUUUAGGUUGUUUUCUCCACAC |
| dsNA-196 | 670 | UGGAGAAAACAACCUAAAUGA | 916 | UCAUUUAGGUUGUUUUCUCCACA |
| dsNA-197 | 671 | GGAGAAAACAACCUAAAUGGU | 917 | ACCAUUUAGGUUGUUUUCUCCAC |
| dsNA-198 | 672 | GAGAAAACAACCUAAAUGGUA | 918 | UACCAUUUAGGUUGUUUUCUCCA |
| dsNA-199 | 673 | GAAAACAACCUAAAUGGUAAA | 919 | UUUACCAUUUAGGUUGUUUUCUC |
| dsNA-200 | 674 | AUCAAGAUUUGCUAUGUUAGA | 920 | UCUAACAUAGCAAAUCUUGAUUU |
| dsNA-201 | 675 | UGUAAAAAUUUUAGCCAAUGA | 921 | UCAUUGGCUAAAAUUUUUACAUC |
| dsNA-202 | 676 | GUAAAAAUUUUAGCCAAUGGA | 922 | UCCAUUGGCUAAAAUUUUUACAU |
| dsNA-203 | 677 | UAAAAAUUUUAGCCAAUGGCA | 923 | UGCCAUUGGCUAAAAUUUUUACA |
| dsNA-204 | 678 | AAAAAUUUUAGCCAAUGGCCU | 924 | AGGCCAUUGGCUAAAAUUUUUAC |
| dsNA-205 | 679 | AAAUUUUAGCCAAUGGCCUCA | 925 | UGAGGCCAUUGGCUAAAAUUUUU |
| dsNA-206 | 680 | AAUUUUAGCCAAUGGCCUCCU | 926 | AGGAGGCCAUUGGCUAAAAUUUU |
| dsNA-207 | 681 | UUAGCCAAUGGCCUCCUUCAA | 927 | UUGAAGGAGGCCAUUGGCUAAAA |
| dsNA-208 | 682 | UAGCCAAUGGCCUCCUUCAGU | 928 | ACUGAAGGAGGCCAUUGGCUAAA |
| dsNA-209 | 683 | GGGCCAAAUUAAUGACAUAUU | 929 | AAUAUGUCAUUAAUUUGGCCCUU |
| dsNA-210 | 684 | GGCCAAAUUAAUGACAUAUUU | 930 | AAAUAUGUCAUUAAUUUGGCCCU |
| dsNA-211 | 685 | GCCAAAUUAAUGACAUAUUUA | 931 | UAAAUAUGUCAUUAAUUUGGCCC |
| dsNA-212 | 686 | CCAAAUUAAUGACAUAUUUCA | 932 | UGAAAUAUGUCAUUAAUUUGGCC |
| dsNA-213 | 687 | CAAAUUAAUGACAUAUUUCAA | 933 | UUGAAAUAUGUCAUUAAUUUGGC |
| dsNA-214 | 688 | AAUUAAUGACAUAUUUCAAAA | 934 | UUUUGAAAUAUGUCAUUAAUUUG |
| dsNA-215 | 689 | AUUAAUGACAUAUUUCAAAAA | 935 | UUUUUGAAAUAUGUCAUUAAUUU |
| dsNA-216 | 690 | ACAUAUUUGAUCAGUCUUUUU | 936 | AAAAAGACUGAUCAAAUAUGUUG |
| dsNA-217 | 691 | CAUAUUUGAUCAGUCUUUUUA | 937 | UAAAAAGACUGAUCAAAUAUGUU |
| dsNA-218 | 692 | AUAUUUGAUCAGUCUUUUUAU | 938 | AUAAAAAGACUGAUCAAAUAUGU |
| dsNA-219 | 693 | UAUUUGAUCAGUCUUUUUAUA | 939 | UAUAAAAAGACUGAUCAAAUAUG |
| dsNA-220 | 694 | UUUGAUCAGUCUUUUUAUGAU | 940 | AUCAUAAAAAGACUGAUCAAAUA |
| dsNA-221 | 695 | GCCAAGAGCACCAAGAACUAA | 941 | UUAGUUCUUGGUGCUCUUGGCUU |
| dsNA-222 | 696 | AGAGCACCAAGAACUACUCCA | 942 | UGGAGUAGUUCUUGGUGCUCUUG |
| dsNA-223 | 697 | GGUGAACAUACAAGUGGCAUA | 943 | UAUGCCACUUGUAUGUUCACCUC |
| dsNA-224 | 698 | GUUUUGGGAGGCUUGAUGGAA | 944 | UUCCAUCAAGCCUCCCAAAACCA |
| dsNA-225 | 699 | GAGAAUUUUGGUUGGGCCUAA | 945 | UUAGGCCCAACCAAAAUUCUCCA |
| dsNA-226 | 700 | GAAUUUUGGUUGGGCCUAGAA | 946 | UUCUAGGCCCAACCAAAAUUCUC |
| dsNA-227 | 701 | AUUUUGGUUGGGCCUAGAGAA | 947 | UUCUCUAGGCCCAACCAAAAUUC |
| dsNA-228 | 702 | UUUUGGUUGGGCCUAGAGAAA | 948 | UUUCUCUAGGCCCAACCAAAAUU |
| dsNA-229 | 703 | GUUGGGCCUAGAGAAGAUAUA | 949 | UAUAUCUUCUCUAGGCCCAACCA |
| dsNA-230 | 704 | UUGGGCCUAGAGAAGAUAUAA | 950 | UUAUAUCUUCUCUAGGCCCAACC |
| dsNA-231 | 705 | UGGGCCUAGAGAAGAUAUACU | 951 | AGUAUAUCUUCUCUAGGCCCAAC |
| dsNA-232 | 706 | AAAACAACCUAAAUGGUAAAU | 952 | AUUUACCAUUUAGGUUGUUUUCU |
| dsNA-233 | 707 | AAACAACCUAAAUGGUAAAUA | 953 | UAUUUACCAUUUAGGUUGUUUUC |
| dsNA-234 | 708 | AACAACCUAAAUGGUAAAUAU | 954 | AUAUUUACCAUUUAGGUUGUUUU |
| dsNA-235 | 709 | ACAACCUAAAUGGUAAAUAUA | 955 | UAUAUUUACCAUUUAGGUUGUUU |
| dsNA-236 | 710 | CAACCUAAAUGGUAAAUAUAA | 956 | UUAUAUUUACCAUUUAGGUUGUU |
| dsNA-237 | 711 | ACCUAAAUGGUAAAUAUAACA | 957 | UGUUAUAUUUACCAUUUAGGUUG |
| dsNA-238 | 712 | CUAAAUGGUAAAUAUAACAAA | 958 | UUUGUUAUAUUUACCAUUUAGGU |
| dsNA-239 | 713 | UAAAUGGUAAAUAUAACAAAA | 959 | UUUUGUUAUAUUUACCAUUUAGG |
| dsNA-240 | 714 | CAAAAUCAAGAUUUGCUAUGU | 960 | ACAUAGCAAAUCUUGAUUUUGGC |
| dsNA-241 | 715 | GUCACUUGAACUCAACUCAAA | 961 | UUUGAGUUGAGUUCAAGUGACAU |
| dsNA-242 | 716 | AAUUAAUGACAUAUUUCAAAA | 962 | UUUUGAAAUAUGUCAUUAAUUUG |
| dsNA-243 | 717 | ACAUAUUUGAUCAGUCUUUUU | 963 | AAAAAGACUGAUCAAAUAUGUUG |
| dsNA-244 | 718 | AACAACCUAAAUGGUAAAUAU | 964 | AUAUUUACCAUUUAGGUUGUUUU |
| dsNA-245 | 719 | UAAAUGGUAAAUAUAACAAAA | 965 | UUUUGUUAUAUUUACCAUUUAGG |
| dsNA-246 | 720 | AAAAUCAAGAUUUGCUAUGUU | 966 | AACAUAGCAAAUCUUGAUUUUGG |
| dsNA-247 | 721 | CUAAAUGGUAAAUAUAACAAA | 967 | UUUGUUAUAUUUACCAUUUAGGU |
| dsNA-248 | 722 | CAAAUUAAUGACAUAUUUCAA | 968 | UUGAAAUAUGUCAUUAAUUUGGC |
| dsNA-249 | 723 | UUUGAUCAGUCUUUUUAUGAU | 969 | AUCAUAAAAAGACUGAUCAAAUA |
| dsNA-250 | 724 | AACAACCUAAAUGGUAAAUAU | 970 | AUAUUUACCAUUUAGGUUGUUUU |
| dsNA-251 | 725 | AACAACCUAAAUGGUAAAUAU | 971 | AUAUUUACCAUUUAGGUUGUUUU |
| dsNA-252 | 726 | AACAACCUAAAUGGUAAAUAU | 972 | AUAUUUACCAUUUAGGUUGUUUU |
| dsNA-253 | 727 | AACAACCUAAAUGGUAAAUAU | 973 | AUAUUUACCAUUUAGGUUGUUUU |
| dsNA-254 | 728 | AACAACCUAAAUGGUAAAUAU | 974 | AUAUUUACCAUUUAGGUUGUUUU |
| dsNA-255 | 729 | AACAACCUAAAUGGUAAAUAU | 975 | AUAUUUACCAUUUAGGUUGUUUU |
| dsNA-256 | 730 | AACAACCUAAAUGGUAAAUAU | 976 | AUAUUUACCAUUUAGGUUGUUUU |
| dsNA-257 | 731 | AACAACCUAAAUGGUAAAUAU | 977 | AUAUUUACCAUUUAGGUUGUUUU |
| dsNA-258 | 732 | AACAACCUAAAUGGUAAAUAU | 978 | AUAUUUACCAUUUAGGUUGUUUU |
| dsNA-259 | 733 | AACAACCUAAAUGGUAAAUAU | 979 | AUAUUUACCAUUUAGGUUGUUUU |
| dsNA-260 | 734 | AACAACCUAAAUGGUAAAUAU | 980 | AUAUUUACCAUUUAGGUUGUUUU |
| dsNA-261 | 735 | AACAACCUAAAUGGUAAAUAU | 981 | AUAUUUACCAUUUAGGUUGUUUU |
| dsNA-262 | 736 | CAAAAUCAAGAUUUGCUAUGU | 982 | ACAUAGCAAAUCUUGAUUUUGGC |
| dsNA-263 | 737 | CAAAAUCAAGAUUUGCUAUGU | 983 | ACAUAGCAAAUCUUGAUUUUGGC |
| dsNA-264 | 738 | CAAAAUCAAGAUUUGCUAUGU | 984 | ACAUAGCAAAUCUUGAUUUUGGC |
| dsNA-265 | 739 | CAAAAUCAAGAUUUGCUAUGU | 985 | ACAUAGCAAAUCUUGAUUUUGGC |
| dsNA-266 | 740 | CAAAAUCAAGAUUUGCUAUGU | 986 | ACAUAGCAAAUCUUGAUUUUGGC |
| dsNA-267 | 741 | AACAACCUAAAUGGUAAAUAU | 987 | AUAUUUACCAUUUAGGUUGUUUU |
| dsNA-268 | 742 | AACAACCUAAAUGGUAAAUAU | 988 | AUAUUUACCAUUUAGGUUGUUUU |
| dsNA-269 | 743 | AACAACCUAAAUGGUAAAUAU | 989 | AUAUUUACCAUUUAGGUUGUUUU |
| dsNA-270 | 744 | CAAAAUCAAGAUUUGCUAUGU | 990 | ACAUAGCAAAUCUUGAUUUUGGC |
| dsNA-271 | 745 | CAAAAUCAAGAUUUGCUAUGU | 991 | ACAUAGCAAAUCUUGAUUUUGGC |
| dsNA-272 | 746 | CAAAAUCAAGAUUUGCUAUGU | 992 | ACAUAGCAAAUCUUGAUUUUGGC |
| dsNA-273 | 747 | CAAAAUCAAGAUUUGCUAUGU | 993 | ACAUAGCAAAUCUUGAUUUUGGC |
| dsNA-274 | 748 | CAAAAUCAAGAUUUGCUAUGU | 994 | ACAUAGCAAAUCUUGAUUUUGGC |
| dsNA-275 | 749 | CAAAAUCAAGAUUUGCUAUGU | 995 | ACAUAGCAAAUCUUGAUUUUGGC |
| dsNA-276 | 750 | CAAAAUCAAGAUUUGCUAUGU | 996 | ACAUAGCAAAUCUUGAUUUUGGC |
| dsNA-277 | 751 | CAAAAUCAAGAUUUGCUAUGU | 997 | ACAUAGCAAAUCUUGAUUUUGGC |
| dsNA-278 | 752 | CAAAAUCAAGAUUUGCUAUGU | 998 | ACAUAGCAAAUCUUGAUUUUGGC |
| dsNA-279 | 753 | CAAAAUCAAGAUUUGCUAUGU | 999 | ACAUAGCAAAUCUUGAUUUUGGC |
| dsNA-280 | 754 | CAAAAUCAAGAUUUGCUAUGU | 1001 | ACAUAGCAAAUCUUGAUUUUGGC |
| dsNA-281 | 755 | CAAAAUCAAGAUUUGCUAUGU | 1002 | ACAUAGCAAAUCUUGAUUUUGGC |

TABLE 4
ANGPTL3 siNA SS and AS Sequences with Modified Nucleotides

| | SEQ | | SEQ | |
|---|---|---|---|---|
| | ID | | ID | |
| Identifier | NO: | SS 5′->3′ | NO: | AS 5′->3′ |
| dsNA-159 | 756 | mCpsmApsmAmAmAmUfCmAfAfGfA | 1003 | vmApsfCpsmAmUmAfGmCmAmAmAmUm |
| | | mUmUmUmGmCmUmAmUmGpsmU | | CmUfUmGfAmUmUmUmUmGpsmGpsmC |
| dsNA-160 | 757 | mApsmApsmAmAmUmCfAmAfGfAfU | 1004 | vmApsfApsmCmAmUfAmGmCmAmAmAm |
| | | mUmUmGmCmUmAmUmGmUpsmU | | UmCfUmUfGmAmUmUmUmUpsmGpsmG |
| dsNA-161 | 758 | mUpsmGpsmGmUmCmUfUmAfAfAfG | 1005 | vmUpsfGpsmGmAmCfAmAmAmGmUmCm |
| | | mAmCmUmUmUmGmUmCmCpsmA | | UmUfUmAfAmGmAmCmCmApsmUpsmG |
| dsNA-162 | 759 | mGpsmUpsmCmUmUmAfAmAfGfAfC | 1006 | vmUpsfApsmUmGmGfAmCmAmAmAmGm |
| | | mUmUmUmGmUmCmCmAmUpsmA | | UmCfUmUfUmAmAmGmAmCpsmCpsmA |
| dsNA-163 | 760 | mUpsmCpsmUmUmAmAfAmGfAfCfU | 1007 | vmUpsfUpsmAmUmGfGmAmCmAmAmAm |
| | | mUmUmGmUmCmCmAmUmApsmA | | GmUfCmUfUmUmAmAmGmApsmCpsmC |
| dsNA-164 | 76 | mCpsmUpsmUmAmAmAfGmAfCfUfU | 1008 | vmUpsfUpsmUmAmUfGmGmAmCmAmAm |
| | | mUmGmUmCmCmAmUmAmApsmA | | AmGfUmCfUmUmUmAmAmGpsmApsmC |
| dsNA-165 | 762 | mUpsmUpsmAmAmAmGfAmCfUfUfU | 1009 | vmUpsfCpsmUmUmAfUmGmGmAmCmAm |
| | | mGmUmCmCmAmUmAmAmGpsmA | | AmAfGmUfCmUmUmUmAmApsmGpsmA |
| dsNA-166 | 763 | mUpsmApsmAmAmGmAfCmUfUfUfG | 1010 | vmUpsfUpsmCmUmUfAmUmGmGmAmCm |
| | | mUmCmCmAmUmAmAmGmApsmA | | AmAfAmGfUmCmUmUmUmApsmApsmG |
| dsNA-167 | 764 | mApsmApsmAmGmAmCfUmUfUfGfU | 1011 | vmUpsfGpsmUmCmUfUmAmUmGmGmAm |
| | | mCmCmAmUmAmAmGmAmCpsmA | | CmAfAmAfGmUmCmUmUmUpsmApsmA |
| dsNA-168 | 765 | mCpsmApsmAmCmAmUfAmUfUfUfG | 1012 | vmApsfApsmAmGmAfCmUmGmAmUmCm |
| | | mAmUmCmAmGmUmCmUmUpsmU | | AmAfAmUfAmUmGmUmUmGpsmApsmG |
| dsNA-169 | 766 | mApsmCpsmCmAmGmUfGmAfAfAfU | 1013 | vmUpsfUpsmCmUmUfCmUmUmUmGmAm |
| | | mCmAmAmAmGmAmAmGmApsmA | | UmUfUmCfAmCmUmGmGmUpsmUpsmU |
| dsNA-170 | 767 | mGpsmGpsmUmAmAmAfGmAfAfUfA | 1014 | vmUpsfCpsmAmAmGfUmGmAmCmAmUm |
| | | mUmGmUmCmAmCmUmUmGpsmA | | AmUfUmCfUmUmUmAmCmCpsmUpsmC |
| dsNA-171 | 768 | mGpsmUpsmAmAmAmGfAmAfUfAfU | 1015 | vmUpsfUpsmCmAmAfGmUmGmAmCmAm |
| | | mGmUmCmAmCmUmUmGmApsmA | | UmAfUmUfCmUmUmUmAmCpsmCpsmU |
| dsNA-172 | 769 | mUpsmApsmAmAmGmAfAmUfAfUfG | 1016 | vmUpsfUpsmUmCmAfAmGmUmGmAmCm |
| | | mUmCmAmCmUmUmGmAmApsmA | | AmUfAmUfUmCmUmUmUmApsmCpsmC |
| dsNA-173 | 770 | mApsmApsmAmGmAmAfUmAfUfGfU | 1017 | vmApsfGpsmUmUmCfAmAmGmUmGmAm |
| | | mCmAmCmUmUmGmAmAmCpsmU | | CmAfUmAfUmUmCmUmUmUpsmApsmC |
| dsNA-174 | 771 | mApsmApsmGmAmAmUfAmUfGfUfC | 1018 | vmUpsfApsmGmUmUfCmAmAmGmUmGm |
| | | mAmCmUmUmGmAmAmCmUpsmA | | AmCfAmUfAmUmUmCmUmUpsmUpsmA |
| dsNA-175 | 772 | mGpsmApsmAmUmAmUfGmUfCfAfC | 1019 | vmUpsfUpsmGmAmGfUmUmCmAmAmGm |
| | | mUmUmGmAmAmCmUmCmApsmA | | UmGfAmCfAmUmAmUmUmCpsmUpsmU |
| dsNA-176 | 773 | mApsmApsmUmAmUmGfUmCfAfCfU | 1020 | vmUpsfUpsmUmGmAfGmUmUmCmAmAm |
| | | mUmGmAmAmCmUmCmAmApsmA | | GmUfGmAfCmAmUmAmUmUpsmCpsmU |
| dsNA-177 | 774 | mApsmUpsmAmUmGmUfCmAfCfUfU | 1021 | vmApsfGpsmUmUmGfAmGmUmUmCmAm |
| | | mGmAmAmCmUmCmAmAmCpsmU | | AmGfUmGfAmCmAmUmAmUpsmUpsmC |
| dsNA-178 | 775 | mUpsmApsmUmGmUmCfAmCfUfUfG | 1022 | vmUpsfApsmGmUmUfGmAmGmUmUmCm |
| | | mAmAmCmUmCmAmAmCmUpsmA | | AmAfGmUfGmAmCmAmUmApsmUpsmU |
| dsNA-179 | 776 | mUpsmGpsmUmCmAmCfUmUfGfAfA | 1023 | vmUpsfUpsmGmAmGfUmUmGmAmGmUm |
| | | mCmUmCmAmAmCmUmCmApsmA | | UmCfAmAfGmUmGmAmCmApsmUpsmA |
| dsNA-180 | 777 | mGpsmUpsmCmAmCmUfUmGfAfAfC | 1024 | vmUpsfUpsmUmGmAfGmUmUmGmAmGm |
| | | mUmCmAmAmCmUmCmAmApsmA | | UmUfCmAfAmGmUmGmAmCpsmApsmU |
| dsNA-181 | 778 | mUpsmCpsmAmCmUmUfGmAfAfCfU | 1025 | vmUpsfUpsmUmUmGfAmGmUmUmGmAm |
| | | mCmAmAmCmUmCmAmAmApsmA | | GmUfUmCfAmAmGmUmGmApsmCpsmA |
| dsNA-182 | 779 | mApsmGpsmAmAmAmUfAmGfAfAfA | 1026 | vmUpsfUpsmGmAmGfCmUmGmAmUmUm |
| | | mAmUmCmAmGmCmUmCmApsmA | | UmUfCmUfAmUmUmUmCmUpsmUpsmU |
| dsNA-183 | 780 | mGpsmApsmAmAmUmAfGmAfAfAfA | 1027 | vmUpsfCpsmUmGmAfGmCmUmGmAmUm |
| | | mUmCmAmGmCmUmCmAmGpsmA | | UmUfUmCfUmAmUmUmUmCpsmUpsmU |
| dsNA-184 | 781 | mApsmApsmUmAmGmAfAmAfAfUfC | 1028 | vmUpsfUpsmUmCmUfGmAmGmCmUmGm |
| | | mAmGmCmUmCmAmGmAmApsmA | | AmUfUmUfUmCmUmAmUmUpsmUpsmC |
| dsNA-185 | 782 | mCpsmCpsmAmAmGmAfGmCfAfCfC | 1029 | vmApsfGpsmUmAmGfUmUmCmUmUmGm |
| | | mAmAmGmAmAmCmUmAmCpsmU | | GmUfGmCfUmCmUmUmGmGpsmCpsmU |
| dsNA-186 | 783 | mApsmApsmGmAmGmCfAmCfCfAfA | 1030 | vmUpsfGpsmAmGmUfAmGmUmUmCmUm |
| | | mGmAmAmCmUmAmCmUmCpsmA | | UmGfGmUfGmCmUmCmUmUpsmGpsmG |
| dsNA-187 | 784 | mApsmGpsmCmAmCmCfAmAfGfAfA | 1031 | vmApsfApsmGmGmGfAmGmUmAmGmUm |
| | | mCmUmAmCmUmCmCmCmUpsmU | | UmCfUmUfGmGmUmGmCmUpsmCpsmU |
| dsNA-188 | 785 | mApsmGpsmGmUmGmAfAmCfAfUfA | 1032 | vmApsfUpsmGmCmCfAmCmUmUmGmUm |
| | | mCmAmAmGmUmGmGmCmApsmU | | AmUfGmUfUmCmAmCmCmUpsmCpsmU |
| dsNA-189 | 786 | mGpsmUpsmGmAmAmCfAmUfAfCfA | 1033 | vmApsfCpsmAmUmGfCmCmAmCmUmUm |
| | | mAmGmUmGmGmCmAmUmGpsmU | | GmUfAmUfGmUmUmCmAmCpsmCpsmU |
| dsNA-190 | 787 | mUpsmGpsmAmAmCmAfUmAfCfAfA | 1034 | vmUpsfApsmCmAmUfGmCmCmAmCmUm |
| | | mGmUmGmGmCmAmUmGmUpsmA | | UmGfUmAfUmGmUmUmCmApsmCpsmC |
| dsNA-191 | 788 | mGpsmApsmAmCmAmUfAmCfAfAfG | 1035 | vmApsfUpsmAmCmAfUmGmCmCmAmCm |
| | | mUmGmGmCmAmUmGmUmApsmU | | UmUfGmUfAmUmGmUmUmCpsmApsmC |
| dsNA-192 | 789 | mGpsmGpsmUmUmUmUfGmGfGfAfG | 1036 | vmUpsfCpsmCmAmUfCmAmAmGmCmCm |
| | | mGmCmUmUmGmAmUmGmGpsmA | | UmCfCmCfAmAmAmAmCmCpsmApsmU |
| dsNA-193 | 790 | mUpsmGpsmGmGmAmGfGmCfUfUfG | 1037 | vmApsfApsmUmUmCfUmCmCmAmUmCm |
| | | mAmUmGmGmAmGmAmAmUpsmU | | AmAfGmCfCmUmCmCmCmApsmApsmA |
| dsNA-194 | 791 | mUpsmGpsmUmGmGmAfGmAfAfAfA | 1038 | vmApsfUpsmUmUmAfGmGmUmUmGmUm |
| | | mCmAmAmCmCmUmAmAmApsmU | | UmUfUmCfUmCmCmAmCmApsmCpsmU |
| dsNA-195 | 792 | mGpsmUpsmGmGmAmGfAmAfAfAfC | 1039 | vmUpsfApsmUmUmUfAmGmGmUmUmGm |
| | | mAmAmCmCmUmAmAmAmUpsmA | | UmUfUmUfCmUmCmCmAmCpsmApsmC |
| dsNA-196 | 793 | mUpsmGpsmGmAmGmAfAmAfAfCfA | 1040 | vmUpsfCpsmAmUmUfUmAmGmGmUmUm |
| | | mAmCmCmUmAmAmAmUmGpsmA | | GmUfUmUfUmCmUmCmCmApsmCpsmA |
| dsNA-197 | 794 | mGpsmGpsmAmGmAmAfAmAfCfAfA | 1041 | vmApsfCpsmCmAmUfUmUmAmGmGmUm |
| | | mCmCmUmAmAmAmUmGmGpsmU | | UmGfUmUfUmUmCmUmCmCpsmApsmC |
| dsNA-198 | 795 | mGpsmApsmGmAmAmAfAmCfAfAfC | 1042 | vmUpsfApsmCmCmAfUmUmUmAmGmGm |
| | | mCmUmAmAmAmUmGmGmUpsmA | | UmUfGmUfUmUmUmCmUmCpsmCpsmA |
| dsNA-199 | 796 | mGpsmApsmAmAmAmCfAmAfCfCfU | 1043 | vmUpsfUpsmUmAmCfCmAmUmUmUmAm |
| | | mAmAmAmUmGmGmUmAmApsmA | | GmGfUmUfGmUmUmUmUmCpsmUpsmC |
| dsNA-200 | 797 | mApsmUpsmCmAmAmGfAmUfUfUfG | 1044 | vmUpsfCpsmUmAmAfCmAmUmAmGmCm |
| | | mCmUmAmUmGmUmUmAmGpsmA | | AmAfAmUfCmUmUmGmAmUpsmUpsmU |
| dsNA-201 | 798 | mUpsmGpsmUmAmAmAfAmAfUfUfU | 1045 | vmUpsfCpsmAmUmUfGmGmCmUmAmAm |
| | | mUmAmGmCmCmAmAmUmGpsmA | | AmAfUmUfUmUmUmAmCmApsmUpsmC |
| dsNA-202 | 799 | mGpsmUpsmAmAmAmAfAmUfUfUfU | 1046 | vmUpsfCpsmCmAmUfUmGmGmCmUmAm |
| | | mAmGmCmCmAmAmUmGmGpsmA | | AmAfAmUfUmUmUmUmAmCpsmApsmU |
| dsNA-203 | 800 | mUpsmApsmAmAmAmAfUmUfUfUfA | 1047 | vmUpsfGpsmCmCmAfUmUmGmGmCmUm |
| | | mGmCmCmAmAmUmGmGmCpsmA | | AmAfAmAfUmUmUmUmUmApsmCpsmA |
| dsNA-204 | 801 | mApsmApsmAmAmAmUfUmUfUfAfG | 1048 | vmApsfGpsmGmCmCfAmUmUmGmGmCm |
| | | mCmCmAmAmUmGmGmCmCpsmU | | UmAfAmAfAmUmUmUmUmUpsmApsmC |
| dsNA-205 | 802 | mApsmApsmAmUmUmUfUmAfGfCfC | 1049 | vmUpsfGpsmAmGmGfCmCmAmUmUmGm |
| | | mAmAmUmGmGmCmCmUmCpsmA | | GmCfUmAfAmAmAmUmUmUpsmUpsmU |
| dsNA-206 | 803 | mApsmApsmUmUmUmUfAmGfCfCfA | 1050 | vmApsfGpsmGmAmGfGmCmCmAmUmUm |
| | | mAmUmGmGmCmCmUmCmCpsmU | | GmGfCmUfAmAmAmAmUmUpsmUpsmU |
| dsNA-207 | 804 | mUpsmUpsmAmGmCmCfAmAfUfGfG | 1051 | vmUpsfUpsmGmAmAfGmGmAmGmGmCm |
| | | mCmCmUmCmCmUmUmCmApsmA | | CmAfUmUfGmGmCmUmAmApsmApsmA |
| dsNA-208 | 805 | mUpsmApsmGmCmCmAfAmUfGfGfC | 1052 | vmApsfCpsmUmGmAfAmGmGmAmGmGm |
| | | mCmUmCmCmUmUmCmAmGpsmU | | CmCfAmUfUmGmGmCmUmApsmApsmA |
| dsNA-209 | 806 | mGpsmGpsmGmCmCmAfAmAfUfUfA | 1053 | vmApsfApsmUmAmUfGmUmCmAmUmUm |
| | | mAmUmGmAmCmAmUmAmUpsmU | | AmAfUmUfUmGmGmCmCmCpsmUpsmU |
| dsNA-210 | 807 | mGpsmGpsmCmCmAmAfAmUfUfAfA | 1054 | vmApsfApsmAmUmAfUmGmUmCmAmUm |
| | | mUmGmAmCmAmUmAmUmUpsmU | | UmAfAmUfUmUmGmGmCmCpsmCpsmU |
| dsNA-211 | 808 | mGpsmCpsmCmAmAmAfUmUfAfAfU | 1055 | vmUpsfApsmAmAmUfAmUmGmUmCmAm |
| | | mGmAmCmAmUmAmUmUmUpsmA | | UmUfAmAfUmUmUmGmGmCpsmCpsmC |
| dsNA-212 | 809 | mCpsmCpsmAmAmAmUfUmAfAfUfG | 1056 | vmUpsfGpsmAmAmAfUmAmUmGmUmCm |
| | | mAmCmAmUmAmUmUmUmCpsmA | | AmUfUmAfAmUmUmUmGmGpsmCpsmC |
| dsNA-213 | 810 | mCpsmApsmAmAmUmUfAmAfUfGfA | 1057 | vmUpsfUpsmGmAmAfAmUmAmUmGmUm |
| | | mCmAmUmAmUmUmUmCmApsmA | | CmAfUmUfAmAmUmUmUmGpsmGpsmC |
| dsNA-214 | 811 | mApsmApsmUmUmAmAfUmGfAfCfA | 1058 | vmUpsfUpsmUmUmGfAmAmAmUmAmUm |
| | | mUmAmUmUmUmCmAmAmApsmA | | GmUfCmAfUmUmAmAmUmUpsmUpsmG |
| dsNA-215 | 812 | mApsmUpsmUmAmAmUfGmAfCfAfU | 1059 | vmUpsfUpsmUmUmUfGmAmAmAmUmAm |
| | | mAmUmUmUmCmAmAmAmApsmA | | UmGfUmCfAmUmUmAmAmUpsmUpsmU |
| dsNA-216 | 813 | mApsmCpsmAmUmAmUfUmUfGfAfU | 1060 | vmApsfApsmAmAmAfGmAmCmUmGmAm |
| | | mCmAmGmUmCmUmUmUmUpsmU | | UmCfAmAfAmUmAmUmGmUpsmUpsmG |
| dsNA-217 | 814 | mCpsmApsmUmAmUmUfUmGfAfUfC | 1061 | vmUpsfApsmAmAmAfAmGmAmCmUmGm |
| | | mAmGmUmCmUmUmUmUmUpsmA | | AmUfCmAfAmAmUmAmUmGpsmUpsmU |
| dsNA-218 | 815 | mApsmUpsmAmUmUmUfGmAfUfCfA | 1062 | vmApsfUpsmAmAmAfAmAmGmAmCmUm |
| | | mGmUmCmUmUmUmUmUmApsmU | | GmAfUmCfAmAmAmUmAmUpsmGpsmU |
| dsNA-219 | 816 | mUpsmApsmUmUmUmGfAmUfCfAfG | 1063 | vmUpsfApsmUmAmAfAmAmAmGmAmCm |
| | | mUmCmUmUmUmUmUmAmUpsmA | | UmGfAmUfCmAmAmAmUmApsmUpsmG |
| dsNA-220 | 817 | mUpsmUpsmUmGmAmUfCmAfGfUfC | 1064 | vmApsfUpsmCmAmUfAmAmAmAmAmGm |
| | | mUmUmUmUmUmAmUmGmApsmU | | AmCfUmGfAmUmCmAmAmApsmUpsmA |
| dsNA-221 | 818 | mGpsmCpsmCmAmAmGfAmGfCfAfC | 1065 | vmUpsfUpsmAmGmUfUmCmUmUmGmGm |
| | | mCmAmAmGmAmAmCmUmApsmA | | UmGfCmUfCmUmUmGmGmCpsmUpsmU |
| dsNA-222 | 819 | mApsmGpsmAmGmCmAfCmCfAfAfG | 1066 | vmUpsfGpsmGmAmGfUmAmGmUmUmCm |
| | | mAmAmCmUmAmCmUmCmCpsmA | | UmUfGmGfUmGmCmUmCmUpsmUpsmG |
| dsNA-223 | 820 | mGpsmGpsmUmGmAmAfCmAfUfAfC | 1067 | vmUpsfApsmUmGmCfCmAmCmUmUmGm |
| | | mAmAmGmUmGmGmCmAmUpsmA | | UmAfUmGfUmUmCmAmCmCpsmUpsmC |
| dsNA-224 | 821 | mGpsmUpsmUmUmUmGfGmGfAfGfG | 1068 | vmUpsfUpsmCmCmAfUmCmAmAmGmCm |
| | | mCmUmUmGmAmUmGmGmApsmA | | CmUfCmCfCmAmAmAmAmCpsmCpsmA |
| dsNA-225 | 822 | mGpsmApsmGmAmAmUfUmUfUfGfG | 1069 | vmUpsfUpsmAmGmGfCmCmCmAmAmCm |
| | | mUmUmGmGmGmCmCmUmApsmA | | CmAfAmAfAmUmUmCmUmCpsmCpsmA |
| dsNA-226 | 823 | mGpsmApsmAmUmUmUfUmGfGfUfU | 1070 | vmUpsfUpsmCmUmAfGmGmCmCmCmAm |
| | | mGmGmGmCmCmUmAmGmApsmA | | AmCfCmAfAmAmAmUmUmCpsmUpsmC |
| dsNA-227 | 824 | mApsmUpsmUmUmUmGfGmUfUfGfG | 1071 | vmUpsfUpsmCmUmCfUmAmGmGmCmCm |
| | | mGmCmCmUmAmGmAmGmApsmA | | CmAfAmCfCmAmAmAmAmUpsmUpsmC |
| dsNA-228 | 825 | mUpsmUpsmUmUmGmGfUmUfGfGfG | 1072 | vmUpsfUpsmUmCmUfCmUmAmGmGmCm |
| | | mCmCmUmAmGmAmGmAmApsmA | | CmCfAmAfCmCmAmAmAmApsmUpsmU |
| dsNA-229 | 826 | mGpsmUpsmUmGmGmGfCmCfUfAfG | 1073 | vmUpsfApsmUmAmUfCmUmUmCmUmCm |
| | | mAmGmAmAmGmAmUmAmUpsmA | | UmAfGmGfCmCmCmAmAmCpsmCpsmA |
| dsNA-230 | 827 | mUpsmUpsmGmGmGmCfCmUfAfGfA | 1074 | vmUpsfUpsmAmUmAfUmCmUmUmCmUm |
| | | mGmAmAmGmAmUmAmUmApsmA | | CmUfAmGfGmCmCmCmAmApsmCpsmC |
| dsNA-231 | 828 | mUpsmGpsmGmGmCmCfUmAfGfAfG | 1075 | vmApsfGpsmUmAmUfAmUmCmUmUmCm |
| | | mAmAmGmAmUmAmUmAmCpsmU | | UmCfUmAfGmGmCmCmCmApsmApsmC |
| dsNA-232 | 829 | mApsmApsmAmAmCmAfAmCfCfUfA | 1076 | vmApsfUpsmUmUmAfCmCmAmUmUmUm |
| | | mAmAmUmGmGmUmAmAmApsmU | | AmGfGmUfUmGmUmUmUmUpsmCpsmU |
| dsNA-233 | 830 | mApsmApsmAmCmAmAfCmCfUfAfA | 1077 | vmUpsfApsmUmUmUfAmCmCmAmUmUm |
| | | mAmUmGmGmUmAmAmAmUpsmA | | UmAfGmGfUmUmGmUmUmUpsmUpsmC |
| dsNA-234 | 831 | mApsmApsmCmAmAmCfCmUfAfAfA | 1078 | vmApsfUpsmAmUmUfUmAmCmCmAmUm |
| | | mUmGmGmUmAmAmAmUmApsmU | | UmUfAmGfGmUmUmGmUmUpsmUpsmU |
| dsNA-235 | 832 | mApsmCpsmAmAmCmCfUmAfAfAfU | 1079 | vmUpsfApsmUmAmUfUmUmAmCmCmAm |
| | | mGmGmUmAmAmAmUmAmUpsmA | | UmUfUmAfGmGmUmUmGmUpsmUpsmU |
| dsNA-236 | 833 | mCpsmApsmAmCmCmUfAmAfAfUfG | 1080 | vmUpsfUpsmAmUmAfUmUmUmAmCmCm |
| | | mGmUmAmAmAmUmAmUmApsmA | | AmUfUmUfAmGmGmUmUmGpsmUpsmU |
| dsNA-237 | 834 | mApsmCpsmCmUmAmAfAmUfGfGfU | 1081 | vmUpsfGpsmUmUmAfUmAmUmUmUmAm |
| | | mAmAmAmUmAmUmAmAmCpsmA | | CmCfAmUfUmUmAmGmGmUpsmUpsmG |
| dsNA-238 | 835 | mCpsmUpsmAmAmAmUfGmGfUfAfA | 1082 | vmUpsfUpsmUmGmUfUmAmUmAmUmUm |
| | | mAmUmAmUmAmAmCmAmApsmA | | UmAfCmCfAmUmUmUmAmGpsmGpsmU |
| dsNA-239 | 836 | mUpsmApsmAmAmUmGfGmUfAfAfA | 1083 | vmUpsfUpsmUmUmGfUmUmAmUmAmUm |
| | | mUmAmUmAmAmCmAmAmApsmA | | UmUfAmCfCmAmUmUmUmApsmGpsmG |
| dsNA-240 | 837 | mCpsmApsmAmAmAmUfCmAfAfGfA | 1084 | vmApsfCpsmAmUmAfGmCmAmAmAmUm |
| | | mUmUmUmGmCmUmAmUmGmUp(ps) | | CmUfUmGfAmUmUmUmUmGpsmGpsmC |
| | | 2GalNAc4 | | |
| dsNA-241 | 838 | mGpsmUpsmCmAmCmUfUmGfAfAfC | 1085 | vmUpsfUpsmUmGmAfGmUmUmGmAmGm |
| | | mUmCmAmAmCmUmCmAmAmA | | UmUfCmAfAmGmUmGmAmCpsmApsmU |
| | | p(ps)2GalNAc4 | | |
| dsNA-242 | 839 | mApsmApsmUmUmAmAfUmGfAfCfA | 1086 | vmUpsfUpsmUmUmGfAmAmAmUmAmUm |
| | | mUmAmUmUmUmCmAmAmAmA | | GmUfCmAfUmUmAmAmUmUpsmUpsmG |
| | | p(ps)2GalNAc4 | | |
| dsNA-243 | 840 | mApsmCpsmAmUmAmUfUmUfGfAfU | 1087 | vmApsfApsmAmAmAfGmAmCmUmGmAm |
| | | mCmAmGmUmCmUmUmUmUmU | | UmCfAmAfAmUmAmUmGmUpsmUpsmG |
| | | p(ps)2GalNAc4 | | |
| dsNA-244 | 841 | mApsmApsmCmAmAmCfCmUfAfAfA | 1088 | vmApsfUpsmAmUmUfUmAmCmCmAmUm |
| | | mUmGmGmUmAmAmAmUmAmU | | UmUfAmGfGmUmUmGmUmUpsmUpsmU |
| | | p(ps)2GalNAc4 | | |
| dsNA-245 | 842 | mUpsmApsmAmAmUmGfGmUfAfAfA | 1089 | vmUpsfUpsmUmUmGfUmUmAmUmAmUm |
| | | mUmAmUmAmAmCmAmAmAmA | | UmUfAmCfCmAmUmUmUmApsmGpsmG |
| | | p(ps)2GalNAc4 | | |
| dsNA-246 | 843 | mApsmApsmAmAmUmCfAmAfGfAfU | 1090 | vmApsfApsmCmAmUfAmGmCmAmAmAm |
| | | mUmUmGmCmUmAmUmGmUmU | | UmCfUmUfGmAmUmUmUmUpsmGpsmG |
| | | p(ps)2GalNAc4 | | |
| dsNA-247 | 844 | mCpsmUpsmAmAmAmUfGmGfUfAfA | 1091 | vmUpsfUpsmUmGmUfUmAmUmAmUmUm |
| | | mAmUmAmUmAmAmCmAmAmA | | UmAfCmCfAmUmUmUmAmGpsmGpsmU |
| | | p(ps)2GalNAc4 | | |
| dsNA-248 | 845 | mCpsmApsmAmAmUmUfAmAfUfGfA | 1092 | vmUpsfUpsmGmAmAfAmUmAmUmGmUm |
| | | mCmAmUmAmUmUmUmCmAmA | | CmAfUmUfAmAmUmUmUmGpsmGpsmC |
| | | p(ps)2GalNAc4 | | |
| dsNA-249 | 846 | mUpsmUpsmUmGmAmUfCmAfGfUfC | 1093 | vmApsfUpsmCmAmUfAmAmAmAmAmGm |
| | | mUmUmUmUmUmAmUmGmAmU | | AmCfUmGfAmUmCmAmAmApsmUpsmA |
| | | p(ps)2GalNAc4 | | |
| dsNA-250 | 847 | mApsmApsmCmAmAmCfCmUfAfAfA | 1094 | vmApsfUpsmAmUmUfUmAmCmCmAmUm |
| | | mUmGmGfUmAmAmAmUmAmU | | UmUfAmGfGmUmUmGmUmUpsmUpsmU |
| | | p(ps)2GalNAc4 | | |
| dsNA-251 | 848 | mApsmApsmCmAmAmCfCmUfAfAfA | 1095 | vmApsfUpsmAmUmUfUmAmCmCmAmUm |
| | | mUmGmGmUmAmAfAmUmAmU | | UmUfAmGfGmUmUmGmUmUpsmUpsmU |
| | | p(ps)2GalNAc4 | | |
| dsNA-252 | 849 | mApsmApsmCmAmAmClfCmUfAfAfA | 1096 | vmApsfUpsmAmUmUfUmAmCmCmAmUm |
| | | mUmGmGmUmAmAmAmUmAmU | | UmUfAmGfGmUmUmGmUmUpsmUpsmU |
| | | p(ps)2GalNAc4 | | |
| dsNA-253 | 850 | mApsmApsmCmAmAmCfCmUfAfAfA | 1097 | vmApsfUpsfAmUmUfUmAmCmCmAmUm |
| | | mUmGmGfUmAmAmAmUmAmU | | UmUfAmGfGmUmUmGmUmUpsmUpsmU |
| | | p(ps)2GalNAc4 | | |
| dsNA-254 | 851 | mApsmApsmCmAmAmCfCmUfAfAfA | 1098 | vmApsfUpsmAmUmUfUmAmCfCmAmUm |
| | | mUmGmGmUmAmAfAmUmAmU | | UmUfAmGfGmUmUmGmUmUpsmUpsmU |
| | | p(ps)2GalNAc4 | | |
| dsNA-255 | 852 | mApsmApsmCmAmAmCfCmUfAfAfA | 1099 | vmApsfUpsmAmUmUfUmAmCmCmAmUm |
| | | mUmGmGmUmAmAfAmUmAmU | | UmUfAmGfGmUmUmGmUfUpsmUpsmU |
| | | p(ps)2GalNAc4 | | |
| dsNA-256 | 853 | mApsmApsmCmAmAmCfCmUfAfAfA | 1100 | vmApsfUpsfAmUmUfUmAmCmCmAmUm |
| | | mUmGmGmUmAmAmAmUmAmU | | UmUfAmGfGmUmUmGmUmUpsmUpsmU |
| | | p(ps)2GalNAc4 | | |
| dsNA-257 | 854 | mApsmApsmCmAmAmCfCmUfAfAfA | 1101 | vmApsfUpsmAmUmUfUmAmCfCmAmUm |
| | | mUmGmGmUmAmAmAmUmAmU | | UmUfAmGfGmUmUmGmUmUpsmUpsmU |
| | | p(ps)2GalNAc4 | | |
| dsNA-258 | 855 | mApsmApsmCmAmAmCfCmUfAfAfA | 1102 | vmApsfUpsmAmUmUfUmAmCmCmAmUm |
| | | mUmGmGmUmAmAmAmUmAmU | | UmUfAmGfGmUmUmGmUfUpsmUpsmU |
| | | p(ps)2GalNAc4 | | |
| dsNA-259 | 856 | mApsmApsmCmAmAmCfCmUfAfAfA | 1103 | vmApsfUpsmAmUmUlfUmAmCmCmAmU |
| | | mUmGmGmUmAmAmAmUmAmU | | mUmUfAmGfGmUmUmGmUmUpsmUpsm |
| | | p(ps)2GalNAc4 | | U |
| dsNA-260 | 857 | mApsmApsmCmAmAmCfCmUfAfAfA | 1104 | vmApsfUpsmAmUmUfUmAmCfCmAmUm |
| | | mUmGmGmUmAmAmAmUmAmU | | UmUfAmGfGmUmUmGmUmUpslmUpsmU |
| | | p(ps)2GalNAc4 | | |
| dsNA-261 | 858 | mApsmApsmCmAmAmCfCmUfAfAfA | 1105 | vmApsfUpsmAmUmUfun34UmAmCfCmAm |
| | | mUmGmGmUmAmAmAmUmAmU | | UmUmUfAmGfGmUmUmGmUmUpsmUps |
| | | p(ps)2GalNAc4 | | mU |
| dsNA-262 | 859 | mCpsmApsmAmAmAmUfCmAfAfGfA | 1106 | vmApsfCpsmAmUmAfGunCmAmAmAmUm |
| | | mUmUmUmGmCmUmAmUmGmU | | CmUfUmGfAmUmUmUmUmGpsmGpsmC |
| | | p(ps)2GalNAc4 | | |
| dsNA-263 | 860 | mCpsmApsmAmAmAmUfCmAfAfGfA | 1107 | vmApsfCpsmAmUmAfGmun34CmAmAmA |
| | | mUmUmUmGmCmUmAmUmGmU | | mUmCmUfUmGfAmUmUmUmUmGpsmGp |
| | | p(ps)2GalNAc4 | | smC |
| dsNA-264 | 861 | mCpsmApsmAmAmAmUfCmAfAfGfA | 1108 | vmApsfCpsmAmUmAfG3ohCmAmAmAmU |
| | | mUmUmUmGmCmUmAmUmGmU | | mCmUfUmGfAmUmUmUmUmGpsmGpsmC |
| | | p(ps)2GalNAc4 | | |
| dsNA-265 | 862 | mCpsmApsmAmAmAmUfCmAfAfGfA | 1109 | vmApsfCpsmAmUmAfG3ocpCmAmAmAm |
| | | mUmUmUmGmCmUmAmUmGmU | | UmCmUfUmGfAmUmUmUmUmGpsmGps |
| | | p(ps)2GalNAc4 | | mC |
| dsNA-266 | 863 | mCpsmApsmAmAmAmUfCmAfAfGfA | 1110 | vmApsfCpsmAmUmAlfGmCmAmAmAmU |
| | | mUmUmUmGmCmUmAmUmGmU | | mCmUfUmGfAmUmUmUmUmGpsmGpsmC |
| | | p(ps)2GalNAc4 | | |
| dsNA-267 | 864 | mApsmApsmCmAmAmCfCmUfAfAfA | 1111 | vmUpsfUpsmAmUmUfUmAmCmCmAmUm |
| | | mUmGmGmUmAmAmAmUmAmU | | UmUfAmGfGmUmUmGmUmUpsmUpsmU |
| | | p(ps)2GalNAc4 | | |
| dsNA-268 | 865 | mApsmApsmCmAmAmCfCmUfAfAfA | 1112 | vm(5mim)UpsfUpsmAmUmUfUmAmCmCm |
| | | mUmGmGmUmAmAmAmUmAmU | | AmUmUmUfAmGfGmUmUmGmUmUpsmU |
| | | p(ps)2GalNAc4 | | psmU |
| dsNA-269 | 866 | mApsmApsmCmAmAmCfCmUfAfAfA | 1113 | vm(56mido)UpsfUpsmAmUmUfUmAmCmC |
| | | mUmGmGmUmAmAmAmUmAmU | | mAmUmUmUfAmGfGmUmUmGmUmUpsm |
| | | p(ps)2GalNAc4 | | UpsmU |
| dsNA-270 | 867 | mCpsmApsmAmAmAmUfCmAfAfGfA | 1114 | vmApsfCpsmAmUmAunGmCmAmAmAmU |
| | | mUmUmUmGmCmUmAmUmGmU | | mCmUfUmGfAmUmUmUmUmGpsmGpsmC |
| | | p(ps)2GalNAc4 | | |
| dsNA-271 | 868 | mCpsmApsmAmAmAmUfCmAfAfGfA | 1115 | vmApsfCpsmAmUmAmun34GmCmAmAmA |
| | | mUmUmUmGmCmUmAmUmGmU | | mUmCmUfUmGfAmUmUmUmUmGpsmGp |
| | | p(ps)2GalNAc4 | | smC |
| dsNA-272 | 869 | mCpsmApsmAmAmAmUfCmAfAfGfA | 1116 | vmApsfCpsmAmUmA3ocpGmCmAmAmAm |
| | | mUmUmUmGmCmUmAmUmGmU | | UmCmUfUmGfAmUmUmUmUmGpsmGps |
| | | p(ps)2GalNAc4 | | mC |
| dsNA-273 | 870 | mCpsmApsmAmAmAmUfCmAfAfGfA | 1117 | vmApsfCpsmAmUmA30hGmCmAmAmAm |
| | | mUmUmUmGmCmUmAmUmGmU | | UmCmUfUmGfAmUmUmUmUmGpsmGps |
| | | p(ps)2GalNAc4 | | mC |
| dsNA-274 | 871 | mCpsmApsmAmAmAmUfCmAfAfGfA | 1118 | vmApsfCpsmAmUmAganr?GmCmAmAmA |
| | | mUmUmUmGmCmUmAmUmGmU | | mUmCmUfUmGfAmUmUmUmUmGpsmGp |
| | | p(ps)2GalNAc4 | | smC |
| dsNA-275 | 872 | mCpsmApsmAmAmAmUfCmAfAfGfA | 1119 | vmApsfCpsmAmUmAgans?GmCmAmAmA |
| | | mUmUmUmGmCmUmAmUmGmU | | mUmCmUfUmGfAmUmUmUmUmGpsmGp |
| | | p(ps)2GalNAc4 | | smC |
| dsNA-276 | 873 | mCpsmApsmAmAmAmUfCmAfAfGfA | 1120 | vmApsfCpsmAmUmAun23entGmCmAmAm |
| | | mUmUmUmGmCmUmAmUmGmU | | AmUmCmUfUmGfAmUmUmUmUmGpsmG |
| | | p(ps)2GalNAc4 | | psmC |
| dsNA-277 | 874 | mCpsmApsmAmAmAmUfCmAfAfGfA | 1121 | vmApslfCpsmAmUmAfGmCmAmAmAmU |
| | | mUmUmUmGmCmUmAmUmGmU | | mCmUfUmGfAmUmUmUmUmGpsmGpsmC |
| | | p(ps)2GalNAc4 | | |
| dsNA-278 | 875 | mCpsmApsmAmAmAmUfCmAfAfGfA | 1122 | vmApsfCpsmAmUmAfGmCmAmAmAmUm |
| | | mUmUmUmGmCmUmAmUmGmU | | CmUlfUmGfAmUmUmUmUmGpsmGpsmC |
| | | p(ps)2GalNAc4 | | |
| dsNA-279 | 876 | mCpsmApsmAmAmAmUfCmAfAfGfA | 1123 | vmApsfCpsmAmUmAfGmCmAmAmAmUm |
| | | mUmUmUmGmCmUmAmUmGmU | | CmUfUmGlfAmUmUmUmUmGpsmGpsmC |
| | | p(ps)2GalNAc4 | | |
| dsNA-280 | 877 | mCpsmApsmAmAmAmUfCmAfAfGfA | 1124 | vmApsfCpsmAmUmAfGmCmAmAmAmUm |
| | | mUmUmUmGmCmUmAmUmGmU | | CmUfUmGfAmUmUmUmUmGpslmGpsmC |
| | | p(ps)2GalNAc4 | | |
| dsNA-281 | 878 | mCpsmApsmAmAmAmUfCmAfAfGfA | 1125 | vmApsfCpsmAmUmAfGmCmAfAmAmUmC |
| | | mUmUmUmGmCmUmAmUmGmU | | mUfUmGfAmUmUmUmUmGpslmGpsmC |
| | | p(ps)2GalNAc4 | | |

Abbreviations: f is a 2′-fluoro nucleotide,
m is a 2′-O-methyl nucleotide,
ps is phosphorothioate internucleotide
linkage, v is a 5′-vinyl phosphonate, vm is 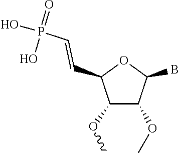
wherein B is a nucleobase or derivative thereof,
vm(5mim) is 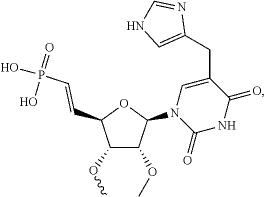
vm(56mido) is 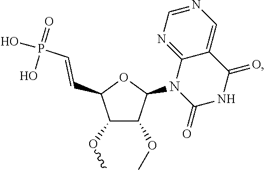
fun34U is 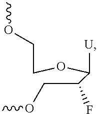
mun34 is 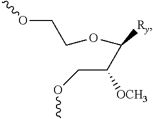
3ocp is 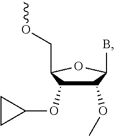
ganr\hat{ } is 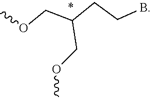
gans\hat{ } is 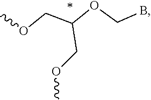
1f is 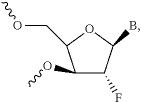
1m is 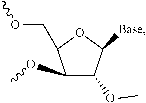
23entG is 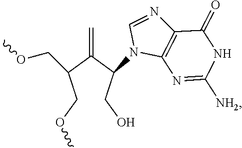
and
p(ps)2GalNAc4 is

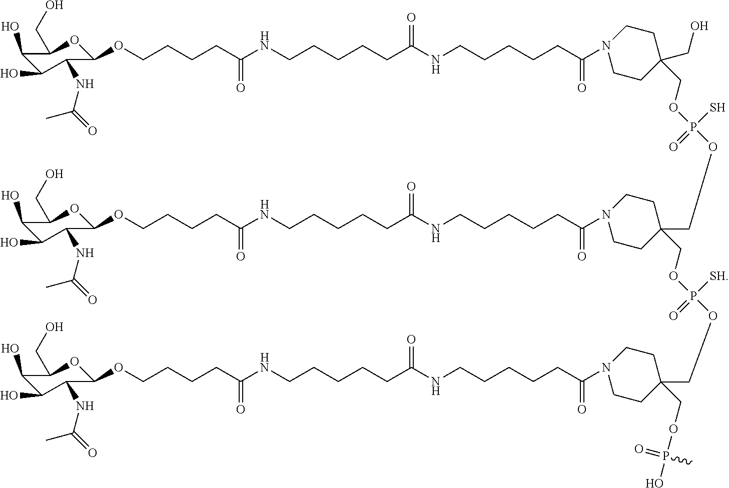

### Pharmaceutical Compositions

The present disclosure also encompasses pharmaceutical compositions comprising siNAs of the present disclosure. In some embodiments is a pharmaceutical composition comprising one or more siNA of the present disclosure, and a pharmaceutically acceptable diluent or carrier. The disclosed siNA can be formulated in a pharmaceutical composition to individually target or inhibit or reduce expression of ANGPTL3 or ANGPTL8, or the disclosed siNA can be formulated in a pharmaceutical composition to simultaneously target or inhibit or reduce expression of both ANGPTL3 and ANGPTL8.

Accordingly, a pharmaceutical composition may comprise at least one siNA comprising a sense strand comprising any one of SEQ ID NOs: 1-316 and an antisense strand comprising any one of SEQ ID NOs: 317-632. A pharmaceutical composition may comprise at least one siNA comprising a sense strand comprising any one of SEQ ID NOs: 633-878 and an antisense strand comprising any one of SEQ ID NOs: 879-1125. In instances in which the goal is to simultaneously target or inhibit or reduce expression of both ANGPTL3 and ANGPTL8, a pharmaceutical composition may comprise at least one siNA comprising a sense strand comprising any one of SEQ ID NOs: 1-316 and an antisense strand comprising any one of SEQ ID NOs: 317-632 and at least one siNA comprising a sense strand comprising any one of SEQ ID NOs: 633-878 and an antisense strand comprising any one of SEQ ID NOs: 879-1125. Alternatively, in instances in which the goal is to simultaneously target or inhibit or reduce expression of both ANGPTL3 and ANGPTL8, two separate pharmaceutical compositions may be administered to a subject concurrently or sequentially, wherein one pharmaceutical composition comprises at least one siNA comprising a sense strand comprising any one of SEQ ID NOs: 1-316 and an antisense strand comprising any one of SEQ ID NOs: 317-632 and the other pharmaceutical composition comprises at least one siNA comprising a sense strand comprising any one of SEQ ID NOs: 633-878 and an antisense strand comprising any one of SEQ ID NOs: 879-1125.

In some embodiments, the pharmaceutical compositions comprising any of the siNA molecules, sense strands, antisense strands, first nucleotide sequences, or second nucleotide sequences described herein. The compositions may comprise 1, 2, 3, or more siNA molecules described herein. In some embodiments, the composition comprises a double-stranded short interfering nucleic acid (ds-siNA) molecule comprises (a) a sense strand comprising a first nucleotide sequence, wherein the first nucleotide sequence is 15 to 30 nucleotides in length and comprises a nucleotide sequence as set forth in SEQ ID NOs: 1-316; and (b) an antisense strand comprising a second nucleotide sequence, wherein the second nucleotide sequence is 15 to 30 nucleotides in length and comprises a nucleotide sequence that is the reverse complement of the first nucleotide sequence. In some embodiments, the first nucleotide sequence comprises a nucleotide sequence as set forth in SEQ ID NOs: 1-316; and (b) an antisense strand that is the reverse complement of the first nucleotide sequence, wherein the antisense strand comprises a second nucleotide sequence, wherein the second nucleotide sequence is 15 to 30 nucleotides in length and comprises a nucleotide sequence as set forth in SEQ ID NOs: 317-632.

In some embodiments, the pharmaceutical compositions comprising any of the siNA molecules, sense strands, antisense strands, first nucleotide sequences, or second nucleotide sequences described herein. The compositions may comprise 1, 2, 3, or more siNA molecules described herein. In some embodiments, the composition comprises a double-stranded short interfering nucleic acid (ds-siNA) molecule comprises (a) a sense strand comprising a first nucleotide sequence, wherein the first nucleotide sequence is 15 to 30 nucleotides in length and comprises a nucleotide sequence as set forth in SEQ ID NOs: 633-878; and (b) an antisense strand comprising a second nucleotide sequence, wherein the second nucleotide sequence is 15 to 30 nucleotides in length and comprises a nucleotide sequence that is the reverse complement of the first nucleotide sequence. In some embodiments, the first nucleotide sequence comprises a nucleotide sequence as set forth in SEQ ID NOs: 633-878; and (b) an antisense strand that is the reverse complement of the first nucleotide sequence, wherein the antisense strand comprises a second nucleotide sequence, wherein the second nucleotide sequence is 15 to 30 nucleotides in length and comprises a nucleotide sequence as set forth in SEQ ID NOs: 879-1125.

In some embodiments, the composition comprises a double-stranded short interfering nucleic acid (ds-siNA) molecule comprising (a) a sense strand comprising a nucleotide sequence identical to SEQ ID NOs: 1-316, and (b) an antisense strand. In some embodiments, the present disclosure provides a double-stranded short interfering nucleic acid (ds-siNA) molecule comprising (a) an antisense strand comprising a nucleotide sequence identical to SEQ ID NOs: 317-632; and (b) a sense strand.

In some embodiments, the composition comprises a double-stranded short interfering nucleic acid (ds-siNA) molecule comprising (a) a sense strand comprising a nucleotide sequence identical to SEQ ID NOs: 633-878, and (b) an antisense strand. In some embodiments, the present disclosure provides a double-stranded short interfering nucleic acid (ds-siNA) molecule comprising (a) an antisense strand comprising a nucleotide sequence identical to SEQ ID NOs: 879-1125; and (b) a sense strand.

In some embodiments, the composition comprises a double-stranded short interfering nucleic acid (ds-siNA) molecule comprising (a) a sense strand as set forth in is SEQ ID NOs: 633, 634, 642, 653, 654, 674, 687, 688, 690-694, 708-713, 756-757, 765, 776, 777, 797, 810, 811, 813-817, or 831-836, and (b) an antisense strand complementary to the sense strand as set forth in SEQ ID NOs: 879, 880, 888, 899, 900, 920, 933, 934, 936-940, 954-959, 1003-1004, 1012, 1023, 1024, 1044, 1057, 1058, 1060-1064, or 1078-1083, respectively. In some embodiments, the composition comprises a double-stranded short interfering nucleic acid (ds-siNA) molecule comprising (a) a sense strand according to SEQ ID NOs: 644, 654, 688, 690, 708, 713, 756, 777, 811, 813, 831, or 836, and (b) an antisense strand complementary to the sense strand according to SEQ ID NOs: 879, 900, 934, 936, 954, 959, 1003, 1024, 1058, 1060, 1078, or 1083, respectively.

Alternatively or additionally, the pharmaceutical compositions may comprise (a) a phosphorylation blocker; and (b) an oligomer. In some embodiments, the phosphorylation blocker is any of the phosphorylation blockers disclosed herein. In some embodiments, the oligomer is any of the oligomers disclosed herein. In some embodiments, the oligomer comprises any of the sense strands, antisense strands, first nucleotide sequences, or second nucleotide sequences described herein. In some embodiments, the oligomer comprises any of the sense strands, antisense strands, first nucleotide sequences, or second nucleotide sequences described herein. In some embodiments, the oligomer comprises one or more modified nucleotides. In some embodiments, the one or more modified nucleotides are independently selected from a 2′-fluoro nucleotide and a 2′-O-methyl nucleotide. In some embodiments, the 2′-fluoro nucleotide or the 2′-O-methyl nucleotide is independently selected from any of the 2′-fluoro or 2′-O-methyl nucleotide mimics disclosed herein. In some embodiments, the oligomer comprises a nucleotide sequence comprising any of the modification patterns disclosed herein.

In some embodiments, the pharmaceutical composition comprises (a) a 5′-stabilized end cap; and (b) an oligomer. In some embodiments, the 5′-stabilized end cap is any of the 5-stabilized end caps disclosed herein. In some embodiments, the oligomer is any of the oligomers disclosed herein. In some embodiments, the oligomer comprises any of the sense strands, antisense strands, first nucleotide sequences, or second nucleotide sequences described herein. In some embodiments, the oligomer comprises one or more modified nucleotides. In some embodiments, the one or more modified nucleotides are independently selected from a 2′-fluoro nucleotide and a 2′-O-methyl nucleotide. In some embodiments, the 2′-fluoro nucleotide or the 2′-O-methyl nucleotide is independently selected from any of the 2′-fluoro or 2′-O-methyl nucleotide mimics disclosed herein. In some embodiments, the oligomer comprises a nucleotide sequence comprising any of the modification patterns disclosed herein.

In some embodiments, the pharmaceutical composition comprises (a) at least one phosphorylation blocker or 5′-stabilized end cap; and (b) an oligomer. In some embodiments, the phosphorylation blocker is any of the phosphorylation blockers disclosed herein. In some embodiments, the conjugated moiety is any of the galactosamines disclosed herein. In some embodiments, the 5′-stabilized end cap is any of the 5-stabilized end caps disclosed herein. In some embodiments, the oligomer is any of the oligomers disclosed herein. In some embodiments, the oligomer comprises any of the sense strands, antisense strands, first nucleotide sequences, or second nucleotide sequences described herein. In some embodiments, the oligomer comprises one or more modified nucleotides. In some embodiments, the one or more modified nucleotides are independently selected from a 2′-fluoro nucleotide and a 2′-O-methyl nucleotide. In some embodiments, the 2′-fluoro nucleotide or the 2′-O-methyl nucleotide is independently selected from any of the 2′-fluoro or 2′-O-methyl nucleotide mimics disclosed herein. In some embodiments, the oligomer comprises a nucleotide sequence comprising any of the modification patterns disclosed herein.

In some embodiments, the pharmaceutical composition containing the siNA of the present disclosure is formulated for systemic administration via parenteral delivery. Parenteral administration includes intravenous, intra-arterial, subcutaneous, intraperitoneal or intramuscular injection or infusion; also subdermal administration, e.g., via an implanted device. In a preferred embodiment, the pharmaceutical composition containing the siNA of the present disclosure is formulated for subcutaneous (SC) or intravenous (IV) delivery. Formulations for parenteral administration may include sterile aqueous solutions, which may also contain buffers, diluents and other pharmaceutically acceptable additives as understood by the skilled artisan. For intravenous use, the total concentration of solutes may be controlled to render the preparation isotonic.

When the pharmaceutical composition comprises two or more siNAs, the siNAs may be present in varying amounts. For example, in some embodiments, the weight ratio of first siNA to second siNA is 1:10 to 10:1, e.g., 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1. In some embodiments, the molar ratio of first siNA to second siNA is 1:10 to 10:1, e.g., 1:10 to 10:1, e.g., 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1.

In some embodiments, the pharmaceutical composition comprises an amount of one or more of the siNA molecules described herein formulated with one or more pharmaceutically acceptable carriers (additives) and/or diluents. The pharmaceutical compositions may be specially formulated for administration in solid or liquid form, including those adapted for the following: (1) parenteral administration, for example, by subcutaneous, intramuscular, intravenous or epidural injection as, for example, a sterile solution or suspension, or sustained-release formulation; (2) topical application, for example, as a cream, ointment, or a controlled-release patch or spray applied to the skin; (3) intravaginally or intrarectally, for example, as a pessary, cream or foam; (4) sublingually; (5) ocularly; (6) transdermally; or (7) nasally.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present disclosure include those suitable for nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound (e.g., siNA molecule) which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In some embodiments, a formulation of the present disclosure comprises an excipient selected from the group consisting of cyclodextrins, celluloses, liposomes, micelle forming agents, e.g., bile acids, and polymeric carriers, e.g., polyesters and polyanhydrides; and a compound (e.g., siNA molecule) of the present disclosure.

Methods of preparing these formulations or compositions include the step of bringing into association a compound (e.g., siNA molecule) of the present disclosure with the carrier and, optionally, one or more accessory ingredients. In general, the formulations are prepared by uniformly and intimately bringing into association a compound (e.g., siNA molecule) of the present disclosure with liquid carriers, or finely divided solid carriers, or both, and then, if necessary, shaping the product.

Formulations of the disclosure suitable for a suspension in an aqueous or non-aqueous liquid, or as an oil-in-water or water-in-oil liquid emulsion, or as an elixir or syrup, each containing a predetermined amount of a compound (e.g., siNA molecule) of the present disclosure as an active ingredient. A compound (e.g., siNA molecule) of the present disclosure may also be administered as a bolus, electuary, or paste.

In dosage forms of the disclosure, the active ingredient may be mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose.

The disclosed dosage forms may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms of the compounds (e.g., siNA molecules) of the disclosure include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (I particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the compositions can also include adjuvants such as wetting agents, emulsifying and suspending agents, sweetening, flavoring, coloring, perfuming and preservative agents.

Suspensions, in addition to the active compounds (e.g., siNA molecules), may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the disclosure for rectal or vaginal administration may be presented as a suppository, which may be prepared by mixing one or more compounds (e.g., siNA molecules) of the disclosure with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound (e.g., siNA molecule).

Formulations of the present disclosure which are suitable for vaginal administration also include pessaries, tampons, creams, gels, pastes, foams or spray formulations containing such carriers as are known in the art to be appropriate.

Dosage forms for the topical or transdermal administration of a compound (e.g., siNA molecule) of this disclosure include powders, sprays, ointments, pastes, creams, lotions, gels, solutions, patches and inhalants. The active compound (e.g., siNA molecule) may be mixed under sterile conditions with a pharmaceutically acceptable carrier, and with any preservatives, buffers, or propellants which may be required.

The ointments, pastes, creams and gels may contain, in addition to an active compound (e.g., siNA molecule) of this disclosure, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound (e.g., siNA molecule) of this disclosure, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound (e.g., siNA molecule) of the present disclosure to the body. Such dosage forms can be made by dissolving or dispersing the compound (e.g., siNA molecule) in the proper medium. Absorption enhancers can also be used to increase the flux of the compound (e.g., siNA molecule) across the skin. The rate of such flux can be controlled by either providing a rate controlling membrane or dispersing the compound (e.g., siNA molecule) in a polymer matrix or gel.

Ophthalmic formulations, eye ointments, powders, solutions and the like, are also contemplated as being within the scope of this invention.

Pharmaceutical compositions of this disclosure suitable for parenteral administration comprise one or more compounds (e.g., siNA molecules) of the disclosure in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds (e.g., siNA molecules) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

In some embodiments, the present disclosure provides a pharmaceutical composition comprising the siNAs disclosed herein conjugated to a ligand targeting receptors expressed in hepatocytes to treat Cardiovascular Diseases (CVD) and MASH The ligands that could be conjugated to the siNAs include but are not limited to GalNAc, Cholesterol, Palmitic Acid etc.

In some embodiments, the pharmaceutical composition comprises a liposome that encapsulates the siNAs disclosed herein to form a lipid nanoparticle (LNP) targeting hepatocellular carcinoma (HCC) or other secondary cancers metastasized to the liver.

In some embodiments, the present disclosure provides a pharmaceutical composition comprising the siNAs disclosed herein conjugated to a ligand targeting receptors expressed in HCC. The ligands that could be conjugated to the siNAs include but are not limited to GalNAc, Folate, Cholesterol, Palmitic Acid etc.

When the compounds (e.g., siNA molecules) of the present disclosure are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier. In some embodiments, the pharmaceutical acceptable carriers is a liposome that encapsulates the ds-siNA molecules.

### Methods of Treatment and Administration

The siNA molecules of the present disclosure may be used to treat or prevent a disease in a subject in need thereof. In some embodiments, a method of treating or preventing a disease in a subject in need thereof comprises administering to the subject any of the siNA molecules disclosed herein. In some embodiments, a method of treating or preventing a disease in a subject in need thereof comprises administering to the subject any of the compositions disclosed herein.

In some embodiments, the subject is a mammal. In some embodiments, the subject is a human. In some embodiments, the subject is a non-human primate. In some embodiments, the subject is a cat. In some embodiments, the subject is a camel. In preferred embodiments in which the subject is a human, the subject may be at least 40 years old, at least 45 years old, at least 50 years old, at least 55 years old, at least 60 years old, at least 65 years old, at least 70 years old, at least 75 years old, or at least 80 years old or older. In some embodiments, the subject is a pediatric subject (i.e., less than 18 years old).

The preparations (e.g., siNA molecules or pharmaceutical compositions thereof) of the present disclosure may be given parenterally, topically, or rectally or administered in the form of an inhalant. They are, of course, given in forms suitable for each administration route. For example, they are administered in tablets or capsule form, administration by injection, infusion, or inhalation; topical by lotion or ointment; rectal by suppositories. Injection, infusion, or inhalation are preferred. In some embodiments, the siNA molecules are given in the form of nanoparticle where a liposome encapsulates the siNA molecules.

These compounds may be administered to humans and other animals for therapy or as a prophylactic by any suitable route of administration, including nasally (as by, for example, a spray), rectally, intravaginally, parenterally, intracisternally and topically, as by powders, ointments or drops, including buccally and sublingually. In some embodiments, the compounds or compositions are inhaled, as by, for example, an inhaler, a nebulizer, or in an aerosolized form.

Regardless of the route of administration selected, the compounds (e.g., siNA molecules) of the present disclosure, which may be used in a suitable hydrated form, and/or the pharmaceutical compositions of the present disclosure, are formulated into pharmaceutically-acceptable dosage forms by conventional methods known to those of skill in the art.

In some embodiments, the present disclosure provides methods of treating or preventing a disease in a subject in need thereof, comprising administering the subject the ds-siNA molecules disclosed herein to reduce ANGPTL8 and/or ANGPTL3 activity or level. In some embodiments, the method of treating or preventing the disease comprises administering the subject the pharmaceutical composition comprising the ds-siNA molecules disclosed herein to reduce ANGPTL8 and/or ANGPTL3 activity or level.

In some embodiments, the disease is a ANGPTL8 and/or ANGPTL3 associated disease or disorder. In some embodiments, the ANGPTL8 and/or ANGPTL3 associated disease or disorder comprises cardiovascular disease or nonalcoholic fatty liver disease (NAFLD), such as metabolic dysfunction-associated steatohepatitis (MASH). In some embodiments, the cardiovascular disease is hypertriglyceridemia (HTG) or familial hypercholesterolemia (FH).

Actual dosage levels of the active ingredients (e.g., siNA molecules) in the pharmaceutical compositions of this disclosure may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound (e.g., siNA molecule) of the present disclosure employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds (e.g., siNA molecules) of the disclosure employed in the pharmaceutical composition at levels lower than that required in order to achieve the desired therapeutic effect and gradually increase the dosage until the desired effect is achieved.

In general, a suitable daily dose of a compound (e.g., siNA molecule) of the disclosure is the amount of the compound that is the lowest dose effective to produce a therapeutic effect. Such an effective dose generally depends upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. In some embodiments, the compound is administered at a dose equal to or greater than 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.35, 0.40, 0.45, 0.50, 0.55, 0.60, 0.65, 0.7, 0.75, 0.8, 0.85, 0.9, 0.95, or 1 mg/kg. In some embodiments, the compound is administered at a dose equal to or less than 200, 190, 180, 170, 160, 150, 140, 130, 120, 110, 100, 95, 90, 85, 80, 75, 70, 65, 60, 55, 50, 45, 40, 35, 30, 25, 20, or 15 mg/kg. In some embodiments, the total daily dose of the compound is equal to or greater than 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 100 mg.

When the compounds (e.g., siNA molecules) described herein are co-administered with another, the effective amount may be less than when the compound is used alone.

If desired, the effective daily dose of the active compound (e.g., siNA molecule) may be administered as two, three, four, five, six or more sub-doses administered separately at appropriate intervals throughout the day, optionally, in unit dosage forms. Preferred dosing is one administration per day. In some embodiments, the compound is administered at least 1, 2, 3, 4, 5, 6, 7, 8, 9, 10, 11, 12, 13, 14, 15, 16, 17, 18, 19, 20, or 21 times a week. In some embodiments, the compound is administered at least 1, 2, 3, 4, 5, 6, 7, 8, 9, 10, 11, 12, 13, 14, 15, 16, 17, 18, 19, 20, or 21 times a month. In some embodiments, the compound is administered once every 1, 2, 3, 4, 5, 6, 7, 8, 9, 10, 11, 12, 13, 14, 15, 16, 17, 18, 19, 20, or 21 days. In some embodiments, the compound is administered once every 1, 2, 3, 4, 5, 6, 7, or 8 weeks.

In some embodiments, the disclosed siNAs and pharmaceutical compositions comprising the disclosed siNAs are combined with approved or late stage development drugs in cardiovascular disease. Non-limiting examples include statins including atorvastatin, fluvastatin, lovastatin, pitavastatin, pravastatin, rosuvastatin and simvastatin; PCSK9 inhibitors including alirocumab, evolocumab, and inclisiran; as well as cholesterol uptake inhibitor Ezetimibe. In some embodiments, the disclosed siNAs and pharmaceutical compositions comprising the disclosed siNAs are combined with approved or late stage development drugs in nonalcoholic fatty liver disease. Non-limiting examples include Resmetirom, ALG-055009, VK2809, efruxifermin, Saroglitazar, Obeticholic acid, Cenicriviroc, Aramchol, Dapagliflozin, Semaglutide, Belapectin, MSDC0602K, Lanifibranor and Tesamorelin.

In some embodiments, the pharmaceutical composition comprises at least a first siNA that reduces ANGPTL3 expression and a second siNA that reduces ANGPTL8 expression as disclosed herein and a pharmaceutically acceptable carrier. In some embodiments, the a weight ratio of the first siNA and the second siNA is selected from 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1. In some embodiments, a molar ration of the first siNA and the second ds-siNA is selected from 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1. In some embodiments, the first siNA and the second siNA are physically linked by a linker. Non-limiting examples of linkers are nucleotide based linkers or non-nucleotide based linkers. In some embodiments, the nucleotide based linker is between about 1 to about 15 nucleotides in length. In some embodiments, the nucleotide based linker is 1 or more, 2 or more, 3 or more, 4 or more, 5 or more, 6 or more, 7 or more, 8 or more, 9 or more, 10 or more, 11 or more, 12 or more, 13 or more, 14 or more, or 15 or more nucleotides in length.

In some embodiments, the linker is at least one or more one N-acetylgalactosamine (e.g., a GalNAc) as disclosed herein.

The siNAs of the disclosure optionally can be used in combination with modulators of other genes and/or gene products associated with the maintenance or development of diseases or disorders associated with aberrant ANGPTL8 and/or ANGPTL3 expression.

### EXAMPLES

These examples are provided for illustrative purposes only and not to limit the scope of the claims provided herein. For the purposes of these examples, the dsNA identifiers refer to the modified sequences provided in Tables 2 and 4.

Comparative control siNAs for the examples below have the sequences set forth in Table 5.

TABLE 5
Comparative Control siNAs Sequences

| | | SEQ ID | | SEQ ID | |
|---|---|---|---|---|---|
| | Identifier | NO: | SS 5′->3′ | NO: | AS 5′->3′ |
| ANGPTL3 | | 1126 | GCUCAACAUAUUU | 1132 | UACUGAUCAAAUA |
| Comparative | | | GAUCAGUA | | UGUUGAGC |
| Control siNA | | | | | |
| Unmodified | | | | | |
| ANGPTL3 | dsNA-282 | 1127 | mGmCmUmCmAmA | 1133 | mUpsfApsmCpsfUmGf |
| Comparative | | | mCmAfUfAfUmUmU | | AmUfCmAfAmAfUm |
| Control siNA | | | mGmAmUmCmAmG | | AfUmGfUmUfGmAfG |
| Modified | | | mUmA | | psmC |
| ANGPTL3 | dsNA-283 | 1128 | Aro-Gal- | 1134 | mUpsfApsmCpsfUmGf |
| Comparative | | | psinv XpsmGmCmUm | | AmUfCmAfAmAfUm |
| Control siNA | | | CmAmAmCmAfUfAf | | AfUmGfUmUfGmAfG |
| Modified + | | | UmUmUmGmAmUm | | psmC |
| GalNAc | | | CmAmGmUmApsinvX | | |
| ANGPTL8 | | 1129 | CCGAGAAUUUGAG | 1135 | UAAGACCUCAAAU |
| Comparative | | | GUCUUA | | UCUCGGAA |
| Control siNA | | | | | |
| Unmodified | | | | | |
| ANGPTL8 | dsNA-284 | 1130 | ln(5m)Cln(5m)CmGm | 1136 | mUpsfApsmAfGmAm |
| Comparative | | | AfGmAfAfUfUmUmG | | CmCmUfCmAmAmA |
| Control siNA | | | mAmGmGmUmCmU | | mUfUmCfUmCmGmG |
| Modified | | | mUmA | | psmApsm.A |
| ANGPTL8 | dsNA-285 | 1131 | (1gT3)3-p- | 1137 | mUpsfApsmAfGmAm |
| Comparative | | | ln(5m)Cln(5m)CmGm | | CmCmUfCmAmAmA |
| Control siNA | | | AfGmAfAfUfUmUmG | | mUfUmCfUmCmGmG |
| Modified + | | | mAmGmGmUmCmU | | psmApsmA |
| GalNAc | | | mUmAmor4hTmor4hT | | |

Abbreviations: f is a 2′-fluoro nucleotide,
m is a 2′-O-methyl nucleotide,
ps is a phosphorothioate internucleotide linkage, (1gT3)3 has a structure of 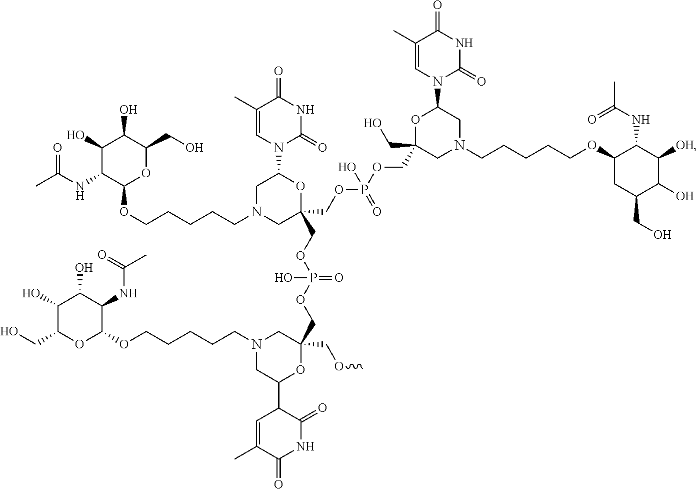
mor4hT (1T4) has a structure of 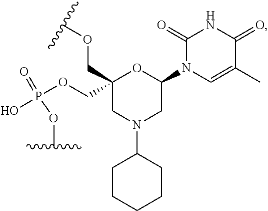 [SMILES: *OC[C@]1(COP(=O)(O)O*)CN(C2CCCCC2)C[C@H](n2cc(C)c(=O)[nH]c2=O)O1]
1nX has the structure of 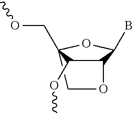
wherein B is a nucleobase or a derivative thereof, Aro-Gal has a structure of 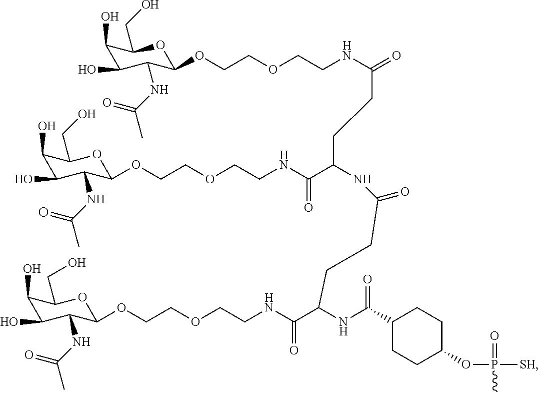
and invX has a structure of

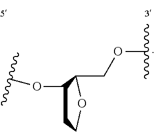

### Example 1. Evaluation of ANGPTLA3 siNAs Using a Three-Point Luciferase Reporter Assay

In the psiCHECK™-2 vector, Renilla luciferase was used as the primary reporter gene with the ANGPTL3 gene (NM_014495.4) cloned downstream of its translational stop codon. A second reporter gene, firefly luciferase, was expressed and used as a negative transfection control. The plasmid was first transfected using Lipofectamine 3000 (Invitrogen, L3000001) into COS-7 cells (ATCC, CRL-1651) seeded into 96-well microplates. The cells were then transfected with 10, 1, or 0.1 nM siNAs using Lipofectamine RNAiMAX (Invitrogen, 13778100). A mock, no-drug negative control, which consisted of transfecting 1× phosphate-buffered saline was included. Parental siNA dsNA-240 was used as the positive comparative control. After 72 hours of siNA treatment, the Dual-Glo® Luciferase Assay System (Promega, E2940) was used according to the manufacturer's protocol to quantify firefly and Renilla luciferase activity. All luminescence was measured on an En Vision plate reader (Perkin Elmer). The firefly: Renilla luminescence ratio was calculated for each well. The ratios from siNA-treated wells were then normalized to ratios of the mock-treated wells and percentage inhibition was calculated (Table 6).

TABLE 6
Results of Three-Point Luciferase Reporter
Assay for ANGPTL3 in COS-7 Cells

| | SEQ ID | SEQ ID | | | |
|---|---|---|---|---|---|
| | NOS | NOS | % inhibition of reporter activity | | |
| Identifier | (SS) | (AS) | 10 nM | 1 nM | 0.1 nM |
| dsNA-159 | 756 | 1003 | 92.66544 | 90.23598 | 29.90765 |
| dsNA-160 | 757 | 1004 | 90.80039 | 77.63515 | 14.84185 |
| dsNA-161 | 758 | 1005 | 25.52548 | −0.446654 | −11.49662 |
| dsNA-162 | 759 | 1006 | 27.02324 | 0.873961 | −12.21051 |
| dsNA-163 | 760 | 1007 | 14.17102 | −2.674181 | −18.51729 |
| dsNA-164 | 761 | 1008 | 6.287476 | −8.26073 | −18.94885 |
| dsNA-165 | 762 | 1009 | −7.16839 | −0.078109 | −18.23525 |
| dsNA-166 | 763 | 1010 | −7.622698 | −6.813788 | −13.74762 |
| dsNA-167 | 764 | 1011 | −10.03329 | −4.146742 | −19.22519 |
| dsNA-168 | 765 | 1012 | 90.16943 | 80.72835 | 25.06606 |
| dsNA-169 | 766 | 1013 | 90.74708 | 79.16289 | 12.81499 |
| dsNA-170 | 767 | 1014 | 73.74018 | 55.62508 | 4.575679 |
| dsNA-171 | 768 | 1015 | 63.53961 | 44.65217 | 1.511342 |
| dsNA-172 | 769 | 1016 | 56.84337 | 27.68057 | −9.09529 |
| dsNA-173 | 770 | 1017 | 52.0949 | 30.62673 | −13.76392 |
| dsNA-174 | 771 | 1018 | 58.83054 | 38.4638 | −6.07889 |
| dsNA-175 | 772 | 1019 | 69.74378 | 55.53058 | 10.5012 |
| dsNA-176 | 773 | 1020 | 65.95357 | 58.03511 | 17.99122 |
| dsNA-177 | 774 | 1021 | 86.10503 | 54.93573 | −8.83371 |
| dsNA-178 | 775 | 1022 | 65.76941 | 28.03964 | −26.1211 |
| dsNA-179 | 776 | 1023 | 93.93257 | 79.40837 | −3.105691 |
| dsNA-180 | 777 | 1024 | 91.56635 | 73.48417 | −2.51586 |
| dsNA-181 | 778 | 1025 | 85.3664 | 62.3963 | −13.04656 |
| dsNA-182 | 779 | 1026 | 83.30719 | 69.68169 | −7.279398 |
| dsNA-183 | 780 | 1027 | 87.15252 | 72.3972 | −4.630884 |
| dsNA-184 | 781 | 1028 | 59.4048 | 31.79845 | −23.07776 |
| dsNA-185 | 782 | 1029 | 49.95508 | 14.92497 | −21.02264 |
| dsNA-186 | 783 | 1030 | 18.76326 | 4.402678 | −9.331931 |
| dsNA-187 | 784 | 1031 | 17.09909 | 5.296831 | −9.131516 |
| dsNA-188 | 785 | 1032 | 13.91719 | 9.904148 | −6.183564 |
| dsNA-189 | 786 | 1033 | 23.11106 | 10.92668 | −5.811119 |
| dsNA-190 | 787 | 1034 | 0.783721 | 4.652944 | −2.811438 |
| dsNA-191 | 788 | 1035 | 7.858755 | 10.45698 | −7.402064 |
| dsNA-192 | 789 | 1036 | 4.169318 | 9.327082 | −0.504418 |
| dsNA-193 | 790 | 1037 | 15.01824 | 5.688844 | −10.25192 |
| dsNA-194 | 791 | 1038 | 74.89367 | 51.11305 | −2.726957 |
| dsNA-195 | 792 | 1039 | 59.05723 | 29.92962 | −3.423218 |
| dsNA-196 | 793 | 1040 | 75.14681 | 45.45653 | −3.363916 |
| dsNA-197 | 794 | 1041 | 55.80831 | 22.77133 | −4.357794 |
| dsNA-198 | 795 | 1042 | 83.62066 | 63.9082 | 1.76832 |
| dsNA-199 | 796 | 1043 | 80.50559 | 48.94628 | −2.478516 |
| dsNA-200 | 797 | 1044 | 90.99619 | 78.22267 | 5.817226 |
| dsNA-201 | 798 | 1045 | 80.70006 | 53.91147 | −13.29569 |
| dsNA-202 | 799 | 1046 | 60.11171 | 29.43151 | −23.1396 |
| dsNA-203 | 800 | 1047 | 64.26869 | 36.34756 | −17.94062 |
| dsNA-204 | 801 | 1048 | 18.6656 | 0.311395 | 1.753766 |
| dsNA-205 | 802 | 1049 | 14.84531 | 6.309918 | −16.04493 |
| dsNA-206 | 803 | 1050 | 16.48941 | 3.737368 | −26.98408 |
| dsNA-207 | 804 | 1051 | 14.84758 | 0.193324 | −25.64271 |
| dsNA-208 | 805 | 1052 | 6.691558 | 1.286914 | −33.82667 |
| dsNA-209 | 806 | 1053 | 50.18374 | 21.50079 | −22.96691 |
| dsNA-210 | 807 | 1054 | 75.48207 | 52.52478 | −9.893261 |
| dsNA-211 | 808 | 1055 | 67.65847 | 48.03154 | −9.264855 |
| dsNA-212 | 809 | 1056 | 77.95893 | 60.20422 | 4.655984 |
| dsNA-213 | 810 | 1057 | 93.2188 | 91.29268 | 51.8368 |
| dsNA-214 | 811 | 1058 | 91.99619 | 84.60418 | 32.35249 |
| dsNA-215 | 812 | 1059 | 82.35955 | 59.80283 | −8.103808 |
| dsNA-216 | 813 | 1060 | 91.63036 | 81.74269 | 17.43654 |
| dsNA-217 | 814 | 1061 | 89.08794 | 70.19488 | −5.678417 |
| dsNA-218 | 815 | 1062 | 84.79333 | 73.05998 | 4.683909 |
| dsNA-219 | 816 | 1063 | 89.5327 | 79.45698 | 10.69112 |
| dsNA-220 | 817 | 1064 | 92.77851 | 88.15634 | 21.27691 |
| dsNA-221 | 818 | 1065 | 23.9862 | 16.51074 | −9.736262 |
| dsNA-222 | 819 | 1066 | 0.932813 | 2.683007 | −14.05353 |
| dsNA-223 | 820 | 1067 | 30.11258 | 20.21437 | −14.7539 |
| dsNA-224 | 821 | 1068 | 26.54681 | 14.89556 | −13.53555 |
| dsNA-225 | 822 | 1069 | 27.01983 | 17.64549 | −8.132288 |
| dsNA-226 | 823 | 1070 | 29.1757 | 22.82461 | −6.46017 |
| dsNA-227 | 824 | 1071 | 28.82275 | 17.41885 | −15.41678 |
| dsNA-228 | 825 | 1072 | 26.23087 | 16.34608 | −10.14718 |
| dsNA-229 | 826 | 1073 | 35.98825 | 26.27576 | −1.038507 |
| dsNA-230 | 827 | 1074 | 58.63784 | 27.14577 | 13.23283 |
| dsNA-231 | 828 | 1075 | 65.12184 | 33.99818 | −3.603306 |
| dsNA-232 | 829 | 1076 | 34.04601 | 14.04026 | −16.29055 |
| dsNA-233 | 830 | 1077 | 81.86511 | 51.19784 | 4.381946 |
| dsNA-234 | 831 | 1078 | 92.08608 | 78.37043 | 37.71091 |
| dsNA-235 | 832 | 1079 | 86.879 | 66.84533 | 16.15206 |
| dsNA-236 | 833 | 1080 | 85.6122 | 69.18959 | 13.04727 |
| dsNA-237 | 834 | 1081 | 90.47951 | 74.4492 | 13.12664 |
| dsNA-238 | 835 | 1082 | 91.01311 | 72.51536 | 23.7666 |
| dsNA-239 | 836 | 1083 | 87.22198 | 76.69057 | 23.83387 |

### Example 2. Evaluation of ANGPTL3 siNAs Using Dose-Response Assays

Select candidates were defined in Example 1 as showing ≥65% inhibition at 1 nM and ≥85% inhibition at 10 nM. Dose-response assays were conducted, with serial concentrations of siNAs starting at 10 nM (1:5 dilutions) for each select candidate siNA using the Dual-Glo® Luciferase Assay System for ANGPTL3 as described in Example 1. Dose-response curves were fitted by nonlinear regression with variable slope, and EC₅₀ values and maximum percentage inhibition were calculated (Table 7).

TABLE 7
Results of Dose-Response Luciferase Reporter
Assay for ANGPTL3 in COS-7 Cells

| | | | | % inhibition of |
|---|---|---|---|---|
| | SEQ ID | SEQ ID | | reporter activity |
| | NO: | NO: | EC₅₀ | of reporter |
| Identifier | (SS) | (AS) | (nM) | activity |
| dsNA-282 | 1127 | 1133 | 0.1464 | 97.51 |
| (Comparative Control) | | | | |
| dsNA-159 | 756 | 1003 | 0.02697 | 84.4 |
| dsNA-160 | 757 | 1004 | 0.02512 | 91.17 |
| dsNA-200 | 797 | 1044 | 0.02279 | 94.04 |
| dsNA-213 | 810 | 1057 | 0.007122 | 90.74 |
| dsNA-214 | 811 | 1058 | 0.01152 | 86.63 |
| dsNA-168 | 765 | 1012 | 0.05978 | 97.79 |
| dsNA-216 | 813 | 1060 | 0.002508 | 94.59 |
| dsNA-217 | 814 | 1061 | 0.02256 | 93.92 |
| dsNA-218 | 815 | 1062 | 0.02948 | 91.9 |
| dsNA-219 | 816 | 1063 | 0.02394 | 93.44 |
| dsNA-220 | 817 | 1064 | 0.01387 | 95.36 |
| dsNA-179 | 776 | 1023 | 0.02525 | 94.21 |
| dsNA-180 | 777 | 1024 | 0.006316 | 94.05 |
| dsNA-234 | 831 | 1078 | 0.02211 | 89.72 |
| dsNA-235 | 832 | 1079 | 0.02322 | 90.05 |
| dsNA-236 | 833 | 1080 | 0.02254 | 86.82 |
| dsNA-237 | 834 | 1081 | 0.05534 | 92.19 |
| dsNA-238 | 835 | 1082 | 0.01654 | 93.78 |
| dsNA-239 | 836 | 1083 | 0.004324 | 91.84 |

For the RT-qPCR assay, hepatoma-derived Huh-7 cells (JCRB Cell Bank, JCRB0403) were transfected with serially diluted siNA starting at 10 nM (1:5 dilutions) and Opti-MEM™ using Lipofectamine RNAiMAX (Invitrogen, 13778100). A negative mock transfection control, which consisted of transfecting 1× phosphate-buffered saline, was included. After about 48 hours of siNA treatment, the Huh-7 cells were processed with the TaqMan Fast Advanced Cells-to-Ct Kit (Invitrogen, A35378), according to the manufacturer's protocol. The cell lysates were used for reverse transcription. Gene expression was measured using TaqMan Fast Advanced Master Mix (Applied Biosystems, 4444964) and the following TaqMan Gene Expression assays (Applied Biosystems, 4331182): ACTB (Hs01060665_g1) and ANGPTL3 (Hs00205581_m1). ACTB served as the endogenous control gene. RT-qPCR reactions were run on the QuantStudio™ 6 Pro Real-Time PCR System (Applied Biosystems). The RQ of gene expression was calculated via the 2^{-ΔΔCt} (RQ) method. Results are presented as expression relative to the expression levels of negative mock control samples. Dose-response curves were fitted by nonlinear regression with variable slope, EC₅₀ values, and maximum percentage inhibition calculated (Table 8).

TABLE 8
Results of Dose-Response RT-qPCR
Assay for ANGPTL3 in Huh-7 Cells

| | | | | Max. % |
|---|---|---|---|---|
| | SEQ ID | SEQ ID | | ANGPTL3 |
| | NO: | NO: | EC₅₀ | RNA |
| Identifier | (SS) | (AS) | (nM) | inhibition |
| dsNA-282 | 1127 | 1133 | 0.0313 | 91.84 |
| (Comparative | | | | |
| Control) | | | | |
| dsNA-159 | 756 | 1003 | 0.0081 | 87.25 |
| dsNA-160 | 757 | 1004 | 0.0159 | 84.01 |
| dsNA-200 | 797 | 1044 | 0.0286 | 89.97 |
| dsNA-213 | 810 | 1057 | 0.017 | 90.71 |
| dsNA-214 | 811 | 1058 | 0.0025 | 93.45 |
| dsNA-168 | 765 | 1012 | 0.0132 | 91.54 |
| dsNA-216 | 813 | 1060 | 0.0094 | 92.63 |
| dsNA-217 | 814 | 1061 | 0.1158 | 87.71 |
| dsNA-218 | 815 | 1062 | 0.0382 | 89.64 |
| dsNA-219 | 816 | 1063 | 0.0397 | 92.63 |
| dsNA-220 | 817 | 1064 | — | 87.25 |
| dsNA-179 | 776 | 1023 | — | 84.01 |
| dsNA-180 | 777 | 1024 | — | 89.97 |
| dsNA-234 | 831 | 1078 | 0.0001 | 90.71 |
| dsNA-235 | 832 | 1079 | 0.0232 | 81.19 |
| dsNA-236 | 833 | 1080 | 0.0855 | 80.44 |
| dsNA-237 | 834 | 1081 | 0.0236 | 87.03 |
| dsNA-238 | 835 | 1082 | 0.0279 | 92.25 |
| dsNA-239 | 836 | 1083 | 0.0314 | 87.49 |

For the RT-qPCR assay, hepatoma-derived Hep3B cells (ATCC, HB-8064) were transfected with serially diluted siNA starting at 10 nM (1:8 dilutions) and Opti-MEM™ using Lipofectamine RNAiMAX (Invitrogen, 13778100). A negative mock transfection control, which consisted of transfecting 1× phosphate-buffered saline, was included. After about 48 hours of siNA treatment, the Hep3B cells were processed with the TaqMan Fast Advanced Cells-to-Ct Kit (Invitrogen, A35378), according to the manufacturer's protocol. The cell lysates were used for reverse transcription. Gene expression was measured using TaqMan Fast Advanced Master Mix (Applied Biosystems, 4444964) and the following TaqMan Gene Expression assays (Applied Biosystems, 4331182): ACTB (Hs01060665_g1) and ANGPTL3 (Hs00205581_m1). ACTB served as the endogenous control gene. RT-qPCR reactions were run on the QuantStudio™ 6 Pro Real-Time PCR System (Applied Biosystems). The RQ of gene expression was calculated via the 2-ΔΔCt (RQ) method. Results are presented as expression relative to the expression levels of negative mock control samples. Dose-response curves were fitted by nonlinear regression with variable slope, EC50 values, and maximum percentage inhibition calculated (Table 9).

TABLE 9
Results of Dose-Response RT-qPCR
Assay for ANGPTL3 in Hep3B Cells

| | SEQ ID NO: | SEQ ID NO: | EC₅₀ | Max. % |
|---|---|---|---|---|
| Identifier | (SS) | (AS) | (nM) | ANGPTL3 |
| dsNA-282 | 1127 | 1133 | 0.04 | 92.7 |
| (Comparative | | | | |
| Control) | | | | |
| dsNA-240 | 837 | 1084 | 0.0092 | 87.78 |
| dsNA-241 | 838 | 1085 | 0.0088 | 86.09 |
| dsNA-242 | 839 | 1086 | 0.0081 | 82.62 |
| dsNA-243 | 840 | 1087 | 0.0061 | 86.42 |
| dsNA-244 | 841 | 1088 | 0.0102 | 87.83 |
| dsNA-245 | 842 | 1089 | 0.0180 | 81.42 |
| dsNA-250 | 843 | 1090 | 0.012 | 88.12 |
| dsNA-251 | 844 | 1091 | 0.011 | 89.62 |
| dsNA-252 | 845 | 1092 | 0.008 | 91.09 |
| dsNA-253 | 846 | 1093 | 0.007 | 90.76 |
| dsNA-254 | 847 | 1094 | 0.014 | 88.25 |
| dsNA-255 | 848 | 1095 | 0.019 | 89.52 |
| dsNA-256 | 849 | 1096 | 0.372 | 85.95 |
| dsNA-257 | 850 | 1097 | 0.026 | 85.62 |
| dsNA-258 | 851 | 1098 | 0.030 | 71.02 |
| dsNA-259 | 852 | 1099 | 0.087 | 68.86 |
| dsNA-260 | 853 | 1100 | 0.013 | 71.93 |
| dsNA-261 | 854 | 1101 | >10 | 58.59 |
| dsNA-262 | 855 | 1102 | 0.009 | 90.08 |
| dsNA-263 | 856 | 1103 | 0.004 | 89.45 |
| dsNA-264 | 857 | 1104 | 0.004 | 88.24 |
| dsNA-265 | 858 | 1105 | 0.005 | 87.60 |
| dsNA-266 | 859 | 1106 | 0.011 | 88.82 |
| dsNA-267 | 860 | 1107 | 0.014 | 85.39 |
| dsNA-268 | 861 | 1108 | 0.016 | 85.27 |
| dsNA-269 | 862 | 1109 | 0.010 | 86.62 |
| dsNA-270 | 863 | 1110 | 0.021 | 77.53 |
| dsNA-271 | 864 | 1111 | 0.041 | 74.92 |
| dsNA-272 | 865 | 1112 | 0.071 | 77.54 |
| dsNA-273 | 866 | 1113 | 0.027 | 75.16 |
| dsNA-274 | 867 | 1114 | 0.015 | 79.56 |
| dsNA-275 | 868 | 1115 | 0.017 | 79.03 |
| dsNA-276 | 869 | 1116 | 0.017 | 83.53 |
| dsNA-277 | 870 | 1117 | 0.035 | 82.54 |
| dsNA-278 | 871 | 1118 | 0.024 | 85.92 |
| dsNA-279 | 872 | 1119 | 0.020 | 82.50 |
| dsNA-280 | 873 | 1120 | 0.011 | 83.93 |
| dsNA-281 | 874 | 1121 | 0.017 | 86.13 |

The human ANGPTL3 siNAs dsNA-240, dsNA-241, dsNA-242, dsNA-243, dsNA-244, dsNA-245, dsNA-250, dsNA-251, dsNA-252, dsNA-253, dsNA-254, dsNA-255, dsNA-260, dsNA-262, dsNA-263, dsNA-264, dsNA-265, dsNA-266, dsNA-267, dsNA-268, dsNA-269, dsNA-274, dsNA-275, dsNA-276, dsNA-280 and dsNA-281 showed better in vitro potency than the comparative control siNA, while dsNA-257, dsNA-258, dsNA-270, dsNA-271, dsNA-272, dsNA-273, dsNA-277, dsNA-278 and dsNA-279 showed similar potency to the comparative control siNA.

### Example 3. Toxicity Assay and Cross-Species Conservation for ANGPTL3 siNAs

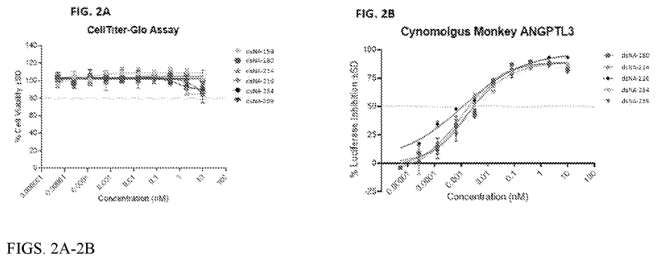

The candidates were further narrowed to six select siNAs, dsNA-159, dsNA-180, dsNA-214, dsNA-216, dsNA-234, and dsNA-239. CellTiter-Glo® Luminescent Cell Viability Assays were performed in siNA-treated COS-7 cells under conditions as described in Example 1. Assays were performed according to the manufacturer's protocol and luminescence was measured on an En Vision plate reader. The luminescence from siNA-treated wells were then normalized to luminescence of mock-treated wells and percentage viability was calculated. (FIG. 2A). No siNAs exhibited cytotoxic effects and all had a CC₅₀ of >10 nM. All six candidates were also assessed for ANGPTL3 inhibition against the cynomolgus monkey ANGPTL3 using a psiCHECK™-2 system similarly described in Example 1, but with the Macaca fascicularis gene (XM_005543185.3) inserted. The activity of the candidates was highly conserved between species (FIG. 2B).

### Example 4. Evaluation of ANGPTLA8 siNAs Using a Three-Point RT-qPCR Reporter Assay

For the RT-qPCR assay, hepatoma-derived Hep3B cells (ATCC, HB-8064) were transfected with siNA at 10.0, 1.0, or 0.1 nM and Opti-MEM™ using Lipofectamine RNAiMAX (Invitrogen, 13778100). A negative mock transfection control, which consisted of transfecting 1× phosphate-buffered saline, was included. After about 48 hours of siNA treatment, the Hep3B cells were processed with the TaqMan Fast Advanced Cells-to-Ct Kit (Invitrogen, A35378), according to the manufacturer's protocol. The cell lysates were used for reverse transcription. Gene expression was measured using TaqMan Fast Advanced Master Mix (Applied Biosystems, 4444964) and the following TaqMan Gene Expression assays (Applied Biosystems, 4331182): ACTB (Hs01060665_g1) and ANGPTL8 (Hs00218820_m1). ACTB served as the endogenous control gene. RT-qPCR reactions were run on the QuantStudio™ 6 Pro Real-Time PCR System (Applied Biosystems). The RQ of gene expression was calculated via the 2-ΔΔCt (RQ) method. Results are presented as expression relative to the expression levels of negative mock control samples. Percentage inhibition at each dose was calculated (Table 10).

TABLE 10
Results of Three-Point RT-qPCR Assay
for ANGPTL8 in Hep3B Cells.

RT-qPCR in Hep3B
% ANGPTL8 RNA inhibition

| Identifier | 10 nM | 1 nM | 0.1 nM |
|---|---|---|---|
| dsNA-284 | 76.73 | 71.69 | 53.14 |
| (Comparative Control) | | | |
| dsNA-001 | 5.59 | −17.19 | −32.92 |
| dsNA-002 | 1.44 | −2.49 | −14.78 |
| dsNA-003 | −6.94 | −18.61 | −26.66 |
| dsNA-004 | −53.45 | −18.11 | −28.68 |
| dsNA-005 | 9.36 | −5.93 | −30.24 |
| dsNA-006 | −22.68 | −32.65 | −26.32 |
| dsNA-007 | 59.78 | 31.42 | −15.36 |
| dsNA-008 | 39.34 | −5.38 | 7.32 |
| dsNA-009 | 53.91 | 26.98 | −13.58 |
| dsNA-010 | 52.18 | 43.93 | 3.33 |
| dsNA-011 | 37.10 | 12.82 | 48.56 |
| dsNA-012 | 42.45 | −1.26 | 48.67 |
| dsNA-013 | −29.42 | −26.86 | 26.03 |
| dsNA-014 | −32.78 | −48.11 | 20.96 |
| dsNA-015 | −18.90 | −73.30 | −8.19 |
| dsNA-016 | −49.81 | −59.93 | −5.08 |
| dsNA-017 | −6.04 | −34.38 | −8.69 |
| dsNA-018 | −37.56 | −54.05 | −14.25 |
| dsNA-019 | 25.30 | −12.14 | −22.67 |
| dsNA-020 | 1.76 | −21.06 | −31.82 |
| dsNA-021 | 59.20 | 43.72 | 47.24 |
| dsNA-022 | 58.18 | 56.07 | 44.98 |
| dsNA-023 | 73.98 | 64.77 | 59.59 |
| dsNA-024 | 28.90 | 10.87 | 26.74 |
| dsNA-025 | 79.78 | 52.17 | 50.57 |
| dsNA-026 | 16.95 | −23.29 | 23.72 |
| dsNA-027 | 21.52 | −6.57 | 25.16 |
| dsNA-028 | 85.66 | 76.10 | 73.64 |
| dsNA-029 | −15.04 | −8.38 | 14.73 |
| dsNA-030 | −10.47 | −35.57 | 9.25 |
| dsNA-031 | 45.33 | −0.97 | 40.73 |
| dsNA-032 | −6.08 | −23.70 | 28.93 |
| dsNA-033 | 15.77 | 5.84 | 34.82 |
| dsNA-034 | 19.49 | −18.35 | 17.41 |
| dsNA-035 | −29.48 | −68.35 | 1.59 |
| dsNA-036 | −34.17 | −72.56 | −2.21 |
| dsNA-037 | −45.98 | −72.71 | 12.72 |
| dsNA-038 | 50.73 | 12.07 | 44.86 |
| dsNA-039 | −27.40 | −70.31 | −21.41 |
| dsNA-040 | −32.85 | −56.15 | −46.51 |
| dsNA-041 | 33.84 | 23.13 | 50.21 |
| dsNA-042 | 41.35 | 44.71 | 40.69 |
| dsNA-043 | 55.68 | 43.23 | 58.34 |
| dsNA-044 | 1.88 | −11.43 | 19.94 |
| dsNA-045 | −44.18 | −63.75 | 9.64 |
| dsNA-046 | −25.42 | −59.78 | 4.77 |
| dsNA-047 | 55.35 | 23.20 | 29.56 |
| dsNA-048 | 33.17 | −16.40 | 29.11 |
| dsNA-049 | 17.31 | 23.66 | 0.26 |
| dsNA-050 | 93.66 | 89.95 | 60.22 |
| dsNA-051 | 9.22 | 18.27 | 12.23 |
| dsNA-052 | 50.51 | 45.64 | 18.71 |
| dsNA-053 | 56.30 | 62.27 | 37.46 |
| dsNA-054 | 68.73 | 67.49 | 59.51 |
| dsNA-055 | 73.77 | 79.36 | 64.66 |
| dsNA-056 | 33.15 | 31.68 | 24.17 |
| dsNA-057 | −71.21 | −35.45 | −2.65 |
| dsNA-058 | 57.57 | 56.19 | 24.95 |
| dsNA-059 | 47.50 | 46.92 | 50.79 |
| dsNA-060 | 52.51 | 40.87 | 39.95 |
| dsNA-061 | 64.55 | 62.31 | 62.44 |
| dsNA-062 | 77.51 | 63.94 | 62.64 |
| dsNA-063 | 66.90 | 68.22 | 66.64 |
| dsNA-064 | 26.27 | 19.32 | 23.29 |
| dsNA-065 | 25.23 | 13.19 | 29.82 |
| dsNA-066 | 11.39 | 25.13 | 27.17 |
| dsNA-067 | 30.87 | 31.79 | 33.63 |
| dsNA-068 | 58.71 | 55.85 | 56.86 |
| dsNA-069 | 83.41 | 86.03 | 72.32 |
| dsNA-070 | 92.17 | 93.56 | 85.43 |
| dsNA-071 | 94.38 | 92.20 | 75.52 |
| dsNA-072 | 76.00 | 75.66 | 51.71 |
| dsNA-073 | 81.12 | 77.39 | 45.07 |
| dsNA-074 | 91.26 | 90.81 | 56.68 |
| dsNA-075 | 68.09 | 69.64 | 34.95 |
| dsNA-076 | 93.80 | 92.55 | 69.74 |
| dsNA-077 | 88.71 | 90.25 | 74.61 |
| dsNA-078 | 10.84 | 31.78 | 11.90 |
| dsNA-079 | 5.91 | 20.61 | 18.24 |
| dsNA-080 | 38.08 | 33.13 | 7.87 |
| dsNA-081 | 30.13 | 25.12 | 12.24 |
| dsNA-082 | 49.37 | 44.06 | 18.02 |
| dsNA-083 | 91.25 | 86.96 | 52.91 |
| dsNA-084 | 94.90 | 90.87 | 64.33 |
| dsNA-085 | 44.80 | 46.19 | 10.00 |
| dsNA-086 | 63.85 | 56.08 | 23.99 |
| dsNA-087 | 49.82 | 41.76 | 18.84 |
| dsNA-088 | 6.89 | 29.59 | 2.96 |
| dsNA-089 | 61.72 | 66.68 | 32.74 |
| dsNA-090 | 65.54 | 74.25 | 67.12 |
| dsNA-091 | 96.08 | 95.02 | 71.39 |
| dsNA-092 | 24.11 | 30.12 | 11.71 |
| dsNA-093 | −20.67 | −3.75 | 2.62 |
| dsNA-094 | 74.95 | 69.95 | 32.20 |
| dsNA-095 | 69.10 | 74.56 | 52.89 |
| dsNA-096 | 74.67 | 80.30 | 61.12 |
| dsNA-097 | 32.44 | 20.22 | −11.46 |
| dsNA-098 | 70.52 | 62.55 | 13.49 |
| dsNA-099 | 61.42 | 38.48 | −17.17 |
| dsNA-100 | 33.27 | 29.29 | 1.60 |
| dsNA-101 | 12.08 | −10.45 | −35.47 |
| dsNA-102 | 73.68 | 51.96 | −2.10 |
| dsNA-103 | 52.77 | 52.24 | −10.79 |
| dsNA-104 | 87.68 | 84.10 | 54.48 |
| dsNA-105 | 47.04 | 57.31 | 34.00 |
| dsNA-106 | 74.49 | 66.99 | 23.22 |
| dsNA-107 | 88.82 | 66.36 | 44.65 |
| dsNA-108 | 78.09 | 55.33 | 40.04 |
| dsNA-109 | 74.84 | 62.67 | 38.46 |
| dsNA-110 | 72.05 | 47.14 | 19.52 |
| dsNA-111 | 80.87 | 70.18 | 46.94 |
| dsNA-112 | 52.69 | 25.79 | −11.60 |
| dsNA-113 | 51.24 | 36.90 | −17.09 |
| dsNA-114 | 10.69 | 14.54 | −9.67 |
| dsNA-115 | 21.03 | 9.13 | −50.77 |
| dsNA-116 | 27.12 | −13.86 | −5.45 |
| dsNA-117 | 45.13 | 35.45 | 18.52 |
| dsNA-118 | 56.45 | 43.48 | 16.95 |
| dsNA-119 | 53.95 | 36.87 | 20.86 |
| dsNA-120 | 80.52 | 68.19 | 38.53 |
| dsNA-121 | 58.06 | 35.96 | 11.03 |
| dsNA-122 | 35.72 | 26.46 | 8.15 |
| dsNA-123 | 41.34 | 27.22 | 7.20 |
| dsNA-124 | 20.08 | 16.29 | 4.64 |
| dsNA-125 | 30.81 | 25.69 | 4.98 |
| dsNA-126 | 44.40 | 32.24 | 1.90 |
| dsNA-127 | 63.52 | 56.61 | 32.02 |
| dsNA-128 | 78.02 | 62.96 | 41.23 |
| dsNA-129 | 82.40 | 75.78 | 57.82 |
| dsNA-130 | 76.02 | 69.44 | 50.42 |
| dsNA-131 | 74.63 | 63.13 | 49.71 |
| dsNA-132 | 84.30 | 71.46 | 55.61 |
| dsNA-133 | 71.87 | 60.87 | 42.17 |
| dsNA-134 | 37.39 | 33.25 | 5.59 |
| dsNA-135 | 45.20 | 27.08 | 9.95 |
| dsNA-136 | 36.17 | 8.49 | −9.45 |
| dsNA-137 | 64.94 | 57.26 | 16.75 |
| dsNA-138 | 79.94 | 71.84 | 51.67 |
| dsNA-139 | 76.86 | 69.20 | 43.55 |
| dsNA-140 | 68.41 | 56.01 | 15.18 |
| dsNA-141 | 78.12 | 70.70 | 45.64 |
| dsNA-142 | 39.15 | 22.36 | 5.31 |
| dsNA-143 | 12.00 | 6.28 | −10.40 |
| dsNA-144 | 49.90 | 38.27 | 19.81 |
| dsNA-145 | 75.62 | 74.71 | 73.95 |
| dsNA-146 | 84.35 | 75.19 | 73.18 |
| dsNA-147 | 73.75 | 69.00 | 77.63 |

All dsNAs in this table correspond to the modified dsNA sequences shown in Table 2 above.

### Example 5. Evaluation of ANGPTL8 siNAs Using a Dose-Response RT-qPCT Assay

For the RT-qPCR assay, hepatoma-derived Hep3B cells (ATCC, HB-8064) were transfected with serially diluted siNA starting at 10 nM (1:8 dilutions) and Opti-MEM™ using Lipofectamine RNAiMAX (Invitrogen, 13778100). A negative mock transfection control, which consisted of transfecting 1× phosphate-buffered saline, was included. After about 48 hours of siNA treatment, the Hep3B cells were processed with the TaqMan Fast Advanced Cells-to-Ct Kit (Invitrogen, A35378), according to the manufacturer's protocol. The cell lysates were used for reverse transcription. Gene expression was measured using TaqMan Fast Advanced Master Mix (Applied Biosystems, 4444964) and the following TaqMan Gene Expression assays (Applied Biosystems, 4331182): ACTB (Hs01060665_g1) and ANGPTL8 (Hs00218820_m1). ACTB served as the endogenous control gene. RT-qPCR reactions were run on the QuantStudio™ 6 Pro Real-Time PCR System (Applied Biosystems). The RQ of gene expression was calculated via the 2-ΔΔCt (RQ) method. Results are presented as expression relative to the expression levels of negative mock control samples. Dose-response curves were fitted by nonlinear regression with variable slope, EC50 values, and maximum percentage inhibition calculated (Table 11). Most of the tested siNAs exhibited similar or enhanced potency and maximum percentage of ANGPTL8 RNA inhibition as the comparative control siNA dsNA-284.

TABLE 11
Results of Dose-Response RT-qPCR
Assay for ANGPTL8 in Hep3B Cells

RT-qPCR in Hep3B

| | EC₅₀ | Maximum % ANGPTL8 |
|---|---|---|
| Identifier | (nM) | RNA inhibition |
| dsNA-284 | 0.012 | 91.91 |
| (Comparative | | |
| Control) | | |
| dsNA-28 | 0.0665 | 92.64 |
| dsNA-50 | 0.1005 | 86.97 |
| dsNA-55 | 0.0162 | 75.28 |
| dsNA-69 | 0.0195 | 88.08 |
| dsNA-70 | 0.0082 | 92.64 |
| dsNA-71 | 0.0356 | 96.04 |
| dsNA-74 | 0.0574 | 98.72 |
| dsNA-76 | 0.064 | 95.59 |
| dsNA-77 | 0.054 | 85.31 |
| dsNA-84 | 0.111 | 96.54 |
| dsNA-91 | 0.052 | 96.73 |
| dsNA-96 | 0.020 | 70.55 |
| dsNA-104 | 0.028 | 95.89 |
| dsNA-129 | 0.050 | 95.10 |
| dsNA-144 | >1.000 | ND |
| dsNA-145 | <0.001 | 84.37 |
| dsNA-146 | <0.001 | 90.58 |
| dsNA-148 | 0.340 | 93.24 |
| dsNA-149 | 0.308 | 92.49 |
| dsNA-150 | 0.073 | 92.80 |
| dsNA-151 | 0.118 | 88.65 |
| dsNA-152 | 0.075 | 98.86 |
| dsNA-153 | 0.023 | 96.41 |
| dsNA-154 | 0.082 | 97.18 |
| dsNA-155 | 0.046 | 84.92 |
| dsNA-156 | 0.119 | 92.54 |
| dsNA-157 | 0.250 | 93.98 |
| dsNA-158 | 0.032 | 89.28 |

All dsNAs in this table correspond to the modified dsNA sequences shown in Table 2 above.

### Example 6. AAV-hANGPTL3 Mouse Study Design

To evaluate certain ANGPTL3 siNA agents, an AAV-ANGPTL3 mouse model was used. Mice (n=4-5/group) were injected with an adeno-associated virus (AAV) expressing human ANGPTL3. Fourteen days after AAV injection, mice were subcutaneously administered a single dose (5 mg/kg) of siNA test article. Fourteen days after administration, the animals were sacrificed; the right, lateral liver lobe of each animal was collected for RT-qPCR and blood of each animal was collected for ELISA.

For RT-qPCR, RNA was extracted using the Rneasy Mini Kit (Qiagen, 74106), according to the manufacturer's protocol. RNA quantity and quality was analyzed with a NanoDrop™ Lite Spectrophotometer (Thermo Scientific), and cDNA was synthesized using the SuperScript IV VILO Master Mix (Invitrogen, 11756500), according to the manufacturer's protocol. Gene expression was measured using TaqMan Fast Advanced Master Mix (Applied Biosystems, 4444964), and the following TaqMan Gene Expression assays (Applied Biosystems, 4331182): Actb (Mm00607939_s1), ANGPTL3 (Hs00205581_m1), B2m (Mm00437762_m1), and Hmbs (Mm01143545_m1). Actb, B2m, and Hmbs served as the endogenous control genes. RT-qPCR reactions were run on the QuantStudio™ 6 Pro Real-Time PCR System (Applied Biosystems). The RQ of gene expression was calculated via the 2-AACt (RQ) method. Results are presented as percent change in expression relative to the expression levels of negative vehicle control samples.

For ELISA, serum was separated from blood samples. Levels human ANGPTL3 protein in sera were quantified using ELISA (R&D Systems, DANL30) according to the manufacturer's protocol. Results are presented as percent change in expression relative to the expression levels of negative vehicle control samples (Table 12). Note, the body weights of mice in all test article groups were normal and showed no toxicity from treatment.

TABLE 12
Day 28 Results of AAV-hANGPTL3 Mouse Study

| | 1 × PBS | dsNA-283 | | | | |
|---|---|---|---|---|---|---|
| | Negative | (Comparative | dsNA- | dsNA- | dsNA- | dsNA- |
| | Control | Control) | 240 | 241 | 243 | 244 |
| % Change | 0 | −24% | −49% | −32% | −36% | −40% |
| in Human | | | | | | |
| ANGPTL3 | | | | | | |
| RNA | | | | | | |
| % Change | 0 | −62% | −79% | −93% | −80% | −79% |
| in Human | | | | | | |
| ANGPTL3 | | | | | | |
| Protein | | | | | | |

Based on these results, dsNA-240, dsNA-241, dsNA-243 and dsNA-244 showed better human ANGPTL3 RNA and protein knockdown than comparative control dsNA-283.

### Example 7. AAV-hANGPTL3 Study Design

To evaluate certain ANGPTL3 siNA agents, an AAV-ANGPTL3 mouse model was used. Mice (n=4/group) were injected with an adeno-associated virus (AAV) expressing human ANGPTL3. Seven days after AAV injection, mice were subcutaneously administered a single dose (0.3, 1.5, or 5.0 mg/kg) of an siNA (dsNA-283; dsNA-244) or a single dose of 5 mg/kg (dsNA-247; dsNA-248; dsNA-249). Twenty-eight days after administration, the animals were sacrificed; the right, lateral liver lobe of each animal was collected for RT-qPCR and blood of each animal was collected for ELISA. (Table 13).

TABLE 13
Example 7 Design Summary Table
AAV-ANGPTL3 28-Day Study Design

| | Oligo | | | | |
|---|---|---|---|---|---|
| | ANGPTL3 | | | | |
| | Gene | | Group | Animals | Dose |
| | Location | Identifier | Number | (n) | (mg/kg) |
| Set A AAV- | | Vehicle-only | 1 | 4 | — |
| hANGPTL3 | | Negative | | | |
| | | Control | | | |
| | 1282 | dsNA-247 | 2 | 4 | 5.0 |
| | 244 | dsNA-248 | 3 | 4 | 5.0 |
| | 277 | dsNA-249 | 4 | 4 | 5.0 |
| | 1276 | dsNA-244 | 5 | 4 | 5.0 |
| | | | 6 | 4 | 1.5 |
| | | | 7 | 4 | 0.3 |
| | 270 | dsNA-283 | 8 | 3 | 5.0 |
| | | (Comparative | 9 | 4 | 1.5 |
| | | Control) | 10 | 4 | 0.3 |

For RT-qPCR, RNA was extracted using the Rneasy Mini Kit (Qiagen, 74106), according to the manufacturer's protocol. RNA quantity and quality was analyzed with a NanoDrop™ Lite Spectrophotometer (Thermo Scientific), and cDNA was synthesized using the SuperScript IV VILO Master Mix (Invitrogen, 11756500), according to the manufacturer's protocol. Gene expression was measured using TaqMan Fast Advanced Master Mix (Applied Biosystems, 4444964), and the following TaqMan Gene Expression assays (Applied Biosystems, 4331182): Actb (Mm00607939_s1), ANGPTL3 (Hs00205581_m1), Angpt13 (Mm00803820_m1), B2m (Mm00437762_m1), and Hmbs (Mm01143545_m1). Actb, B2m, and Hmbs served as the endogenous control genes. RT-qPCR reactions were run on the QuantStudio™ 6 Pro Real-Time PCR System (Applied Biosystems). The RQ of gene expression was calculated via the 2-AACt (RQ) method. Results are presented as percent change in expression relative to the expression levels of negative vehicle control samples.

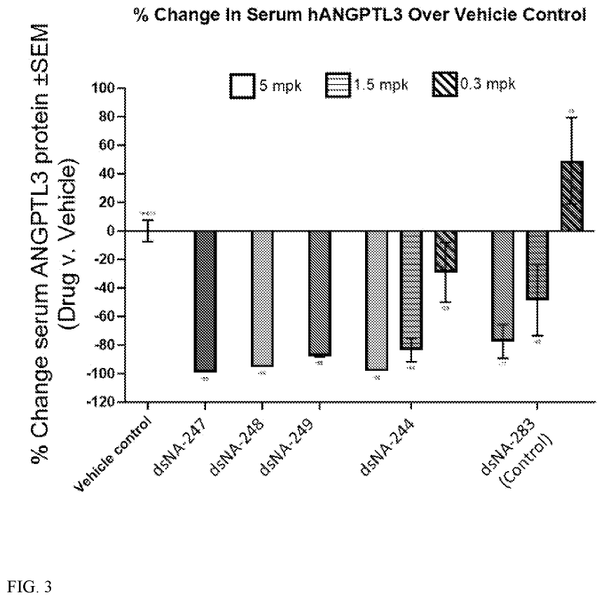

For ELISA, serum was separated from blood samples. Levels human ANGPTL3 protein in sera were quantified using ELISA (R&D Systems, DANL30) according to the manufacturer's protocol. Results are presented as percent change in expression relative to the expression levels of negative vehicle control samples (FIG. 3)

The results indicated that dsNA-244 showed greater target protein knockdown than comparative control dsNA-283 at all 3 dose levels (0.3, 1.5, or 5.0 mg/kg) and that dsNA-247, dsNA-248 or dsNA-249 also were more capable of knocking down the target protein at 5 mg/kg than comparative control dsNA-283.

### Example 8. PD Study in Human ANGPTL3 Knock-In Mice

To evaluate certain ANGPTL3 siNA agents, a knock-in mouse model that expresses human ANGPTL3 (KI-ANGPTL3) was used. Mice (n=6/group) were subcutaneously administered a single dose (5 mg/kg) of siNA test article. Blood was collected at various timepoints throughout the study for ELISA and lipid quantification. Twenty-eight days after administration, the animals were sacrificed; the right, lateral liver lobe of each animal was collected for RT-qPCR and blood of each animal was collected for ELISA and lipid quantification. (Table 14 and 15).

TABLE 14
Treatment Groups for Example 8.

| | | Dose | Dose Volume | Animals |
|---|---|---|---|---|
| Group | Identifier | (mg/kg) | (mL/kg) | (n) |
| 1 | Vehicle-only | — | 5 | 6 |
| | Negative | | | |
| | Control | | | |
| 2 | dsNA-283 | 5 | 5 | 6 |
| | (Comparative | | | |
| | Control) | | | |
| 3 | dsNA-240 | 5 | 5 | 6 |
| 4 | dsNA-243 | 5 | 5 | 6 |
| 5 | dsNA-244 | 5 | 5 | 6 |
| 6 | dsNA-245 | 5 | 5 | 6 |
| 7 | dsNA-246 | 5 | 5 | 6 |
| 8 | dsNA-247 | 5 | 5 | 6 |

TABLE 15
Experimental Timeline for Example 8.

| Day | Sample | Data |
|---|---|---|
| −2 | Pre-dose serum sample | Baseline TG and LDL-C |
| | (15 μL) | |
| 0 | Pre-dose serum sample | TG and LCL-C; ELISA; TC |
| | (25 μL) | and HDL-C |
| 7 | Serum sample at 168 hours | TG and LCL-C; ELISA; TC |
| | (30 μL) | and HDL-C |
| 14 | Serum sample at 336 hours | TG and LDL-C; ELISA; TC |
| | (30 μL) | and HDL-C; ALT/AST |
| 21 | Serum sample at 504 hours | TG and LCL-C; ELISA; TC |
| | (30 μL) | and HDL-C |
| 28 | Serum sample at 672 hours | TG and LDL-C; ELISA; TC |
| | Terminal collection to | and HDL-C; ALT/AST |
| | collect as much volume as | |
| | possible | |
| | Liver sample | TG; gene expression |

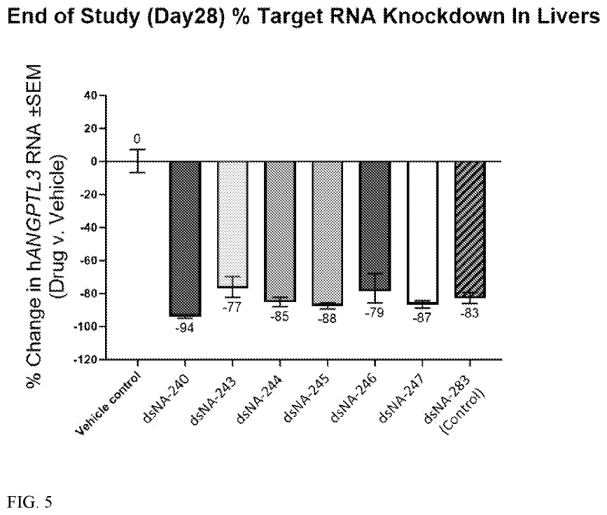

For RT-qPCR, RNA was extracted from the liver samples and cDNA was synthesized. Gene expression was measured using TaqMan Fast Advanced Master Mix (Applied Biosystems, 4444964), and the following TaqMan Gene Expression assays (Applied Biosystems, 4331182): Actb (Mm00607939_s1), ANGPTL3 (Hs00205581_m1), B2m (Mm00437762_m1), and Hmbs (Mm01143545_m1). Actb, B2m, and Hmbs served as the endogenous control genes. RT-qPCR reactions were run on the QuantStudio™ 6 Pro Real-Time PCR System (Applied Biosystems). The RQ of gene expression was calculated via the 2−ΔΔCt (RQ) method. Results are presented as percent change in expression relative to the expression levels of negative vehicle control samples. Knockdown of ANGPTL3 RNA in the liver samples is shown in FIG. 5.

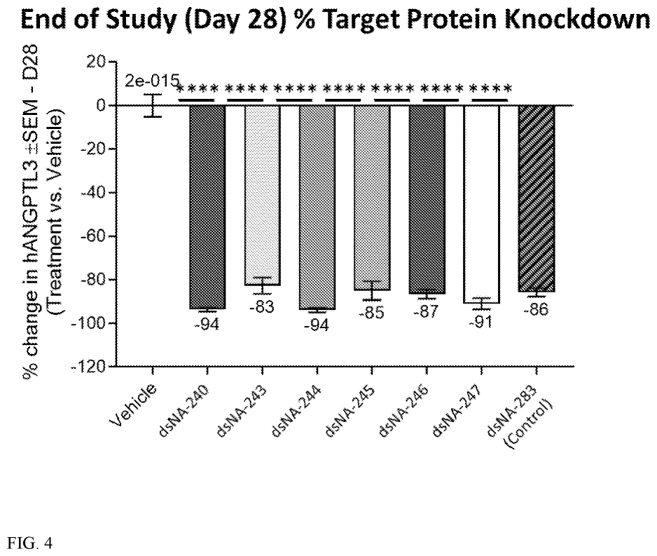

Serum was separated from blood samples. Human ANGPTL3 protein levels in the serum were quantified using ELISA (Abcam, ab254510) (FIG. 4). Triglycerides and total cholesterol levels in the serum were also quantified using a Roche Cobas 6000 c501 Chemistry Analyzer. Absolute protein and lipid concentrations are reported (Table 16; FIG. 4).

TABLE 16
Maximum Percent Serum Lipid Reduction vs. Vehicle-Only
Negative Control During 28-Day Treatment Period

| | dsNA-283 | | | | | | |
|---|---|---|---|---|---|---|---|
| | (Comparative | dsNA- | dsNA- | dsNA- | | | |
| | Control) | 240 | 243 | 244 | dsNA-245 | dsNA-246 | dsNA-247 |
| Maximum | 48% | 53% | 42% | 54% | 57% | 45% | 51% |
| % | | | | | | | |
| Triglyceride | | | | | | | |
| (TG) | | | | | | | |
| Knockdown | | | | | | | |
| vs. Vehicle- | | | | | | | |
| Only | | | | | | | |
| Negative | | | | | | | |
| Control | | | | | | | |
| Maximum | 29% | 35% | 24% | 32% | 37% | 37% | 44% |
| % Low- | | | | | | | |
| Density | | | | | | | |
| Lipoprotein- | | | | | | | |
| Cholesterol | | | | | | | |
| (LDL-C) | | | | | | | |
| Knockdown | | | | | | | |
| vs. Vehicle- | | | | | | | |
| Only | | | | | | | |
| Negative | | | | | | | |
| Control | | | | | | | |
| Maximum | 43% | 46% | 41% | 43% | 41% | 41% | 40% |
| % Total | | | | | | | |
| Cholesterol | | | | | | | |
| (TC) | | | | | | | |
| Knockdown | | | | | | | |
| vs. Vehicle- | | | | | | | |
| Only | | | | | | | |
| Negative | | | | | | | |
| Control | | | | | | | |

The results of the liver ANGPTL3 RNA knockdown experiment showed that dsNA-240, dsNA-244, dsNA-245 and dsNA-247 showed greater target RNA knockdown than comparative control dsNA-283, while dsNA-243 and dsNA-246 showed less target RNA knockdown than the comparative control siNA. In addition, the results of the serum ANGPTL3 protein knockdown exhibited a similar trend with dsNA-240, dsNA-244 and dsNA-247 showing a higher percentage of target protein knockdown than comparative control dsNA-283, while dsNA-243, dsNA-245 and dsNA-246 showed similar target protein knockdown as the comparative control. Finally, the results of the serum lipid reduction study showed that dsNA-240, dsNA-244 and dsNA-245 had overall better lipid reduction profiles than that of comparative control dsNA-283, but dsNA-243, dsNA-246 and dsNA-247 did not show significant improvement when compared with comparative control dsNA-283.

### Example 9. RNA-Seq Off-Target Analysis

To assess the off-target profile, 10 nM siNA was transfected into PHH and after 24 hours, total RNA was isolated. On target RNA knockdown (human ANGPTL3) in total RNA was confirmed by RT-PCR and standard RNA-Seq was carried out by Genewiz/Azenta. The raw FASTQ files were trimmed using Trimmomatic (v. 0.39) to eliminate adapter sequences, then reads were aligned and mapped to genes from the Homo sapiens GRCh38.104 Ensembl annotation using STAR. The aligned/mapped reads were counted/summarized using featureCounts. Differential expressed gene (DEG) analysis was carried out using DESeq2 in R. Genes with fold change greater than 2 and adjusted p-value less than 0.05 were counted as DE genes. The results of this analysis are shown in Table 17.

TABLE 17
Quantification of RNA-Seq Off-Target Analysis

PHH (10 nM)

| | Human | Number of DE |
|---|---|---|
| | ANGPTL3% RNA | Genes in RNA- |
| Identifier | Knockdown | Seq (DEG) |
| dsNA-283 | 88% | 7 |
| (Comparative | | |
| Control) | | |
| dsNA-240 | 90% | 45 |
| dsNA-241 | 92% | 5 |
| dsNA-242 | 92% | 30 |
| dsNA-243 | 91% | 3 |
| dsNA-244 | 88% | 1 |
| dsNA-245 | 86% | 153 |
| dsNA-246 | 87% | 109 |
| dsNA-247 | 87% | 12 |

All siNA showed good human ANGPTL3 target RNA knockdown at 10 nM in Primary Human Hepatocytes (PHH). However, dsNA-244 (DEG=1), dsNA-243 (DEG=3) and dsNA-241 (DEG=5) have fewer off targets than comparative control dsNA-283 (DEG=7).

While dsNA-240 has good potency and knockdown capability, it does not have ideal off target profile as it has 45 DE genes identified in the RNA-Seq analysis. Thus, the seed region of dsNA-240 would be an ideal candidate for testing chemical modifications to reduce off targets while preserving the potency.

### Example 10. Modifying siNA Candidate dsNA-240 by Applying Aligos Chemistries to Destabilize the Seed Region

In order to improve dsNA-240 to maintain potency while reducing off-target binding, a seed destabilizing strategy was applied. This strategy involves incorporation of various nucleotide modifications, including UNA, seco, 3-OH, 3-OCP, or xylo-F chemistries, into key positions in the antisense seed region, which include positions 6 and 7 from the 5′ end of the antisense strand. When the modified siNAs generated via this strategy were tested, all candidates maintained in vitro potency (Table 18). Additional seed destabilized siNAs were generated in a second subsequent round (Table 19), This second round of testing further confirmed that seed destabilization does not significantly affect compound potency.

TABLE 18
Seed Destabilized siNA Round 1 EC₅₀ Results

| Identifier | EC₅₀ (pM) |
|---|---|
| dsNA-240 | 9 |
| Parental siNA) | |
| dsNA-262 | 8.7 |
| dsNA-263 | 4 |
| dsNA-264 | 3.9 |
| dsNA-265 | 5 |
| dsNA-266 | 9 |

TABLE 19
Seed Destabilized siNA Round 2 EC₅₀ Results

| Identifier | EC₅₀ (pM) |
|---|---|
| dsNA-240 | 15.5 |
| Parental siNA) | |
| dsNA-270 | 20.1 |
| dsNA-271 | 40.8 |
| dsNA-272 | 68.9 |
| dsNA-273 | 23.8 |
| dsNA-274 | 13.6 |
| dsNA-275 | 13.6 |
| dsNA-276 | 16.6 |
| dsNA-277 | 33.9 |
| dsNA-278 | 24.3 |
| dsNA-279 | 20.3 |
| dsNA-280 | 10.9 |
| dsNA-281 | 15.7 |

### Example 11. Plasmid Based On- and Off-Target Luciferase Assay

Two plasmids were constructed by inserting either on-target or off target sequence in the region of multiple cloning sites downstream of Renilla luciferase gene in psiCheck plasmid (C8021, Promega). On-target plasmid contains 87 nucleotide short sequence that is the complete sequence match (CM) of human ANGPTL3 with the target site of the specific siNA under investigation. The off-target plasmid contains the sequence match of the seed region (SM) of the siNA under investigation. Seed region of siNA is referring to nucleotide #2 to #8 of the antisense strand numbering from 5′ end. To enhance the signal generated from off target effect, we put in 4 tandem repeats of siNA seed region in psiCheck. On the day of experiment siNA under investigation with serial dilutions were transfected into Cos7 cells along with constant concentration of either on-target plasmid or off-target plasmid. Cos-7 cells containing on- or off-target plasmid, treated with siNA were incubated for 72 hours. Following the incubation, Renilla luciferase assay was carried out according to Promega's protocol. Prism software was used to plot on-target (CM) and off-target (SM) dose response curves.

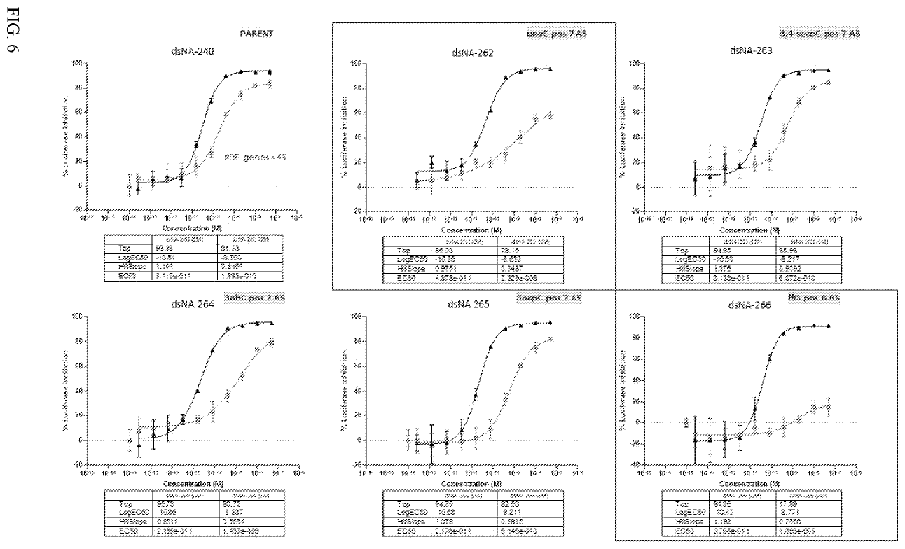

The results of the luciferase assay demonstrate that UNA at position 7 on AS (dsNA-262) and xylo-F at position 6 on AS (dsNA-266) resulted in greatest decrease in seed-related, off-target activity, while conserving on-target potency in comparison to parent compound dsNA-240 (FIG. 6).

### Example 12. AAV-hANGPTL8 Mouse Study Design

To evaluate certain ANGPTL8 siNA agents, an AAV-ANGPTL8 mouse model was used. Mice (n=4/group) were injected with an adeno-associated virus (AAV) expressing human ANGPTL8. Seven days after AAV injection, mice were subcutaneously administered a single dose (5 mg/kg) of an siNA. Twenty-eight days after administration, the animals were sacrificed; the right, lateral liver lobe of each animal was collected for RT-qPCR.

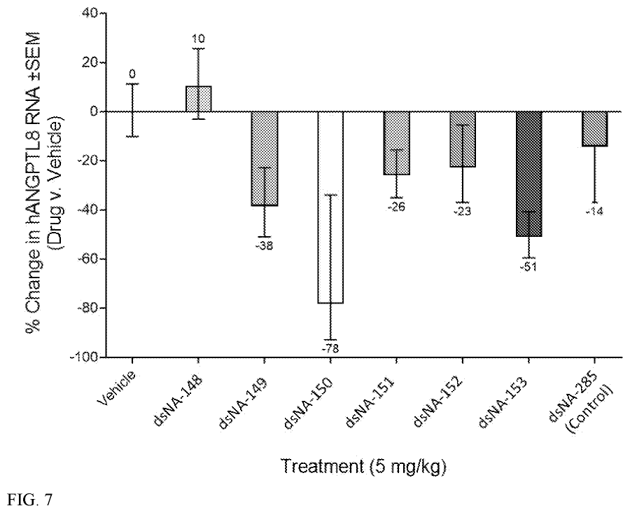

For RT-qPCR, RNA was extracted using the Rneasy Mini Kit (Qiagen, 74106), according to the manufacturer's protocol. RNA quantity and quality was analyzed with a NanoDrop™ Lite Spectrophotometer (Thermo Scientific), and cDNA was synthesized using the SuperScript IV VILO Master Mix (Invitrogen, 11756500), according to the manufacturer's protocol. Gene expression was measured using TaqMan Fast Advanced Master Mix (Applied Biosystems, 4444964), and the following TaqMan Gene Expression assays (Applied Biosystems, 4331182): Actb (Mm00607939_s1), ANGPTL8 (Hs00218820_m1), B2m (Mm00437762_m1), and Hmbs (Mm01143545_m1). Actb, B2m, and Hmbs served as the endogenous control genes. RT-qPCR reactions were run on the QuantStudio™ 6 Pro Real-Time PCR System (Applied Biosystems). The RQ of gene expression was calculated via the 2-AACt (RQ) method. Results are presented as percent change in expression relative to the expression levels of negative vehicle control samples (FIG. 7).

The results of the experiment showed that ANGPTL8 siNA dsNA-149, dsNA-150, dsNA-151, dsNA-152, dsNA-153 provided greater human ANGPTL8 RNA knockdown than comparative control siNA dsNA-285 (FIG. 7).

### Example 13. HANGPTL3 siNA and hANGPTL8 siNA Combination in Human ANGPTL3/8 Double Knock-In Mouse Study

To evaluate certain ANGPTL3 and ANGPTL8 siNA agents, a knock-in mouse model that expresses both human ANGPTL3 and ANGPTL8 (KI-ANGPTL3/8) was used. Mice (n=4/group) were subcutaneously administered a single dose (1 or 5 mg/kg) of one siNA or a combination of two siNAs. Thus, combination of ANGPTL3 and ANGPTL8 siNA was achieved by co-formulation of two siNAs in a single injection in this example. Blood was collected at various timepoints throughout the study for lipid quantification. Twenty-eight days after administration, the animals were sacrificed; the right, lateral liver lobe of each animal was collected for RT-qPCR and blood of each animal was collected for ELISA and lipid quantification. (Tables 20 and 21)

TABLE 20
Treatment Groups for Example 13.

| | | Dose | Dose Volume | Animals |
|---|---|---|---|---|
| Group | Identifier | (mg/kg) | (mL/kg) | (n) |
| 1 | Vehicle-only | — | 5 | 4 |
| | Negative | | | |
| | Control | | | |
| 2 | dsNA-283 | 1 | 5 | 4 |
| | (Comparative | | | |
| | Control) | | | |
| 3 | dsNA-150 | 5 | 5 | 4 |
| 4 | dsNA-152 | 5 | 5 | 4 |
| 5 | dsNA-150 + | 5 + 1 | 5 | 4 |
| | dsNA-244 | | | |
| 6 | dsNA-152 + | 5 + 1 | 5 | 4 |
| | dsNA-244 | | | |
| 7 | dsNA-150 + | 5 + 1 | 5 | 4 |
| | dsNA-241 | | | |
| 8 | dsNA-152 + | 5 + 1 | 5 | 4 |
| | dsNA-241 | | | |
| 9 | dsNA-244 | 1 | 5 | 4 |
| 10 | dsNA-241 | 1 | 5 | 4 |

TABLE 21
Experimental Timeline for Example 13.

| Day | Sample | Data |
|---|---|---|
| 0 | Pre-dose serum sample | TG and LCL-C; ELISA; TC |
| | (25 μL) | and HDL-C |
| 7 | Serum sample at 168 hours | TG and LCL-C; ELISA; TC |
| | (30 μL) | and HDL-C |
| 14 | Serum sample at 336 hours | TG and LDL-C; ELISA; TC |
| | (30 μL) | and HDL-C; ALT/AST |
| 21 | Serum sample at 504 hours | TG and LCL-C; ELISA; TC |
| | (30 μL) | and HDL-C |
| 28 | Serum sample at 672 hours | TG and LDL-C; ELISA; TC |
| | Terminal collection to | and HDL-C; ALT/AST |
| | collect as much volume as | |
| | possible | |
| | Liver sample | TG; gene expression |

For RT-qPCR, RNA was extracted from the liver samples and cDNA was synthesized. Gene expression was measured using TaqMan Fast Advanced Master Mix (Applied Biosystems, 4444964), and the following TaqMan Gene Expression assays (Applied Biosystems, 4331182): Actb (Mm00607939_s1), ANGPTL3 (Hs00205581_m1), ANGPTL8 (Hs00218820_m1), B2m (Mm00437762_m1), and Hmbs (Mm01143545_m1). Actb, B2m, and Hmbs served as the endogenous control genes. RT-qPCR reactions were run on the QuantStudio™ 6 Pro Real-Time PCR System (Applied Biosystems). The RQ of gene expression was calculated via the 2−ΔΔCt (RQ) method. Results are presented as percent change in expression relative to the expression levels of negative vehicle control samples.

Serum was separated from blood samples. Human ANGPTL3 protein levels in the serum were quantified using ELISA (R&D Systems, DANL30). Results are presented as percent change in expression of each treatment group at day 28 relative to each respective group's expression level pre-dose. Triglycerides and total cholesterol levels in the serum were also quantified using a Roche Cobas 6000 c501 Chemistry Analyzer. Absolute lipid concentrations are reported.

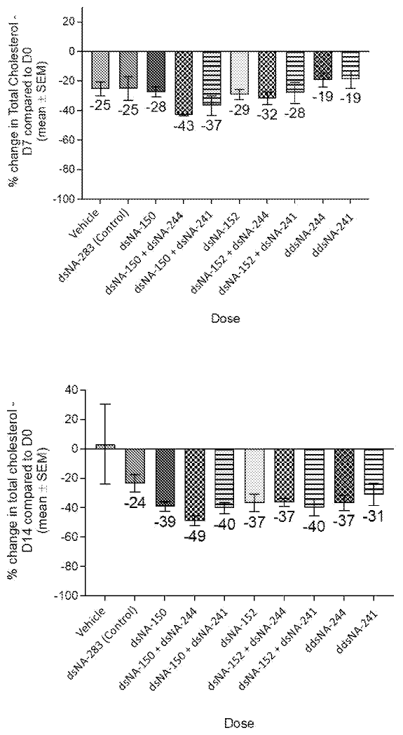

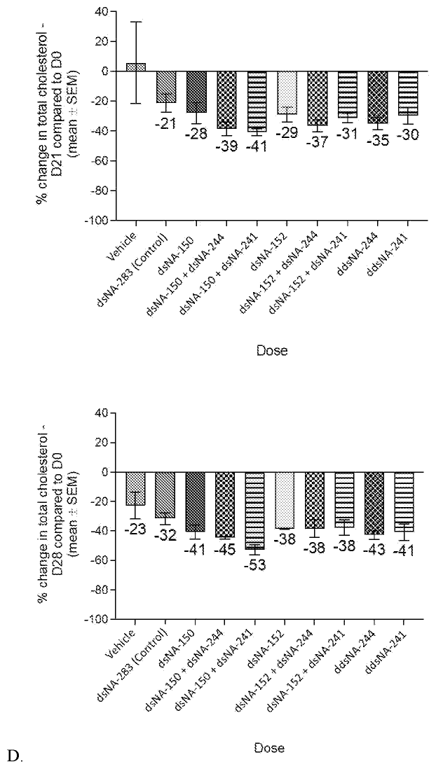

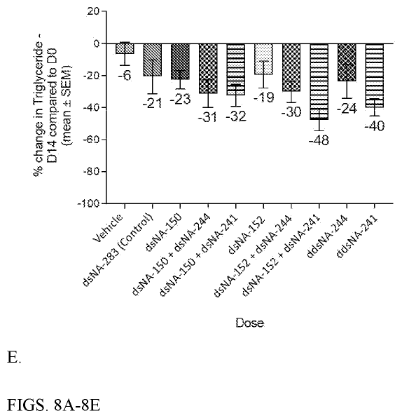

The results of the serum tests for total cholesterol and triglycerides are shown in FIG. 8. Some combinations of the ANGPTL3 and ANGPTL8 siNAs exhibited a greater reduction in total cholesterol levels as compared to the comparative control siNA and single siNA treatments on Day 7 post-dose (FIG. 8A). The combination of dsNA-244 and dsNA-152 showed a reduction in total cholesterol levels of 32%, while dsNA-244 alone only exhibited 19% reduction and dsNA-152 alone exhibited 29% reduction. This combination's reduction was also significantly more than the comparative control dsNA-283 alone, which exhibited a reduction of only 25%. Similarly, the combination of dsNA-244 and dsNA-150 together reduced total cholesterol levels 43%, which is significantly more than dsNA-283 and greater than the reduction seen for dsNA-244 alone or dsNA-150 alone (28%). Finally, the combination of dsNA-241 and dsNA-150 resulted in a 37% reduction of total cholesterol levels, which is also significantly different from the comparative control and greater than the reduction seen with only dsNA-241 (19%) or dsNA-150.

Like the results seen at Day 7 post-dose, certain combinations displayed reductions in total cholesterol levels on Day 14 post-dose (FIG. 8B). The combination treatment of dsNA-241 and dsNA-152 had a reduction of total cholesterol levels of 40%, which is a greater percent reduction as compared to dsNA-241 alone (31%) and ds-NA-152 alone (37%). The combination of dsNA-244 and ds-152 also had a higher reduction of total cholesterol (40%) than either dsNA-244 (37%) or dsNA-152 alone. Finally, knockdown of both ANGPTL3 and ANGPTL8 by dsNA-244 and dsNA-150 respectively showed a 49% reduction in total cholesterol, as compared to dsNA-244 alone or to dsNA-150 alone (39%). All three combinations were significantly better at reducing total cholesterol than the comparative control dsNA-283 (24%).

A similar trend for total cholesterol levels observed at Day 21 post-dose (FIG. 8C). The combination of dsNA-241 and dsNA-150 resulted in a 41% reduction in total cholesterol levels, while dsNA-241 alone had only 30% reduction and dsNA-150 alone had 28% reduction. The combination of dsNA-244 and dsNA-152 together had a reduction of 37% of total cholesterol levels, which is greater than only dsNA-244 (35%) or dsNA-152 (29%). Finally, the combination of dsNA-244 and dsNA-150 result in a likewise greater reduction in total cholesterol (39%) than either dsNA-244 or dsNA-150 alone. All of these combinations had a significantly greater reduction in total cholesterol levels than the comparative control dsNA-283 (21%).

The increase in the ability of combinations of siNAs to reduce total cholesterol levels was also seen at the final timepoint of Day 28 post-dose (FIG. 8D). The combination of dsNA-241 and dsNA-150 reduced total cholesterol levels by 53% at Day 28, whereas dsNA-241 alone and dsNA-150 alone both showed 41%. dsNA-244 and dsNA-150 together also had an increased reduction in total cholesterol levels (45%) as compared to dsNA-244 alone (43%) and ds-150 alone. Both combinations showed significantly greater reductions in total cholesterol than comparative control dsNA-283 alone (−32%).

A similar effect is seen for the combination siNA treatments when serum triglyceride levels were analyzed at Day 14 post-dose (FIG. 8E). The combination of dsNA-241 and dsNA-152 resulted in a 48% reduction in serum triglyceride levels as compared to dsNA-241 alone (40%) or dsNA-152 alone (19%). Treatment with both dsNA-244 and dsNA-152 also had a grater reduction in triglyceride levels (30%) as compared to only dsNA-244 (24%) and dsNA-152. Finally, the combination of dsNA-244 and ds-150 resulted in a 31% reduction, which is greater than the reduction seen for dsNA-244 or dsNA-150 (23%) alone. These combinations all exhibited significantly higher reduction in triglyceride levels than the comparative control dsNA-283 alone (−21%).

### Example 14. Base Modification of 5′-Vinyl Phosphonate End Cap

In order to further enhance in vitro potency, the 5′-vinyl phosphonate end cap was modified. The vm (5mim) U modification was capable of slightly improving the in vitro potency as compared to the parent siNA (dsNA-244) when tested via the same method as in Example 3 (Table 22).

TABLE 22
In Vitro Activities of siNA with Base-Modified End Cap

| | Hep3B hANGPTL3 |
|---|---|
| | RNA Knockdown |
| Identifier | (EC₅₀) |
| dsNA-244 | 43 pM |
| (Parental siNA) | |
| dsNA-267 | 19 pM |
| dsNA-268 | 13 pM |
| dsNA-269 | 17 pM |

### Example 15. Production of Example 15 Monomer

### The Report of Production Process for Intermediate 1b

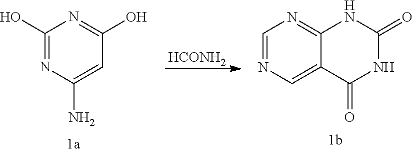

Preparation of (1b): To a solution of 1a (100.0 g, 787.4 mmol) in HCONH₂ (800 mL) was stirred at 150° C. for 8 hour. LC-MS and TLC show SM was completely consumed. The mixture was cooling to 0~5° C., and was added water (400 mL), the solid was collected by filtration. The filter cake was rinsed with MeOH (2×500 mL), drying by rotary evaporator to give 1b (108.0 g, 80% yield) as a yellow solid without further purified and used directly for the next step. ESI-LCMS m/z 165.0 [M+H]⁺. ¹H NMR (400 MHz, DMSO-d₆) δ 11.09 (s, 2H), 9.03-8.91 (m, 2H).

The Report of Production Process for Example 15 Monomer from Intermediate 1b

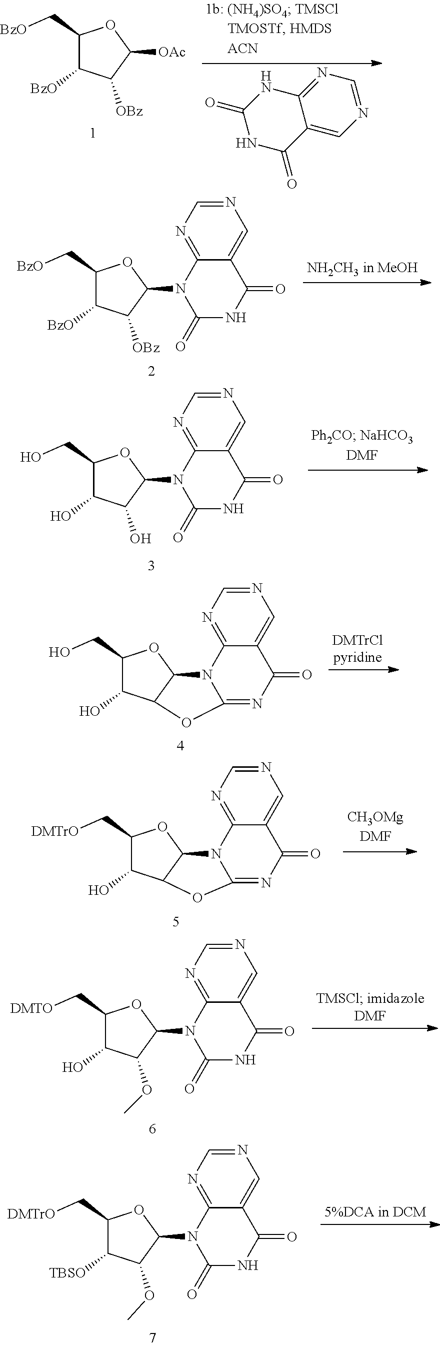

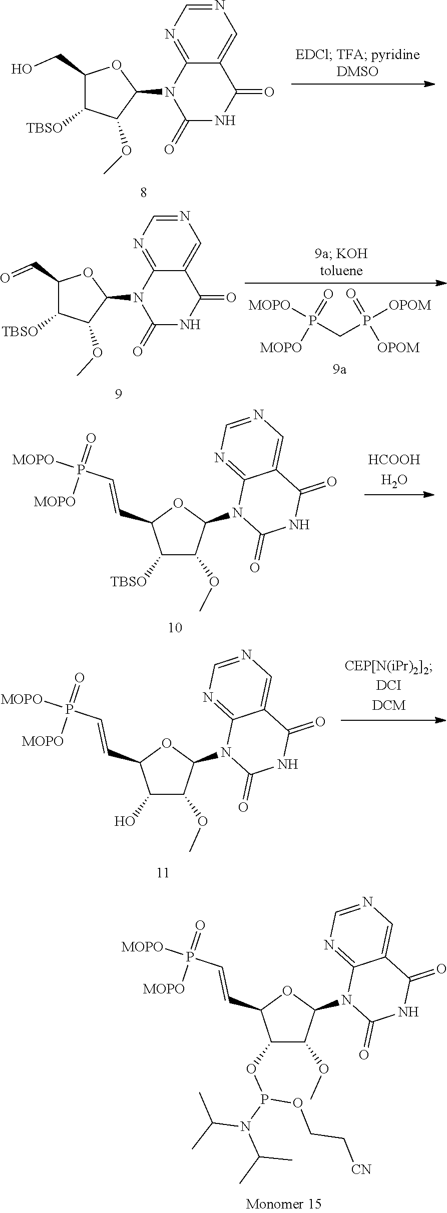

Preparation of (2): To a solution of 1b (130.0 g, 793.7 mmol) in HMDS (1.3 L) was added (NH₄)₂SO₄ (5.2 g, 39.7 mmol) and TMSCl (650.0 g, 5.9 mol) at room temperature. The mixture solution was stirred at 140° C. for 4 h. The mixture solution was removed under reduced pressure, the residue was removed with toluene three times under reduced pressure. The residue in ACN (4.0 L) was added 1 (400.0 g, 793.7 mmol) at room temperature. The mixture solution was cooled to 0° C., the mixture was added TMSOTf (264.3 g, 1.2 mol) drop wise at 0° C. The mixture solution was stirred at r.t for 3 h. TLC and LC-MS show SM was completely consumed. Then the solution was added Na₂HCO₃ (2000 mL), the product was extracted with EA, and was washed with water. Then washed the organic phase once with saturated brine and dried over by Na₂SO₄. Then the solution was concentrated under reduced pressure. The residue was isolated by silica gel column chromatography (eluent, PE/EA=2:1~1:1). This resulted in 2 (180.0 g, 296.0 mmol, 36% yield) as a yellow solid. ESI-LCMS: m/z 609.1 [M+H]⁻. ¹H NMR (400 MHZ, CDCl₃-d) § 9.33 (s, 1H), 9.13-9.00 (m, 2H), 8.07-7.84 (m, 6H), 7.61-7.27 (m, 9H), 7.04 (s, 1H), 6.35-6.13 (m, 2H), 4.91-4.60 (m, 3H).

Preparation of (3): To a solution of 2 (180.0 g, 296.0 mmol) was dissolved in 33% CH₃NH₂ in MeOH (1.5 L) at room temperature. The mixture solution was stirred at r.t for 4 h. TLC and LC-MS show 3 was completely consumed. Then the solution was removed under reduced pressure. The residue was purified by slurry with EA/PE=1:1. This resulted in 3 (80.0 g) as a white solid. ESI-LCMS: m/z 318.1 [M+Na]⁻. ¹H NMR (400 MHz, DMSO-d₆) δ 11.28 (s, 1H), 9.26-9.04 (m, 2H), 6.55 (s, 1H), 5.38-4.15 (m, 5H), 3.79-3.41 (m, 3H).

Preparation of (4): To a solution of 3 (75.0 g, 253.4 mmol) in dry DMF (750 mL) was added Ph2CO (60.0 g, 329.4 mmol) and NaHCO₃ (1.4 g, 17.7 mmol) at room temperature. The mixture solution was stirred at 140° C. for 1 h. LC-MS show 4 was completely consumed. The mixture solution was concentrated under reduced pressure. This resulting product was purified by silica gel column chromatograph (eluent, DCM/MeOH=30:1~10:1). This resulted in 6 (22.0 g, 27% yield) as a brown solid. ESI-LCMS: m/z 279.1 [M+H]⁻.

Preparation of (5): To a solution of 4 (21.0 g, 75.5 mmol) in pyridine (200 mL) was added DMTrCl (30.6 g, 90.6 mmol) at 25° C. The mixture solution was stirred at 25° C. for 2 h. LC-MS show SM was completely consumed. The mixture was diluted with EA (500 mL) and was washed with water. Then washed the organic phase once with saturated brine and dried over by Na₂SO₄. Then the solution was concentrated under reduced pressure. The residue was purified by silica gel column chromatograph (eluent, EA/PE=1:1). This resulted in 5 (31.0 g, 53.4 mmol, 66% yield) as a yellow solid. ESI-LCMS: m/z 581.1 [M+H]⁻. ¹H NMR (400 MHz, DMSO-d₆) δ 9.38 (s, 1H), 9.23 (s, 1H), 7.28-6.98 (m, 10H), 6.77-6.63 (m, 4H), 6.09-6.01 (m, 1H), 5.40-5.32 (m, 1H), 4.48-4.30 (m, 2H), 3.75-3.661 (m, 6H), 3.12-3.04 (m, 1H), 2.81-2.73 (m, 1H).

Preparation of (6): To the solution of 5 (30.0 g, 21.7 mmol) in dry DMF (300 mL) was added (MeO) 2 Mg (26.7 g, 310.3 mmol) at room temperature. And the reaction mixture was stirred at 100° C. for 2 hr. With ice-bath cooling, the reaction was quenched with saturated aq. NH₄Cl and the product was extracted into EA (500 mL). The combined organic layer was washed with water and brine, dried over Na₂SO₄, and concentrated to give a residue which was purified by silica gel column chromatograph (eluent, EA/PE=2:1). This resulted in 6 (23.0 g, 37.5 mmol, 72%) as a white solid. ESI-LCMS: m/z 611.1 [M−H]—. ¹H NMR (400 MHz, DMSO-d₆) δ 12.04 (s, 1H), 9.16 (s, 1H), 9.09 (s, 1H), 7.46-6.46 (m, 14H), 5.02-4.87 (m, 1H), 4.48-3.86 (m, 3H), 3.71 (s, 6H), 3.35 (s, 3H), 3.18 (s, 2H).

Preparation of (7): To a solution of 6 (18.0 g, 29.4 mmol) in DMF (180 mL) was added imidazole (4.0 g, 58.8 mmol) and TBSCl (5.3 g, 35.3 mmol) at room temperature. The mixture solution was stirred at 25° C. for 15 h. LC-MS show 6 was completely consumed. Then the mixture solution was added water, and the product was extracted with EA. And the organic phase was washed with water and saturated brine and dried over by Na₂SO₄. Then the solution was concentrated under reduced pressure. This resulted in crude 7 (19.5 g) as a white solid without further purified and used directly for the next step. ESI-LCMS: m/z 725.2 [M−H]⁻.

Preparation of (8): To a solution of crude 7 (19.5 g) was dissolved in 4% DCA in DCM (190 mL) at room temperature. The mixture solution was stirred at r.t for 20 min. LC-MS show SM was completely consumed. Then the solution was diluted with NaHCO₃·aq (200 mL) and the product was extracted with DCM and washed with water. Then washed the organic phase once with saturated brine and dried over by Na₂SO₄. Then the solution was concentrated under reduced pressure. The residue was purified by silica gel column chromatograph (eluent, DCM/MeOH=50:1). This resulted in giving 8 (6.5 g, 15.3 mmol, 40% yield over 2 steps) as a white solid. ESI-LCMS: m/z 425.1 [M+H]⁻. ¹H NMR (400 MHz, DMSO-d₆) δ 11.95 (s, 1H), 9.12-9.13 (m, 2H), 6.49 (s, 1H), 4.59-4.16 (m, 3H), 3.68-3.28 (m, 3H), 3.19 (s, 3H), 0.80 (s, 9H), 0.03-−003 (m, 6H).

Preparation of (9): To a solution of 8 (6.0 g, 14.1 mmol) and EDCI (8.1 g, 42.3 mmol) in DMSO (60 mL) was added Pyridine (1.3 g, 15.6 mmol) and TFA (888 mg, 7.8 mmol) at 0-5° C. The mixture was stirred at room temperature for 2 h, LC-MS showed the 8 was consumed completely and the reaction was quenched by water at 0° to obtain a mixture was diluted with EA which was separated by a funnel and aqueous phase was extracted with EA (100 mL*2) and was washed with water. Then washed the organic phase once with saturated brine and dried over by Na₂SO₄. Then the solution was concentrated under reduced pressure. This resulted in a crude 4 (6.5 g) as yellow solid was used directly in the next step. ESI-LCMS: m/z 423.1 [M+H]⁻.

Preparation of (10): To a solution of 9 (6.5 g) in toluene (65 mL) was added KOH (1.3 g, 24.0 mmol) and POM ester (8.1 g, 12.9 mmol) at room temperature under N₂ atmosphere. The reaction mixture was stirred at room temperature, for 2.5 h. LC-MS showed the 9 was consumed completely. Then the solution was diluted with EA (100 mL) and the product was extracted with EA and washed with water. Then washed the organic phase once with saturated brine and dried over by Na₂SO₄. Then the solution was concentrated under reduced pressure. The residue was purified by silica gel column chromatography (eluent, PE:EA=1:1~1:2) to give 10 (5.0 g, 6.9 mmol E/Z=76:23 by PNMR) as a yellow solid. ESI-LCMS: m/z 729.2 [M+H]⁻. ¹H NMR (400 MHz, DMSO-d₆) δ 12.09 (s, 1H), 9.18 (s, 2H), 6.80-6.43 (m, 2H), 6.18-5.91 (m, 1H), 5.68-5.47 (m, 4H), 4.93-4.23 (m, 3H), 3.40-3.20 (m, 3H), 1.23-1.05 (m, 18H), 0.89 (s, 9H), 0.08 (s, 6H). ³¹P NMR (600 MHz, DMSO-d₆) δ 16.887, 14.34.

Preparation of (11): A solution of 10 (5.0 g, 6.9 mmol) in HCOOH (25 mL) and water (25 mL) was stirred at room temperature overnight. LC-MS showed 10 was consumed completely. Con. NH₄OH was added to the mixture at 0° C. to quench the reaction until the pH=7.5. The product was extracted into ethyl acetate (100 mL). The product was extracted with EA and was washed with water. Then washed the organic phase once with saturated brine and dried over by Na₂SO₄. Then the solution was concentrated under reduced pressure. The residue was purified twice by silica gel column chromatography (eluent, DCM:MeOH=50:1~30:1) to give 11 (1.6 g, 2.6 mmol, 18% yield over 3 steps, >99% purity by PNMR) as a white solid. ESI-LCMS: m/z 616.1 [M+H]⁻. ¹H NMR (400 MHz, DMSO-d₆) δ 12.08 (s, 1H), 9.17 (s, 2H), 6.89-6.50 (m, 2H), 6.12-5.91 (m, 1H), 5.65-5.51 (m, 4H), 5.43-5.23 (m, 1H), 4.58-4.23 (m, 3H), 3.37 (s, 3H), 1.12 (s, 18H). ³¹P NMR (600 MHz, DMSO-d₆) δ 17.60.

Preparation of (Example 15 monomer): To a solution of 11 (1.6 g, 2.6 mmol) in DCM (20 mL) was added DCI (261 mg, 2.2 mmol) and CEP [N (iPr)₂]₂ (945 mg, 3.1 mmol) at r.t at N₂. The mixture was stirred at r.t at N₂ for 2 h. LC-MS showed all precursor was consumed completely. The mixture was added NaHCO₃ aqueous (100 mL) and extracted with DCM (100 mL). Then the organic layer was washed with water (200 mL) and brine (200 mL) and dried over Na₂SO₄. The solution was filtered, and the filtrate was concentrated to give the crude. The crude was purified by Flash-Prep-HPLC with the following conditions (IntelFlash-1): Column, C18 silica gel; mobile phase, CH₃CN/H₂O (0.05% NH₄HCO₃)=3/2 increasing to CH₃CN/H₂O (0.05% NH₄HCO₃)=1/0 within 20 min, the eluted product was collected at CH₃CN/H₂O (0.05% NH₄HCO₃)=1/0; Detector, UV 254 nm. This resulted in Example 15 monomer (1.6 g, 1.9 mmol, 75% yield, 99% purity) as a white solid. ESI-LCMS: m/z 815.2 [M+H]⁻. ¹H NMR (400 MHz, DMSO-d₆) δ 12.09 (s, 1H), 9.27-9.06 (m, 2H), 6.98-6.54 (m, 2H), 6.21-6.00 (m, 1H), 5.67-5.45 (m, 4H), 4.92-4.35 (m, 3H), 3.89-3.51 (m, 4H), 3.43-3.34 (m, 3H), 2.87-2.74 (m, 2H), 1.24-1.06 (m, 30H). ³¹P NMR (600 MHz, DMSO-d₆) δ 148.79, 147.97, 17.28, 16.92.

### Example 16. Production of Example 16 Monomer

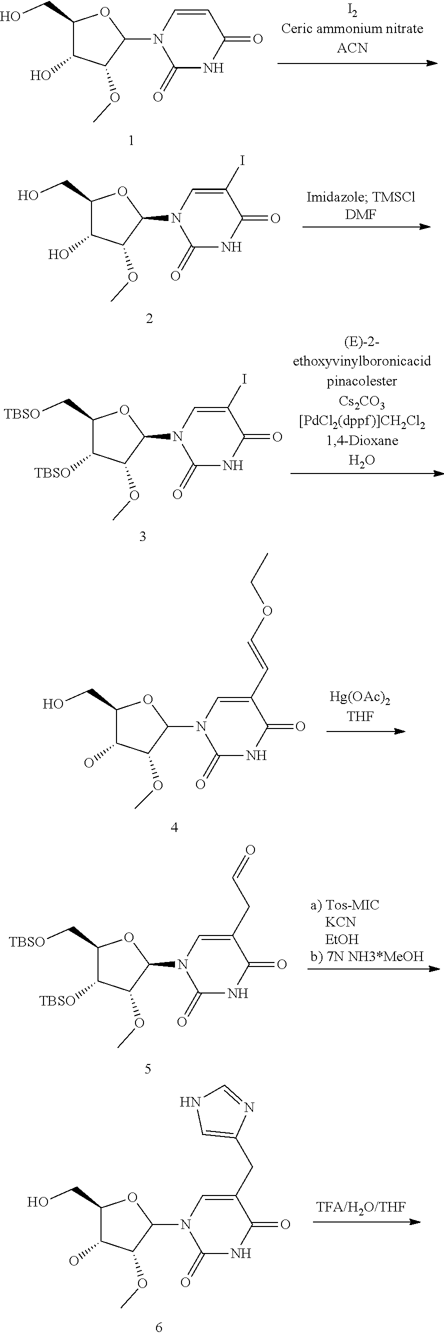

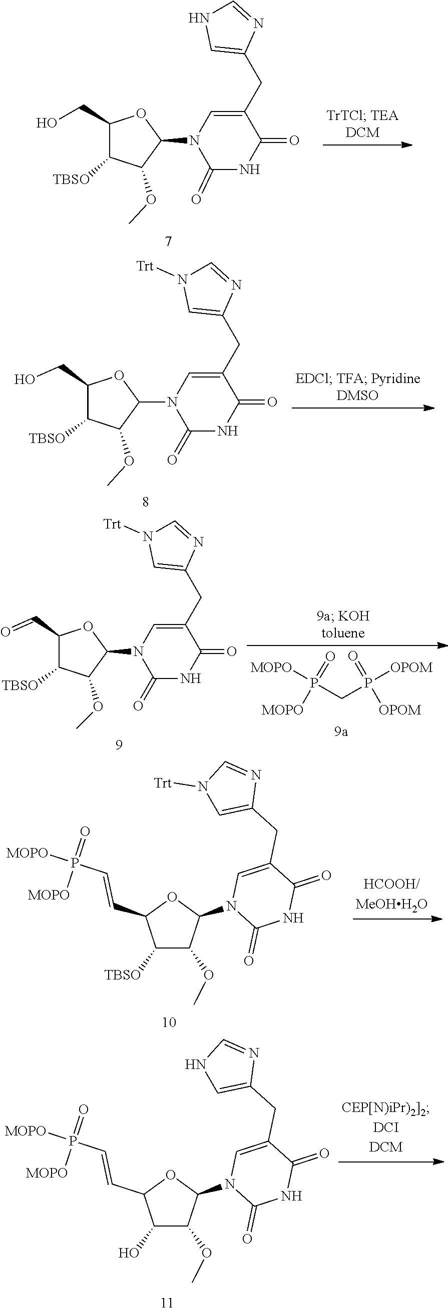

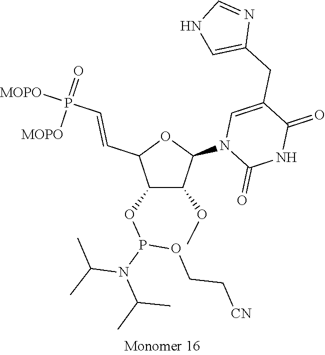

Preparation of (2): To a solution of 1 (100.0 g, 387.3 mmol) in ACN (1.0 L). Then the mixture was added Ceric ammonium nitrate (106.2 g, 193.6 mmol) and I₂ (59.0 g, 232.4 mmol). The mixture was stirred at r.t for 3 h, the solid was collected by filtration. The filter cake was rinsed with ACN (2×500 mL), drying by rotary evaporator to give crude 2 (120.0 g) as a white solid without further purified and used directly for the next step. ESI-LCMS: m/z 384.9 [M+H]⁺.

Preparation of (3): To a solution of 2 (120.0 g, 312.5 mmol) in DMF (1.0 L). Then the mixture was added Imidazole (63.8 g, 937.6 mmol), TBSCl (98.9 g, 656.3 mmol) under N₂ atmosphere. The mixture was stirred at room temperature for 16 h. Then the solution was diluted with EA. And the organic phase was washed with water and saturated brine and dried over by Na₂SO₄. Then the solution was concentrated under reduced pressure. The residue was purified by silica gel column chromatograph (eluent, PE/EA=100:1~1:1). This resulted in 3 (150.0 g, 245.1 mmol, 78.4% yield over two steps) as a solid. ESI-LCMS: m/z 613.1 [M+H]⁺.

Preparation of (4): To a stirred solution of 3 (150.0 g, 245.1 mmol) and (E)-2-ethoxyvinylboronic acid pinacol ester (97.1 g, 490.2 mmol) in 1, 4-Dioxane (1.5 L) and H₂O (187 mL) was added Cs₂CO₃ (175.7 g, 539.2 mmol), [PdCl₂ (dppf)] CH₂Cl₂ (19.8 g, 24.5 mmol). The resulting mixture was stirred overnight at 50° C. under argon atmosphere. The reaction was cooled to room temperature and was extracted with EA (2×1.0 L). And the organic phase was washed with water and saturated brine and dried over by Na₂SO₄. Then the solution was concentrated under reduced pressure. The residue was purified by silica gel column chromatograph (eluent, PE/EA=100:1~ 1:1). This resulted in giving 4 (107.0 g, 192.1 mmol) as a solid. ESI-LCMS: m/z 557.2 [M+H]⁺.

Preparation of (5): To the solution of 4 (107.0 g, 192.1 mmol) in THF (1.0 L) followed by the addition of Hg (OAc) 2 (110.0 g, 345.8 mmol) was dissolved in ice-water (500 mL). The resulting mixture was stirred for 6 h at 0° C. The reaction was quenched with sat. NH₄Cl (aq.) at 0° C. The resulting mixture was extracted with EA. And the organic phase was washed with water and saturated brine and dried over by Na₂SO₄. Then the solution was concentrated under reduced pressure. The residue was purified by silica gel column chromatograph (eluent, PE/EA=100:1~ 1:1). This resulted in 5 (41.0 g, 77.5 mmol) as a solid. ESI-LCMS: m/z 529.2 [M+H]⁺; ¹H NMR (400 MHz, DMSO-d₆) δ: 11.59 (s, 1H), 9.59 (d, J=1.3 Hz, 1H), 7.56 (s, 1H), 5.85 (d, J=5.5 Hz, 1H), 4.23 (q, J=6.9, 5.6 Hz, 1H), 3.94-3.75 (m, 2H), 3.75-3.65 (m, 1H), 3.40-3.32 (m, 4H), 0.89 (t, J=4.0 Hz, 18H), 0.16-0.03 (m, 12H).

Preparation of (6): To a stirred solution 5 (41.0 g, 77.5 mmol) in dry EtOH (800 mL) was added KCN (756 mg, 11.6 mmol) and Tos-MIC (16.6 g, 85.2 mmol) at room temperature for 30 min. The reaction was concentrated under reduced pressure. The residue was dissolved in 7N NH₃·MeOH (400 mL) in a High-pressure kettle. The resulting mixture was stirred for 4 h at 100° C. The reaction was quenched with sat. NH₄Cl (aq.) at 0° C. The resulting mixture was extracted with EA. And the organic phase was washed with water and saturated brine and dried over by Na₂SO₄. Then the solution was concentrated under reduced pressure. The residue was purified by silica gel column chromatograph (eluent, PE/EA=100:1~ 1:1). The residue was purified by Flash-Prep-HPLC with the following conditions (IntelFlash-1): Column, C18 silica gel; mobile phase, CH₃CN/H₂O (0.5% NH₄HCO₃)=1/3 increasing to CH₃CN/H₂O (0.5% NH₄HCO₃)=1/0 within 25 min, the eluted product was collected at CH₃CN/H₂O (0.5% NH₄HCO₃)=58/42; Detector, UV 254 nm. This resulted in giving 6 (10.3 g, 18.2 mmol) as a solid. ESI-LCMS: m/z 567.2 [M+H]⁺.

Preparation of (7): To a solution of 6 (10.3 g, 18.2 mmol) in THF (100 mL) and was added TFA/H₂O=1/1 (40 ml). The mixture was stirred at 0° C. for 1 h. TLC showed 6 was consumed completely. NaHCO₃ (aq.) was added to the mixture at 0° C. Then the solution diluted with EA. The organic layer was washed with brine, dried over anhydrous Na₂SO₄. After filtration, the filtrate was concentrated under reduced pressure. The residue was purified by silica gel column chromatography (eluent, DCM:MeOH=100:1~ 30:1). The residue was purified by Flash-Prep-HPLC with the following conditions (IntelFlash-1): Column, C18 silica gel; mobile phase, CH₃CN/H₂O (0.5% NH₄HCO₃)=1/3 increasing to CH₃CN/H₂O (0.5% NH₄HCO₃)=1/0 within 25 min, the eluted product was collected at CH₃CN/H₂O (0.5% NH₄HCO₃)=58/42; Detector, UV 254 nm. This resulted in giving 7 (3.0 g, 6.6 mmol) as a white solid. ESI-LCMS: m/z 453.3 [M+H]⁺.

Preparation of (8): To a stirred mixture of 7 (3.0 g, 6.6 mmol) in DCM (30 mL) was added TEA (1.3 g, 13.2 mmol) and TrTCl (2.2 g, 7.9 mmol) at room temperature under N₂ atmosphere. The resulting mixture was stirred for 2 h at room temperature under argon atmosphere. The reaction was quenched by the addition of sat. NaHCO₃ (aq.). The resulting mixture was extracted with EA. The organic layer was washed with brine, dried over anhydrous Na₂SO₄. After filtration, the filtrate was concentrated under reduced pressure. The residue was purified by silica gel column chromatography (eluent, PE (0.5% TEA)/EA=50:1~2:3). This resulted in giving 8 (2.8 g, 4.0 mmol, 61% yield) as an off-white solid. ESI-LCMS: m/z 695.2 [M+H]⁺.

Preparation of (9): To a stirred mixture of 8 (2.8 g, 4.0 mmol) in DMSO (30 mL) was added EDCI (2.3 g, 12.0 mmol), pyridine (0.35 g, 4.4 mmol) and was dropped TFA (0.25 g, 2.2 mmol) at 0° C. under N₂ atmosphere. The resulting mixture was stirred for 2 h at room temperature under argon atmosphere. The reaction was quenched by the addition of sat. NaHCO₃ (aq.). The resulting mixture was extracted with EA. The organic layer was washed with brine, dried over anhydrous Na₂SO₄. After filtration, the filtrate was concentrated under reduced pressure. This resulted in crude 9 (4.0 g) as yellow solid used directly for the next step. ESI-LCMS: m/z 711.2 [M+H₂O]⁺.

Preparation of (10): To a stirred mixture of 9 (4.0 g, 4.0 mmol) in Toluene (30 mL) was added KOH (0.41 g, 6.8 mmol) and POM (2.8 g, 4.4 mmol) at 0° C. under N₂ atmosphere. The resulting mixture was stirred for 2 h at room temperature under argon atmosphere. The reaction was quenched by the addition of sat. NaHCO₃ (aq.). The resulting mixture was extracted with EA. The organic layer was washed with brine, dried over anhydrous Na₂SO₄. After filtration, the filtrate was concentrated under reduced pressure. The residue was purified by Flash-Prep-HPLC with the following conditions (IntelFlash-1): Column, C18 silica gel; mobile phase, CH₃CN/H₂O (0.5% NH₄HCO₃)=1/3 increasing to CH₃CN/H₂O (0.5% NH₄HCO₃)=1/0 within 25 min, the eluted product was collected at CH₃CN/H₂O (0.5% NH₄HCO₃)=85/15; Detector, UV 254 nm. This resulted in giving 10 (3.1 g, 3.1 mmol) as a white solid. ESI-LCMS: m/z 999.2 [M+H]⁺.

Preparation of (11): To a stirred mixture of 10 (3.1 g, 3.1 mmol) in THF (6 mL) and MeOH (4 mL) was added Formic acid (15 mL) and H₂O (15 mL) at 0° C. The resulting mixture was stirred for 1 h at 0° C. The reaction was quenched by the addition of sat. NaHCO₃ (aq.). The resulting mixture was extracted with EA. The organic layer was washed with brine, dried over anhydrous Na₂SO₄. After filtration, the filtrate was concentrated under reduced pressure. The residue was purified by Flash-Prep-HPLC with the following conditions (IntelFlash-1): Column, C18 silica gel; mobile phase, CH₃CN/H₂O (0.5% NH₄HCO₃)=1/3 increasing to CH₃CN/H₂O (0.5% NH₄HCO₃)=1/0 within 25 min, the eluted product was collected at CH₃CN/H₂O (0.5% NH₄HCO₃)=75/25; Detector, UV 254 nm. The residue was purified by SFC. This resulted in giving 11 (1.4 g, 2.1 mmol) as a white solid. ESI-LCMS: m/z 643.5 [M+H]⁺; ¹H NMR (400 MHz, DMSO-d₆) δ: 11.78 (s, 1H), 11.44 (s, 1H), 7.54 (d, J=20.3 Hz, 2H), 6.90-6.59 (m, 2H), 6.10 (ddd, J=22.2, 17.1, 1.5 Hz, 1H), 5.80 (d, J=4.0 Hz, 1H), 5.62 (dt, J=13.7, 1.0 Hz, 4H), 5.53 (d, J=6.7 Hz, 1H), 4.39-4.30 (m, 1H), 3.89 (dd, J=5.4, 4.0 Hz, 1H), 3.45 (s, 2H), 3.38 (s, 3H), 3.33 (s, 2H), 1.14 (d, J=3.8 Hz, 18H); ³¹P NMR (162 MHz, DMSO-d₆) δ: 17.22.

Preparation of (Example 16 Monomer): To a solution of 11 (1.4 g, 2.1 mmol) in DCM (20 mL) was added DCI (210 mg, 1.7 mmol) and CEP [N (iPr)₂]₂ (0.76 g, 2.5 mmol) under N₂. The mixture was stirred at 25° C. for 2.5 h. LCMS showed 11 was consumed completely. The product was extracted with DCM. The organic layer was washed with H₂O and brine. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/3 increasing to CH₃CN/H₂O (0.5% NH₄HCO₃)=1/0 within 25 min, the eluted product was collected at CH₃CN/H₂O (0.5% NH₄HCO₃)=1/0; Detector, UV 254 nm. This resulted in Example 16 Monomer (600 mg, 6.4 mmol) as a white solid. ESI-LCMS: m/z 843.2 [M+H]⁺; ¹H NMR (400 MHz, DMSO-d₆) δ: 11.78 (s, 1H), 11.45 (s, 1H), 7.57 (d, J=15.1 Hz, 2H), 6.81-6.73 (m, 1H), 6.16 (ddt, J=21.8, 17.1, 1.3 Hz, 1H), 5.82-5.73 (m, 1H), 5.67-5.55 (m, 4H), 4.58-4.44 (m, 1H), 4.35 (ddt, J=43.0, 11.0, 5.6 Hz, 1H), 4.11 (q, J=4.5 Hz, 1H), 3.85-3.67 (m, 1H), 3.65-3.53 (m, 1H), 3.38 (q, J=17.0, 16.0 Hz, 8H), 2.86-2.74 (m, 2H), 1.17-1.10 (m, 30H); ³¹P NMR (162 MHz, DMSO-d₆) δ: 149.63, 149.07, 16.92, 16.62.

### Example 17. Production of Example 17 Monomer

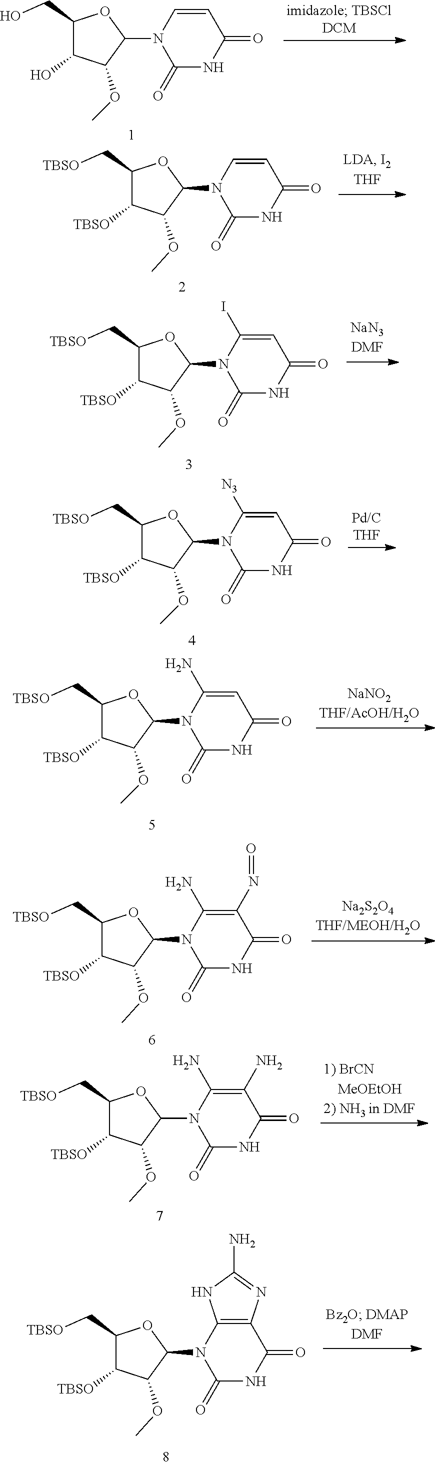

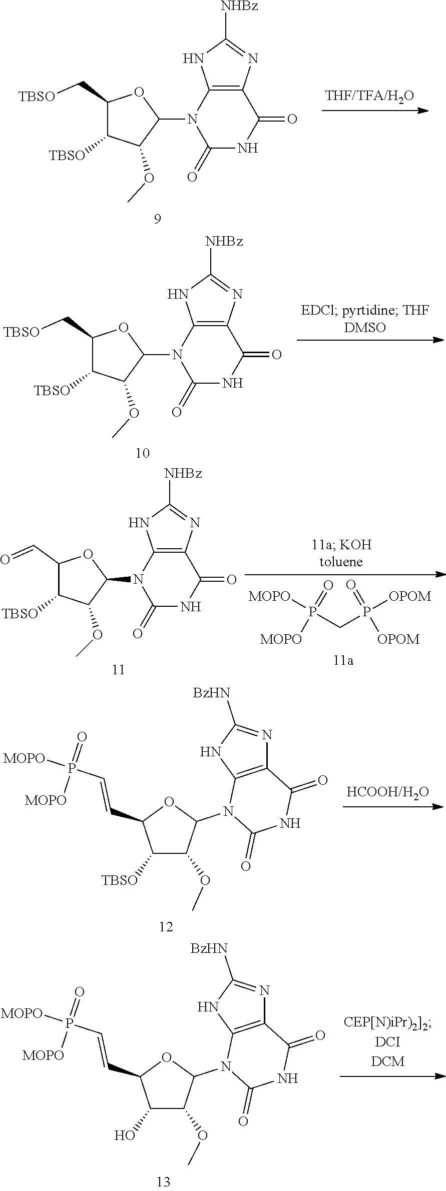

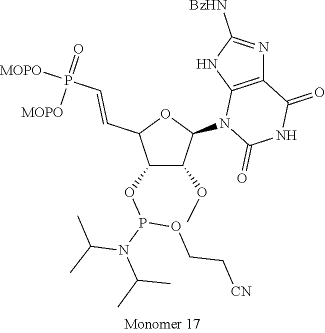

Preparation of (2): To a solution of 1 (300.0 g, 1.1 mol) in DMF (3.0 L) was added imidazole (408.0 g, 6.0 mol) and TBSCl (453.0 g, 3.0 mol) at room temperature. The mixture solution was stirred at 50° C. for 15 h. LC-MS show 1 was completely consumed. Then the mixture solution was added water, and the product was extracted with EA. And the organic phase was washed with water and saturated brine and dried over by Na₂SO₄. Then the solution was concentrated under reduced pressure. The residue was purified by silica gel column chromatograph (eluent, PE/EA=5:1). This resulted in giving 2 (500.0 g, 1.0 mol, 90% yield) as a white solid. ESI-LCMS: m/z 487.3 [M+H]⁻. ¹H NMR (400 MHz, DMSO-d₆) δ 11.30 (s, 1H), 7.71 (d, J=8.1 MHz, 1H), 5.71 (d, J=8.2 MHz, 1H), 5.47 (d, J=12.1 MHz, 1H), 4.19-4.09 (m, 1H), 3.87-3.54 (m, 4H), 3.28 (s, 3H), 0.86-0.74 (m, 18H), 0.02-−0.01 (m, 12H).

Preparation of (3): To a solution of 2 (530.0 g, 1.1 mol) in dry THF (2.5 L) was added 2M LDA (1.7 L, 3.3 mol) drop wise at −78° C. The mixture solution was stirred at −78° C. for 1 h. The mixture was added I₂ (1.1 kg, 4.4 mol) drop wise at −78° C. The mixture solution was stirred at −78° C. for 2 h. TLC and LC-MS show SM was completely consumed. Then the solution was added NH₄Cl·aq (500 mL), the product was extracted with EA, and was washed with water. Then washed the organic phase once with saturated brine and dried over by Na₂SO₄. Then the solution was concentrated under reduced pressure. The residue was to give crude 3 (635.0 g) as a brown solid without further purified and used directly for the next step. ESI-LCMS: m/z 613.13 [M+H]⁻.

Preparation of (4): To a solution of 3 (635.0 g) in DMF (6.5 L) was added NaN₃ (107.0 g, 1.7 mol) at 25° C. The mixture solution was stirred at r.t for 15 h. LC-MS show SM was completely consumed. Then the solution was added H₂O (500 mL), the product was extracted with EA, and was washed with water. Then washed the organic phase once with saturated brine and dried over by Na₂SO₄. Then the solution was concentrated under reduced pressure. The residue was to give crude 4 (580.0 g) as a yellow solid without further purified and used directly for the next step. ESI-LCMS: m/z 528.1 [M+H]⁻.

Preparation of (5): To a solution of 4 (580.0 g) in THF (5.6 L) was added 10% Pd/C (58.4 g) at 25° C. The mixture solution was stirred at r.t at H₂ for 5 h. LC-MS show SM was completely consumed. Filtered out the solid, and the solution was concentrated under reduced pressure. The residue was purified by silica gel column chromatograph (eluent, PE/EA=2:1~1:1). This resulted in giving 5 (60.0 g, 119.8 mmol, 12% yield over 3 steps) as a yellow solid. ESI-LCMS: m/z 503.0 [M+H]⁻. ¹H NMR (400 MHz, DMSO-d₆) δ 10.65-10.54 (m, 1H), 6.59 (s, 2H), 6.13-6.02 (m, 1H), 4.70 (d, J=4.0 MHz, 1H), 4.42-4.02 (m, 2H), 3.89-3.66 (m, 3H), 3.26 (s, 3H), 0.92-0.82 (m, 18H), 0.15-0.03 (m, 12H).

Preparation of (6): To a solution of 4 (60.0 g, 119.8 mmol) in THF/AcOH/H₂O=1:1:1 (600 mL) was added NaNO₂ (12.4 g, 179.8 mmol) at 25° C. The mixture solution was stirred at 50° C. for 1 h. LC-MS show SM was completely consumed. The solution was diluted with EA (600 mL) and washed with water. Then washed the organic phase once with saturated brine and dried over by Na₂SO₄. Then the solution was concentrated under reduced pressure. The residue was to give crude 4 (60.0 g) as a yellow solid without further purified and used directly for the next step. ESI-LCMS: m/z 531.0 [M+H]⁻.

Preparation of (7): To a solution of 6 (60.0 g) in THE/MeOH/H₂O=1:1:1 (600 mL) was added Na₂S₂O₄ (89.64 g, 515.8 mmol) at 25° C. The mixture solution was stirred at r.t for 1 h. LC-MS show SM was completely consumed. The solution was diluted with EA (600 mL) and washed with water. Then washed the organic phase once with saturated brine and dried over by Na₂SO₄. Then the solution was concentrated under reduced pressure. The residue was purified by silica gel column chromatograph (eluent, PE/EA=1:1~1:2). This resulted in giving 7 (31.0 g, 60.0 mmol, 50% yield over 2 steps) as a yellow solid. ESI-LCMS: m/z 517.2 [M+H]⁻. ¹H NMR (400 MHz, DMSO-d₆) δ 10.87 (s, 1H), 6.09 (d, J=8.0 MHz, 1H), 5.74 (s, 2H), 4.48-4.37 (m, 1H), 4.12-3.67 (m, 4H), 3.25 (s, 3H), 3.23-2.97 (m, 1H), 0.88 (s, 18H), 0.10-0.05 (m, 12H).

Preparation of (8): To a solution of 7 (30.0 g, 58.0 mmol) in MeOEtOH (300 mL) was added BrCN (29.9 L, 250.0 mmol) at 0° C. The mixture solution was stirred at 25° C. for 15 min. The mixture was added water, and the mixture was filtered, and the filter residue was concentrated. The residue was dissolved in DMF (300 mL). The mixture was supplied with NH₃ gas, and the mixture solution was stirred at 25° C. for 15 h. TLC and LC-MS show SM was completely consumed. Then the solution was added NH₄Cl·aq (500 mL), the product was extracted with EA, and was washed with water. Then washed the organic phase once with saturated brine and dried over by Na₂SO₄. Then the solution was concentrated under reduced pressure. This resulted was purified by silica gel column chromatograph (eluent, PE:EA=2:1~1:2). The residue was purified by Flash-Prep-HPLC with the following conditions (IntelFlash-1): Column, C18 silica gel; mobile phase, CH₃CN/H₂O (0.05% NH₄HCO₃)=1/3 increasing to CH₃CN/H₂O (0.05% NH₄HCO₃)=4/1 within 25 min, the eluted product was collected at CH₃CN/H₂O (0.05% NH₄HCO₃)=3/2; Detector, UV 254 nm. This resulted in giving 8 (4.5 g, 8.3 mmol, 14% yield) as a yellow solid. ESI-LCMS: m/z 540.2 [M+H]⁻. 1H NMR (400 MHz, DMSO-d₆) δ 11.36 (s, 1H), 10.65 (s, 1H), 6.78-5.87 (m, 3H), 4.66-4.46 (m, 2H), 3.88-3.54 (m, 3H), 3.27 (s, 3H), 1.75 (s, 1H), 0.95-0.77 (m, 18H), 0.14-−0.05 (m, 12H).

Preparation of (9): To a solution of 8 (4.5 g, 8.3 mmol) in DMF (45 mL) was added Bz₂O (2.1 g, 9.1 mmol) and DMAP (101 mg, 0.8 mmol) at room temperature. The mixture was stirred at r.t for 4 h. TLC and LC-MS show the reaction was complete. The mixture was poured into NaHCO₃·aq, the product was extracted with EA. Then washed the organic phase once with saturated brine and dried over by Na₂SO₄. Then the solution was concentrated under reduced pressure. This resulted was purified by Flash-Prep-HPLC with the following conditions (IntelFlash-1): Column, C18 silica gel; mobile phase, CH₃CN/H₂O (0.05% NH₄HCO₃)=2/3 increasing to CH₃CN/H₂O (0.05% NH₄HCO₃)=1/0 within 30 min, the eluted product was collected at CH₃CN/H₂O (0.05% NH₄HCO₃)=4/1; Detector, UV 254 nm. This resulted in 9 (3.3 g, 5.1 mmol, 61% yield, 98% purity) as a white solid. ESI-LCMS: m/z 646.2 [M+H]⁻. ¹H NMR (400 MHz, DMSO-d₆) δ 12.70 (s, 1H), 11.83 (s, 1H), 11.13 (s, 1H), 8.12-7.44 (m, 5H), 6.15-6.01 (m, 1H), 4.81-4.50 (m, 2H), 3.91-3.25 (m, 6H), 0.98-0.77 (m, 18H), 0.17-−0.05 (m, 12H).

Preparation of (10): To a solution of 9 (3.3 g, 5.1 mmol) in THF (10 mL) and was added TFA/H₂O=1/1 (20 ml). The mixture was stirred at 0° C. for 1 h. TLC showed 9 was consumed completely. NaHCO₃ (aq.) was added to the mixture at 0° C. Then the solution diluted with EA. The organic layer was washed with brine, dried over anhydrous Na₂SO₄. After filtration, the filtrate was concentrated under reduced pressure. The residue was purified by silica gel column chromatography (eluent, PE:EA=3:1~1:1). The residue was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=1/0 within 25 min, the eluted product was collected at CH₃CN/H₂O (0.5% NH₄HCO₃)=58/42; Detector, UV 254 nm. This resulted in 10 (1.9 g, 3.6 mmol) as a white solid. ESI-LCMS: m/z 530.3 [M+H]⁻. ¹H NMR (400 MHz, DMSO-d₆) δ 12.74 (s, 1H), 11.83 (s, 1H), 11.17 (s, 1H), 8.08-7.50 (m, 5H), 6.15-6.01 (m, 1H), 4.84-4.45 (m, 3H), 3.84-3.22 (m, 6H), 0.90 (s, 18H), 0.12-0.0 (m, 6H).

Preparation of (11); To a solution of 10 (1.9 g, 3.6 mmol) and EDCI (2.1 g, 10.7 mmol) in DMSO (20 mL) was added pyridine (310 mg, 3.9 mmol) and TFA (223 mg, 1.9 mmol) at 0-5° C. The mixture was stirred at room temperature for 2 h, LC-MS showed the 10 was consumed completely and the reaction was quenched by water at 0° to obtain a mixture was diluted with EA which was separated by a funnel and aqueous phase was extracted with EA (100 mL*2) and was washed with water. Then washed the organic phase once with saturated brine and dried over by Na₂SO₄. Then the solution was concentrated under reduced pressure. This resulted in a crude 11 (1.8 g) as yellow solid, which was used directly to next step. ESI-LCMS: m/z 528.3 [M+H]⁻.

Preparation of (12): To a solution of 11 (1.8 g) in toluene (20 mL) was added KOH (323 g, 5.8 mmol) and POM seter (2.4 g, 3.7 mmol) at room temperature under N₂ atmosphere. The reaction mixture was stirred at room temperature for 2.5 h. LC-MS showed the 11 was consumed completely. Then the solution was diluted with EA (100 mL) and the product was extracted with EA and washed with water. Then washed the organic phase once with saturated brine and dried over by Na₂SO₄. Then the solution was concentrated under reduced pressure. The residue was purified by silica gel column chromatography (eluent, PE:EA=2:1~1:1). The residue was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=1/0 within 25 min, the eluted product was collected at CH₃CN/H₂O (0.5% NH₄HCO₃)=4/1; Detector, UV 254 nm. This resulted in 12 (600 mg, 0.7 mmol, E/Z>99% by PNMR) as a white solid. ESI-LCMS: m/z 834.2 [M−H]⁻. ¹H NMR (400 MHz, DMSO-d₆) δ 12.73 (s, 1H), 11.81 (s, 1H), 11.18 (s, 1H), 8.13-7.47 (m, 5H), 6.98-6.67 (m, 1H), 6.21-6.00 (m, 2H), 5.67-5.48 (m, 4H), 4.69-4.28 (m, 3H), 3.34 (s, 3H), 1.13-1.09 (m, 18H), 0.89 (s, 9H), 0.11-0.06 (m, 6H). ³¹P NMR (600 MHz, DMSO-d₆) δ 17.28.

Preparation of (13): A solution of 12 (600 mg, 0.7 mmol) in HCOOH (3 mL) and water (3 mL) was stirred at room temperature overnight. LC-MS showed 12 was consumed completely. Con. NH₄OH was added to the mixture at 0° C. to quench the reaction until the pH=7.5. The product was extracted into ethyl acetate (100 mL). The product was extracted with EA and was washed with water. Then washed the organic phase once with saturated brine and dried over by Na₂SO₄. Then the solution was concentrated under reduced pressure. The residue was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=1/0 within 25 min, the eluted product was collected at CH₃CN/H₂O (0.5% NH₄HCO₃)=4/1; Detector, UV 254 nm. This resulted in 13 (330 mg, 0.4 mmol, 57% yield, >98% purity by HPLC & PNMR) as a white solid. ESI-LCMS: m/z 720.2 [M−H]⁻. ¹H NMR (400 MHz, DMSO-d₆) δ 12.67 (s, 1H), 11.78 (s, 1H), 11.18 (s, 1H), 8.13-7.45 (m, 5H), 6.99-6.77 (m, 1H), 6.21-6.00 (m, 2H), 5.64-5.28 (m, 5H), 4.50 (s, 2H), 4.33 (s, 1H), 3.37 (s, 3H), 1.15-1.02 (m, 18H). ³¹P NMR (600 MHz, DMSO-d₆) δ 17.91.

Preparation of (Example 17 Monomer): To a solution of 13 (330 mg, 0.4 mmol) in DCM (20 mL) was added DCI (46 mg, 0.4 mmol) and CEP [N (iPr)₂]₂ (165 mg, 0.5 mmol) under N₂. The mixture was stirred at 25° C. for 2.5 h. LCMS showed 13 was consumed completely. The product was extracted with DCM. The organic layer was washed with H₂O and brine. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/3 increasing to CH₃CN/H₂O (0.5% NH₄HCO₃)=1/0 within 25 min, the eluted product was collected at CH₃CN/H₂O (0.5% NH₄HCO₃)=1/0; Detector, UV 254 nm. This resulted in Example 17 Monomer (310 mg, 0.3 mmol) as a white solid. ESI-LCMS: m/z 920.1 [M−H]⁻. ¹H NMR (400 MHz, DMSO-d₆) δ 12.68 (s, 1H), 11.78 (s, 1H), 11.19 (s, 1H), 8.14-7.48 (m, 5H), 7.10-6.71 (m, 1H), 6.26-6.03 (m, 2H), 5.67-5.48 (m, 4H), 4.79-4.40 (m, 3H), 3.89-3.35 (m, 7H), 2.87-2.72 (m, 2H), 1.25-1.02 (m, 30H). ³¹P NMR (600 MHz, DMSO-d₆) δ 149.24, 148.66, 17.64, 17.26.

These examples are provided for illustrative purposes only and not to limit the scope of the claims provided herein.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof, inclusive of the endpoints. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

What is claimed is:

1. A short interfering nucleic acid (siNA) molecule comprising:
(a) a sense strand comprising a first nucleotide sequence that is at least about 60%, 65%, 70%, 75%, 80%, 85%, 90%, 95%, or 100% identical to an RNA corresponding to a target gene, wherein the first nucleotide sequence:
(i) is 15 to 30 nucleotides in length; and
(ii) comprises 15 or more modified nucleotides independently selected from a 2′-O-methyl nucleotide and a 2′-fluoro nucleotide, wherein at least one modified nucleotide is a 2′-O-methyl nucleotide and the nucleotide at position 3, 5, 7, 8, 9, 10, 11, 12, 14, 17, and/or 19 from the 5′ end of the first nucleotide sequence is a 2′-fluoro nucleotide or wherein at least one modified nucleotide is a 2′-O-methyl nucleotide and at least one modified nucleotide is a 2′-fluoro nucleotide; and
an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence:
(iii) is 15 to 30 nucleotides in length; and
(iv) comprises 15 or more modified nucleotides independently selected from a 2′-O-methyl nucleotide and a 2′-fluoro nucleotide, wherein at least one modified nucleotide is a 2′-O-methyl nucleotide and at least one modified nucleotide is a 2′-fluoro nucleotide; or
(b) a sense strand comprising a first nucleotide sequence that is at least about 60%, 65%, 70%, 75%, 80%, 85%, 90%, 95%, or 100% identical to an RNA corresponding to a target gene, wherein the first nucleotide sequence:
(i) is 15 to 30 nucleotides in length; and
(ii) comprises 15 or more modified nucleotides independently selected from a 2′-O-methyl nucleotide and a 2′-fluoro nucleotide, wherein at least one modified nucleotide is a 2′-O-methyl nucleotide and at least one modified nucleotide is a 2′-fluoro nucleotide; and
an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence:
(iii) is 15 to 30 nucleotides in length; and (iv) comprises 15 or more modified nucleotides independently selected from a 2′-O-methyl nucleotide and a 2′-fluoro nucleotide, wherein at least one modified nucleotide is a 2′-O-methyl nucleotide and the nucleotide at position 2, 5, 6, 8, 10, 14, 16, 17, and/or 18 from the 5′ end of the second nucleotide sequence is a 2′-fluoro nucleotide;
wherein the target gene is a gene encoding ANGPTL8 or a gene that controls expression of ANGPTL8.

2. A short interfering nucleic acid (siNA) molecule comprising:
(a) a sense strand comprising a first nucleotide sequence that is at least about 60%, 65%, 70%, 75%, 80%, 85%, 90%, 95%, or 100% identical to an RNA corresponding to a target gene, wherein the first nucleotide sequence:
(i) is 15 to 30 nucleotides in length; and
(ii) comprises 15 or more modified nucleotides independently selected from a 2′-O-methyl nucleotide and a 2′-fluoro nucleotide, wherein at least one modified nucleotide is a 2′-O-methyl nucleotide and the nucleotide at position 3, 5, 7, 8, 9, 10, 11, 12, 14, 17, and/or 19 from the 5′ end of the first nucleotide sequence is a 2′-fluoro nucleotide or wherein at least one modified nucleotide is a 2′-O-methyl nucleotide and at least one modified nucleotide is a 2′-fluoro nucleotide; and
an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence:
(iii) is 15 to 30 nucleotides in length; and
(iv) comprises 15 or more modified nucleotides independently selected from a 2′-O-methyl nucleotide and a 2′-fluoro nucleotide, wherein at least one modified nucleotide is a 2′-O-methyl nucleotide and at least one modified nucleotide is a 2′-fluoro nucleotide; or
(b) a sense strand comprising a first nucleotide sequence that is at least about 60%, 65%, 70%, 75%, 80%, 85%, 90%, 95%, or 100% identical to an RNA corresponding to a target gene, wherein the first nucleotide sequence:
(i) is 15 to 30 nucleotides in length; and
(ii) comprises 15 or more modified nucleotides independently selected from a 2′-O-methyl nucleotide and a 2′-fluoro nucleotide, wherein at least one modified nucleotide is a 2′-O-methyl nucleotide and at least one modified nucleotide is a 2′-fluoro nucleotide; and
an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence:
(iii) is 15 to 30 nucleotides in length; and (iv) comprises 15 or more modified nucleotides independently selected from a 2′-O-methyl nucleotide and a 2′-fluoro nucleotide, wherein at least one modified nucleotide is a 2′-O-methyl nucleotide and the nucleotide at position 2, 5, 6, 8, 10, 14, 16, 17, and/or 18 from the 5′ end of the second nucleotide sequence is a 2′-fluoro nucleotide;
wherein the target gene is a gene encoding ANGPTL3 or a gene that controls expression of ANGPTL3.

3. A double-stranded short interfering nucleic acid (ds-siNA) molecule comprising:
(a) a sense strand comprising a first nucleotide sequence, wherein the first nucleotide sequence is 15 to 30 nucleotides in length and comprises a nucleotide sequence as set forth in in any one of SEQ ID NOs: 1-316, and
an antisense strand comprising a second nucleotide sequence, wherein the second nucleotide sequence is 15 to 30 nucleotides in length and comprises a nucleotide sequence that is a substantial reverse complement of the first nucleotide sequence; or
(b) a sense strand comprising a first nucleotide sequence, wherein the first nucleotide sequence is 15 to 30 nucleotides in length and comprises a nucleotide sequence that is a substantial reverse complement of a second nucleotide sequence; and
an antisense strand comprising the second nucleotide sequence, wherein the second nucleotide sequence is 15 to 30 nucleotides in length and comprises a nucleotide sequence as set forth in in any one of SEQ ID NOs: 317-632.

4. A double-stranded short interfering nucleic acid (ds-siNA) molecule comprising:
(a) a sense strand comprising a first nucleotide sequence, wherein the first nucleotide sequence is 15 to 30 nucleotides in length and comprises a nucleotide sequence as set forth in in any one of SEQ ID NOs: 633-878, and
an antisense strand comprising a second nucleotide sequence, wherein the second nucleotide sequence is 15 to 30 nucleotides in length and comprises a nucleotide sequence that is a substantial reverse complement of the first nucleotide sequence; or
(b) a sense strand comprising a first nucleotide sequence, wherein the first nucleotide sequence is 15 to 30 nucleotides in length and comprises a nucleotide sequence that is a substantial reverse complement of a second nucleotide sequence; and
an antisense strand comprising the second nucleotide sequence, wherein the second nucleotide sequence is 15 to 30 nucleotides in length and comprises a nucleotide sequence as set forth in any one of SEQ ID NOs: 879-1125.

5. A double-stranded short interfering nucleic acid (ds-siNA) molecule comprising (a) a sense strand comprising a nucleotide sequence as set forth in any one of SEQ ID NOs: 1-316, and (b) an antisense strand.

6. A double-stranded short interfering nucleic acid (ds-siNA) molecule comprising (a) a sense strand comprising a nucleotide sequence as set forth in any one of SEQ ID NOs: 633-878, and (b) an antisense strand.

7. A double-stranded short interfering nucleic acid (ds-siNA) molecule comprising (a) an antisense strand comprising a nucleotide sequence as set forth in any one of SEQ ID NOS: 317-632, and (b) a sense strand.

8. A double-stranded short interfering nucleic acid (ds-siNA) molecule comprising (a) an antisense strand comprising a nucleotide sequence as set forth in any one of SEQ ID NOs: 879-1125, and (b) a sense strand.

9. The ds-siNA of any one of claim 2, 4, 6 or 8, wherein the sense strand comprises SEQ ID NOs: 633, 634, 642, 653, 654, 674, 687, 688, 690-694, 708-713, 756-757, 765, 776, 777, 797, 810, 811, 813-817, or 831-836, and the antisense strand comprises SEQ ID NOs: 879, 880, 888, 899, 900, 920, 933, 934, 936-940, 954-959, 1003-1004, 1012, 1023, 1024, 1044, 1057, 1058, 1060-1064, or 1078-1083, respectively.

10. The ds-siNA of any one of claims 2, 4, 6, 8, wherein the sense strand comprises SEQ ID NOs: 633, 654, 688, 690, 708, 713, 756, 777, 811, 813, 831, or 836, and the antisense strand comprises SEQ ID NOs: 879, 900, 934, 936, 954, 959, 1003, 1024, 1058, 1060, 1078, or 1083, respectively.

11. The ds-siNA of any one of claims 1-10, wherein the sense and/or antisense strand further comprises a TT sequence at 3′ end.

12. The ds-siNA of any one of claims 1-11, wherein at least one end of the ds-siNA is a blunt end.

13. The ds-siNA of any one of claims 1-11, wherein at least one end of the ds-siNA comprises an overhang, wherein the overhang comprises at least one nucleotide, optionally wherein the at least one nucleotide comprises a 2′-O-methyl nucleotide, a deoxy nucleotide, or a uracil analogue or derivative.

14. The ds-siNA of any one of claims 1-11, wherein both ends of the ds-siNA comprise an overhang, wherein the overhang comprises at least one nucleotide, optionally wherein the at least one nucleotide comprises a 2′-O-methyl nucleotide, a deoxy nucleotide, or a uracil analogue or derivative.

15. The ds-siNA of any one of claims 1-14, wherein the sense strand and/or the antisense strand independently comprise 1 or more mesyl phosphoramidate internucleoside linkages, or 1 or more phosphorothioate internucleoside linkages.

16. The ds-siNA molecule according to any one of claims 1-15, wherein the sense strand comprises at least 1, 2, 3, 4, 5, 6, 7, 8, 9, 10, 11, 12, 13, 14, 15 or more phosphorothioate internucleoside linkages.

17. The ds-siNA molecule of claim 16, wherein:
(i) at least one phosphorothioate internucleoside linkage in the sense strand is between the nucleotides at positions 1 and 2 from the 5′ end of the first nucleotide sequence;
(ii) at least one phosphorothioate internucleoside linkage is between the nucleotides at positions 2 and 3 from the 5′ end of the first nucleotide sequence.

18. The ds-siNA molecule according to any one of claims 1-17, wherein the antisense strand further comprises at least 1, 2, 3, 4, 5, 6, 7, 8, 9, 10, 11, 12, 13, 14, 15 or more phosphorothioate internucleoside linkages.

19. The ds-siNA molecule of claim 18, wherein:
(i) at least one phosphorothioate internucleoside linkage in the antisense strand is between the nucleotides at positions 1 and 2 from the 5′ end of the second nucleotide sequence;
(ii) at least one phosphorothioate internucleoside linkage in the antisense strand is between the nucleotides at positions 2 and 3 from the 5′ end of the second nucleotide sequence;
(iii) at least one phosphorothioate internucleoside linkage in the antisense strand is between the nucleotides at positions 1 and 2 from the 3′ end of the second nucleotide sequence; and/or (iv) at least one phosphorothioate internucleoside linkage is between the nucleotides at positions 2 and 3 from the 3′ end of the second nucleotide sequence.

20. The ds-siNA molecule according to any one of claims 1-19, wherein the sense strand comprises at least 1, 2, 3, 4, 5, 6, 7, 8, 9, 10, 11, 12, 13, 14, 15 or more mesyl phosphoramidate internucleoside linkages.

21. The ds-siNA molecule of claim 20, wherein:
(i) at least one mesyl phosphoramidate internucleoside linkage in the sense strand is between the nucleotides at positions 1 and 2 from the 5′ end of the first nucleotide sequence; (ii) at least one mesyl phosphoramidate internucleoside linkage is between the nucleotides at positions 2 and 3 from the 5′ end of the first nucleotide sequence.

22. The ds-siNA molecule according to any one of claims 1-21, wherein the antisense strand further comprises at least 1, 2, 3, 4, 5, 6, 7, 8, 9, 10, 11, 12, 13, 14, 15 or more mesyl phosphoramidate internucleoside linkages.

23. The ds-siNA molecule of claim 22, wherein:
(i) at least one mesyl phosphoramidate internucleoside linkage in the antisense strand is between the nucleotides at positions 1 and 2 from the 5′ end of the second nucleotide sequence;
(ii) at least one mesyl phosphoramidate internucleoside linkage in the antisense strand is between the nucleotides at positions 2 and 3 from the 5′ end of the second nucleotide sequence;
(iii) at least one mesyl phosphoramidate internucleoside linkage in the antisense strand is between the nucleotides at positions 1 and 2 from the 3′ end of the second nucleotide sequence; and/or (iv) at least one mesyl phosphoramidate internucleoside linkage is between the nucleotides at positions 2 and 3 from the 3′ end of the second nucleotide sequence.

24. The ds-siNA of any one of claims 1-23, wherein the antisense strand comprises one or more modified nucleotides.

25. The ds-siNA of any one of claims 1-24, wherein the sense strand comprises one or more modified nucleotides.

26. The ds-siNA of any one of claims 1-24, wherein the sense strand and the antisense strand each independently comprise one or more modified nucleotides.

27. The ds-siNA of any one of claims 24-26, wherein the modified nucleotides are independently selected from 2′-O-methyl nucleotides and 2′-fluoro nucleotides.

28. The ds-siNA of claim 27, wherein at least one 2′-fluoro nucleotide or 2′-O-methyl nucleotide is a 2′-fluoro or 2-O-methyl nucleotide mimic of Formula (V): 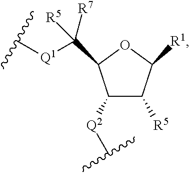
wherein
R¹ is a nucleobase, aryl, heteroaryl, or H,
Q¹ and Q² are independently S or O,
R⁵ is -OCD₃, —F, or —OCH₃, and
R⁶ and R⁷ are independently H or D.

29. The siNA molecule according to any one of the preceding claims, wherein the antisense strand, sense strand, first nucleotide sequence, and/or second nucleotide sequence comprises at least one modified nucleotide selected from: 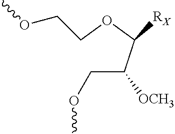
wherein Rx is a nucleobase, aryl, heteroaryl, or H, 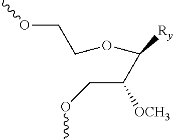
(mun34), wherein R_{y} is a nucleobase, 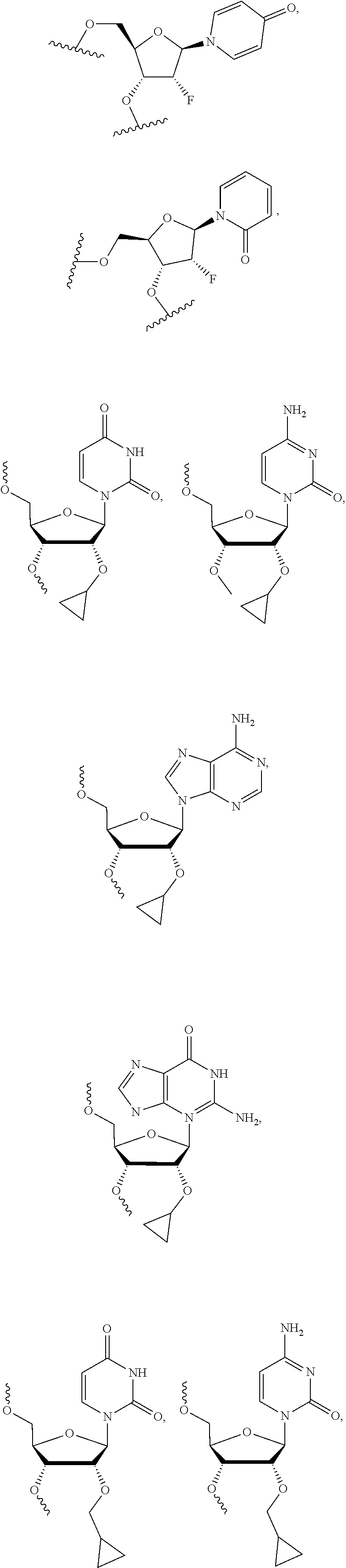 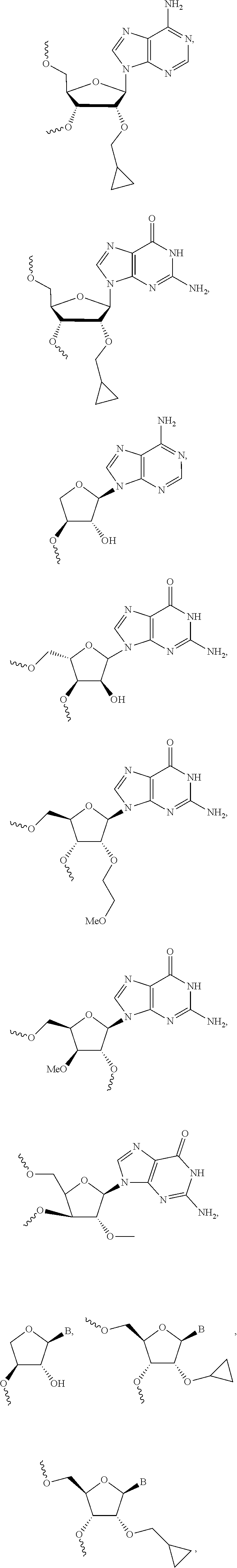 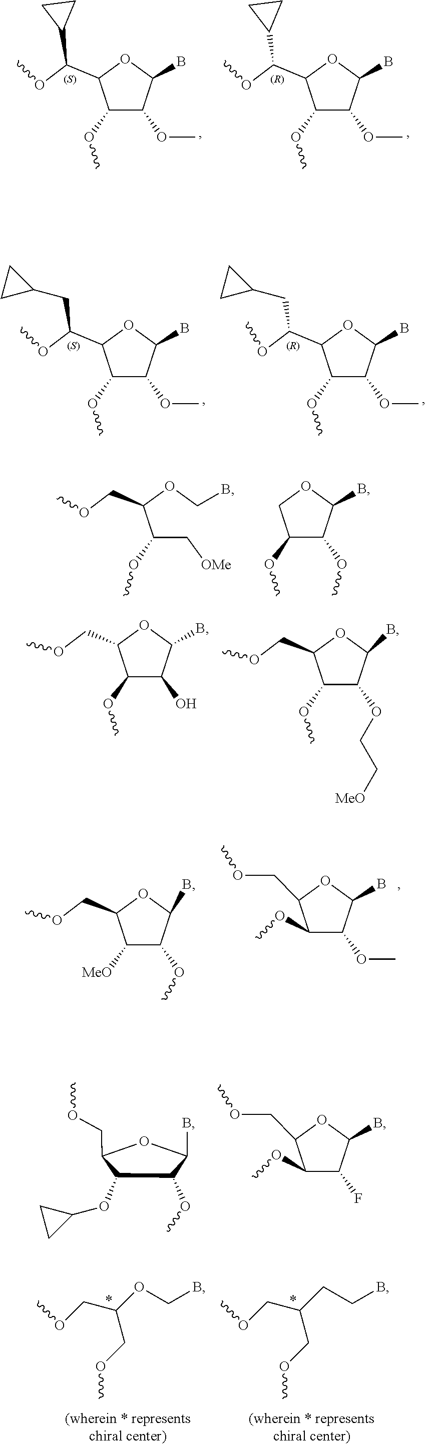 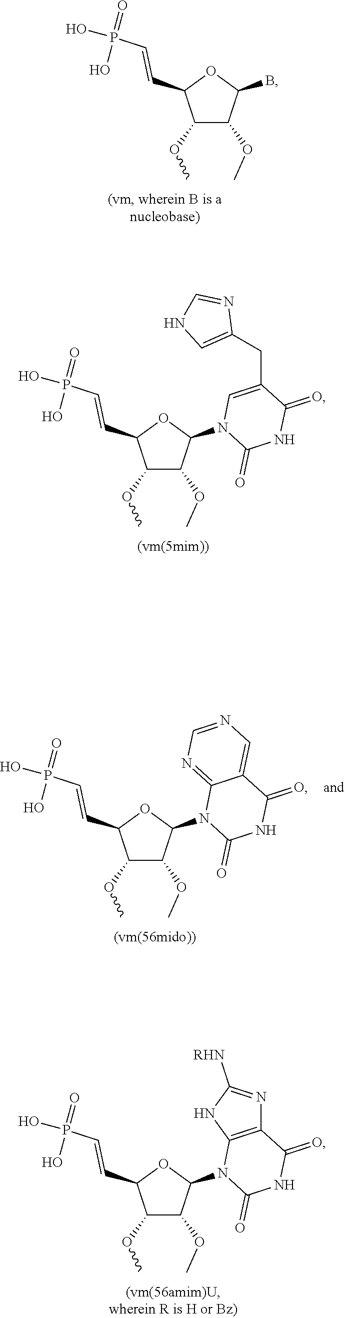
wherein B is a nucleobase, aryl, heteroaryl, or H, and wherein [symbol] or [symbol] represents a phosphodiester linkage, a phosphorothioate linkage, a mesyl phosphoramidate linkage, or H.

30. The ds-siNA of any of claims 1-29, wherein the ds-siNA further comprises a phosphorylation blocker, a 5′-stabilized end cap, or a combination thereof.

31. The ds-siNA of claim 30, wherein the phosphorylation blocker is attached to the 5′ end of the sense strand, and optionally, wherein the phosphorylation blocker is attached to the 5′ end of the sense strand via one or more of a phosphodiester linkage, a phosphorothioate linkage, a mesyl phosphoramidate linkage, or a phosphorodithioate linkage.

32. The ds-siNA of claim 30 or 31, wherein the 5′-stabilized end cap is a 5′ vinyl phosphonate.

33. The ds-siNA of any of claims 30-32, wherein the 5′-stabilized end cap is attached to the 5′ end of the antisense strand, and optionally, wherein the 5′-stabilized end cap is attached to the 5′ end of the antisense strand with one or more of a phosphodiester linkage, a phosphorothioate linkage, a mesyl phosphoramidate linkage, or a phosphorodithioate linkage.

34. The ds-siNA according to any one of the preceding claims, wherein the ds-siNA further comprises a galactosamine.

35. The ds-siNA according to claim 34, wherein the galactosamine is an N-acetylgalactosamine comprising a structure of: 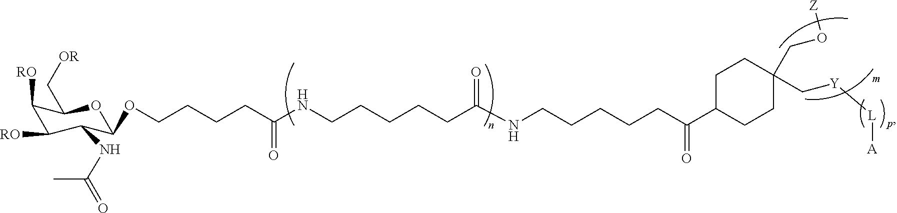
wherein
m is 1, 2, 3, 4, or 5;
each n is independently 1 or 2;
p is 0 or 1;
each R is independently H;
each Y is independently selected from —O—P(═O)(SH)—, —O—P(═O)(O)—, —O—P(═O)(OH)—, and —O—P(S)S—;
Z is H or a second protecting group;
either L is a linker or L and Y in combination are a linker; and
A is H, OH, a third protecting group, an activated group, or an oligonucleotide.

36. The siNA according to claim 34, wherein the galactosamine is an N-acetylgalactosamine comprising a structure of: 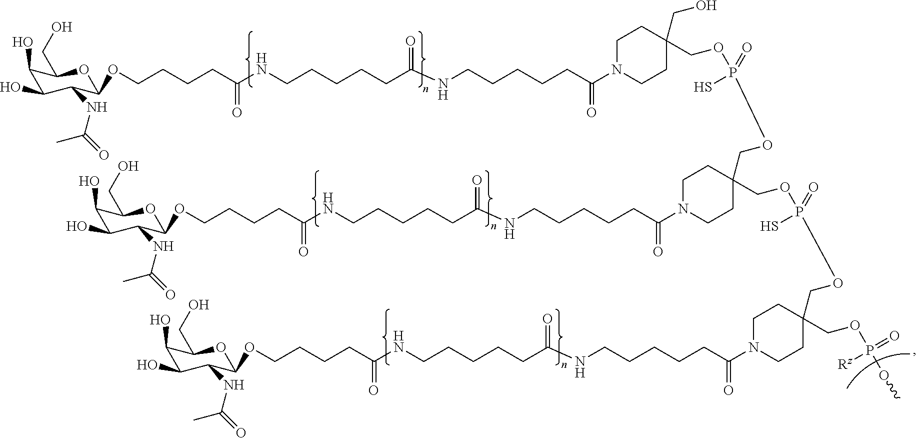
wherein R^{z} is OH or SH; and each n is independently 1 or 2.

37. The ds-siNA of any one of the preceding claims, wherein the sense and/or anti-sense strand of the ds-siNA is conjugated to GalNAc, Folate, Cholesterol, or Palmitic Acid.

38. The ds-siNA of any one of the preceding claims, wherein the ds-siNA reduces ANGPTL8 or ANGPTL3 expression by more than about 10%, more than about 20% more than about 30%, more than about 40%, more than about 50%, more than about 60%, more than about 70%, more than about 80%, more than about 90%, more than about 95%, or 100%.

39. The ds-siNA of any one of the preceding claims, wherein the ds-siNA reduces ANGPTL8 or ANGPTL3 expression or activity with a EC50 value of 50 pM or less, of 40 pM or less, of 30 pM or less, of 20 pM or less, 15 pM or less, 10 pM or less, 5 pM or less, 2 pM or less, or 1 pM or less.

40. The ds-siNA of any one of the preceding claims, wherein the ds-siNA has a CC₅₀ value of more than 1 μM.

41. The ds-siNA of any one of the preceding claims, wherein the ds-siNA comprises RNA nucleotides.

42. A ds-siNA, comprising a sense strand and an antisense strand, wherein the antisense strand comprises at its 5′ end one of the following:

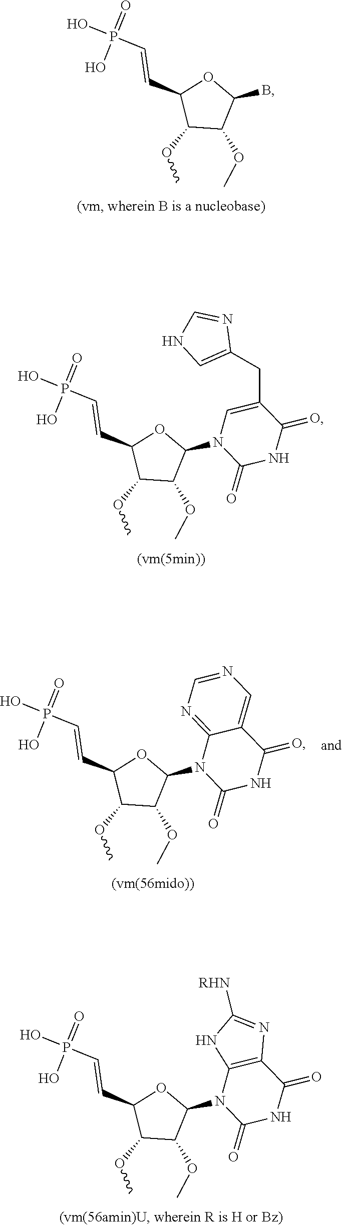

43. A pharmaceutical composition comprising at least one ds-siNA according to any one of claims 1-42 and a pharmaceutically acceptable carrier or diluent.

44. A pharmaceutical composition comprising two or more ds-siNA according to any one of claims 1-42.

45. A pharmaceutical composition comprising at least a first siNA that reduces ANGPTL3 expression and a second siNA that reduces ANGPTL8 expression and a pharmaceutically acceptable carrier.

46. The pharmaceutical composition of claim 45, wherein the first siNA that reduces ANGPTL3 expression comprises a nucleic acid sequence disclosed in Table 3, and the second siNA that reduces ANGPTL8 expression comprises a nucleic acid sequence disclosed in Table 1.

47. The pharmaceutical composition of claim 45 or 46, wherein the first siNA that reduces ANGPTL3 expression is a ds-siNA disclosed in Table 4, and the second siNA that reduces ANGPTL8 expression is a ds-siNA disclosed in Table 2.

48. The pharmaceutical composition of any one of claims 45-47, wherein a weight ratio of the first siNA and the second siNA is selected from 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1.

49. The pharmaceutical composition of any one of claims 45-48, wherein a molar ration of the first siNA and the second siNA is selected from 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1.

50. The pharmaceutical composition of any one of claims 45-49, wherein the first siNA and the second siNA are connected by a linker.

51. The pharmaceutical composition of claim 50, wherein the linker is selected from a nucleotide-based linker or a non-nucleotide based linker.

52. The pharmaceutical composition of claim 51, wherein the nucleotide-based linker comprises between about 1 to about 15 nucleotides.

53. The pharmaceutical composition of claim 51, wherein the linker comprises at least one N-acetylgalactosamine.

54. The pharmaceutical composition of any one of claims 43-53, wherein the pharmaceutical composition is formulated for parenteral, ocular, nasal, transdermal, pulmonary, or topical administration or comprises a liposome that encapsulates the ds-siNA.

55. Use of the ds-siNA according to any one of claims 1-42 in the manufacture of a medicament for treating cardiovascular disease or nonalcoholic fatty liver disease.

56. Use of two or more ds-siNA according to any one of claims 1-42 in the manufacture of a medicament for treating cardiovascular disease or nonalcoholic fatty liver disease.

57. The ds-siNA according to any one of claims 1-42 or the pharmaceutical composition according to any one of claims 43-54 for use in treating cardiovascular disease or nonalcoholic fatty liver disease.

58. Two or more ds-siNA according to any one of claims 1-42 or the pharmaceutical composition according to any one of claims 43-54 for use in treating cardiovascular disease or nonalcoholic fatty liver disease

59. A method of treating a disease in a subject in need thereof, comprising administering to the subject the ds-siNA according to any one of claims 1-42 or the pharmaceutical composition according to any one of claims 43-54, wherein the disease is cardiovascular disease or nonalcoholic fatty liver disease.

60. A method of treating a disease in a subject in need thereof, comprising administering to the subject two or more ds-siNA according to any one of claims 1-42 or the pharmaceutical composition according to any one of claims 43-54, wherein the disease is cardiovascular disease or nonalcoholic fatty liver disease.

61. A method of treating a disease in a subject in need thereof, comprising administering to the subject a first pharmaceutical composition comprising at least a first ds-siNA that reduces ANGPTL3 expression and a second pharmaceutical composition comprising at least a second ds-siNA that reduces ANGPTL8 expression, wherein the disease is cardiovascular disease or nonalcoholic fatty liver disease.

62. The method of any one of claims 59-61, wherein the cardiovascular disease is Hypertriglyceridemia (HTG) or Familial Hypercholesterolemia (FH).

63. The method according to any one of claims 59-62, wherein the subject is a mammal, and optionally wherein the mammal is a human or a non-human primate.

64. The method of any one of claims 59-63, wherein the ds-siNA or pharmaceutical composition(s) are administered intravenously, subcutaneously, or via inhalation.

65. The method of any one of claims 59-64, wherein the subject has been treated with one or more additional therapeutic agents.

66. A method of reducing ANGPTL8 and/or ANGPTL3 expression or activity in a tissue, an organ, or a cell of a subject in need thereof, wherein the method comprises delivering to the subject one or more of the ds-siNA according to any one of claims 1-42 or the pharmaceutical composition according to any one of claims 43-54.

67. A modified nucleoside selected from [chemical structure]
